(19) United States
(12) Patent Application Publication
(10) Pub. No.: US 2021/0254073 A1
(43) Pub. Date: Aug. 19, 2021

(54) METHODS FOR CAPTURE AND ANALYSIS OF MACROMOLECULAR COMPLEXES

(71) Applicant: Cornell University, Ithaca, NY (US)

(72) Inventors: Judhajeet Ray, Ithaca, NY (US); Abdullah Ozer, Ithaca, NY (US); John Lis, Ithaca, NY (US)

(21) Appl. No.: 17/167,991

(22) Filed: Feb. 4, 2021

Related U.S. Application Data
(60) Provisional application No. 62/970,015, filed on Feb. 4, 2020.
(60) Provisional application No. 62/970,035, filed on Feb. 4, 2020.

(51) Int. Cl. C12N 15/115; C12Q 1/6844; C12Q 1/6869; G01N 33/543

(52) U.S. Cl. CPC C12N 15/115; G01N 33/54306; C12Q 1/6869; C12Q 1/6844

(57) ABSTRACT

The present application relates to a method for analyzing a molecular target in a sample. The method involves providing an aptamer, wherein the aptamer is a high affinity binding partner to at least a portion of the molecular target in the sample. The aptamer is contacted with the sample containing the molecular target under conditions effective for the molecular target and aptamer to bind to each other. The molecular target is separated from the sample to form a molecular target enriched sample. The separated molecular target of the enriched sample is then analyzed.

This application claims the benefit of U.S. Provisional Patent Application Ser. Nos. 62/970,035 and 62/970,015, filed Feb. 4, 2020, which are hereby incorporated by reference in their entirety.

### FIELD

The present application relates to methods for capture and analysis of macromolecular complexes.

### BACKGROUND

Characterizing macromolecular complexes (MCs) and their interactions is essential for understanding any biological process at the molecular level. With increased resolution and throughput of mass spectrometry (MS) in the last decade, MS-based proteomic analyses following co-immunoprecipitations have been extensively utilized to identify interacting partners of many proteins (DeBlasio et al., “Insights into the Polerovirus-plant Interactome Revealed by Coimmunoprecipitation and Mass Spectrometry,” Mol. Plant Microbe Interact. 28:467-481 (2015); Budayeva et al., “A Mass Spectrometry View of Stable and Transient Protein Interactions,” Adv. Exp. Med. Biol. 806:263-282 (2014); Bauer et al., “Affinity Purification-mass Spectrometry. Powerful Tools for the Characterization of Protein Complexes,” Eur. J. Biochem. 270: 570-578 (2003)). These assays rely on the availability of antibodies that are well characterized, highly specific, and high-affinity against the protein of interest (POI) or a peptide tag that allow the binding partners of the (tagged)-target protein to be co-precipitated while the antibody is immobilized on beads/resins. Even for a single antibody, significant lot-to-lot variability affects the purity, specificity and yield of (co-) immunoprecipitations. The (co-) immunoprecipitated proteins are subsequently eluted by denaturation (i.e. with heat, SDS or combinations) or by on-bead proteolytic digestion and analyzed by MS. However, contaminating peptides derived from the antibody/serum or Protein-A/G are routinely found at an order of magnitude higher abundance than the POI (DeBlasio et al., “Insights into the Polerovirus-plant Interactome Revealed by Coimmunoprecipitation and Mass Spectrometry,” Mol. Plant Microbe Interact. 28:467-481 (2015); Budayeva et al., “A Mass Spectrometry View of Stable and Transient Protein Interactions,” Adv. Exp. Med. Biol., 806:263-282 (2014)). This can hinder the identification of interacting partners, particularly if they are rare or substoichiometric. In some cases, eluates are further fractionated by gel electrophoresis and individual protein bands are excised to exclude heavy and light chains of the antibody prior to MS. This method limits the number of proteins that can be identified and prevents analysis of proteins below the limit of detection for a given electrophoresis/protein staining technique (Jafari et al., “Comparison of In-gel Protein Separation Techniques Commonly Used for Fractionation in Mass Spectrometry-based Proteomic Profiling,” Electrophoresis 33:2516-2526 (2012)) while increasing the likelihood of keratin contamination and the length of time needed for sample preparation.

The present application is directed to overcoming these and other deficiencies in the art.

### SUMMARY

The present application relates to a method for analyzing a molecular target in a sample. The method involves providing an aptamer, wherein the aptamer is a high affinity binding partner to at least a portion of the molecular target in the sample. The aptamer is contacted with the sample containing the molecular target under conditions effective for the molecular target and aptamer to bind to each other. The molecular target is separated from the sample to form a molecular target enriched sample. The separated molecular target of the enriched sample is then analyzed.

In some embodiments, the separated molecular target is analyzed using mass spectrometry. In some embodiments, the separated molecular target is analyzed using cryo-electron microscopy. In some embodiments, the separated molecular target is analyzed using nucleotide sequencing. In some embodiments, the separated molecular target is analyzed using any combination of these techniques.

To provide an alternative to immunoprecipitations, an RNA aptamer-based affinity purification method has been developed using the highly-specific and high-affinity Green Fluorescent Protein (GFP)-aptamer (Shui et al., “RNA Aptamers that Functionally Interact with Green Fluorescent Protein and its Derivatives,” Nucleic Acids Res. 40:e39 (2012), which is hereby incorporated by reference in its entirety) to co-purify GFP-tagged target proteins and their binding partners for identification by mass spectrometry (MS) (AptA-MS), nucleotide sequencing, and/or cryo-electron microscopy. Nucleic acid aptamers can be selected against a wide variety of targets and synthesized in unlimited quantities by cost-effective methods. These properties, in addition to their high specificity and affinity, make aptamers attractive reagents for affinity purification. Indeed, aptamers have been used for affinity purification of targets from biological mixtures followed by MS, but mainly for target detection and biomarker discovery (Gulbakan, “Oligonucleotide Aptamers: Emerging Affinity Probes for Bioanalytical Mass Spectrometry and Biomarker Discovery,” Anal. Methods 7:7416-7430 (2015), which is hereby incorporated by reference in its entirety). These detection assays were developed with a handful of aptamers and demonstrated to work by proof-of-principle experiments with little or no biological applications. General and simple affinity-capture methods using RNA aptamers are lacking, especially those that allow for quantitative analysis of protein interactions and protein complex formation directly from cellular lysates and can be applied to address a broad array of biological questions in a wide range of species, tissues, and cell types.

The GFP protein in combination with the high-affinity and high-specificity GFP-aptamer (Shui et al., “RNA Aptamers that Functionally Interact with Green Fluorescent Protein and its Derivatives,” Nucleic Acids Res. 40:e39 (2012), which is hereby incorporated by reference in its entirety) serves as a suitable affinity tool to identify, for example, protein-protein interactions by MS, three-dimensional structure by cryo-electron microscopy, and DNA binding sites of proteins within a complex using nucleotide sequencing techniques. The methods described herein allow use with a broad collection of existing GFP-fusion proteins in human cells and other model organisms including Drosophila and yeast. Here, it is demonstrated that AptA-MS is superior to conventional co-immunoprecipitations for subsequent MS analysis, because it is devoid of immunoprecipitation-derived protein contaminants, and provides a dramatic enrichment of the POI. Using AptA-MS several known and novel interactors of human Heat Shock Factor 1 (HSF1) tagged with GFP have been identified, some of which showed an increased association following heat shock (HS). In addition, post-translational modifications (PTMs) of HSF1 and the co-precipitated histones have been identified without additional tailored enrichment steps for these modifications. AptA-MS has also been applied with other aptamers (e.g. NELF-aptamer) (Pagano et al., “Defining NELF-E RNA Binding in HIV-1 and Promoter-proximal Pause Regions,” PLoS Genet. 10:e1004090 (2014), which is hereby incorporated by reference in its entirety) to enrich its target from Drosophila S2 cells. The results indicate that in addition to purifying transiently transfected HSF1-GFP from human cells, the GFP-aptamer is capable of enriching endogenous GFP-tagged RNA polymerase II (Pol II) from yeast, as well as formaldehyde crosslinked GFP from Drosophila S2 cells, thereby making it a versatile tool for affinity purification of GFP-tagged proteins from various sources.

### BRIEF DESCRIPTION OF THE DRAWINGS

FIG. 1 shows the AptA-MS workflow. The polyadenylated GFP-aptamer is annealed to desthiobiotin (dB) labeled oligo dT and immobilized on streptavidin (SA) coated magnetic Dynabeads. Cellular lysate containing Protein of interest (POI)-GFP is incubated with the immobilized aptamer beads that are washed and finally eluted with biotin. Eluate is subjected to MS, and the data is processed through a pipeline for protein identification followed by enrichment analysis, interaction score quantification, and PTM analysis. Figure partially created with BioRender.com using the GFP structure (PDB ID: 4KW4, Barnard et al., “Crystal Structure of Green Fluorescent Protein,” doi: 10.2210/pdb4KW4/pdb (2014), which is hereby incorporated by reference in its entirety).

FIGS. 2A-2D show enrichment of GFP-tagged proteins by AptA-MS.

FIG. 2A is a schematic representation of the experimental design. FIG. 2B shows cellular lysates prepared from HCT116 cells transfected with GFP or HSF1-GFP expressing plasmids that were analyzed by anti-GFP (green) and anti-Actin (red, loading control) western blot. GFP (Abcam, ab290) and Actin (Sigma, MAB1501) antibodies were used at 1:2000 and 1:5000 dilutions, respectively. FIG. 2C shows lysate from cells expressing GFP or HSF1-GFP were precipitated with the GFP- or Control (Ctrl)-aptamer and eluates were analyzed by gel electrophoresis and silver-staining. The bottom panel shows a fluorescence image of the eluates. FIG. 2D shows enrichment analysis of HSF1 in AptA-MS samples from cells expressing GFP or HSF1-GFP, before or after heat shock, pulled-down with the GFP- or the control-aptamer. Plot represents data from five independent biological replicates.

FIGS. 3A-3B show a comparison of aptamer vs antibody affinity purification. FIG. 3A shows cellular lysates prepared from HCT116 cells that were transfected with HSF1-GFP expressing plasmids and were affinity-purified by the GFP-aptamer (Apt) or the GFP-antibody (Ab). Eluates were run after RNase treatment on an SDS-PAGE and visualized by silver-stain. FIG. 3B shows comparative analysis of AptA-MS vs. conventional immunoprecipitation. Identified proteins from HSF1-GFP AptA-MS or CP.RTP immunoprecipitation (DeBlasio et al., “Insights into the polerovirus-plant interactome revealed by coimmunoprecipitation and mass spectrometry,” Mol Plant Microbe Interact 28:467-481 (2015), which is hereby incorporated by reference in its entirety), which were subjected to identical MS analysis, are ranked according to their abundance. Asterisk denotes MS data obtained from DeBlasio et al., “Insights into the polerovirus-plant interactome revealed by coimmunoprecipitation and mass spectrometry,” Mol Plant Microbe Interact 28:467-481 (2015), which is hereby incorporated by reference in its entirety.

FIGS. 4A-4B show interaction and PTM analysis of HSF1. FIG. 4A shows SAINT analysis of proteins pulled-down by HSF1-GFP AptA-MS before or after HS. Dotted horizontal line represents the SAINT score cutoff (0.65). Labeled proteins above the cutoff are called as HSF1 interactors. Fold Change represents the algorithmically calculated fold change A value which takes into account representation among biological replicates. FIG. 4B shows post-translational modifications (PTMs) on HSF1 residues identified in AptA-MS. Red and blue represent phosphorylation and acetylation, respectively. Asterisk denotes newly identified modification.

FIG. 5 shows tandem Affinity Purification of Drosophila HSF and its interacting partners. Lysates prepared from Drosophila S2 cells stably expressing NTAP-dHSF or dHSF-CTAP were subjected to Tandem Affinity Purification. Proteins in the final eluate were fractionated by SDS-PAGE and visualized by silver stain. Protein bands that were specifically co-purified with dHSF were identified by mass spectrometry in Cornell Mass Spectrometry Core Facility.

FIGS. 6A-6D show MS1 and MS2 validation of post-translationally modified HSF1 peptides. As shown in FIG. 6A, to verify post translational modifications predicted by Scaffold PTM, MS1 analysis was performed on a representative HSF1 peptide (#-GHTDTEGRPPSphosPPPTSTphosPEK-372). The normalized peak area of the modified peptide shows a consistent ratio of precursor ion masses among biological replicates regardless of condition. These precursor ion masses are derived from naturally occurring carbon isotopes C12, C13, and C14 which result in an ion with the expected mass (M) or with an addition of +1 or +2 atomic mass units (M+1, M+2). The normalized peak areas for the selected peptide are shown with M, M+1, and M+2 shown in blue, grey, and pink respectively. MS1 analysis was performed using Skyline to quantify the peak areas from a doubly phosphorylated peptide detected using AptA-MS. As shown in FIG. 6B, the MS2 spectrum for the same modified peptide (#-GHTDTEGRPPSphosPPPTSTphosPEK-372) is consistent with the MS1 analysis and shows the expected mass shifts for S363 and T369 phosphorylation of HSF1. MS2 spectra also verify predicted HSF1 acetylation at two novel lysine (K) residues (FIG. 6C) K62 and (FIG. 6D) K162.

FIG. 7 shows total spectral counts over all biological replicates from histone proteins H4, H2A.Z, H2B, and H2A resulting from Mascot searches allowing for lysine acetylation (in blue), serine/threonine phosphorylation (in red), and no modification (in gray). Spectral counts are shown in both non-heat shock and heat shock conditions.

FIGS. 8A-8C shows GO analysis of proteins enriched by AptA-MS. Proteins enriched by the GFP-aptamer from HSF1-GFP expressing cells compared to GFP expressing cells before or after heat shock are primarily classified based on (FIG. 8A) molecular function, (FIG. 8B) nucleic acid binding property, and (FIG. 8C) protein category.

FIGS. 9A-9C shows gene ontology (GO) analysis of proteins enriched by the NELF-aptamer AptA-MS from HCT116 cells expressing HSF1-GFP under non-heat shock or heat shock conditions. Identified proteins are primarily classified based on; molecular function (FIG. 9A), nucleic acid binding property (FIG. 9B), and protein category (FIG. 9C).

FIGS. 10A-10C shows aptamer capture of target proteins from Drosophila S2 cells. Cellular lysates prepared from Drosophila S2 cells stably expressing GFP were prepared without (FIG. 10A) or with (FIG. 10B) 1% formaldehyde crosslinking. Lysates were subjected to pull-down with the GFP- or the control (Ctrl)-aptamer and analyzed by SDS-PAGE with silver-staining. Bottom panel shows the fluorescence image of the eluates. FIG. 10C shows enrichment analysis of dNELF-E protein from AptA-MS with NELF-aptamer from Drosophila S2 cell nuclear extract.

FIGS. 11A-11B shows GFP aptamer capture of GFP-Rpb3 from yeast cells. Cellular lysates prepared from S. pombe cells expressing endogenously tagged GFP-Rpb3 were subjected to pull-down with the GFP- or the control (Ctrl)-aptamer. Eluates were analyzed by SDS-PAGE with Silver stain (FIG. 11A) or western blot (FIG. 11B) with the GFP antibody (Abcam, ab290). Expected molecular weight of GFP-Rpb3 is 61.6 kDa.

FIG. 12 shows an electron micrograph of a graphene oxide grid.

FIG. 13 shows ChAP-seq identifies HSF1 binding sites. Genome browser-shot showing a region around the heat shock gene HSPA1A. Each track is described as follows. EGFP-NHS-GFPpA: EGFP expressing cells in NHS condition pulled down by GFP aptamer; HSF1-GFP-NHS-GFPpA: HSF1-GFP expressing cells in NETS condition pulled down by GFP aptamer; HSF1-GFP-NHSNELFpA: HSF1-GFP expressing cells in NHS condition pulled down by control aptamer; EGFPHS-GFPpA: EGFP expressing cells in HS condition pulled down by GFP aptamer; HSF1-GFPHS-GFPpA: HSF1-GFP expressing cells in HS condition pulled down by GFP aptamer; HSF1-GFP-HS-NELFpA: HSF1-GFP expressing cells in HS condition pulled down by control aptamer.

FIG. 14A shows FA-ChAP-qPCR specifically enriches Pol II binding sites. qPCR results showing percent input enriched by the GFP-aptamer and the NELF(Ctrl)-aptamer pulldown at expected Pol II binding site near the GAPDH TSS (Target site) in comparison to a control non-target site 2 Kb upstream where Pol II is not expected to be strongly bound. FIG. 14B shows FA-ChAP identifies genome-wide binding of Pol II with high sensitivity. DNA sequencing results from GFP-aptamer pulldown showing distribution of Pol II binding sites near 3 representative genes (UBAP2, MYC, PVT1).

FIG. 15 shows one embodiment of sequential affinity purification.

FIG. 16 shows a second embodiment of sequential affinity purification.

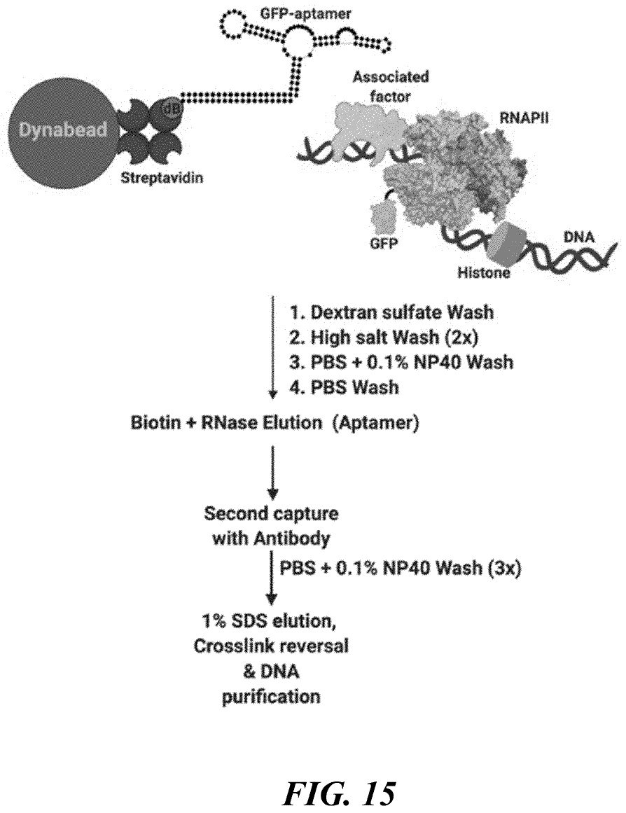

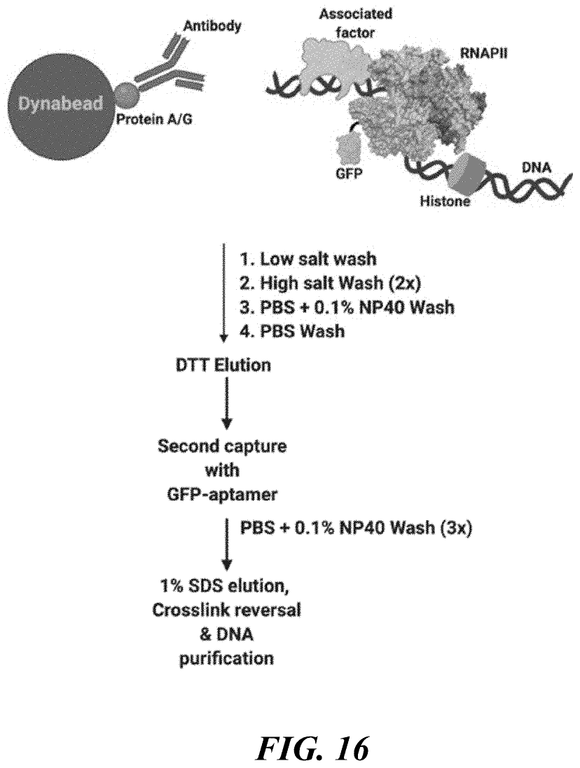

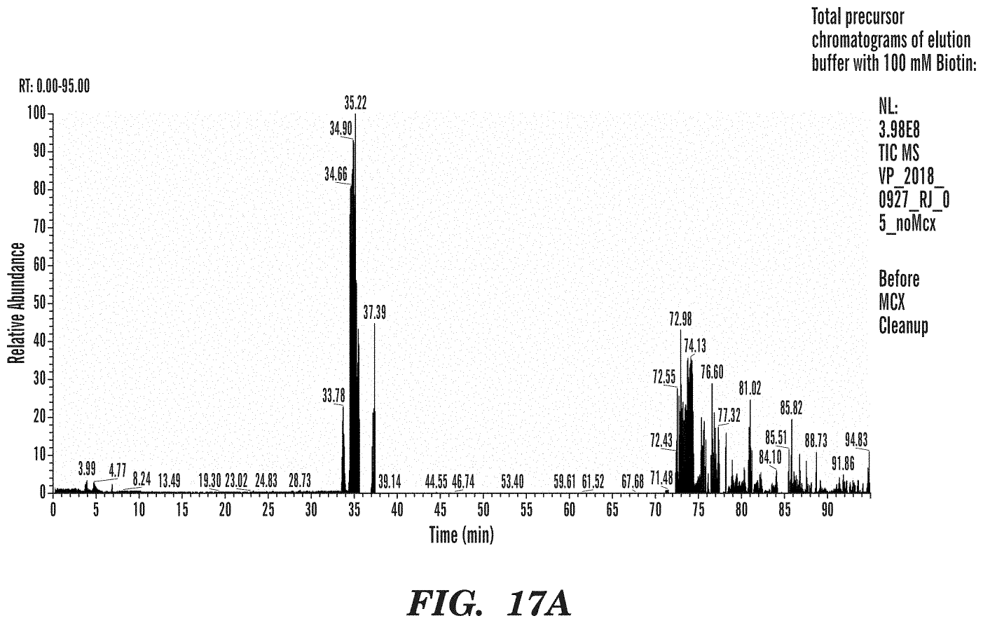

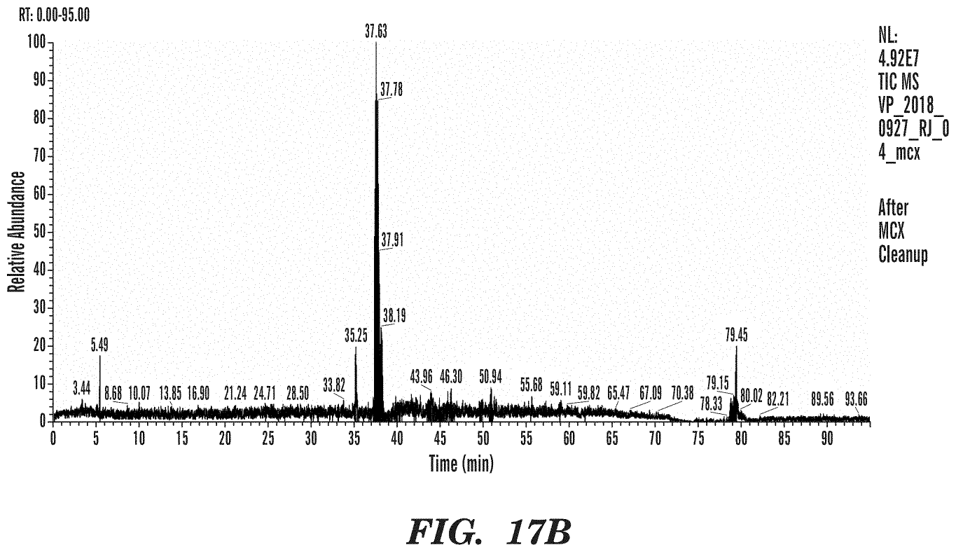

FIGS. 15-16 created with BioRender.com using the Pol II structure (PDB ID: 1I6H, doi: 10.2210/pdb1I6H/pdb, Gnatt et al., “Structural Basis of Transcription: an RNA Polymerase II Elongation Complex at 3.3 A Resolution,” Science 292(5523):1876-82 (2001), which is hereby incorporated by reference in its entirety.

FIGS. 17A-17B shows total precursor chromatograms of elution buffer spiked with 100 mM biotin, before and after MCX cleanup. Two fresh aliquots of buffer containing 20 mM Tris-HCl, pH 7.5, 50 mM NaCl, and 100 mM biotin were dried using a vacuum centrifuge. In FIG. 17A, the sample was directly reconstituted in 0.1% trifluoroacetic acid and 2% acetonitrile in water and subjected to mass spectrometry. In FIG. 17B, the sample was reconstituted in 0.1% formic acid in water, and subjected to an MCX cleanup before mass spectrometry.

### DETAILED DESCRIPTION

The present application is directed to a method for analyzing a molecular target in a sample. The method involves providing an aptamer, wherein the aptamer is a high affinity binding partner to at least a portion of the molecular target in the sample. The aptamer is contacted with the sample containing the molecular target under conditions effective for the molecular target and aptamer to bind to each other. The molecular target is separated from the sample to form a molecular target enriched sample. The separated molecular target of the enriched sample is then analyzed.

The sample may be any biological sample, including and without limitation, a cell or tissue lysate, a plasma sample, a serum sample, a blood sample, an exosome sample, or other biological sample. In some embodiments, the biological sample is derived from a cell sample, e.g., a cell lysate of a population of cells grown in vitro or in vivo. Suitable cells include immortalized cells of a cell line or primary cells. Cells can be non-mammalian or mammalian cells (e.g., a preparation of rodent cells, rabbit cells, guinea pig cells, feline cells, canine cells, porcine cells, equine cells, bovine cell, ovine cells, monkey cells, or human cells). In some embodiments, the sample is derived from preparation of human cells. Suitable cell samples also include primary or immortalized embryonic cells, fetal cells, or adult cells, at any stage of their lineage, e.g., totipotent, pluripotent, multipotent, or differentiated cells.

The molecular target in the sample that is subject to analysis according to the methods described herein may comprise one or more biomolecules. In some embodiments, the molecular target comprises at least two, at least three, at least four, at least five, or greater than five biomolecules in complex together. In some embodiments, the one or more biomolecules of the molecular target are proteins, polypeptides, peptides, ribonucleic acid molecules (RNA), deoxyribonucleic acid molecules (DNA), lipids, carbohydrates, or any combination thereof.

The molecular target in the sample that is subject to analysis according to the methods described herein may comprise one or more biomolecules that are transiently introduced into a sample (e.g., through transient transfection) or that are endogenously present in the sample. Methods of transient transfection are well known to a person of ordinary skill in the art.

In some embodiments, the sample is crosslinked to maintain the integrity of the molecular target therein. Crosslinking the sample is generally carried out prior to contacting the sample with the aptamer. Methods of crosslinking a sample and/or the components of the sample are well known in the art and include, for example, ultraviolet irradiation, chemical and physical (e.g., optical) crosslinking. Non-limiting examples of chemical crosslinking agents include formaldehyde and psoralen (Solomon et al., Proc. Natl. Acad. Sci. USA 82:6470-6474 (1985); Solomon et al., Cell 53:937947 (1988), which are hereby incorporated by reference in their entirety). Cross-linking is performed using any of a number of approaches known in the art, such as by adding a solution comprising formaldehyde, as described in the Examples herein, to a sample comprising nucleic acid molecules and chromatin proteins. In some embodiments, the sample is crosslinked by exposing the sample to a 0.1%, 0.2%, 0.3%, 0.4%, 0.5%, 0.6%, 0.7%, 0.8%, 0.9%, 1% of >1% solution of formaldehyde. Other non-limiting examples of agents that can be used for cross-linking DNA and proteins include, but are not limited to, mitomycin C, nitrogen mustard, melphalan, 1,3-butadiene diepoxide, cis diaminedichloroplatinum(II) and cyclophosphamide.

In other embodiments, the sample is “native”, or not crosslinked, prior to contacting with the aptamer.

As used herein, the term “aptamer” refers to an oligonucleotide molecule, or peptide molecule that binds to a specific molecular target.

In some embodiments, the aptamer is a peptide aptamer. As described in Reverdatto et al., “Peptide Aptamers; Development and Applications,” Curr Top Med Chem 15(12):1082-1101 (2015), which is hereby incorporated by reference in its entirety, peptide aptamers contemplated for use in the method of the present application are small combinatorial proteins that are selected to bind to specific sites on target molecules. Generally, peptide aptamers consist of 5-20 amino acid long residue sequences and are typically embedded as a loop within a stable protein scaffold.

In other embodiments, the aptamer is an oligonucleotide molecule. Oligonucleotide aptamer compositions may be double-stranded or single-stranded, and may include deoxyribonucleotides, ribonucleotides, nucleotide derivatives, or other nucleotide-like molecules. Aptamers assume a variety of shapes forming helices, single stranded loops, stem loops, etc. that provides a tertiary structure responsible for target molecule binding. Aptamer target binding involves three dimensional, shape dependent interactions as well as hydrophobic interactions, base stacking and intercalation.

In accordance with the methods of the present application, the aptamer may be a nucleic acid, preferably an RNA aptamer. In some embodiments, the aptamer is a non-naturally occurring, artificially-engineered aptamer. In other embodiments, the aptamer is a natural aptamer. In some embodiments, the aptamer utilized in the methods herein is an RNA aptamer comprising at least two stem loops, at least three stem loops, or at least four stem loops. In some embodiments, the aptamer utilized in the methods disclosed herein comprises an internal loop and at least two flanking stems (preferably three stems). In some embodiments, the aptamers are designed and synthesized or randomly synthesized. The aptamers can be screened, selected or ranked by affinity binding test with a molecular target. In other embodiments, the aptamers are amplified from a selected sequence or optimized sequence.

The nucleotide components of aptamers suitable for use in accordance with the methods described herein may include modified or non-natural nucleotides to enhance stability or other desired properties. Accordingly, in some embodiments, aptamers of the disclosure comprise one or more modified sugar groups (e.g., modifications at the 2′ position of the ribose to include a 2′-amino, 2′-fluoro, or 2′O-methyl group) to enhance resistance to nucleases and improved stability. In some embodiments, aptamers of the disclosure comprise a phosphodiester linkage modification, e.g., incorporation of phosphorothioate or boranophosphate linkages, to enhance resistance to nucleases. In some embodiments, the aptamers of the disclosure are made of L-ribose-based nucleotides instead of natural D-nucleotides to confer nuclease resistance. In some embodiments, aptamers of the disclosure are composed of locked nucleic acids or analogues thereof. In some embodiments, the aptamers of the disclosure comprise a 3′ end cap to prevent nuclease resistance.

As described herein the aptamer is a high affinity binding partner to at least a portion of the molecular target in the sample. As used herein, a “high affinity binding partner” generally has a low dissociation constant with its molecular target. In some embodiments, the aptamer has high affinity to a molecular target protein or a molecular tag fused with a molecular target. In some embodiments, the high affinity binding is reversible between the aptamer and the molecular target (or molecular target fused with a tag). In some embodiments, the binding can be dissociated by a reagent with higher affinity to the aptamer or a reagent capable of changing the conformation of the aptamer or the molecular target. The binding affinity can be measured by equilibrium dissociation constant (KD) of the aptamer and the target protein. The smaller the KD value, the greater the binding affinity of the aptamer for its target protein. The larger the KD value, the weaker the binding affinity. In some embodiments, the KD of the aptamer and target protein is less than 0.001 nM, 0.01 nM, 0.1 nM, 1 nM, 5 nM, 10 nM, 20 nM, 30 nM, 50 nM, or 100 nM. In some embodiments, the aptamer binds green fluorescent protein tag of the molecular target, and the KD of the aptamer is less than 20 nM, less than 10 nM, less than 5 nM or between 1-50 nM or preferably between 5-20 nM.

In identifying suitable aptamers for use in the methods of the present application, a person of skill in the art would understand that this involves selecting aptamers that bind the molecular target with sufficiently high affinity (e.g., Kd<50 nM) and specificity from a pool of nucleic acids containing a random region of varying or predetermined length (Shi et al., “A Specific RNA Hairpin Loop Structure Binds the RNA Recognition Motifs of the Drosophila SR Protein B52,” Mol. Cell Biol. 17:1649-1657 (1997); Shi, “Perturbing Protein Function with RNA Aptamers,” Thesis, Cornell University, University Microfilms, Inc. (1997), which are hereby incorporated by reference in their entirety).

For example, identifying suitable aptamers of the present application can be carried out using an established in vitro selection and amplification scheme known as SELEX. The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk and Gold, “Systematic Evolution of Ligands by Exponential Enrichment: RNA Ligands to Bacteriophage T4 DNA Polymerase,” Science 249:505-510 (1990), which are hereby incorporated by reference in their entirety. The SELEX procedure can be modified so that an entire pool of aptamers with binding affinity can be identified by selectively partitioning the pool of aptamers. This procedure is described in U.S. Patent Application Publication No. 2004/0053310, which is hereby incorporated by reference in its entirety.

In some embodiments, the portion of the molecular target bound by the aptamer is a tag moiety. In some embodiments, the aptamer is a high affinity binding partner to at least a portion of the tag moiety of the molecular target. The tag moiety can be fused or conjugated to a protein, an RNA, a DNA, a lipid, or a carbohydrate component of the molecular target.

Exemplary tag moieties that can be fused or conjugated to the molecular target in accordance with the methods disclosed herein include, without limitation, a fluorescent protein or fragment thereof, a poly-His tag, a maltose binding protein tag, an albumin-binding protein tag, a calmodulin binding peptide tag, a glutathione S-transferase tag, a chitin binding protein tag, FLAG-tag, HA-tag, Protein A tag, and combinations thereof. Aptamers to various tag moieties described herein and are known in the art. For example, WO 2014/185802 to Bartnicki et al. describes DNA aptamers that bind to His tags which are suitable for use in accordance with the methods of the disclosure, and U.S. Patent Application Publication No. 2010/0036106 to Yoshida et al. describes high affinity RNA aptamers that bind with high affinity to GST tags, which are also suitable for use in accordance with the methods of the disclosure. Fusing, conjugating, or labeling a protein, an RNA, a DNA, a lipid, or a carbohydrate with a tag moiety can be carried out using standard molecular biology techniques that are well known to those of skill in the art

In some embodiments, the molecular target is fused or conjugated to a fluorescent protein tag. Fluorescent protein tags can be a naturally occurring protein or an engineered protein, such as a derivative of the naturally occurring fluorescent protein.

Exemplary fluorescent proteins include, without limitation, Aequorea-derived proteins such as Green Fluorescent Protein (“GFP”), enhanced Green Fluorescent Protein (“eGFP”), Yellow Fluorescent Protein (“YFP”), enhanced Yellow Fluorescent Protein (“EYFP”), and Cyan Fluorescent Protein (“CFP”), Enhanced Cyan Fluorescent Protein (“ECFP”), their variants or the combination thereof, as well as proteins derived from coral species including, but not limited to, Discosoma and Trachyphyllia geoffroyi. Other proteins having fluorescent or other signaling properties that are known in the art and commercially available can also be used.

Exemplary modified fluorescent proteins that can also be utilized as tag moieties in the methods of the present disclosure include those that contain one or more of the following modifications: circular permutation (Baird et al., “Circular Permutation and Receptor Insertion Within Green Fluorescent Proteins,” Proc. Natl. Acad. Sci. USA 96:11241-11246 (1999), which is hereby incorporated by reference in its entirety), splitting (Zhang et al., “Combinatorial Marking of Cells and Organelles with Reconstituted Fluorescent Proteins,” Cell 119:137-144 (2004), which is hereby incorporated by reference in its entirety), enhanced folding (Pedelacq et al., “Engineering and Characterization of a Superfolder Green Fluorescent Protein,” Nat. Biotechnol. 24:79-88 (2006), which is hereby incorporated by reference in its entirety), or other modifications (Zhang et al., “Creating New Fluorescent Probes for Cell Biology,” Nat. Rev. Mol. Cell Biol. 3:906-918 (2002), which is hereby incorporated by reference in its entirety).

Specific examples of fluorescent proteins suitable for use in accordance with the methods disclosed herein (and their encoding nucleic acids) are well known in the art including, without limitation, those reported as Genbank Accessions AB195239, DD431502-DD431504, DD420089-DD420091, AY013821, AY013824-AY013827, EF064258-EF064259, AF435-427-AF435-434, DQ092360-DQ092365, DQ525024-DQ525025, X83959-X83960, AY533296, AB041904, X96418, BD136947-BD136949, U73901, AX250563-AX250571, AF302837, AF183395, AF058694-AF058695, U50963, L29345, M62653-M62654, DQ301560, AY679106-AY679108, AY678264-AY678271, AF168419-AF168420, AF272711, AY786536-AY786537, AF545828, AF506025-AF506027, AF420593, BAC20344, BD440518-BD440519, and AB085641, each of which is hereby incorporated by reference in its entirety.

In some embodiments of the present disclosure, the aptamer is a nucleic acid aptamer that binds to a fluorescent protein, e.g., GFP, eGFP, eCFP, and eYFP. In accordance with these embodiments, the fluorescent protein is a tag moiety associated with the molecular target being analyzed in the method of the present application. Exemplary nucleic acid aptamers that bind fluorescent proteins that are suitable for use in the methods described herein include those disclosed in U.S. Pat. No. 8,445,655 to Kotlikoff et al., and Shui et al., “RNA Aptamers that Functionally Interact with Green Fluorescent Protein and its Derivatives,” Nucleic Acids Res. 40(5): e39 (2012), which are hereby incorporated by reference in their entirety. These nucleic acid aptamers have a core region sequence that is the primary portion of the aptamer having fluorescent protein binding activity. Accordingly, suitable aptamers that bind with high affinity to green fluorescent proteins for use in the methods disclosed herein include aptamers having any one of the core sequences provided in Table 1 below.

TABLE 1
Exemplary GFP Aptamer Core Region Nucleotide
Sequences as disclosed in
U.S. Patent No. 8,445,655 to Kotlikoff et al.

| SEQ | |
|---|---|
| ID NO: | Core Region Nucleotide Sequence |
| 1 | AUCGAAUUGUNNGGUAGAAAGUCCUUUGAGAGNAAC |
| | CNGGGNGGAUACUG, |
| | where N is any nucleotide (A, U, C, or G). |
| 2 | AUCGAAUUGUUAGGUAGAAAGUCCUUUGAGAGGAAC |
| | CUGGGAGGAUACUG |
| 3 | AUCGAAUUGUGUGGUAGAAAGUCCUUUGAGAGAAAC |
| | CAGGGGGGAUACUG |
| 4 | NGNGAAUUGNGUGGGGAAAGUCCUNAAAAGAGGGCC |
| | ACNGCCGAAACGCC, |
| | where N is any nucleotide (A, U, C, or G) |
| 5 | UGUGAAUUGGGUGGGGAAAGUCCUGAAAAGAGGGCC |
| | ACCGCCGAAACGCC |
| 6 | UGCGAAUUGGGUGGGGAAAGUCCUGAAAAGAGGGCC |
| | ACCGCCGAAACGCC |
| 7 | UGUGAAUUGAGUGGGGAAAGUCCUGAAAAGAGGGCC |
| | ACUGCCGAAACGCC |
| 8 | UGUGAAUUGGGUGGGGAAAGUCCUUAAAAGAGGGCC |
| | ACCGCCGAAACGCC |
| 9 | GGUGAAUUGGGUGGGGAAAGUCCUUAAAAGAGGGCC |
| | ACCGCCGAAACGCC |
| 10 | UGUGAAUUGAGUGGGGAAAGUCCUGAAAAGAGGGCC |
| | ACCGCCGAAACGCC |
| 11 | UGUGAAUUGAGUGGGGAAAGUCCUGAAAAGAGGGCC |
| | ACAGCCGAAACGCC |
| 12 | UUGGGGUGGGGUGGGGAAAGUCCUUAAAA |
| | GAGGGCCACCACAGAAGCAAU |
| 13 | UUGGGGUGGGGUGGGGAAAGUCCUUAAAA |
| | GAGGGCCACCACAGAAGCAAU |

The RNA aptamers described above, may further comprise a first primer region located 5′ to the core region, a second primer region located 3′ to the core region; and one or both of a 5′ random nucleotide sequence region and a 3′ random nucleotide sequence region. These nucleic acid aptamers assume a secondary structure comprising three stems and a central loop.

In another embodiment, the aptamer is a nucleic acid aptamer that binds to a fluorescent protein and is based on nucleic acid sequences described in Tome et al., “Comprehensive analysis of RNA-protein interactions by high-throughput sequencing-RNA affinity profiling,” Nat Methods (11): 683-688 (2014), which is hereby incorporated by reference in its entirety. Accordingly, an exemplary nucleic acid aptamer for use in the methods described herein includes the GFP aptamer having the sequence of:

(SEQ ID NO: 14)
GGGAGCTTCTGGACTGCGATGGGAGCACGAAACGTCGTGGCGCAATTGG
GTGGGGAAAGTCCTTAAAAGAGGGCCACCACAGAAGC.

In another embodiment, the nucleic acid aptamer is an aptamer that binds to negative elongation factor (NELF). Suitable nucleic acid aptamers that bind NELF include those disclosed in Tome et al., “Comprehensive analysis of RNA-protein interactions by high-throughput sequencing-RNA affinity profiling,” Nat Methods (11): 683-688 (2014), which is hereby incorporated by reference in its entirety. An exemplary nucleic acid aptamer that binds to NELF for use in the methods described herein has the sequence of:

(SEQ ID NO: 15)
GGGAATGGATCCACATCTACGAATTCCCAACGACTGCCGAGCGAGATTA
CGCTTGAGCGCCCCACTGAGGATGCCCACGGGCGATTGGGGCACGGC,
TTCACTGCAGACTTGACGAAGCTT.

In accordance with the methods described herein, a suitable aptamer is contacted with the sample containing the molecular target under conditions effective for the molecular target and aptamer to bind to each other. The molecular target bound to the aptamer is then separated from the sample to form a molecular target enriched sample.

In some embodiments, the aptamer (e.g., a nucleic acid aptamer) is immobilized on a solid support. Suitable solid supports for use in the methods of the present application include, without limitation, a bead, a particle, a column, a fluidic device, a microfluidic device, a microarray, a strand, a gel, a sheet, a tube, a sphere, a capillary, a pad, a film, a plate, a disc, and a membrane. The solid support may have any convenient shape, such as a disc, square, circle, etc., and may contain raised or depressed regions suitable for immobilization of aptamers of the present disclosure. By way of example, the aptamer is immobilized on a surface and/or the interior of a bead, substrate, matrix or network via high affinity binding complex. The bead, substrate, matrix or network can be coated with one part of a high affinity binding complex (e.g., streptavidin), and the aptamer is conjugated to the other part of the high affinity binding complex (e.g., biotin). The aptamer and molecular target is then eluted from the solid support. The elution buffer may comprise the other part of the high affinity binding complex.

As noted above, immobilization of the aptamer to a suitable solid support can be achieved by covalent linkage or by noncovalent interaction (e.g., biotinylated DNA bound to avidin coated beads). Accordingly, the surface of the solid support is coated with a first binding partner of a binding complex and the aptamer may be coupled to a second binding partner of the binding complex, and the aptamer is immobilized on the solid support via binding between the first and second binding partners of the binding complex. Suitable first and second binding partners for immobilizing an aptamer to a solid support in accordance with this embodiment of the present application include, without limitation, biotin and streptavidin, desthiobiotin and streptavidin, maltose and maltose binding protein (MBP), chitin and chitin binding protein, amylose and MBP, glutathione and glutathione-S-transferase, and Ni2+-NTA and His-tag, and integrin and integrin binding peptide. Methods of covalently attaching oligonucleotides to a solid support are well known in the art, see e.g., Gosh and Musso, “Covalent Attachment of Oligonucleotides to Solid Supports,” Nucleic Acids Res. 15(13): 5353-5372 (1987), Joos et al., “Covalent Attachment of Hybridizable Oligonucleotides to Glass Supports,” Anal. Biochem. 247(1):96-101 (1997); Lund et al., “Assessment of Methods for Covalent Binding of Nucleic Acids to Magnetic Beads, Dynabeads, and the Characteristics of the Bound Nucleic Acids in Hybridization Reactions,” Nucleic Acids Res. 16(22):10861-80 (1988), which are hereby incorporated by reference in their entirety.

In some embodiments, the aptamer is reusable or can be rejuvenated or regenerated. The aptamer (e.g. immobilized aptamer) can be used for at least 2 runs (or rounds), at least 3, 4, 5, 10 runs (or rounds) for purifying molecular targets.

The molecular targets collected by using aptamer separation or purification is free or substantially free of other non-specific proteins or contaminating proteins without additional purification process to remove the contaminant proteins. The molecular target collected after the aptamer separation forms a molecular target enriched sample. In some embodiments, the molecular target of this enriched sample is subject to analysis as described here. In some embodiments, the method further comprises an additional enrichment step. This enrichment step may be added either before or after binding of the aptamer to the molecular target. Addition of such a step to the method of the present application allows for a sequential affinity-purification to enrich for a particular molecular target of interest, e.g., a molecular target known to comprise two or more biomolecules. This technique is particularly useful when the aptamer bound portion of the molecular target is a known constituent of more than one macromolecular complex, and it is desirable to analyze a particular macromolecular complex. The technique is also useful to simply improve the purity of the molecular target prior to analysis.

One embodiment of the sequential enrichment step is depicted in FIG. 15. Briefly, FIG. 15 depicts the use of an aptamer (e.g., GFP-aptamer) to carry out a first enrichment of a macromolecular complex from a crosslinked cellular lysate that consists of tagged protein (in this example GFP-tagged RNA Polymerase II), an associated factor (which can be any protein), and histone proteins all bound to a DNA sequence. Following a series of washes the aptamer bound complex is eluted and the same eluate is used in the sequential enrichment step to further purify the molecular target via immunoprecipitation of the associated factor or histones associated with the target using an additional binding agent. Immunoprecipitation results in recovery of the macromolecular complex of interest. This macromolecular complex can subsequently be analyzed to identify the protein constituents of the complex, the three dimensional structure, and/or DNA sequence(s) associated with the proteins of the complex.

Accordingly, the method of the present application may further involve providing, after separating the molecular target from the sample to form a molecular target enriched sample, a binding agent that binds to a different portion of the molecular target than bound by the aptamer. The molecular target enriched sample is contacted with the binding agent under conditions effective for the molecular target and binding agent to bind to each other. The binding agent is then immunoprecipitated, thereby enriching/isolating the bound molecular target.

Alternatively, as depicted in FIG. 16, the method of the present application may include a sequential enrichment step that is added before binding of the aptamer to the molecular target. In this embodiment, a binding agent is provided that binds to a different portion of the molecular target than bound by the aptamer. The binding agent is introduced into the sample, prior to contacting the sample with the aptamer, under conditions effective for the molecular target and binding agent to bind to each other. The binding agent is then immunoprecipitated, thereby enriching/isolating the bound molecular target, and the immunoprecipitated molecular target is subjected to contacting with the aptamer and the methods following, which are described supra. In some embodiments, the sample is crosslinked prior to carrying out the sequential enrichment step, e.g., prior to introducing the binding agent. In some embodiments, the sample is not subject to crosslinking prior to introducing the binding agent.

In accordance with these embodiments, suitable binding agents for use in the sequential enrichments steps of the methods described herein include any binding agent typically used for immunoprecipitation, including, without limitation, full antibodies, epitope binding fragments of whole antibodies, antibody derivative, antibody mimics, aptamers, etc.

In some embodiments, the binding agent is a full antibody, composed of two light chains and two heavy chain, where the variable regions of each chain (i.e., V_{L} and V_{H}) form the epitope binding region of the antibody. In some embodiments, the binding agent is an epitope binding fragment of an antibody. Examples of the epitope-binding fragments that can be utilized in the methods described herein include Fab′ or Fab fragments (i.e., monovalent fragments containing the V_{L}, V_{H}, C_{L} and C_{H}1 domains); F(ab′)₂ fragments (i.e., bivalent fragments comprising two Fab fragments linked by a disulfide bridge at the hinge region; Fd fragments (consisting essentially of the V_{H} and C_{H}1 domains); Fv fragments consisting essentially of a V_{L} and V_{H} domain; and dAb fragments, which consist essentially of a V_{H} or V_{L} domain. In some embodiments, the binding agent is an antibody derivative, which is a molecule that contains at least one epitope binding domain of any antibody. An exemplary antibody derivative for use in the methods described herein is a single chain Fv (“scFv”) antibody, which comprises covalently linked V_{H}::V_{L} heterodimer to form a single antigen binding domain.

When used to sequentially enrich the molecular target as described herein, the antibody, binding fragment thereof, or derivative thereof binds to portion of the molecular target that is different than the portion bound by the aptamer. This portion may be a structural or sequence portion of the molecular target itself or another tag moiety as described infra. For example, as shown in FIGS. 15 and 16, an antibody and aptamer bind to different proteins that are complexed within the same molecular target.

Once the molecular target has been sufficiently separated from the sample, it is analyzed using any one of a variety of methods well known in the art including, without limitation, mass spectrometry, cryo-electron microscopy, and nucleotide sequencing.

In some embodiments, the molecular target comprises two or more associated biomolecules and analysis of these associated biomolecules involves identifying each of the two or more associated biomolecules of the molecular target.

In accordance with this embodiment, the separated or immunoprecipitated molecular target may be subjected to mass spectrometry to detect each of the two or more associated biomolecules. The use of mass spectrometry for identification of associated biomolecules is well known in the art and described in, for example, Yugandhar et al., “Inferring Protein-Protein Interaction Networks From Mass Spectrometry-Based Proteomic Approaches: a Mini-Review,” Comput Struct Biotechnol J 17:805-811 (2019), which is hereby incorporated by reference in its entirety.

In some embodiments, the molecular target is directly analyzed using MS without the incorporation of any additional separation process to remove contaminant proteins. In some embodiments, the molecular target is subject to one or more additional purification steps to remove contaminant proteins.

Identification of low-abundant proteins by MS becomes possible due to high specificity and enrichment by the aptamer. This also helps in identifying post-translational modifications of the captured proteins without the need for any modification-specific enrichment step. Thus, in some embodiments, the separated or immunoprecipitated molecular target of the method described herein is subjected to mass spectrometry to detect one or more posttranslational modifications of the molecular target. Posttranslational modifications of the molecular target that can be detected using MS include, without limitation, methylation, acetylation, phosphorylation, sumoylation, or combinations thereof. The presence of covalent modifications in proteins affects the molecular weight of the modified amino acids, and the mass increment or deficit can be detected by MS. Accordingly, when practicing the methods of the present application, MS analysis of the molecular target can be used for very high sensitivity detection of post translational modifications in the molecular target, to identify the site of the post translational modification within the molecular target, discover the presence of novel post translational modifications in the molecular target and associated protein sand finally to quantify the relative changes in post translational modification occupancy at distinct sites in the molecular target (Larsen et al., “Analysis of Posttranslational Modification of Proteins by Tandem Mass Spectrometry,” Biotechniques 40(6):790-797 (2018), which is hereby incorporated by reference in its entirety). Methods for identification of post-translational modifications by MS are described in, for example, Larsen et al., “Analysis of Posttranslational Modification of Proteins by Tandem Mass Spectrometry,” Biotechniques 40(6):790-797 (2018), which is hereby incorporated by reference in its entirety.

In some embodiments, analysis of the molecular target is performed using cryo-electron microscopy. The separated or immunoprecipitated molecular target is subjected to cryo-electron microscopy to determine the three-dimensional structure of the molecular target. In some embodiments, the three-dimensional structure of two or more associated biomolecules is determined.

In electron cryo-microscopy (cryoEM), the purified molecular target (e.g., target protein or protein complex) is preserved in vitreous water on sample grids allowing for its native structural state to be maintained (Nogales et al., “Cryo-EM:a Unique Tool for the Visualization of Macromolecular Complexity,” Mol. Cell 58:677-689 (2015), which is hereby incorporated by reference in its entirety). The use of cryoEM in analysis of molecular targets is known in the art and reviewed in, for example, Murata et al., “Cryo-electron Microscopy for Structural Analysis of Dynamic Biological Macromolecules,” Biochimica et Biophysica Acta 1862(2):3240334 (2018), which is hereby incorporated by reference in its entirety.

In some embodiments, the molecular target comprises nucleotide oligomers and the analysis involves isolating the nucleotide oligomers from the immunoprecipitated or separated molecular target and subjecting the isolated nucleotide oligomers to an amplification reaction, a sequencing reaction, or a combination thereof to identify the isolated nucleotide oligomers of the molecular target.

Amplification of nucleotide oligomers that have been isolated from the immunoprecipitated or separated molecular target may be performed using nucleic acid amplification methods well known in the art. These methods include, without limitation, polymerase chain reaction (PCR) (U.S. Pat. No. 5,219,727, which is hereby incorporated by reference in its entirety) and its variants such as in situ polymerase chain reaction (U.S. Pat. No. 5,538,871, which is hereby incorporated by reference in its entirety), quantitative polymerase chain reaction (U.S. Pat. No. 5,219,727, which is hereby incorporated by reference in its entirety), nested polymerase chain reaction (U.S. Pat. No. 5,556,773), self-sustained sequence replication and its variants (Guatelli et al. “Isothermal, In vitro Amplification of Nucleic Acids by a Multienzyme Reaction Modeled after Retroviral Replication,” Proc Natl Acad Sci USA 87(5): 1874-8 (1990), which is hereby incorporated by reference in its entirety), transcriptional amplification and its variants (Kwoh et al. “Transcription-based Amplification System and Detection of Amplified Human Immunodeficiency Virus type 1 with a Bead-Based Sandwich Hybridization Format,” Proc Natl Acad Sci USA 86(4): 1173-7 (1989), which is hereby incorporated by reference in its entirety), Qb Replicase and its variants (Miele et al. “Autocatalytic Replication of a Recombinant RNA.” J Mol Biol 171(3): 281-95 (1983), which is hereby incorporated by reference in its entirety), cold-PCR (Li et al. “Replacing PCR with COLD-PCR Enriches Variant DNA Sequences and Redefines the Sensitivity of Genetic Testing.” Nat Med 14(5): 579-84 (2008), which is hereby incorporated by reference in its entirety) or any other nucleic acid amplification method known in the art. Depending on the amplification technique that is employed, the amplified molecules are detected during amplification (e.g., real-time PCR) or subsequent to amplification using detection techniques known to those of skill in the art. Suitable nucleic acid detection assays include, for example and without limitation, northern blot, microarray, serial analysis of gene expression (SAGE), next-generation RNA sequencing (e.g., deep sequencing, whole transcriptome sequencing, exome sequencing), gene expression analysis by massively parallel signature sequencing (MPSS), immune-derived colorimetric assays, and mass spectrometry (MS) methods (e.g., MassARRAY® System).

Alternatively, sequencing of nucleotide oligomers that have been isolated from the immunoprecipitated or separated molecular target may be performed using methods of nucleotide sequencing that are well known in the art. Illustrative non-limiting examples of nucleic acid sequencing techniques include, but are not limited to, chain terminator (Sanger) sequencing and dye terminator sequencing, as well as “next generation” sequencing techniques. Those of ordinary skill in the art will recognize that because RNA is less stable in the cell and more prone to nuclease attack experimentally RNA is usually, although not necessarily, reverse transcribed to DNA before sequencing.

Additional DNA sequencing techniques suitable for use in the analysis of the molecular target that are known in the art include, without limitation, fluorescence-based sequencing methodologies (See, e.g., Birren et al., Genome Analysis: Analyzing DNA, 1, Cold Spring Harbor, N.Y.; which is hereby by reference in its entirety). In some embodiments, automated sequencing techniques are utilized, or the systems, devices, and methods employ parallel sequencing of partitioned amplicons (PCT Publication No: WO2006084132 to Kevin McKernan et al., which is hereby incorporated by reference in its entirety). DNA sequencing may also be achieved by parallel oligonucleotide extension (See, e.g., U.S. Pat. No. 5,750,341 to Macevicz et al., and U.S. Pat. No. 6,306,597 to Macevicz et al., both of which are hereby incorporated by reference in their entireties). Additional examples of sequencing techniques include the Church polony technology (Mitra et al., Analytical Biochemistry 320, 55-65 (2003); Shendure et al., Science 309, 1728-1732 (2005); U.S. Pat. Nos. 6,432,360, 6,485,944, U.S. Pat. No. 6,511,803; which are hereby incorporated by reference in their entireties) the 454 picotiter pyrosequencing technology (Margulies et al., Nature 437, 376-380 (2005); US 20050130173; herein incorporated by reference in their entireties), the Solexa single base addition technology (Bennett et al., Pharmacogenomics 6:373-382 (2005); U.S. Pat. Nos. 6,787,308; 6,833,246; which are hereby incorporated by reference in their entirety), Illumina Single base sequencing technology, the Lynx massively parallel signature sequencing technology (Brenner et al. Nat. Biotechnol. 18:630-634 (2000); U.S. Pat. Nos. 5,695,934; 5,714,330; which are hereby incorporated by reference in their entirety) and the Adessi PCR colony technology (Adessi et al. Nucleic Acid Res. 28, E87 (2000); WO 00018957, which are hereby incorporated by reference in their entirety).

A set of methods referred to as “next-generation sequencing” techniques (Voelkerding et al., Clinical Chem. 55: 641-658 (2009); MacLean et al., Nature Rev. Microbiol., 7: 287-296, which are hereby incorporated by reference in their entirety) may also be used in accordance with this embodiment. These techniques may be used to sequence the nucleotide oligomers that have been isolated from the immunoprecipitated or separated molecular target. Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems (Voelkerding et al., Clinical Chem. 55: 641-658 (2009); MacLean et al., Nature Rev. Microbiol., 7: 287-296; U.S. Pat. No. 5,912,148; U.S. Pat. No. 6,130,073; which are hereby incorporated by reference in their entirety). Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

Other emerging single molecule sequencing methods useful in the methods of the present application include real-time sequencing by synthesis using a VisiGen platform (Voelkerding et al., Clinical Chem. 55: 641-658 (2009); U.S. Pat. No. 7,329,492; U.S. patent application Ser. No. 11/671,956; U.S. patent application Ser. No. 11/781,166; which are hereby incorporated by reference in their entirety).

While certain embodiments have been set forth, alternative embodiments and various modifications will be apparent form the above description to those skilled in the art. These and other alternatives are considered equivalents and within the spirit and scope of this disclosure.

### EXAMPLES

The examples below are intended to exemplify the practice of embodiments of the disclosure but are by no means intended to limit the scope thereof.

### Materials and Methods

Cell culture and transfection. Human HCT116 cells were grown in McCoy's 5A media (with 10% FBS+P/S) at 37° C. Around 4 million HCT116 cells were plated in McCoy's 5A media (with 10% FBS+P/S) 24 h prior to being transfected with pEGFP-N1 or pHSF1-GFPN3 (Wang et al., “Regulation of Molecular Chaperone Gene Transcription Involves the Serine Phosphorylation, 14-3-3 Epsilon Binding, and Cytoplasmic Sequestration of Heat Shock Factor 1,” Mol. Cell. Biol. 23:6013-6026 (2003), which is hereby incorporated by reference in its entirety) (gift from Stuart Calderwood, Addgene #32538) plasmid and Fugene HD reagent at 3:1 ratio. It should be noted that in this study for HCT116 and S2 cells, GFP or GFP-fusion protein refers to protein containing enhanced (E)-GFP which has equivalent binding affinity to the GFP-aptamer (Shui et al., “RNA Aptamers that Functionally Interact with Green Fluorescent Protein and its Derivatives,” Nucleic Acids Res. 40:e39 (2012), which is hereby incorporated by reference in its entirety). Transfection efficiency was monitored after 20 h (˜90% efficient as judged by GFP fluorescence) and cells were then subjected to instantaneous HS (Mahat et al., “Use of Conditioned Media is Critical for Studies of Regulation in Response to Rapid Heat Shock,” Cell Stress Chaperones 22:155-162 (2017), which is hereby incorporated by reference in its entirety). Cells were scraped after 30 min HS and centrifuged at 500×g for 5 min at 4.0 and washed twice with ice-cold 1× PBS.

Drosophila S2 cells were grown in M3+BPYE media (with 10% FBS) at 25.C. S. pombe cells were cultivated using standard procedures (Moreno et al., “Molecular Genetic Analysis of Fission Yeast Schizosaccharomyces pombe,” Methods Enzymol. 194:795-823 (1991), which is hereby incorporated by reference in its entirety).

Human cellular lysate preparation and aptamer-based affinity purification. Transfected human HCT116 cells before or after HS were resuspended in 0.5 ml ice-cold cellular lysis buffer (1× PBS+0.2% NP40+1× EDTA-free Protease inhibitor cocktail). Cells were incubated on ice for 30 min followed by sonication with Bioruptor Diagenode at High setting (30 s ON/30 s OFF) for 5 min at 4.C. The lysate was centrifuged at 20 000× g for 10 min at 4.0 and the resulting supernatant was transferred to a new tube. The cleared lysate was diluted to a final buffer containing 1×PBS, 0.05% NP40, 5.25 mM MgCl₂, 187.5 ng/μl yeast RNA, 187.5 ng/μl sheared salmon sperm DNA, 200 units SUPERase IN/ml.

RNA preparation and immobilization on beads. The polyadenylated (20 nt ‘A’) GFP or polyadenylated control (NELF)-aptamer was in vitro transcribed using T7 RNA polymerase and purified by phenol/chloroform and polyacrylamide gel extraction. DNA sequences of the GFP and control (NELF)-RNA aptamers are based on sequences used in (Tome et al., “Comprehensive Analysis of RNA-protein Interactions by High-throughput Sequencing-RNA Affinity Profiling,” Nat. Methods 11:683-688 (2014), which is hereby incorporated by reference in its entirety) and are as follows; GFP-aptamer:

(SEQ ID NO: 14)
GGGAGCTTCTGGACTGCGATGGGAGCACGAAA
CGTCGTGGCGCAATTGGGTGGGGAAAGTCCTT
AAAAGAGGGCCACCACAGAAGC,
NELF-aptamer:
(SEQ ID NO: 15)
GGGAATGGATCCACATCTACGAATTCCCAACG
ACTGCCGAGCGAGATTACGCTTGAGCGCCCCA
CTGAGGATGCCCACGGGCGATTGGGGCACGGC
TTCACTGCAGACTTGACGAAGCTT.

200 pmol of polyadenylatedGFP or the control (NELF)-aptamer was annealed to equimolar desthiobiotin-oligodT-20 in 200 of 1× annealing buffer (10 mM Tris-HCl pH 7.5, 50 mM NaCl) by heating at 95.0 for 3 min and slow cooling to room temperature over >1 h. For each pull-down 1 mg of Dynabeads MyOne Streptavidin C1 (Thermo) magnetic beads were washed once with 1 ml and twice with 0.1 ml of Tween wash buffer (5 mM Tris-HCl pH 7.5, 0.5 mM EDTA, 1 M NaCl, 0.05% Tween-20) by placing on a magnetic separator for 2 min and removing the supernatant. To eliminate possible RNase activity, beads were washed once with 0.1 ml of 0.1M NaOH, 0.05M NaCl followed by two washes of 0.1 ml of 0.1 M NaCl with changing tubes in between washes. The beads were resuspended in 200 μl of 2× binding buffer (10 mM Tris-HCl pH 7.5, 1 mM EDTA, 2M NaCl) supplemented with 4 units/ml SUPERase IN. The resulting bead slurry was mixed with the annealed RNA aptamer and incubated on a thermomixer for 1 h at 23.0 with shaking. Aptamer bound beads were washed twice with 0.4 ml of Bead wash buffer (1× PBS, 0.05% NP40, 5 mM MgCl2), supplemented with 4 units/ml SUPERase IN with changing tubes in between washes.

Binding to lysate, washing and elution. The GFP- or control-aptamer bound beads were resuspended in the diluted cellular lysate and incubated at 4.0 with rotation for 2 h. The beads were placed on a magnet and supernatant was removed. The beads were washed twice with 0.5 ml of Bead wash buffer and twice with 0.5 ml of 1× PBS, 5 mM MgCl₂, with changing tubes in between washes. Beads were resuspended in 50 μl of fresh Elution buffer (5 mM Biotin, 50 mM ammonium phosphate, pH 7.5) and incubated in a Thermomixer at 37.0 with shaking for 1 h followed by collection of the eluate into a fresh tube.

Details of the immunoprecipitation method utilizing the GFP-antibody are provided below.

Affinity purification from other organisms. Drosophila S2 cells stably expressing GFP, wild-type Drosophila S2 cells and S. pombe cells expressing endogenous GFP-Rpb3 were cultured for lysate or nuclear extract preparation, and were subjected to aptamer-based purification or Tandem affinity purification (TAP)-tag-based purification. Further details of these methods are provided below.

Proteomics workflow. Mass spectrometry sample preparation. Eluate from human HCT116 cells was solubilized in 8 M urea in 100 mM ammonium bicarbonate (ABC). The sample was reduced in 10 mM dithiothreitol (DTT) at 37.0 for 1 h. Cysteines were blocked in 30 mM methyl methanethiosulfonate (MMTS) at room temperature for 1 h without light. The sample volume was adjusted to reduce the concentration of urea to below 1M using 100 mM ABC, and proteins were digested using 1 μg of Trypsin at 37.0 overnight. Salts, RNA and other contaminants were removed using mixed-mode cation exchange (MCX) columns (Oasis). Elution buffer spiked-in with biotin and analyzed before and after MCX cleanup shows that the biotin signal is drastically reduced after cleanup (FIG. 17). Samples were dried using a speed vacuum.

Mass Spectrometry. All mass spectrometry with samples prepared from human HCT116 cells was performed on a Q-Exactive HF-X (Thermo Fisher Scientific) mass spectrometer with a EasyLC 1200 HPLC and autosampler (Thermo Fisher Scientific). The dried pull-downs were solubilized in 30 μl of loading buffer (0.1% trifluoroacetic acid and 2% acetonitrile in water), and 3 μl was injected via the autosampler onto a 150-μm Kasil fritted trap packed with Reprosil-Pur C18-AQ (3-μm bead diameter) to a bed length of 2 cm at a flow rate of 2 μl/min. After loading and desalting using a total volume of 8 μl of loading buffer, the trap was brought on-line with a pulled fused-silica capillary tip (75 μm i.d.) packed to a length of 25 cm with the same Dr. Maisch beads. The column and trap were mounted to a heated microspray source (CorSolutions) at 50° C. Peptides were eluted off of the column using a gradient of 5-28% acetonitrile in 0.1% formic acid over 25 min, followed by 28-60% acetonitrile over 5 min at a flow rate of 300 nl/min.

The mass spectrometer was operated using electrospray ionization (2 kV) with the heated transfer tube at 250.0 using data dependent acquisition (DDA), whereby one orbitrap mass spectrum (m/z 400-1600) was acquired with up to 20 orbitrap MS/MS spectra. The resolution for MS in the orbitrap was 60 000 at m/z 200, and 15 000 for MS/MS. The automatic gain control targets for MS was 3e6, and 1e5 for MS/MS. The maximum fill times were 45 and 25 ms, respectively. The MS/MS spectra were acquired using quadrupole isolation with an isolation width of 1.6 m/z and HCD collision energy (NCE) of 28%. The precursor ion threshold intensity was set to 2e6 in order to trigger an MS/MS acquisition. Furthermore, MS/MS acquisitions were allowed for precursor charge states of 2-5. Dynamic exclusion (including all isotope peaks) was set for 10 s.

Details of the mass spectrometry method associated with NELF-aptamer pull-down from Drosophila S2 cells are provided below.

Data Analysis. Raw spectral files were converted to mascot generic format using MSGUI, then searched against a database containing human proteins from UniProt with the addition of the protein sequence for GFP using Mascot. The search parameters allowed for fixed cysteine methylthiolation and variable methionine oxidation modifications, with a 10 ppm peptide mass tolerance, 0.5 Da fragment mass tolerance, and one missed tryptic cleavage. Subsequent searches allowed for variable lysine acetylation and serine/threonine phosphorylation, respectively, each with fixed cysteine methylthiolation and variable methionine oxidation allowing for 20 ppm peptide mass tolerance and 0.5 Da fragment mass tolerance and a maximum of three missed tryptic cleavages. Searches were also submitted with the above parameters and a 0.02 Da fragment mass tolerance, which resulted in no substantial changes to the results. Initial analyses of abundance and enrichment were conducted using Scaffold (Searle, “Scaffold: a Bioinformatic Tool for Validating MS/MS-based Proteomic Studies,” Proteomics 10: 1265-1269 (2010), which is hereby incorporated by reference in its entirety). Prediction and scoring of HSF1 interacting partners was done using Significance analysis of interactome (SAINT), and data was presented using the SAINT score and fold change A values (Choi et al., “SAINT: Probabilistic Scoring of Affinity Purification-mass Spectrometry Data,” Nat. Methods 8:70-73 (2011), which is hereby incorporated by reference in its entirety). Rather than performing a traditional fold change calculation, SAINT takes into account representation of each protein among biological replicates. As a control dataset to train the algorithm, the proteins detected in pull-downs using the GFP aptamer from GFP cells were used in addition to pull-downs using the NELF-aptamer from HSF1-GFP cells. This 2-fold control strategy provided proteins that bind non-specifically to RNA and to free GFP. The resulting output was an enrichment calculation (fold change A, the most conservative option) and probability score for interaction with HSF1 (SAINT score). Prediction and assignment of posttranslational modifications was done using Scaffold PTM (Vincent-Maloney et al., “Probabilistically Assigning Sites of Protein Modification with Scaffold PTM,” J. Biomol. Tech. 22:S36-S37 (2011), which is hereby incorporated by reference in its entirety). Site assignments were confirmed using MS1 quantification in Skyline (Pino et al., “The Skyline Ecosystem: Informatics for Quantitative Mass Spectrometry Proteomics,” Mass Spectrom. Rev. 39:229-244 (2017), which is hereby incorporated by reference in its entirety). All mass spectrometry proteome data were deposited to the ProteomeXchange Consortium (Deutsch et al., “The ProteomeXchange Consortium in 2017: Supporting the Cultural Change in Proteomics Public Data Deposition,” Nucleic Acids Res. 45:D1100-D1106 (2017), which is hereby incorporated by reference in its entirety) via the PRIDE repository (Perez-Riverol et al., “The PRIDE Database and Related Tools and Resources in 2019: Improving Support for Quantification Data,” Nucleic Acids Res. 47:D442-D450 (2019), which is hereby incorporated by reference in its entirety) with the dataset identifier PXDO15620.

Gene ontology (GO) analysis. The Protein Analysis Through Evolutionary Relationships (PANTHER) classification system (Mi et al., “Protocol Update for Large-scale Genome and Gene Function Analysis with the PANTHER Classification System (v.14.0),” Nat. Protoc. 14:703-721 (2019), which is hereby incorporated by reference in its entirety) was used to determine the GO classification of enriched proteins. UniProt IDs from proteins statistically enriched (Fishers exact test P<0.05) in HSF1-GFP cells compared to GFP cells with the GFP- or NELF-aptamer were used as query for the PANTHER 15.0 Gene List Analysis tool. Functional classification using the Homo sapiens reference database was performed using this tool. The percent of genes from the query matching to a specific function against the total number of queried genes with function matches was plotted using gg-plot2 (Wickham, H. In: Ggplot2: elegant graphics for data analysis. second edn. Springer, Switzerland (2016), which is hereby incorporated by reference in its entirety).

Immunoprecipitation of HSF1-GFP with the GFP antibody. HCT116 cells were transfected with HSF1-GFP plasmid and harvested after 20 hr of transfection. Cells were incubated in ice-cold cellular lysis buffer (1× PBS+0.2% NP40+1× EDTA-free Protease inhibitor cocktail) on ice for 30 min followed by sonication with Bioruptor Diagenode at High setting (30 s ON/30 s OFF) for 5 min at 4° C. The lysate was centrifuged at 20,000×g for 10 min at 4° C. and the resulting supernatant was transferred to a new tube. The cleared lysate was diluted to a final buffer containing 1× PBS, 0.05% NP40, 5.25 mM MgCl₂. The GFP-antibody (Abcam, ab290) was immobilized on Protein-A Dynabeads for 4 h at 4° C. Beads were washed in antibody wash buffer (1× PBS, 5 mg/ml BSA, 0.05% Tween-20) and finally resuspended in the diluted cellular lysate with rotation at 4° C. for 2 h. The beads were washed twice in 0.5 ml bead wash buffer (1× PBS, 0.05% NP40, 5 mM MgCl₂), with changing tubes in between washes. The beads were washed twice with 0.5 ml of 1× PBS, 5 mM MgCl₂, with changing tubes in between washes. Beads were resuspended in 55 μL of 10 mM Tris-HCl, pH 8.5 and incubated in at 98° C. for 5 min followed by collection of the eluate into a fresh tube.

Aptamer-based GFP purification from Drosophila S2 cells. S2 cells stably expressing GFP were grown in M3+BPYE media (with 10% FBS) at 25° C. Around 100 million cells were collected by centrifugation at 1000×g for 5 min at 4° C. Cells were washed twice in 1× PBS and resuspended in 0.5 ml ice-cold cellular lysis buffer (1× PBS, 0.2% NP40, supplemented with 1× Protease inhibitor cocktail before use) and incubated on ice for 30 min followed by sonication with Bioruptor Diagenode at H setting (30 s ON/60 s OFF) for 15 min at 4° C. The lysate was centrifuged at 20,000×g for 10 min at 4° C. and the resulting supernatant was transferred to a new tube and diluted to a final buffer containing 1× PBS, 0.05% NP40, 5.25 mM MgCl₂, 250 ng/μL yeast RNA, 250 ng/μL sheared salmon sperm DNA (sssDNA), 300 units/ml SUPERase IN.

For crosslinking experiments, 80 million cells were centrifuged at 1000× g for 10 min at 4° C. to remove the media and the pellet was resuspended in 1× PBS. Crosslinking was done by adding 1% Paraformaldehyde and incubated at room temperature for 10 min with occasional mixing. Glycine was added to a final concentration of 147 mM and incubated at room temperature for 5 min with occasional mixing. The crosslinked cells were centrifuged at 1000×g for 5 min at 4° C. and the supernatant was discarded. The pellet was washed with 1× PBS, flash frozen in liquid nitrogen and stored at −80° C. To prepare lysate from the crosslinked cells, the frozen cells were thawed on ice and resuspended in 0.5 ml of ice cold cellular lysis buffer. The cells were incubated on ice for 20 min and centrifuged at 4500×g for 5 min. The pellet was washed once more with 0.5 ml of ice cold cellular lysis buffer. The pellet was resuspended in 0.2 ml of 0.5% SDS and incubated at 37° C. for 15 min. The volume was brought up to 0.5 ml with 1× PBS, 1× Protease inhibitor cocktail and sonicated with Bioruptor Diagenode at H setting (20 s ON/60 s OFF) for 30 min at 4° C. The lysate was centrifuged at 20,000×g for 10 min at 4° C. The resulting supernatant was transferred to a new tube and diluted to a final buffer containing 1× PBS, 250 ng/μL yeast RNA, 250 sssDNA, 0.025% SDS, 0.05% Triton X-100, 5.25 mM MgCl2, 300 units/ml SUPERase IN. The aptamer RNA bead preparation, binding, washing and elution were done similar to the procedure used for human HCT116 cells, except in case of crosslinked cells, the elution was done with RNase A/T1 cocktail treatment.

Drosophila nuclear extract preparation and aptamer-based affinity purification. Nuclear extract was prepared from Drosophila S2 cells following the protocol as described in (Brunner et al., “A High Quality Catalog of the Drosophila melanogaster Proteome,” Nat Biotechnol 25:576-583 (2007), which is hereby incorporated by reference in its entirety). Briefly, cells were grown to confluency in T150 flasks, collected into falcon tubes and were washed 4 times with 1× PBS by centrifuging at 1000× g at 4° C. for 10 min. Cells were lysed with 5 pellet volumes of ice-cold hypotonic lysis buffer (10 mM HEPES pH 7.6, 1.5 mM MgCl₂, 10 mM KCl, supplemented with 1× Protease inhibitor cocktail and 0.5 mM DTT before use) and incubated on ice for 10 min. The cells were dounced on ice for 20 times with a dounce homogenizer and tight pestle. The lysate was centrifuged at 1000×g at 4° C. for 7 min. Supernatant was discarded and the pellet was washed again with ice cold hypotonic lysis buffer. Finally, the pellet was resuspended in 0.5 packed volumes of ice-cold high salt extraction buffer (20 mM HEPES pH 7.6, 25% glycerol, 1.5 mM MgCl₂, 420 mM NaCl, supplemented with 1× Protease inhibitor cocktail and 0.5 mM DTT before use) and the lysate was stirred for 30 min at 4° C. on an ice bath. The lysate was centrifuged at 20,000× g for 20 min at 4° C. and the supernatant containing the nuclear proteins was transferred into a fresh tube, flash frozen in liquid nitrogen and stored at −80° C. Before aptamer precipitation the nuclear extract was diluted to a final buffer containing 25 mM HEPES pH 7.6, 6.25% glycerol, 5 mM MgCl₂, 105 mM NaCl, 25 mM KCl, 250 ng/μL yeast RNA, 250 ng/μL sssDNA and 50 μM Dextran sulfate.

RNA preparation and immobilization on beads: The in vitro transcribed, polyadenylated NELF- or polyadenylated control (GFP)-aptamer was annealed to desthiobiotin oligodT-20 and immobilized on the beads following the steps as described previously for human HCT116 cells by replacing the bead wash buffer with NE bead wash buffer (25 mM Tris-HCl, pH 7.5, 5 mM MgCl₂, 100 mM NaCl, 25 mM KCl, 0.05% NP40).

Binding to lysate, washing & elution: The NELF- or control-aptamer bound beads were resuspended in the diluted nuclear extract and incubated at 4° C. with rotation for 2 hr. The beads were placed on a magnet and supernatant was removed. The beads were washed twice with the NE bead wash buffer and twice with non-NP40 containing NE bead wash buffer with changing tubes in between washes. Beads were resuspended in 50 μL of fresh NE elution buffer (4 mM Biotin, 20 mM Tris-HCl, pH 7.5, 50 mM NaCl) and incubated in a Thermomixer at 37° C. with shaking for 1 hr followed by collection of the eluate into a fresh tube.

Aptamer-based GFP-Rpb3 purification from S. pombe cells. S. pombe strains used in this study has the genotype ade6-m216, ura4-D18, leu1, gfp-rpb3 (Source: National Bio Resource Project, Japan). Lysate preparation from fission yeast was performed as described previously (Kajitani et al., “Ser7 of RNAPIICTD Facilitates Heterochromatin Formation by Linking ncRNA to RNAi,” Proc Natl Acad Sci USA 114: E11208-E11217 (2017), which is hereby incorporated by reference in its entirety) with several modifications. Cells were cultured in 100 ml of YES to 1×10⁷ cells/ml and harvested by centrifugation. Cell pellets were suspended in Lysis Buffer (1× PBS, 0.2% NP40, 1 mM PMSF, 1/50 volume of cOmpleteTM, Mini, EDTA-free Protease Inhibitor Cocktail [Roche], and 1/100 volume of PhosSTOPTM phosphatase inhibitor tablets [Roche]). Cell lysates were prepared by gentle vortexing with zirconia beads (Biospec products) in a FastPrep-24TM (Molecular Bio Products) at room temperature. Crude lysates were cleared by two-step centrifugation at 1100×g for 1 min at 4° C. and at 15,000×g for 10 min at 4° C. Lysates were diluted to a final buffer containing 1× PBS, 187 ng/μL yeast RNA, 187 ng/μL sssDNA, 200 units/ml SUPERase IN, 0.05% NP40, 5 mM MgCl₂. The aptamer RNA bead preparation, binding, washing and elution were done similar to the procedure used for human HCT116 cells. Western blot was performed with GFP antibody (Abcam ab290, 1:2000 dilution) and secondary Licor antibody (anti-rabbit IRDye 800CW).

Tandem affinity purification (TAP)-tag-based purification of Drosophila HSF (dHSF) from Drosophila S2 cells. Drosophila S2 cells stably transfected with plasmids encoding either N- or C terminal TAP-tagged dHSF in pMK33-NTAP or pMK33-CTAP vectors (Veraksa et al., “Analyzing protein complexes in Drosophila with tandem affinity purification-mass spectrometry,” Dev Dyn 232:827-834 (2005), which is hereby incorporated by reference in its entirety) were selected and cultured in M3+BPYE media supplemented with 10% FBS, Penn/Strep, and 300 μg/ml Hygromycin. Protein expression was induced with 0.5 mM CuSO₄ for 6 h at 24° C. Cells were collected by centrifugation at 3000 rpm, 10 min in 4° C., washed with ice-cold 1× PBS, and lysed with TAP Lysis Buffer (1×108 cells/ml: 10% Glycerol, 50 mM HEPESKOH pH 7.6, 150 mM KCl, 2 mM EDTA, 0.3% NP-40, freshly supplemented with 2 mM DTT, 1 mM PMSF, and 1× complete Protease inhibitor cocktail, 10 mM NaF). Cell lysate was incubated on ice for 15 min, passed through 21 gauge needle 10 times, and cleared by centrifugation at 20800×g 15 min at 4° C. Cleared lysate was passed through IgG sepharose column (150 μL resin) twice on ice and column was washed three times with TEV Cleavage Buffer (10 mM HEPES-KOH pH 7.6, 150 mM NaCl, 0.1% NP-40, 0.5 mM EDTA freshly supplemented with 1 mM DTT). Proteins were eluted by TEV protease cleavage (330 μL total volume with 1× TEV Cleavage Buffer with 4.25 μg TEV Protease) at RT for 1 h. The eluate was diluted 4 fold with Calmodulin Binding Buffer (10 mM HEPES-KOH pH 7.6, 150 mM NaCl, 0.1% NP-40, 1 mM Imidazole, 1 mM MgOAc, 2 mM CaCl₂) freshly supplemented with 10 mM 2-Mercaptoethanol), passed twice through 50 μL of pre-washed Ni-NTA superflow resin to remove His-tagged TEV protease, and flowthrough is supplemented with 4 mM CaCl₂) and passed twice over 50 μL of pre-washed Calmodulin resin on ice. Resin was then washed four times with Calmodulin Binding Buffer, and bound proteins were eluted twice with 150 μL Calmodulin Binding Buffer containing 25 mM EGTA. Proteins in the combined eluate were visualized by SDSPAGE/Silver Staining and individual bands were subjected to Mass Spectrometric analysis by Cornell Proteomics Core Facility.

Liquid chromatography-tandem mass spectrometry analysis of NELF-aptamer pull-downs from Drosophila S2 cells. Dried tryptic peptides from pulldowns using the NELF-aptamer from S2 cells were solubilized with 2% acetonitrile (ACN)/0.5% formic acid (FA) for nano LC-MS/MS analysis, which was carried out using an Orbitrap FusionTM TribridTM (Thermo-Fisher Scientific, San Jose, Calif.) mass spectrometer with a nanospray Flex Ion Source, and coupled with a Dionex UltiMate3000RSLCnano system (Thermo, Sunnyvale, Calif.). 5 μL of sample was injected onto a PepMap C-18 RP nano trap column (100 μm×20 mm, 5 μm, Dionex) with nanoViper Fittings via 20 μL/min trapping flow rate for on-line desalting, then separated on a PepMap C-18 RP nano column (75 μm×25 cm, 2 μm) at 35° C., and then eluted by a 60 min gradient of 5-35% ACN in 0.1% FA at 300 nL/min, followed by a 7 min ramp to 90% ACN/0.1% FA, and an 8 min hold at 90% ACN/0.1% FA. The column was re-equilibrated with 5% ACN/0.1% FA for 21 min before each subsequent injection. The Orbitrap Fusion was operated in positive ion mode with spray voltage set at 1.7 kV and source temperature 275° C. External calibration for Fourier transform (FT), ion trap and quadrupole mass analyzers was performed. In data dependent acquisition (DDA) analysis, the instrument was operated using the FT mass analyzer in MS scan to select precursor ions followed by 3 s “Top Speed” data-dependent CID ion trap MS/MS scans at 1.6 m/z quadrupole isolation for precursor peptides with multiply charged ions (+2-7) above a threshold ion count of 5,000 and using normalized collision energy of 30%. MS survey scans were at a resolving power of 120,000 (fwhm at m/z 200) for the mass range of m/z 375-1575. Dynamic exclusion parameters were set at repeat count 1 with a 40 s repeat duration and ±10 ppm exclusion mass width. The activation time was 10 ms for CID analysis. All data were acquired under Xcalibur 3.0 operation software (Thermo-Fisher Scientific).

### Example 1—Basic Strategy

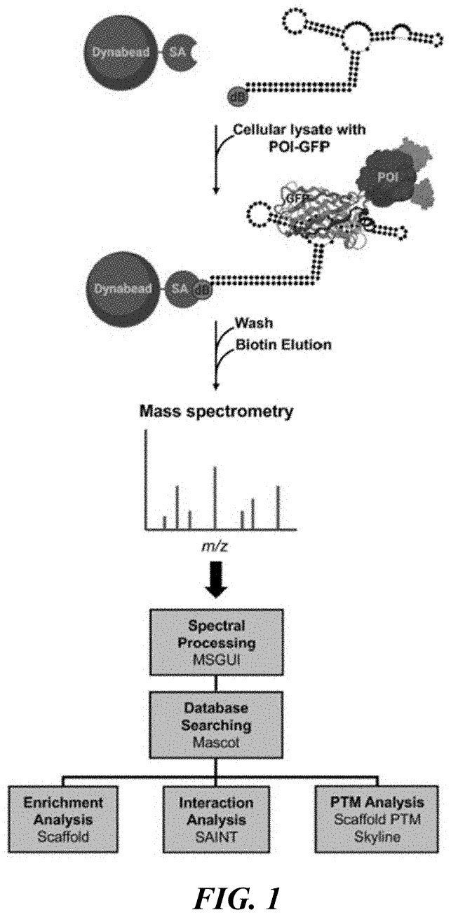

The GFP-aptamer (Shui et al., “RNA Aptamers that Functionally Interact with Green Fluorescent Protein and its Derivatives,” Nucleic Acids Res. 40:e39 (2012), which is hereby incorporated by reference in its entirety) with polyA-tail is immobilized on streptavidin magnetic Dynabeads via hybridization with desthiobiotin-oligodT and is incubated with a cellular lysate expressing GFP-tagged POI. After gently washing the beads, the aptamer-bound proteins can be specifically eluted with excess biotin, which has much higher affinity for streptavidin and readily competes off desthiobiotin. Eluted proteins are then analyzed by MS, leaving non-specifically bound proteins on beads (FIG. 1). The MS spectra are bioinformatically analyzed to determine enrichment of the POI relative to controls, protein interactions, and PTM site assignment.

### Example 2—GFP-Aptamer Provides a Clean Method of Enriching GFP-Tagged Proteins from Cellular Lysates

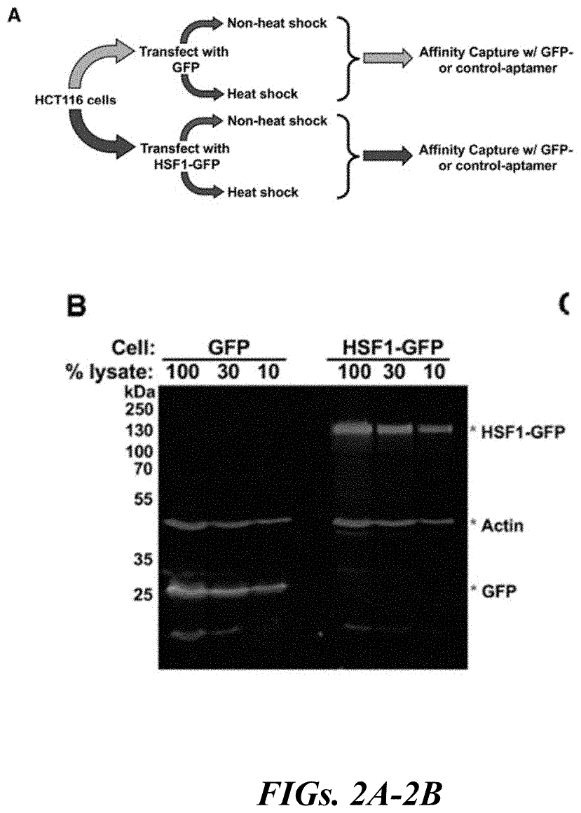

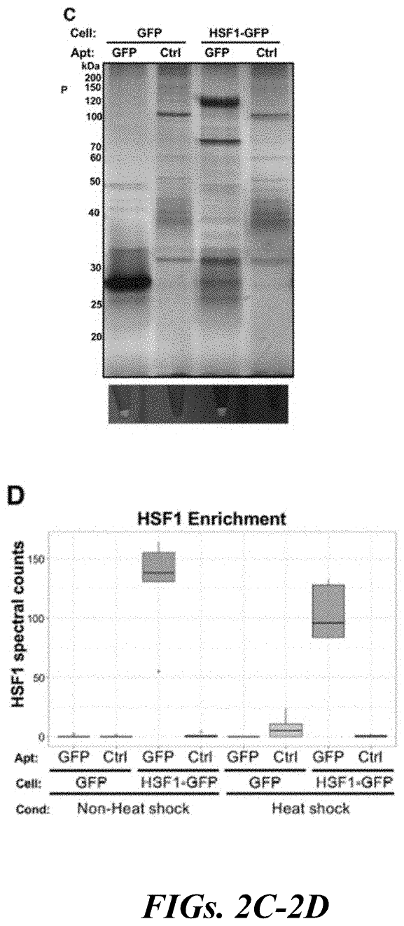

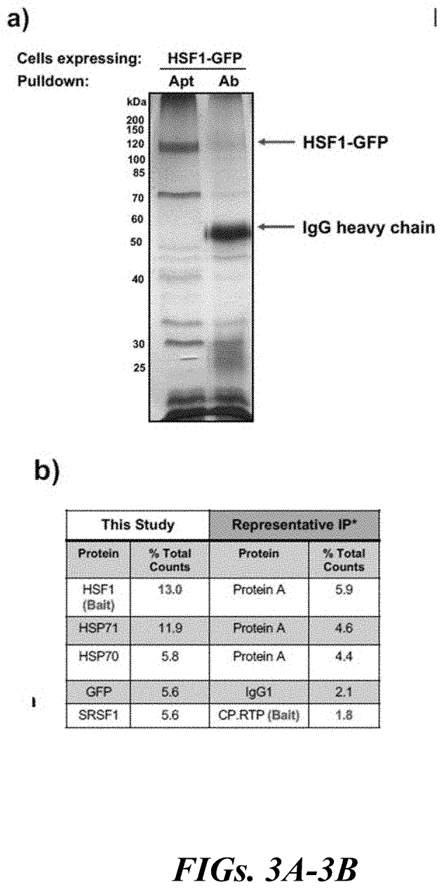

The AptA-MS method was tested by purifying HSF1 and its interacting partners from HCT116 cells transiently expressing HSF1 fused to GFP. HSF1 is a major regulator of stress-induced transcription that binds to hundreds of Heat Shock Elements (HSEs) genome-wide upon activation (Vihervaara et al., “Transcriptional Response to Stress is Pre-wired by Promoter and Enhancer Architecture,” Nat. Commun. 8:255 (2017), which is hereby incorporated by reference in its entirety). In non-heat shock (NETS) conditions, endogenous HSF1 is predominantly in an inactive monomeric state but converts to an active trimeric DNA-binding state upon HS (Vihervaara et al., “Molecular Mechanisms Driving Transcriptional Stress Responses,” Nat. Rev. Genet. 19:385-397 (2018), which is hereby incorporated by reference in its entirety). HSF1 is also regulated by chaperones and PTMs, and the identification of these binding partners and PTMs is critical to the understanding of HSF1's function (Gomez-Pastor et al., “Regulation of Heat Shock Transcription Factors and Their Roles in Physiology and Disease,” Nat. Rev. Mol. Cell Biol. 19:4-19 (2018); Vihervaara et al., “HSF1 at a Glance,” J. Cell Sci. 127:261-266 (2014), which are hereby incorporated by reference in their entirety). HCT116 cells were transfected with plasmid vectors expressing GFP or HSF1-GFP under the same promoter, and subjected to a 30 min heat shock at 42.0 (HS) or kept at 37.0 (NETS) (FIG. 2A). Total cellular lysates prepared from transfected cells were confirmed to be expressing similar levels of GFP and HSF1-GFP proteins by GFP western blot (FIG. 2B). Lysates were used for protein purifications with GFP- or control-aptamer immobilized on magnetic beads. The GFP-, but not the control-aptamer, was able to purify the targeted GFP and HSF1-GFP proteins that appeared as the most abundant bands observed by a silver-stained gel (FIG. 2C). In addition, fluorescent signal from the eluates indicated that GFP remained intact and functional (FIG. 2C, bottom panel). The additional bands detected on the silver-stained gel indicated the copurification of interacting proteins. In comparison, an affinity purification of HSF1-GFP utilizing the GFP antibody revealed IgG heavy chain as the most abundant protein in the eluate as detected by a silver-stained gel (FIG. 3A).

To identify the interactors of the target proteins, the eluates were processed for MS and peptide spectra were analyzed using a bioinformatics pipeline that quantifies protein enrichment and identifies interacting partners as well as PTMs (FIG. 1). HSF1 was the most abundant MS detected protein in both HSF1-GFP pull-downs (NETS and HS) (FIG. 3B). However, analyzing a conventional immunoprecipitation-MS data (DeBlasio et al., “Insights into the Polerovirus-plant Interactome Revealed by Coimmunoprecipitation and Mass Spectrometry,” Mol. Plant Microbe Interact. 28:467-481 (2015), which is hereby incorporated by reference in its entirety) with the same pipeline revealed ProteinA and IgG as the most abundant proteins identified (FIG. 3B). Nonspecifically binding proteins can pose a serious problem for any affinity-purification method. To reduce the presence of these background binders competitors like yeast RNA and sheared salmon sperm DNA have been used during the experiment. In addition, the control pull-downs from the cellular lysates provide information about the non-specific binders. Using the spectral counts from all five independent biological replicates, it was determined that HSF1 spectral counts from HSF1-GFP expressing cells were >300-fold enriched in GFP-aptamer pull-downs relative to the GFP-expressing cells, in which HSF1 was generally below the limit of detection (FIG. 2D).

### Example 3—AptA-MS Specifically Identifies HSF1 Interacting Partners and PTMs

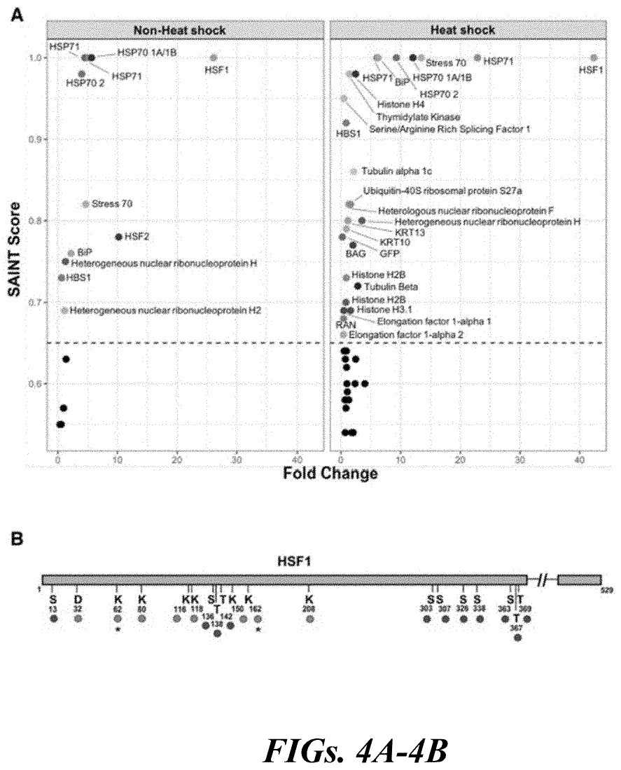

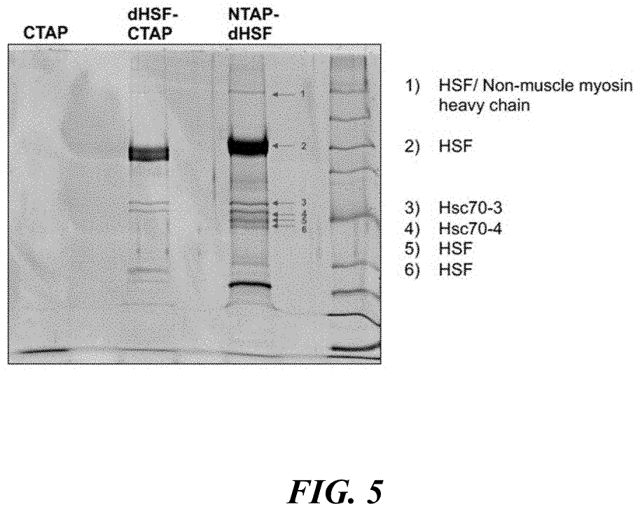

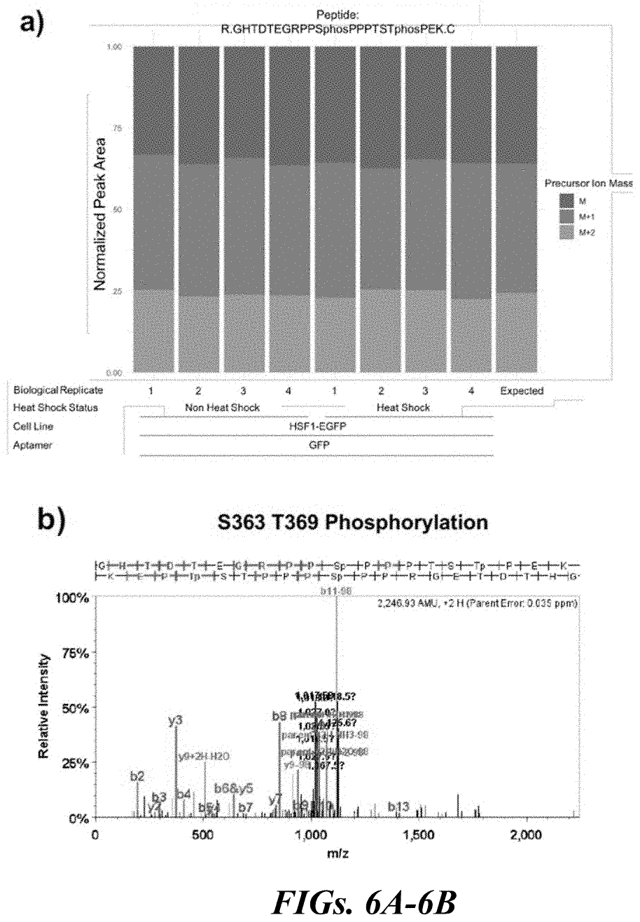

28 proteins were identified that are predicted to interact with HSF1 with high confidence based on SAINT score (Choi et al., “SAINT: Probabilistic Scoring of Affinity Purification-mass Spectrometry Data,” Nat. Methods 8:70-73 (2011), which is hereby incorporated by reference in its entirety), many of which are specific to the HS condition (FIG. 4A). Most of these interactors are molecular chaperones that are expected to associate with HSF1 (Takaki et al., In: Nakai, A. (ed). Heat Shock Factor. Springer, Tokyo, pp. 51-72 (2016); Raychaudhuri et al., “Interplay of Acetyltransferase EP300 and the Proteasome System in Regulating Heat Shock Transcription Factor 1,” Cell 156:975-985 (2014), which are hereby incorporated by reference in their entirety). dHSF has also been purified via the TAP-tag method from S2 cells, where enriched eluates were gel fractionated and individual protein bands were gel excised prior to MS. This independent assay verifies that some of these chaperone interactors are common in metazoans (FIG. 5). TAP-tagged dHSF was expressed near endogenous levels and still found to be associated with chaperones like dHsc70-3 (ortholog of human BiP) and dHsc70-4 (ortholog of human HSPA8) in NHS condition, indicating that HSF1-chaperone interactions detected in HCT116 cells by AptA-MS are not simply due to HSF1-GFP overexpression. HSF1 is bound by chaperones in normal conditions that prevent its transcriptional activity and is converted to an active state upon stress as the chaperones are released to bind to unfolded protein targets (Voellmy et al., “Chaperone Regulation of the Heat Shock Protein Response,” Adv. Exp. Med. Biol. 594:89-99 (2007), which is hereby incorporated by reference in its entirety). The data identifies primarily the HSP70 family of proteins to be interacting with HSF1 with high confidence relative to HSP90, supporting previous findings that report the lack of HSP90 interaction in human cells (Baler et al., “Heat Shock Gene Regulation by Nascent Polypeptides and Denatured Proteins: hsp70 as a Potential Autoregulatory Factor,” J. Cell Biol. 117:1151-1159 (1992), which is hereby incorporated by reference in its entirety), although this interaction has been reported in other organisms (Nadeau et al., “Hsp90 Chaperonins Possess ATPase Activity and Bind Heat Shock Transcription Factors and Peptidyl Prolyl Isomerases,” J. Biol. Chem. 268:1479-1487 (1993), which is hereby incorporated by reference in its entirety). In summary, the general overlap of AptA-MS identified candidates with the previously known HSF1 interacting partners validated the capability of the method to detect protein-protein interactions with high confidence. AptA-MS revealed new HSF1 interacting proteins not detected by TAP purification or other published studies (Table 1).

TABLE 1

| Present study | Published | Notes |
|---|---|---|
| HSF1 | PMID: 8455624, | |
| | PMID: 7935471, | |
| | PMID: 7623826, | |
| | PMID: 9222587, | |
| | PMID: 9727490, | |
| | PMID: 11583998, | |
| | PMID: 26754925, | |
| | PMID: 26727489 | |
| HSP70 | PMID: 7935376, | |
| | PMID: 9222587, | |
| | PMID: 9499401 | |
| HSP71/HSPA8 | PMID: 9499401 | |
| Stress 70/HSP70 | PMID: 7935376, | |
| | PMID: 9222587, | |
| | PMID: 9499401 | |
| BiP/HSP70 | PMID: 7935376, | |
| | PMID: 9222587, | |
| | PMID: 9499401 | |
| HBS1 | No | |
| Thymidylate Kinase | No | |
| Tubulin alpha 1c | No | Interacts with |
| | | Histone H4, |
| | | HSP90 |
| Histone H4 | No | |
| Elongation Factor 1-alpha 1 | PMID: 16554823, | |
| | PMID: 25233275 | |
| Tubulin beta | No | |
| Ubiquitin 40S ribosomal protein | No | |
| S27a | | |
| GTP-binding nuclear protein Ran | No | |
| Histone H2B | No | |
| Nuclear ribonucleoprotein H | No | |
| Serine/Arginine Rich Splicing | No | |
| Factor 1 | | |
| Heterologous nuclear | PMID: 16554823 | |
| ribonucleoprotein F | | |
| Heterogeneous nuclear | PMID: 16554823 | |
| ribonucleoprotein H | | |
| Heterogeneous nuclear | PMID: 16554823 | |
| ribonucleoprotein H2 | | |
| BAG | PMID: 23824909 | |
| KRT13 | No | |
| KRT10 | No | |
| Histone H3.1 | No | |
| RAN | No | |
| Elongation Factor 1-alpha 2 | No | |
| HSF2 | PMID: 25450459 | |

HSF1 has been previously shown to interact with translation elongation factor eEF1A1 based on an immunoprecipitation assay, which is implicated to have a broad regulatory function in the HS response (Vera et al., “The Translation Elongation Factor eEF1A1 Couples Transcription to Translation During Heat Shock Response,” Elife, 3:e03164 (2014)). Interestingly, AptA-MS identified not only eEF1A1, but also eEF1A2 (SAINT scores of 0.69 and 0.66, respectively) as HSF1-interactors. In addition, novel, high-confidence HSF1 interactions with translation elongation factor HBS1 and thymidylate kinase (a nucleotide biosynthesis enzyme) (SAINT scores of 0.92 and 0.98, respectively) have been identified during HS (FIG. 4A). HSF2 was also found to be associated with HSF1 during NHS as evident from the high SAINT score (0.78). However, this high confidence interaction was note detected upon HS, confirming previous reports where HSF1/HSF2 interaction was found to be reduced during HS (Korfanty et al., “Crosstalk Between HSF1 and HSF2 During the Heat Shock Response in Mouse Testes,” Int. J. Biochem. Cell Biol. 57:76-83 (2014), which is hereby incorporated by reference in its entirety). In addition, histones H4, H2B and H3.1 were found to be enriched in HS samples over NHS samples (FIG. 4A), likely reflecting the fact that upon heat stress HSF1 binding to DNA increases and is located near nucleosomes (Vihervaara et al., “Transcriptional Response to Stress is Pre-wired by Promoter and Enhancer Architecture,” Nat. Commun. 8:255 (2017), which is hereby incorporated by reference in its entirety). Detecting histones does not necessarily indicate direct interaction with HSF1, rather they could simply be nearby HSF1 and co-precipitated with HSF1-bound DNA. A stringent SAINT score cutoff of 0.65 was applied to call high confidence HSF1 interactors. However, there are many proteins with slightly lower SAINT scores, which may be bona fide interactors and could be confirmed in future experiments.

HSF1 is well-known to possess multiple PTMs in normal and HS conditions that include acetylation, phosphorylation and sumoylation of specific residues (Xu et al., “Post-translational Modification of Human Heat Shock Factors and Their Functions: a Recent Update by Proteomic Approach,” J. Proteome Res. 11:2625-2634 (2012), which is hereby incorporated by reference in its entirety). The pulldown strategy allowed identification of the acetylation and phosphorylation of HSF1 and other co-precipitants without any specific enrichment for these modifications (FIG. 4B, Table 2).

TABLE 2

| | | Distinct | | | | |
|---|---|---|---|---|---|---|
| | | Peptide | Mascot | Acetyl | Phospho | Phospho |
| Protein Name | Accession Number | Sequences | Score | (K) | (S) | (T) |
| Group of Heat shock factor protein 1 OS = Homo | UniProtKB: Q00613 +3 | 445 | 271.1 | 12 | 7 | 2 |
| sapiens GN = HSF1 PE = 1 SV = 1 (+3) | | | | | | |
| Group of Heat shock 70 kDa protein 1A/1B | UniProtKB: P08107 +6 | 571 | 820.3 | 10 | 1 | 2 |
| OS = Homo sapiens GN = HSPA1A PE = 1 SV = 5 | | | | | | |
| (+6) | | | | | | |
| GFP_AEQVI | GFP_AEQVI | 962 | 1 | 8 | | 1 |
| Desmoplakin OS = Homo sapiens GN = DSP | UniProtKB: P15924 | 16 | 41.36 | 7 | | 2 |
| PE = 1 SV = 3 | | | | | | |
| Golgin subfamily A member 4 OS = Homo | UniProtKB: Q13439 | 22 | 25.65 | 6 | 1 | |
| sapiens GN = GOLGA4 PE = 1 SV = 1 | | | | | | |
| Green fluorescent protein (GFP-Cter-HisTag) | UniProtKB: CON_Q9U6Y5 | 87 | 2 | 6 | | 1 |
| E3 ubiquitin-protein ligase RBBP6 OS = Homo | UniProtKB: Q7Z6E9 | 14 | 127.3 | 5 | | |
| sapiens GN = RBBP6 PE = 1 SV = 1 | | | | | | |
| Nucleolar and coiled-body phosphoprotein 1 | UniProtKB: Q14978 | 4 | 62.09 | 5 | | |
| OS = Homo sapiens GN = NOLC1 PE = 1 SV = 2 | | | | | | |
| Nucleolin OS = Homo sapiens GN = NCL PE = 1 | UniProtKB: P19338 | 7 | 72.76 | 5 | | |
| SV = 3 | | | | | | |
| Group of Serine/arginine-rich splicing factor 1 | UniProtKB: Q07955 +1 | 859 | 35.42 | 4 | 3 | |
| OS = Homo sapiens GN = SRSF1 PE = 1 SV = 2 | | | | | | |
| (+1) | | | | | | |
| Ryanodine receptor 3 OS = Homo sapiens | UniProtKB: Q15413 | 12 | 27.38 | 4 | | |
| GN = RYR3 PE = 1 SV = 2 | | | | | | |
| Small nuclear ribonucleoprotein Sm D2 | UniProtKB: P62316 | 4 | 61.92 | 4 | | |
| OS = Homo sapiens GN = SNRPD2 PE = 1 SV = 1 | | | | | | |
| Thyroid hormone receptor-associated protein 3 | UniProtKB: Q9Y2W1 | 464 | 1 | 4 | 8 | |
| OS = Homo sapiens GN = THRAP3 PE = 1 SV = 2 | | | | | | |
| Treacle protein OS = Homo sapiens GN = TCOF1 | UniProtKB: Q13428 | 6 | 39.66 | 4 | 1 | 1 |
| PE = 1 SV = 3 | | | | | | |
| Tyrosine-protein kinase ABL2 OS = Homo | UniProtKB: P42684 | 3 | 50.66 | 4 | | |
| sapiens GN = ABL2 PE = 1 SV = 1 | | | | | | |
| 40S ribosomal protein S25 OS = Homo sapiens | UniProtKB: P62851 | 4 | 30.89 | 3 | | |
| GN = RPS25 PE = 1 SV = 1 | | | | | | |
| Aquaporin-12B OS = Homo sapiens | UniProtKB: A6NM10 | 1 | 35.76 | 3 | | |
| GN = AQP12B PE = 2 SV = 1 | | | | | | |
| ATP-dependent RNA helicase DHX8 | UniProtKB: Q14562 | 4 | 52.28 | 3 | | |
| OS = Homo sapiens GN = DHX8 PE = 1 SV = 1 | | | | | | |
| Centromere-associated protein E OS = Homo | UniProtKB: Q02224 | 39 | 36.49 | 3 | | |
| sapiens GN = CENPE PE = 1 SV = 2 | | | | | | |
| Envoplakin OS = Homo sapiens GN = EVPL | UniProtKB: Q92817 | 7 | 30.23 | 3 | 1 | |
| PE = 1 SV = 3 | | | | | | |
| Group of ELAV-like protein 4 OS = Homo | UniProtKB: P26378 +1 | 134 | 64.91 | 3 | 1 | 1 |
| sapiens GN = ELAVL4 PE = 1 SV = 2 (+1) | | | | | | |
| Group of Ezrin OS = Homo sapiens GN = EZR | UniProtKB: P15311 +2 | 26 | 292.2 | 3 | | |
| PE = 1 SV = 4 (+2) | | | | | | |
| Group of Heterogeneous nuclear | UniProtKB: O60812 +1 | 12 | 425.7 | 3 | | |
| ribonucleoprotein C-like 1 OS = Homo sapiens | | | | | | |
| GN = HNRNPCL1 PE = 1 SV = 1 (+1) | | | | | | |
| Group of Histone H1.5 OS = Homo sapiens | UniProtKB: P16401 +2 | 18 | 137.9 | 3 | 1 | 2 |
| GN = HIST1H1B PE = 1 SV = 3 (+2) | | | | | | |
| Group of Histone H2B type 1-A OS = Homo | UniProtKB: Q96A08 +1 | 7 | 93.01 | 3 | 1 | 1 |
| sapiens GN = HIST1H2BA PE = 1 SV = 3 (+1) | | | | | | |
| Group of K22E_HUMAN (+21) | K22E_HUMAN +21 | 25 | 744 | 3 | 3 | 3 |
| Group of Squamous cell carcinoma antigen | UniProtKB: Q15020 +1 | 11 | 174.1 | 3 | 1 | |
| recognized by T-cells 3 OS = Homo sapiens | | | | | | |
| GN = SART3 PE = 1 SV = 1 (+1) | | | | | | |
| Histone H1.1 OS = Homo sapiens | UniProtKB: Q02539 | 6 | 42.99 | 3 | | |
| GN = HIST1H1A PE = 1 SV = 3 | | | | | | |
| Histone H4 OS = Homo sapiens GN = HIST1H4A | UniProtKB: P62805 | 8 | 245.9 | 3 | | |
| PE = 1 SV = 2 | | | | | | |
| Kinetochore-associated protein 1 OS = Homo | UniProtKB: P50748 | 5 | 22.48 | 3 | | |
| sapiens GN = KNTC1 PE = 1 SV = 1 | | | | | | |
| Lebercilin OS = Homo sapiens GN = LCA5 PE = 1 | UniProtKB: Q86VQ0 | 6 | 39.09 | 3 | 1 | |
| SV = 2 | | | | | | |
| Midasin OS = Homo sapiens GN = MDN1 PE = 1 | UniProtKB: Q9NU22 | 9 | 31.37 | 3 | | |
| SV = 2 | | | | | | |
| Ribonuclease H2 subunit B OS = Homo sapiens | UniProtKB: Q5TBB1 | 4 | 18.55 | 3 | | |
| GN = RNASEH2B PE = 1 SV = 1 | | | | | | |
| 60S ribosomal protein L22-like 1 OS = Homo | UniProtKB: Q6P5R6 | 2 | 151.3 | 2 | | |
| sapiens GN = RPL22L1 PE = 1 SV = 2 | | | | | | |
| ATP synthase-coupling factor 6, mitochondrial | UniProtKB: P18859 | 1 | 18.19 | 2 | | |
| OS = Homo sapiens GN = ATP5J PE = 1 SV = 1 | | | | | | |
| Bromodomain testis-specific protein OS = Homo | UniProtKB: Q58F21 | 7 | 30.76 | 2 | | |
| sapiens GN = BRDT PE = 1 SV = 4 | | | | | | |
| CAS1_BOVIN | CAS1_BOVIN | 1 | 33.57 | 2 | 1 | |
| CAS2_BOVIN | CAS2_BOVIN | 4 | 49.76 | 2 | 1 | |
| CASB_BOVIN | CASB_BOVIN | 3 | 46.98 | 2 | 1 | |
| Double-stranded RNA-binding protein Staufen | UniProtKB: O95793 | 3 | 42.56 | 2 | | |
| homolog 1 OS = Homo sapiens GN = STAU1 | | | | | | |
| PE = 1 SV = 2 | | | | | | |
| E3 SUMO-protein ligase RanBP2 OS = Homo | UniProtKB: P49792 | 3 | 35.6 | 2 | 3 | |
| sapiens GN = RANBP2 PE = 1 SV = 2 | | | | | | |
| Filaggrin OS = Homo sapiens GN = FLG PE = 1 | UniProtKB: P20930 | 10 | 41.76 | 2 | 4 | |
| SV = 3 | | | | | | |
| Group of ATP-dependent RNA helicase DDX50 | UniProtKB: Q9BQ39 +1 | 14 | 178.2 | 2 | 6 | 1 |
| OS = Homo sapiens GN = DDX50 PE = 1 SV = 1 | | | | | | |
| (+1) | | | | | | |
| Group of Nucleolysin TIA-1 isoform p40 | UniProtKB: P31483 +1 | 17 | 919.2 | 2 | | |
| OS = Homo sapiens GN = TIA1 PE = 1 SV = 3 (+1) | | | | | | |
| Histone H3.2 OS = Homo sapiens | UniProtKB: Q71DI3 | 4 | 68.36 | 2 | | 1 |
| GN = HIST2H3A PE = 1 SV = 3 | | | | | | |
| Histone H3.3C OS = Homo sapiens GN = H3F3C | UniProtKB: Q6NXT2 | 4 | 36.73 | 2 | | 1 |
| PE = 1 SV = 3 | | | | | | |
| Host cell factor 1 OS = Homo sapiens | UniProtKB: P51610 | 5 | 29.27 | 2 | | |
| GN = HCFC1 PE = 1 SV = 2 | | | | | | |
| Interleukin enhancer-binding factor 3 OS = Homo | UniProtKB: Q12906 | 10 | 200.3 | 2 | | |
| sapiens GN = ILF3 PE = 1 SV = 3 | | | | | | |
| Putative 60S ribosomal protein L13a-like | UniProtKB: Q6NVV1 | 5 | 65.36 | 2 | | |
| MGC87657 OS = Homo sapiens PE = 5 SV = 1 | | | | | | |
| Putative ATP-dependent RNA helicase DHX30 | UniProtKB: Q7L2E3 | 2 | 40.41 | 2 | 3 | 1 |
| OS = Homo sapiens GN = DHX30 PE = 1 SV = 1 | | | | | | |
| Serine/arginine repetitive matrix protein 1 | UniProtKB: Q8IYB3 | 5 | 45.21 | 2 | | |
| OS = Homo sapiens GN = SRRM1 PE = 1 SV = 2 | | | | | | |
| Serine/threonine-protein kinase PLK1 | UniProtKB: P53350 | 2 | 35.38 | 2 | | |
| OS = Homo sapiens GN = PLK1 PE = 1 SV = 1 | | | | | | |
| Small nuclear ribonucleoprotein Sm D3 | UniProtKB: P62318 | 3 | 64.86 | 2 | | |
| OS = Homo sapiens GN = SNRPD3 PE = 1 SV = 1 | | | | | | |
| Suprabasin OS = Homo sapiens GN = SBSN PE = 2 | UniProtKB: Q6UWP8 | 5 | 33.14 | 2 | | |
| SV = 1 | | | | | | |
| Transcriptional regulator ATRX OS = Homo | UniProtKB: P46100 | 29 | 31.59 | 2 | | |
| sapiens GN = ATRX PE = 1 SV = 5 | | | | | | |
| TRYP_PIG | TRYP_PIG | 447 | 1 | 2 | | |
| Ubiquitin-60S ribosomal protein L40 OS = Homo | UniProtKB: P62987 | 5 | 86.25 | 2 | | |
| sapiens GN = UBA52 PE = 1 SV = 2 | | | | | | |
| Uncharacterized protein C1orf14 OS = Homo | UniProtKB: Q9BZQ2 | 8 | 26.44 | 2 | | |
| sapiens GN = C1orf14 PE = 1 SV = 2 | | | | | | |
| Uncharacterized protein C17orf85 OS = Homo | UniProtKB: Q53F19 | 6 | 96.29 | 2 | | |
| sapiens GN = C17orf85 PE = 1 SV = 2 | | | | | | |
| Zinc finger protein 668 OS = Homo sapiens | UniProtKB: Q96K58 | 2 | 27.83 | 2 | | |
| GN = ZNF668 PE = 2 SV = 3 | | | | | | |
| 5′ exonuclease Apollo OS = Homo sapiens | UniProtKB: Q9H816 | 1 | 18.11 | 1 | | |
| GN = DCLRE1B PE = 1 SV = 1 | | | | | | |
| 40S ribosomal protein SA OS = Homo sapiens | UniProtKB: P08865 | 6 | 132.9 | 1 | | |
| GN = RPSA PE = 1 SV = 4 | | | | | | |
| 60S ribosomal protein L7 OS = Homo sapiens | UniProtKB: P18124 | 1 | 70.62 | 1 | | 1 |
| GN = RPL7 PE = 1 SV = 1 | | | | | | |
| 60S ribosomal protein L7a OS = Homo sapiens | UniProtKB: P62424 | 11 | 174.9 | 1 | | |
| GN = RPL7A PE = 1 SV = 2 | | | | | | |
| Activating signal cointegrator 1 complex subunit | UniProtKB: Q8N3C0 | 8 | 34.17 | 1 | | |
| 3 OS = Homo sapiens GN = ASCC3 PE = 1 SV = 3 | | | | | | |
| Amyotrophic lateral sclerosis 2 chromosomal | UniProtKB: Q8N187 | 1 | 17.17 | 1 | | |
| region candidate gene 8 protein OS = Homo | | | | | | |
| sapiens GN = ALS2CR8 PE = 2 SV = 2 | | | | | | |
| Ankyrin repeat domain-containing protein 18B | UniProtKB: A2A2Z9 | 6 | 20.41 | 1 | | |
| OS = Homo sapiens GN = ANKRD18B PE = 2 | | | | | | |
| SV = 1 | | | | | | |
| Antigen KI-67 OS = Homo sapiens GN = MKI67 | UniProtKB: P46013 | 10 | 64.15 | 1 | 1 | |
| PE = 1 SV = 2 | | | | | | |
| Apoptotic chromatin condensation inducer in the | UniProtKB: Q9UKV3 | 12 | 191.8 | 1 | 2 | |
| nucleus OS = Homo sapiens GN = ACIN1 PE = 1 | | | | | | |
| SV = 2 | | | | | | |
| ATP-dependent RNA helicase A OS = Homo | UniProtKB: Q08211 | 3 | 63.99 | 1 | 1 | 1 |
| sapiens GN = DHX9 PE = 1 SV = 4 | | | | | | |
| BAG family molecular chaperone regulator 2 | UniProtKB: O95816 | 3 | 51.94 | 1 | 1 | |
| OS = Homo sapiens GN = BAG2 PE = 1 SV = 1 | | | | | | |
| Bcl-2-associated transcription factor 1 | UniProtKB: Q9NYF8 | 201 | 1 | 1 | 12 | |
| OS = Homo sapiens GN = BCLAF1 PE = 1 SV = 2 | | | | | | |
| Beta-lactoglobulin - Bos taurus (Bovine) | UniProtKB: CON_P02754 | 1 | 50.65 | 1 | | |
| BUD13 homolog OS = Homo sapiens | UniProtKB: Q9BRD0 | 3 | 46.76 | 1 | 1 | |
| GN = BUD13 PE = 1 SV = 1 | | | | | | |
| CCAAT/enhancer-binding protein zeta | UniProtKB: Q03701 | 5 | 51.11 | 1 | | |
| OS = Homo sapiens GN = CEBPZ PE = 1 SV = 3 | | | | | | |
| Cell division cycle 5-like protein OS = Homo | UniProtKB: Q99459 | 5 | 42.49 | 1 | | |
| sapiens GN = CDC5L PE = 1 SV = 2 | | | | | | |
| Chloride transport protein 6 OS = Homo sapiens | UniProtKB: P51797 | 2 | 19.04 | 1 | | |
| GN = CLCN6 PE = 1 SV = 2 | | | | | | |
| Chondroitin sulfate proteoglycan 4 OS = Homo | UniProtKB: Q6UVK1 | 2 | 28.11 | 1 | | |
| sapiens GN = CSPG4 PE = 1 SV = 2 | | | | | | |
| Cohesin subunit SA-2 OS = Homo sapiens | UniProtKB: Q8N3U4 | 3 | 42.22 | 1 | 1 | 1 |
| GN = STAG2 PE = 1 SV = 3 | | | | | | |
| Coiled-coil domain-containing protein C6orf97 | UniProtKB: Q8IYT3 | 9 | 35.82 | 1 | | |
| OS = Homo sapiens GN = C6orf97 PE = 2 SV = 3 | | | | | | |
| CTD small phosphatase-like protein 2 | UniProtKB: Q05D32 | 1 | 32.07 | 1 | | |
| OS = Homo sapiens GN = CTDSPL2 PE = 1 SV = 2 | | | | | | |
| E3 ubiquitin-protein ligase UBR5 OS = Homo | UniProtKB: O95071 | 6 | 33.52 | 1 | | |
| sapiens GN = UBR5 PE = 1 SV = 2 | | | | | | |
| ETS translocation variant 3 OS = Homo sapiens | UniProtKB: P41162 | 2 | 19.55 | 1 | | |
| GN = ETV3 PE = 1 SV = 2 | | | | | | |
| Eukaryotic translation initiation factor 5B | UniProtKB: O60841 | 10 | 48.26 | 1 | 2 | 1 |
| OS = Homo sapiens GN = EIF5B PE = 1 SV = 4 | | | | | | |
| Exosome component 10 OS = Homo sapiens | UniProtKB: Q01780 | 8 | 38 | 1 | | |
| GN = EXOSC10 PE = 1 SV = 2 | | | | | | |
| Glutamate [NMDA] receptor subunit epsilon-2 | UniProtKB: Q13224 | 3 | 21.55 | 1 | | |
| OS = Homo sapiens GN = GRIN2B PE = 1 SV = 3 | | | | | | |
| Group of 14-3-3 protein theta OS = Homo | UniProtKB: P27348 +3 | 9 | 122.6 | 1 | | |
| sapiens GN = YWHAQ PE = 1 SV = 1 (+3) | | | | | | |
| Group of 40S ribosomal protein S4, Y isoform 1 | UniProtKB: P22090 +1 | 9 | 130.6 | 1 | | |
| OS = Homo sapiens GN = RPS4Y1 PE = 2 SV = 2 | | | | | | |
| (+1) | | | | | | |
| Group of 40S ribosomal protein S17 OS = Homo | UniProtKB: P08708 +1 | 7 | 271.2 | 1 | | |
| sapiens GN = RPS17 PE = 1 SV = 2 (+1) | | | | | | |
| Group of 60S ribosomal protein L3 OS = Homo | UniProtKB: P39023 +1 | 10 | 93.74 | 1 | | |
| sapiens GN = RPL3 PE = 1 SV = 2 (+1) | | | | | | |
| Group of ADP/ATP translocase 1 OS = Homo | UniProtKB: P12235 +2 | 8 | 53.43 | 1 | | |
| sapiens GN = SLC25A4 PE = 1 SV = 4 (+2) | | | | | | |
| Group of Alpha-actinin-4 OS = Homo sapiens | UniProtKB: O43707 +2 | 25 | 100.2 | 1 | | |
| GN = ACTN4 PE = 1 SV = 2 (+2) | | | | | | |
| Group of Elongation factor 1-alpha 1 OS = Homo | UniProtKB: P68104 +1 | 18 | 498.5 | 1 | | |
| sapiens GN = EEF1A1 PE = 1 SV = 1 (+1) | | | | | | |
| Group of Heterogeneous nuclear | UniProtKB: O43390 +1 | 8 | 185.4 | 1 | | 1 |
| ribonucleoprotein R OS = Homo sapiens | | | | | | |
| GN = HNRNPR PE = 1 SV = 1 (+1) | | | | | | |
| Group of Histone H2A type 1-B/E OS = Homo | UniProtKB: P04908 +2 | 3 | 92.3 | 1 | | |
| sapiens GN = HIST1H2AB PE = 1 SV = 2 (+2) | | | | | | |
| Group of Histone H2B type 1-K OS = Homo | UniProtKB: O60814 +6 | 7 | 115.3 | 1 | | 1 |
| sapiens GN = HIST1H2BK PE = 1 SV = 3 (+6) | | | | | | |
| Group of Histone-binding protein RBBP4 | UniProtKB: Q09028 +1 | 4 | 72.2 | 1 | | |
| OS = Homo sapiens GN = RBBP4 PE = 1 SV = 3 | | | | | | |
| (+1) | | | | | | |
| Group of K1C10_HUMAN (+18) | K1C10_HUMAN +18 | 22 | 798.3 | 1 | 1 | 1 |
| Group of K1H2_HUMAN (+6) | K1H2_HUMAN +6 | 10 | 91.39 | 1 | | |
| Group of Polyadenylate-binding protein 4-like | UniProtKB: POCB38 +6 | 23 | 502.4 | 1 | 1 | 1 |
| OS = Homo sapiens GN = PABPC4L PE = 2 SV = 1 | | | | | | |
| (+6) | | | | | | |
| Group of Probable ATP-dependent RNA | UniProtKB: P17844 +1 | 9 | 117.7 | 1 | 1 | |
| helicase DDX5 OS = Homo sapiens GN = DDX5 | | | | | | |
| PE = 1 SV = 1 (+1) | | | | | | |
| Group of RNA-binding motif protein, X-linked- | UniProtKB: O75526 +1 | 7 | 51.72 | 1 | 1 | |
| like-2 OS = Homo sapiens GN = RBMXL2 PE = 1 | | | | | | |
| SV = 3 (+1) | | | | | | |
| Group of Splicing factor, proline- and | UniProtKB: P23246 +1 | 6 | 73.78 | 1 | | |
| glutamine-rich OS = Homo sapiens GN = SFPQ | | | | | | |
| PE = 1 SV = 2 (+1) | | | | | | |
| Group of Tax_Id = 9606 Gene_Symbol = KRT6B | UniProtKB: CON_P04259 +9 | 20 | 298.9 | 1 | | |
| Keratin, type II cytoskeletal 6B (+9) | | | | | | |
| Group of Tax_Id = 9606 Gene_Symbol = KRT14 | UniProtKB: CON_P02533 +5 | 15 | 220 | 1 | | 1 |
| Keratin, type I cytoskeletal 14 (+5) | | | | | | |
| Group of Tax_Id = 9606 Gene_Symbol = PRSS1 | UniProtKB: CON_P07477 +2 | 4 | 279.6 | 1 | | 1 |
| Trypsin-1 precursor (+2) | | | | | | |
| Group of Thymosin beta-4-like protein 3 | UniProtKB: A8MW06 +2 | 6 | 152.1 | 1 | | |
| OS = Homo sapiens GN = TMSL3 PE = 2 SV = 1 | | | | | | |
| (+2) | | | | | | |
| Group of Zinc finger CCCH domain-containing | UniProtKB: O75152 +12 | 160 | 893.1 | 1 | 1 | 1 |
| protein 11A OS = Homo sapiens GN = ZC3H11A | | | | | | |
| PE = 1 SV = 3 (+12) | | | | | | |
| Homeobox protein Hox-B9 OS = Homo sapiens | UniProtKB: P17482 | 3 | 35.31 | 1 | | |
| GN = HOXB9 PE = 1 SV = 2 | | | | | | |
| Malate dehydrogenase, mitochondrial | UniProtKB: P40926 | 5 | 83.78 | 1 | | |
| OS = Homo sapiens GN = MDH2 PE = 1 SV = 3 | | | | | | |
| Mitochondrial import receptor subunit TOM70 | UniProtKB: O94826 | 2 | 22.92 | 1 | | |
| OS = Homo sapiens GN = TOMM70A PE = 1 | | | | | | |
| SV = 1 | | | | | | |
| Multimerin-1 OS = Homo sapiens GN = MMRN1 | UniProtKB: Q13201 | 3 | 22.87 | 1 | | |
| PE = 1 SV = 3 | | | | | | |
| Myosin-IIIa OS = Homo sapiens GN = MYO3A | UniProtKB: Q8NEV4 | 5 | 22.38 | 1 | | |
| PE = 1 SV = 2 | | | | | | |
| N-acetyl serotonin O-methyltransferase-like | UniProtKB: O95671 | 2 | 28.29 | 1 | | |
| protein OS = Homo sapiens GN = ASMTL PE = 1 | | | | | | |
| SV = 3 | | | | | | |
| Neuronal acetylcholine receptor subunit alpha-9 | UniProtKB: Q9UGM1 | 3 | 20.18 | 1 | | |
| OS = Homo sapiens GN = CHRNA9 PE = 1 SV = 2 | | | | | | |
| Nuclear fragile X mental retardation-interacting | UniProtKB: Q7Z417 | 6 | 42.1 | 1 | | |
| protein 2 OS = Homo sapiens GN = NUFIP2 PE = 1 | | | | | | |
| SV = 1 | | | | | | |
| Nucleoside-triphosphatase C1orf57 OS = Homo | UniProtKB: Q9BSD7 | 2 | 43.54 | 1 | | |
| sapiens GN = C1orf57 PE = 1 SV = 1 | | | | | | |
| Pre-mRNA-processing factor 40 homolog B | UniProtKB: Q6NWY9 | 4 | 14.68 | 1 | | |
| OS = Homo sapiens GN = PRPF40B PE = 1 SV = 1 | | | | | | |
| Prelamin-A/C OS = Homo sapiens GN = LMNA | UniProtKB: P02545 | 10 | 61.01 | 1 | | |
| PE = 1 SV = 1 | | | | | | |
| Protein disulfide-isomerase OS = Homo sapiens | UniProtKB: P07237 | 7 | 204.9 | 1 | | |
| GN = P4HB PE = 1 SV = 3 | | | | | | |
| Protein flightless-1 homolog OS = Homo sapiens | UniProtKB: Q13045 | 5 | 22.61 | 1 | | |
| GN = FLII PE = 1 SV = 2 | | | | | | |
| Puratrophin-1 OS = Homo sapiens | UniProtKB: Q58EX7 | 2 | 26.31 | 1 | | |
| GN = PLEKHG4 PE = 1 SV = 1 | | | | | | |
| Putative RNA-binding protein 15 OS = Homo | UniProtKB: Q96T37 | 4 | 61.21 | 1 | | |
| sapiens GN = RBM15 PE = 1 SV = 2 | | | | | | |
| RanBP2-like and GRIP domain-containing | UniProtKB: A6NKT7 | 19 | 33.97 | 1 | | |
| protein 3 OS = Homo sapiens GN = RGPD3 PE = 2 | | | | | | |
| SV = 1 | | | | | | |
| Regulation of nuclear pre-mRNA domain- | UniProtKB: Q5VT52 | 4 | 35.54 | 1 | | |
| containing protein 2 OS = Homo sapiens | | | | | | |
| GN = RPRD2 PE = 1 SV = 1 | | | | | | |
| Rho GTPase-activating protein 11A OS = Homo | UniProtKB: Q6P4F7 | 10 | 17.08 | 1 | | |
| sapiens GN = ARHGAP11A PE = 1 SV = 2 | | | | | | |
| Rho GTPase-activating protein 28 OS = Homo | UniProtKB: Q9P2N2 | 3 | 39.66 | 1 | | |
| sapiens GN = ARHGAP28 PE = 2 SV = 3 | | | | | | |
| Ribosome biogenesis protein BMS1 homolog | UniProtKB: Q14692 | 7 | 61.5 | 1 | | |
| OS = Homo sapiens GN = BMS1 PE = 1 SV = 1 | | | | | | |
| Ribosome biogenesis protein BRX1 homolog | UniProtKB: Q8TDN6 | 7 | 23.91 | 1 | | |
| OS = Homo sapiens GN = BRIX1 PE = 1 SV = 2 | | | | | | |
| Ribosome production factor 2 homolog | UniProtKB: Q9H7B2 | 6 | 78.38 | 1 | | |
| OS = Homo sapiens GN = RPF2 PE = 1 SV = 2 | | | | | | |
| Ribosome-binding protein 1 OS = Homo sapiens | UniProtKB: Q9P2E9 | 50 | 62.07 | 1 | 1 | 1 |
| GN = RRBP1 PE = 1 SV = 4 | | | | | | |
| RNA-binding protein 28 OS = Homo sapiens | UniProtKB: Q9NW13 | 11 | 141.9 | 1 | | 1 |
| GN = RBM28 PE = 1 SV = 3 | | | | | | |
| RNA-binding protein FUS OS = Homo sapiens | UniProtKB: P35637 | 3 | 13.52 | 1 | | |
| GN = FUS PE = 1 SV = 1 | | | | | | |
| RNA-binding protein Raly OS = Homo sapiens | UniProtKB: Q9UKM9 | 4 | 64.67 | 1 | 1 | 1 |
| GN = RALY PE = 1 SV = 1 | | | | | | |
| Scaffold attachment factor B1 OS = Homo | UniProtKB: Q15424 | 6 | 47.2 | 1 | | |
| sapiens GN = SAFB PE = 1 SV = 4 | | | | | | |
| Serine/arginine-rich splicing factor 3 OS = Homo | UniProtKB: P84103 | 4 | 174.7 | 1 | | |
| sapiens GN = SRSF3 PE = 1 SV = 1 | | | | | | |
| Serine/threonine-protein kinase Chk1 OS = Homo | UniProtKB: O14757 | 7 | 26.68 | 1 | | |
| sapiens GN = CHEK1 PE = 1 SV = 1 | | | | | | |
| Serine/threonine-protein phosphatase 6 | UniProtKB: O15084 | 1 | 18.41 | 1 | | |
| regulatory ankyrin repeat subunit A OS = Homo | | | | | | |
| sapiens GN = ANKRD28 PE = 1 SV = 4 | | | | | | |
| Sickle tail protein homolog OS = Homo sapiens | UniProtKB: Q5T5P2 | 7 | 34.21 | 1 | | |
| GN = SKT PE = 1 SV = 2 | | | | | | |
| Small nuclear ribonucleoprotein G-like protein | UniProtKB: A8MWD9 | 1 | 18.68 | 1 | | 1 |
| OS = Homo sapiens PE = 3 SV = 2 | | | | | | |
| SNW domain-containing protein 1 OS = Homo | UniProtKB: Q13573 | 4 | 43.77 | 1 | | |
| sapiens GN = SNW1 PE = 1 SV = 1 | | | | | | |
| Splicing factor 3B subunit 1 OS = Homo sapiens | UniProtKB: O75533 | 1 | 81.84 | 1 | 1 | |
| GN = SF3B1 PE = 1 SV = 3 | | | | | | |
| Splicing factor U2AF 65 kDa subunit | UniProtKB: P26368 | 10 | 543.4 | 1 | 1 | |
| OS = Homo sapiens GN = U2AF2 PE = 1 SV = 4 | | | | | | |
| Stress-70 protein, mitochondrial OS = Homo | UniProtKB: P38646 | 308 | 1 | 1 | | |
| sapiens GN = HSPA9 PE = 1 SV = 2 | | | | | | |
| Synaptonemal complex central element protein | UniProtKB: Q6PIF2 | 3 | 40.51 | 1 | | |
| 2 OS = Homo sapiens GN = SYCE2 PE = 2 SV = 2 | | | | | | |
| THO complex subunit 4 OS = Homo sapiens | UniProtKB: Q86V81 | 5 | 130.1 | 1 | | |
| GN = THOC4 PE = 1 SV = 3 | | | | | | |
| Transcription factor HES-1 OS = Homo sapiens | UniProtKB: Q14469 | 1 | 35.4 | 1 | | |
| GN = HES1 PE = 1 SV = 1 | | | | | | |
| Triadin OS = Homo sapiens GN = TRDN PE = 1 | UniProtKB: Q13061 | 5 | 26.62 | 1 | | |
| SV = 3 | | | | | | |
| Trifunctional enzyme subunit beta, | UniProtKB: P55084 | 4 | 35.81 | 1 | | |
| mitochondrial OS = Homo sapiens GN = HADHB | | | | | | |
| PE = 1 SV = 3 | | | | | | |
| Tubby-related protein 4 OS = Homo sapiens | UniProtKB: Q9NRJ4 | 2 | 15.54 | 1 | | |
| GN = TULP4 PE = 1 SV = 2 | | | | | | |
| Tumor necrosis factor receptor superfamily | UniProtKB: O00300 | 2 | 36.43 | 1 | | |
| member 11B OS = Homo sapiens | | | | | | |
| GN = TNFRSF11B PE = 1 SV = 2 | | | | | | |
| U2 small nuclear ribonucleoprotein A′ | UniProtKB: P09661 | 5 | 29.45 | 1 | | |
| OS = Homo sapiens GN = SNRPA1 PE = 1 SV = 2 | | | | | | |
| Ubiquitin carboxyl-terminal hydrolase 7 | UniProtKB: Q93009 | 3 | 16.94 | 1 | | |
| OS = Homo sapiens GN = USP7 PE = 1 SV = 2 | | | | | | |
| UBX domain-containing protein 4 OS = Homo | UniProtKB: Q92575 | 2 | 42.99 | 1 | | |
| sapiens GN = UBXN4 PE = 1 SV = 2 | | | | | | |
| Uncharacterized protein KIAA0195 OS = Homo | UniProtKB: Q12767 | 1 | 28.25 | 1 | | |
| sapiens GN = KIAA0195 PE = 1 SV = 1 | | | | | | |
| Uncharacterized protein KIAA1370 OS = Homo | UniProtKB: Q32MH5 | 5 | 16.77 | 1 | | |
| sapiens GN = KIAA1370 PE = 2 SV = 2 | | | | | | |
| WD repeat and FYVE domain-containing | UniProtKB: Q96P53 | 2 | 34.2 | 1 | | |
| protein 2 OS = Homo sapiens GN = WDFY2 PE = 2 | | | | | | |
| SV = 2 | | | | | | |
| X-ray repair cross-complementing protein 6 | UniProtKB: P12956 | 9 | 70.77 | 1 | | |
| OS = Homo sapiens GN = XRCC6 PE = 1 SV = 2 | | | | | | |
| Zinc finger C3H1 domain-containing protein | UniProtKB: O60293 | 6 | 24.08 | 1 | | |
| OS = Homo sapiens GN = ZFC3H1 PE = 1 SV = 3 | | | | | | |
| Zinc finger CCCH domain-containing protein 6 | UniProtKB: P61129 | 2 | 33.49 | 1 | | |
| OS = Homo sapiens GN = ZC3H6 PE = 2 SV = 1 | | | | | | |
| Zinc finger protein 618 OS = Homo sapiens | UniProtKB: Q5T7W0 | 1 | 37.02 | 1 | | |
| GN = ZNF618 PE = 1 SV = 1 | | | | | | |
| 3′-5′ exoribonuclease 1 OS = Homo sapiens | UniProtKB: Q8IV48 | 2 | 39.59 | | | |
| GN = ERI1 PE = 1 SV = 3 | | | | | | |
| 3,2-trans-enoyl-CoA isomerase, mitochondrial | UniProtKB: P42126 | 3 | 111.8 | | | |
| OS = Homo sapiens GN = DCI PE = 1 SV = 1 | | | | | | |
| 4F2 cell-surface antigen heavy chain OS = Homo | UniProtKB: P08195 | 7 | 137.8 | | | |
| sapiens GN = SLC3A2 PE = 1 SV = 3 | | | | | | |
| 6-phosphofructokinase type C OS = Homo | UniProtKB: Q01813 | 10 | 117.9 | | | |
| sapiens GN = PFKP PE = 1 SV = 2 | | | | | | |
| 6-phosphogluconate dehydrogenase, | UniProtKB: P52209 | 7 | 55.75 | | | |
| decarboxylating OS = Homo sapiens GN = PGD | | | | | | |
| PE = 1 SV = 3 | | | | | | |
| 10 kDa heat shock protein, mitochondrial | UniProtKB: P61604 | 6 | 86.86 | | | |
| OS = Homo sapiens GN = HSPE1 PE = 1 SV = 2 | | | | | | |
| 26S protease regulatory subunit 6A OS = Homo | UniProtKB: P17980 | 6 | 117.8 | | | |
| sapiens GN = PSMC3 PE = 1 SV = 3 | | | | | | |
| 26S protease regulatory subunit 6B OS = Homo | UniProtKB: P43686 | 4 | 40.6 | | | |
| sapiens GN = PSMC4 PE = 1 SV = 2 | | | | | | |
| 26S protease regulatory subunit 10B OS = Homo | UniProtKB: P62333 | 10 | 147.7 | | | |
| sapiens GN = PSMC6 PE = 1 SV = 1 | | | | | | |
| 26S proteasome non-ATPase regulatory subunit | UniProtKB: Q99460 | 12 | 56.9 | | | |
| 1 OS = Homo sapiens GN = PSMD1 PE = 1 SV = 2 | | | | | | |
| 26S proteasome non-ATPase regulatory subunit | UniProtKB: Q13200 | 5 | 146.9 | | | |
| 2 OS = Homo sapiens GN = PSMD2 PE = 1 SV = 3 | | | | | | |
| 26S proteasome non-ATPase regulatory subunit | UniProtKB: P55036 | 5 | 86.51 | | | |
| 4 OS = Homo sapiens GN = PSMD4 PE = 1 SV = 1 | | | | | | |
| 26S proteasome non-ATPase regulatory subunit | UniProtKB: P51665 | 7 | 37.94 | | | |
| 7 OS = Homo sapiens GN = PSMD7 PE = 1 SV = 2 | | | | | | |
| 26S proteasome non-ATPase regulatory subunit | UniProtKB: O00231 | 8 | 89.93 | | | |
| 11 OS = Homo sapiens GN = PSMD11 PE = 1 | | | | | | |
| SV = 3 | | | | | | |
| 39S ribosomal protein L12, mitochondrial | UniProtKB: P52815 | 4 | 63.79 | | | |
| OS = Homo sapiens GN = MRPL12 PE = 1 SV = 2 | | | | | | |
| 39S ribosomal protein L13, mitochondrial | UniProtKB: Q9BYD1 | 2 | 29.39 | | | 1 |
| OS = Homo sapiens GN = MRPL13 PE = 1 SV = 1 | | | | | | |
| 39S ribosomal protein L38, mitochondrial | UniProtKB: Q96DV4 | 2 | 42.57 | | | |
| OS = Homo sapiens GN = MRPL38 PE = 1 SV = 2 | | | | | | |
| 40S ribosomal protein S2 OS = Homo sapiens | UniProtKB: P15880 | 6 | 134.8 | | | |
| GN = RPS2 PE = 1 SV = 2 | | | | | | |
| 40S ribosomal protein S3 OS = Homo sapiens | UniProtKB: P23396 | 2 | 36.08 | 1 | | |
| GN = RPS3 PE = 1 SV = 2 | | | | | | |
| 40S ribosomal protein S3a OS = Homo sapiens | UniProtKB: P61247 | 6 | 50.59 | | | |
| GN = RPS3A PE = 1 SV = 2 | | | | | | |
| 40S ribosomal protein S4, Y isoform 2 | UniProtKB: Q8TD47 | 4 | 99.06 | | | |
| OS = Homo sapiens GN = RPS4Y2 PE = 1 SV = 3 | | | | | | |
| 40S ribosomal protein S5 OS = Homo sapiens | UniProtKB: P46782 | 4 | 63.66 | | | |
| GN = RPS5 PE = 1 SV = 4 | | | | | | |
| 40S ribosomal protein S6 OS = Homo sapiens | UniProtKB: P62753 | 4 | 70.33 | | | |
| GN = RPS6 PE = 1 SV = 1 | | | | | | |
| 40S ribosomal protein S7 OS = Homo sapiens | UniProtKB: P62081 | 4 | 35.8 | | | |
| GN = RPS7 PE = 1 SV = 1 | | | | | | |
| 40S ribosomal protein S8 OS = Homo sapiens | UniProtKB: P62241 | 1 | 84.81 | | | |
| GN = RPS8 PE = 1 SV = 2 | | | | | | |
| 40S ribosomal protein S9 OS = Homo sapiens | UniProtKB: P46781 | 6 | 110 | | | |
| GN = RPS9 PE = 1 SV = 3 | | | | | | |
| 40S ribosomal protein S11 OS = Homo sapiens | UniProtKB: P62280 | 4 | 52.89 | | | |
| GN = RPS11 PE = 1 SV = 3 | | | | | | |
| 40S ribosomal protein S12 OS = Homo sapiens | UniProtKB: P25398 | 1 | 46.49 | | | |
| GN = RPS12 PE = 1 SV = 3 | | | | | | |
| 40S ribosomal protein S13 OS = Homo sapiens | UniProtKB: P62277 | 5 | 42.79 | | | |
| GN = RPS13 PE = 1 SV = 2 | | | | | | |
| 40S ribosomal protein S14 OS = Homo sapiens | UniProtKB: P62263 | 2 | 36.39 | | | |
| GN = RPS14 PE = 1 SV = 3 | | | | | | |
| 40S ribosomal protein S15 OS = Homo sapiens | UniProtKB: P62841 | 2 | 30.66 | | | |
| GN = RPS15 PE = 1 SV = 2 | | | | | | |
| 40S ribosomal protein S15a OS = Homo sapiens | UniProtKB: P62244 | 2 | 50.38 | | | |
| GN = RPS15A PE = 1 SV = 2 | | | | | | |
| 40S ribosomal protein S16 OS = Homo sapiens | UniProtKB: P62249 | 7 | 72.5 | | | |
| GN = RPS16 PE = 1 SV = 2 | | | | | | |
| 40S ribosomal protein S18 OS = Homo sapiens | UniProtKB: P62269 | 7 | 132 | | | |
| GN = RPS18 PE = 1 SV = 3 | | | | | | |
| 40S ribosomal protein S19 OS = Homo sapiens | UniProtKB: P39019 | 8 | 208 | | | |
| GN = RPS19 PE = 1 SV = 2 | | | | | | |
| 40S ribosomal protein S20 OS = Homo sapiens | UniProtKB: P60866 | 5 | 131.7 | | | |
| GN = RPS20 PE = 1 SV = 1 | | | | | | |
| 40S ribosomal protein S21 OS = Homo sapiens | UniProtKB: P63220 | 2 | 59.66 | | | |
| GN = RPS21 PE = 1 SV = 1 | | | | | | |
| 40S ribosomal protein S23 OS = Homo sapiens | UniProtKB: P62266 | 3 | 79.81 | | | |
| GN = RPS23 PE = 1 SV = 3 | | | | | | |
| 40S ribosomal protein S24 OS = Homo sapiens | UniProtKB: P62847 | 8 | 72.29 | | | |
| GN = RPS24 PE = 1 SV = 1 | | | | | | |
| 40S ribosomal protein S26 OS = Homo sapiens | UniProtKB: P62854 | 4 | 39.88 | | | |
| GN = RPS26 PE = 1 SV = 3 | | | | | | |
| 40S ribosomal protein S27 OS = Homo sapiens | UniProtKB: P42677 | 3 | 187.2 | | | |
| GN = RPS27 PE = 1 SV = 3 | | | | | | |
| 40S ribosomal protein S27-like OS = Homo | UniProtKB: Q71UM5 | 2 | 83.06 | | | |
| sapiens GN = RPS27L PE = 1 SV = 3 | | | | | | |
| 40S ribosomal protein S28 OS = Homo sapiens | UniProtKB: P62857 | 1 | 47.5 | | | |
| GN = RPS28 PE = 1 SV = 1 | | | | | | |
| 40S ribosomal protein S29 OS = Homo sapiens | UniProtKB: P62273 | 2 | 22.83 | | | |
| GN = RPS29 PE = 1 SV = 2 | | | | | | |
| 40S ribosomal protein S30 OS = Homo sapiens | UniProtKB: P62861 | 1 | 24.28 | | | |
| GN = FAU PE = 1 SV = 1 | | | | | | |
| 60 kDa heat shock protein, mitochondrial | UniProtKB: P10809 | 5 | 124.3 | | | 1 |
| OS = Homo sapiens GN = HSPD1 PE = 1 SV = 2 | | | | | | |
| 60S acidic ribosomal protein P0 OS = Homo | UniProtKB: P05388 | 10 | 280.6 | | | |
| sapiens GN = RPLP0 PE = 1 SV = 1 | | | | | | |
| 60S acidic ribosomal protein P0-like OS = Homo | UniProtKB: Q8NHW5 | 4 | 86.97 | | | |
| sapiens GN = RPLP0P6 PE = 5 SV = 1 | | | | | | |
| 60S ribosomal protein L4 OS = Homo sapiens | UniProtKB: P36578 | 5 | 38.07 | | | |
| GN = RPL4 PE = 1 SV = 5 | | | | | | |
| 60S ribosomal protein L5 OS = Homo sapiens | UniProtKB: P46777 | 8 | 186.8 | | | |
| GN = RPL5 PE = 1 SV = 3 | | | | | | |
| 60S ribosomal protein L6 OS = Homo sapiens | UniProtKB: Q02878 | 6 | 139.2 | | | |
| GN = RPL6 PE = 1 SV = 3 | | | | | | |
| 60S ribosomal protein L8 OS = Homo sapiens | UniProtKB: P62917 | 5 | 265.4 | | | |
| GN = RPL8 PE = 1 SV = 2 | | | | | | |
| 60S ribosomal protein L9 OS = Homo sapiens | UniProtKB: P32969 | 4 | 138.5 | | | |
| GN = RPL9 PE = 1 SV = 1 | | | | | | |
| 60S ribosomal protein L10a OS = Homo sapiens | UniProtKB: P62906 | 3 | 45.75 | | | |
| GN = RPL10A PE = 1 SV = 2 | | | | | | |
| 60S ribosomal protein L11 OS = Homo sapiens | UniProtKB: P62913 | 4 | 173 | | | |
| GN = RPL11 PE = 1 SV = 2 | | | | | | |
| 60S ribosomal protein L12 OS = Homo sapiens | UniProtKB: P30050 | 3 | 174.3 | | | |
| GN = RPL12 PE = 1 SV = 1 | | | | | | |
| 60S ribosomal protein L13 OS = Homo sapiens | UniProtKB: P26373 | 5 | 160.8 | | | |
| GN = RPL13 PE = 1 SV = 4 | | | | | | |
| 60S ribosomal protein L13a OS = Homo sapiens | UniProtKB: P40429 | 3 | 70.97 | | | |
| GN = RPL13A PE = 1 SV = 2 | | | | | | |
| 60S ribosomal protein L14 OS = Homo sapiens | UniProtKB: P50914 | 6 | 35.31 | | | |
| GN = RPL14 PE = 1 SV = 4 | | | | | | |
| 60S ribosomal protein L15 OS = Homo sapiens | UniProtKB: P61313 | 5 | 126.6 | | | |
| GN = RPL15 PE = 1 SV = 2 | | | | | | |
| 60S ribosomal protein L17 OS = Homo sapiens | UniProtKB: P18621 | 3 | 69.71 | | | |
| GN = RPL17 PE = 1 SV = 3 | | | | | | |
| 60S ribosomal protein L18 OS = Homo sapiens | UniProtKB: Q07020 | 1 | 74.54 | | | |
| GN = RPL18 PE = 1 SV = 2 | | | | | | |
| 60S ribosomal protein Ll8a OS = Homo sapiens | UniProtKB: Q02543 | 4 | 82.02 | | | |
| GN = RPL18A PE = 1 SV = 2 | | | | | | |
| 60S ribosomal protein L19 OS = Homo sapiens | UniProtKB: P84098 | 8 | 50.74 | | | |
| GN = RPL19 PE = 1 SV = 1 | | | | | | |
| 60S ribosomal protein L21 OS = Homo sapiens | UniProtKB: P46778 | 6 | 152.3 | | | |
| GN = RPL21 PE = 1 SV = 2 | | | | | | |
| 60S ribosomal protein L22 OS = Homo sapiens | UniProtKB: P35268 | 1 | 63.08 | | | |
| GN = RPL22 PE = 1 SV = 2 | | | | | | |
| 60S ribosomal protein L23 OS = Homo sapiens | UniProtKB: P62829 | 4 | 133.6 | | | |
| GN = RPL23 PE = 1 SV = 1 | | | | | | |
| 60S ribosomal protein L23a OS = Homo sapiens | UniProtKB: P62750 | 4 | 84.27 | | | |
| GN = RPL23A PE = 1 SV = 1 | | | | | | |
| 60S ribosomal protein L24 OS = Homo sapiens | UniProtKB: P83731 | 3 | 70.95 | | 1 | |
| GN = RPL24 PE = 1 SV = 1 | | | | | | |
| 60S ribosomal protein L26 OS = Homo sapiens | UniProtKB: P61254 | 11 | 37.53 | | | |
| GN = RPL26 PE = 1 SV = 1 | | | | | | |
| 60S ribosomal protein L26-like 1 OS = Homo | UniProtKB: Q9UNX3 | 11 | 128.4 | | | |
| sapiens GN = RPL26L1 PE = 1 SV = 1 | | | | | | |
| 60S ribosomal protein L27 OS = Homo sapiens | UniProtKB: P61353 | 5 | 65.61 | | | |
| GN = RPL27 PE = 1 SV = 2 | | | | | | |
| 60S ribosomal protein L27a OS = Homo sapiens | UniProtKB: P46776 | 2 | 108 | | | |
| GN = RPL27A PE = 1 SV = 2 | | | | | | |
| 60S ribosomal protein L28 OS = Homo sapiens | UniProtKB: P46779 | 2 | 34.05 | | | |
| GN = RPL28 PE = 1 SV = 3 | | | | | | |
| 60S ribosomal protein L29 OS = Homo sapiens | UniProtKB: P47914 | 7 | 96.57 | | | |
| GN = RPL29 PE = 1 SV = 2 | | | | | | |
| 60S ribosomal protein L30 OS = Homo sapiens | UniProtKB: P62888 | 10 | 204.9 | | | |
| GN = RPL30 PE = 1 SV = 2 | | | | | | |
| 60S ribosomal protein L31 OS = Homo sapiens | UniProtKB: P62899 | 6 | 100.3 | | | |
| GN = RPL31 PE = 1 SV = 1 | | | | | | |
| 60S ribosomal protein L34 OS = Homo sapiens | UniProtKB: P49207 | 5 | 85.68 | | | |
| GN = RPL34 PE = 1 SV = 3 | | | | | | |
| 60S ribosomal protein L35 OS = Homo sapiens | UniProtKB: P42766 | 3 | 42.98 | | | |
| GN = RPL35 PE = 1 SV = 2 | | | | | | |
| 60S ribosomal protein L35a OS = Homo sapiens | UniProtKB: P18077 | 2 | 20.5 | | | |
| GN = RPL35A PE = 1 SV = 2 | | | | | | |
| 60S ribosomal protein L36 OS = Homo sapiens | UniProtKB: Q9Y3U8 | 2 | 28.32 | | | |
| GN = RPL36 PE = 1 SV = 3 | | | | | | |
| 60S ribosomal protein L37 OS = Homo sapiens | UniProtKB: P61927 | 1 | 34.93 | | | |
| GN = RPL37 PE = 1 SV = 2 | | | | | | |
| 60S ribosomal protein L37a OS = Homo sapiens | UniProtKB: P61513 | 3 | 34.26 | | | |
| GN = RPL37A PE = 1 SV = 2 | | | | | | |
| 60S ribosomal protein L38 OS = Homo sapiens | UniProtKB: P63173 | 2 | 44.34 | | | |
| GN = RPL38 PE = 1 SV = 2 | | | | | | |
| 116 kDa U5 small nuclear ribonucleoprotein | UniProtKB: Q15029 | 9 | 207 | | | |
| component OS = Homo sapiens GN = EFTUD2 | | | | | | |
| PE = 1 SV = 1 | | | | | | |
| (Bos taurus) Fibrinogen alpha chain precursor | UniProtKB: CON_P02672 | 3 | 43.52 | | 1 | |
| (Bos taurus) similar to Complement C4-A | UniProtKB: CON_P01030 | 14 | 23.23 | | | |
| precursor | | | | | | |
| (Bos taurus) Similar to Immunoglobulin | UniProtKB: CON_Q1RMN8 | 1 | 50.55 | | | |
| lambda-like polypeptide 1 | | | | | | |
| A-kinase anchor protein 12 OS = Homo sapiens | UniProtKB: Q02952 | 21 | 157.3 | | | |
| GN = AKAP12 PE = 1 SV = 3 | | | | | | |
| Acetyl-CoA acetyltransferase, mitochondrial | UniProtKB: P24752 | 6 | 148.7 | | | |
| OS = Homo sapiens GN = ACAT1 PE = 1 SV = 1 | | | | | | |
| Acidic leucine-rich nuclear phosphoprotein 32 | UniProtKB: Q9BTT0 | 1 | 67.89 | | | |
| family member E OS = Homo sapiens | | | | | | |
| GN = ANP32E PE = 1 SV = 1 | | | | | | |
| Actin-related protein 2/3 complex subunit 3 | UniProtKB: O15145 | 4 | 32.95 | | | |
| OS = Homo sapiens GN = ARPC3 PE = 1 SV = 3 | | | | | | |
| Activating signal cointegrator 1 OS = Homo | UniProtKB: Q15650 | 3 | 21.31 | | | |
| sapiens GN = TRIP4 PE = 1 SV = 4 | | | | | | |
| Activator of basal transcription 1 OS = Homo | UniProtKB: Q9ULW3 | 4 | 59.39 | | | |
| sapiens GN = ABT1 PE = 1 SV = 1 | | | | | | |
| Acylamino-acid-releasing enzyme OS = Homo | UniProtKB: P13798 | 2 | 55.79 | | | |
| sapiens GN = APEH PE = 1 SV = 4 | | | | | | |
| Adenine phosphoribosyltransferase OS = Homo | UniProtKB: P07741 | 1 | 30.65 | | | |
| sapiens GN = APRT PE = 1 SV = 2 | | | | | | |
| Adenosylhomocysteinase OS = Homo sapiens | UniProtKB: P23526 | 4 | 141.5 | | | |
| GN = AHCY PE = 1 SV = 4 | | | | | | |
| Adenylate kinase 2, mitochondrial OS = Homo | UniProtKB: P54819 | 10 | 103.4 | | | |
| sapiens GN = AK2 PE = 1 SV = 2 | | | | | | |
| Adenylate kinase domain-containing protein 1 | UniProtKB: Q5TCS8 | 4 | 33.86 | | | |
| OS = Homo sapiens GN = AKD1 PE = 1 SV = 2 | | | | | | |
| Adenylate kinase isoenzyme 1 OS = Homo | UniProtKB: P00568 | 5 | 22.56 | | | |
| sapiens GN = AK1 PE = 1 SV = 3 | | | | | | |
| Adenylyl cyclase-associated protein 1 | UniProtKB: Q01518 | 4 | 88.42 | | | |
| OS = Homo sapiens GN = CAP1 PE = 1 SV = 5 | | | | | | |
| Adipose most abundant gene transcript 2 protein | UniProtKB: Q15847 | 2 | 134.7 | | | |
| OS = Homo sapiens GN = APM2 PE = 1 SV = 1 | | | | | | |
| ADP-sugar pyrophosphatase OS = Homo sapiens | UniProtKB: Q9UKK9 | 1 | 24.62 | | | |
| GN = NUDT5 PE = 1 SV = 1 | | | | | | |
| ADP/ATP translocase 2 OS = Homo sapiens | UniProtKB: P05141 | 3 | 27.9 | | | |
| GN = SLC25A5 PE = 1 SV = 6 | | | | | | |
| Alanyl-tRNA synthetase, cytoplasmic | UniProtKB: P49588 | 7 | 108 | | | |
| OS = Homo sapiens GN = AARS PE = 1 SV = 2 | | | | | | |
| ALBU_BOVIN | ALBU_BOVIN | 1 | 56.01 | | 1 | |
| Aldehyde dehydrogenase family 1 member A3 | UniProtKB: P47895 | 4 | 35.83 | | | |
| OS = Homo sapiens GN = ALDH1A3 PE = 1 SV = 2 | | | | | | |
| Aldose reductase OS = Homo sapiens | UniProtKB: P15121 | 5 | 38.07 | | | |
| GN = AKR1B1 PE = 1 SV = 3 | | | | | | |
| Alpha-2B adrenergic receptor OS = Homo | UniProtKB: P18089 | 2 | 32.85 | | 1 | |
| sapiens GN = ADRA2B PE = 2 SV = 2 | | | | | | |
| Alpha-endosulfine OS = Homo sapiens | UniProtKB: O43768 | 2 | 35.26 | | | |
| GN = ENSA PE = 1 SV = 1 | | | | | | |
| Alstrom syndrome protein 1 OS = Homo sapiens | UniProtKB: Q8TCU4 | 8 | 26.12 | | | |
| GN = ALMS1 PE = 1 SV = 3 | | | | | | |
| Aminoacyl tRNA synthase complex-interacting | UniProtKB: Q13155 | 2 | 41.49 | | | |
| multifunctional protein 2 OS = Homo sapiens | | | | | | |
| GN = AIMP2 PE = 1 SV = 2 | | | | | | |
| Aminopeptidase Q OS = Homo sapiens | UniProtKB: Q6Q4G3 | 2 | 35.48 | | 1 | |
| GN = AQPEP PE = 1 SV = 4 | | | | | | |
| AMY-1-associating protein expressed in testis 1 | UniProtKB: Q7Z4T9 | 1 | 34.53 | | | |
| OS = Homo sapiens GN = AAT1 PE = 1 SV = 2 | | | | | | |
| Annexin A1 OS = Homo sapiens GN = ANXA1 | UniProtKB: P04083 | 6 | 246 | | | |
| PE = 1 SV = 2 | | | | | | |
| Annexin A3 OS = Homo sapiens GN = ANXA3 | UniProtKB: P12429 | 2 | 24.04 | | | |
| PE = 1 SV = 3 | | | | | | |
| Annexin A7 OS = Homo sapiens GN = ANXA7 | UniProtKB: P20073 | 3 | 62.78 | | | 1 |
| PE = 1 SV = 3 | | | | | | |
| ANXA5_HUMAN | ANXA5_HUMAN | 8 | 200.8 | | | |
| AP-1 complex subunit gamma-like 2 OS = Homo | UniProtKB: O75843 | 2 | 23.05 | | | |
| sapiens GN = AP1G2 PE = 1 SV = 1 | | | | | | |
| AP-2 complex subunit beta OS = Homo sapiens | UniProtKB: P63010 | 9 | 26.03 | | | |
| GN = AP2B1 PE = 1 SV = 1 | | | | | | |
| Apolipoprotein B-100 OS = Homo sapiens | UniProtKB: P04114 | 13 | 30.47 | | 2 | 2 |
| GN = APOB PE = 1 SV = 2 | | | | | | |
| Arginyl-tRNA synthetase, cytoplasmic | UniProtKB: P54136 | 9 | 76.57 | | | |
| OS = Homo sapiens GN = RARS PE = 1 SV = 2 | | | | | | |
| Arylamine N-acetyltransferase 1 OS = Homo | UniProtKB: P18440 | 2 | 20.62 | | | |
| sapiens GN = NAT1 PE = 1 SV = 2 | | | | | | |
| Asparagine synthetase [glutamine-hydrolyzing] | UniProtKB: P08243 | 7 | 56.92 | | | |
| OS = Homo sapiens GN = ASNS PE = 1 SV = 4 | | | | | | |
| Asparaginyl-tRNA synthetase, cytoplasmic | UniProtKB: O43776 | 8 | 176.3 | | | |
| OS = Homo sapiens GN = NARS PE = 1 SV = 1 | | | | | | |
| Aspartate aminotransferase, cytoplasmic | UniProtKB: P17174 | 6 | 80.3 | | | |
| OS = Homo sapiens GN = GOT1 PE = 1 SV = 3 | | | | | | |
| Aspartate aminotransferase, mitochondrial | UniProtKB: P00505 | 9 | 48.97 | | | |
| OS = Homo sapiens GN = GOT2 PE = 1 SV = 3 | | | | | | |
| Ataxin-2 OS = Homo sapiens GN = ATXN2 PE = 1 | UniProtKB: Q99700 | 4 | 28.6 | | | |
| SV = 2 | | | | | | |
| Ataxin-2-like protein OS = Homo sapiens | UniProtKB: Q8WWM7 | 5 | 36.75 | | | |
| GN = ATXN2L PE = 1 SV = 2 | | | | | | |
| ATP synthase subunit alpha, mitochondrial | UniProtKB: P25705 | 8 | 169.6 | | | |
| OS = Homo sapiens GN = ATP5A1 PE = 1 SV = 1 | | | | | | |
| ATP synthase subunit beta, mitochondrial | UniProtKB: P06576 | 10 | 159 | | | |
| OS = Homo sapiens GN = ATP5B PE = 1 SV = 3 | | | | | | |
| ATP synthase subunit d, mitochondrial | UniProtKB: O75947 | 7 | 64.26 | | | |
| OS = Homo sapiens GN = ATP5H PE = 1 SV = 3 | | | | | | |
| ATP synthase subunit delta, mitochondrial | UniProtKB: P30049 | 1 | 32.79 | | | |
| OS = Homo sapiens GN = ATP5D PE = 1 SV = 2 | | | | | | |
| ATP synthase subunit e, mitochondrial | UniProtKB: P56385 | 4 | 23 | | | |
| OS = Homo sapiens GN = ATP5I PE = 1 SV = 2 | | | | | | |
| ATP synthase subunit gamma, mitochondrial | UniProtKB: P36542 | 8 | 79.46 | | | |
| OS = Homo sapiens GN = ATP5C1 PE = 1 SV = 1 | | | | | | |
| ATP synthase subunit O, mitochondrial | UniProtKB: P48047 | 7 | 75.76 | | | |
| OS = Homo sapiens GN = ATP5O PE = 1 SV = 1 | | | | | | |
| ATP-binding cassette sub-family E member 1 | UniProtKB: P61221 | 7 | 64.12 | | | |
| OS = Homo sapiens GN = ABCE1 PE = 1 SV = 1 | | | | | | |
| ATP-citrate synthase OS = Homo sapiens | UniProtKB: P53396 | 15 | 113.6 | | | |
| GN = ACLY PE = 1 SV = 3 | | | | | | |
| ATP-dependent RNA helicase DDX3X | UniProtKB: O00571 | 2 | 65.32 | | | 1 |
| OS = Homo sapiens GN = DDX3X PE = 1 SV = 3 | | | | | | |
| ATP-dependent RNA helicase DDX18 | UniProtKB: Q9NVP1 | 3 | 40.36 | | | |
| OS = Homo sapiens GN = DDX18 PE = 1 SV = 2 | | | | | | |
| ATP-dependent RNA helicase DDX24 | UniProtKB: Q9GZR7 | 5 | 34.95 | | | |
| OS = Homo sapiens GN = DDX24 PE = 1 SV = 1 | | | | | | |
| ATP-dependent RNA helicase DDX54 | UniProtKB: Q8TDD1 | 5 | 76.82 | | | |
| OS = Homo sapiens GN = DDX54 PE = 1 SV = 2 | | | | | | |
| ATP-dependent RNA helicase DDX55 | UniProtKB: Q8NHQ9 | 4 | 39.83 | | | |
| OS = Homo sapiens GN = DDX55 PE = 1 SV = 3 | | | | | | |
| ATP-dependent zinc metalloprotease YME1L1 | UniProtKB: Q96TA2 | 3 | 28.66 | | 1 | |
| OS = Homo sapiens GN = YME1L1 PE = 1 SV = 2 | | | | | | |
| ATPase inhibitor, mitochondrial OS = Homo | UniProtKB: Q9UII2 | 3 | 34.51 | | | |
| sapiens GN = ATPIF1 PE = 1 SV = 1 | | | | | | |
| Axin interactor, dorsalization-associated protein | UniProtKB: Q96BJ3 | 1 | 14.36 | | | |
| OS = Homo sapiens GN = AlDA PE = 1 SV = 1 | | | | | | |
| B-cell lymphoma/leukemia 11B OS = Homo | UniProtKB: Q9C0K0 | 1 | 18.59 | | | |
| sapiens GN = BCL11B PE = 1 SV = 1 | | | | | | |
| B-cell receptor-associated protein 31 OS = Homo | UniProtKB: P51572 | 8 | 43.02 | | | |
| sapiens GN = BCAP31 PE = 1 SV = 3 | | | | | | |
| Bactericidal/permeability-increasing protein-like | UniProtKB: Q8NFQ5 | 3 | 27.67 | | | |
| 3 OS = Homo sapiens GN = BPIL3 PE = 2 SV = 1 | | | | | | |
| BAG family molecular chaperone regulator 5 | UniProtKB: Q9UL15 | 2 | 46.9 | | | |
| OS = Homo sapiens GN = BAG5 PE = 1 SV = 1 | | | | | | |
| Band 4.1-like protein 5 OS = Homo sapiens | UniProtKB: Q9HCM4 | 3 | 26.97 | | 1 | |
| GN = EPB41L5 PE = 1 SV = 3 | | | | | | |
| Basic leucine zipper and W2 domain-containing | UniProtKB: Q7L1Q6 | 10 | 44.79 | | | |
| protein 1 OS = Homo sapiens GN = BZW1 PE = 1 | | | | | | |
| SV = 1 | | | | | | |
| Basigin OS = Homo sapiens GN = BSG PE = 1 | UniProtKB: P35613 | 7 | 156.2 | | | |
| SV = 2 | | | | | | |
| Bifunctional aminoacyl-tRNA synthetase | UniProtKB: P07814 | 17 | 35.22 | | | |
| OS = Homo sapiens GN = EPRS PE = 1 SV = 5 | | | | | | |
| Bifunctional purine biosynthesis protein PURH | UniProtKB: P31939 | 5 | 95.29 | | | |
| OS = Homo sapiens GN = ATIC PE = 1 SV = 3 | | | | | | |
| Brain-specific angiogenesis inhibitor 2 | UniProtKB: O60241 | 11 | 20.58 | | | |
| OS = Homo sapiens GN = BAI2 PE = 2 SV = 2 | | | | | | |
| Bystin OS = Homo sapiens GN = BYSL PE = 1 | UniProtKB: Q13895 | 3 | 35.98 | | | |
| SV = 3 | | | | | | |
| C2 domain-containing protein 3 OS = Homo | UniProtKB: Q4AC94 | 2 | 44.27 | | | |
| sapiens GN = C2CD3 PE = 1 SV = 4 | | | | | | |
| C-1-tetrahydrofolate synthase, cytoplasmic | UniProtKB: P11586 | 11 | 107.5 | | | |
| OS = Homo sapiens GN = MTHFD1 PE = 1 SV = 3 | | | | | | |
| CAD protein OS = Homo sapiens GN = CAD | UniProtKB: P27708 | 8 | 42.24 | | | |
| PE = 1 SV = 3 | | | | | | |
| Cadherin-7 OS = Homo sapiens GN = CDH7 | UniProtKB: Q9ULB5 | 4 | 31.77 | | 2 | |
| PE = 2 SV = 2 | | | | | | |
| CAH2_HUMAN | CAH2_HUMAN | 2 | 40.1 | | | |
| Calcyclin-binding protein OS = Homo sapiens | UniProtKB: Q9HB71 | 8 | 81.06 | | | |
| GN = CACYBP PE = 1 SV = 2 | | | | | | |
| Calmodulin OS = Homo sapiens GN = CALM1 | UniProtKB: P62158 | 5 | 68.64 | | | |
| PE = 1 SV = 2 | | | | | | |
| Calnexin OS = Homo sapiens GN = CANX PE = 1 | UniProtKB: P27824 | 12 | 136.2 | | | |
| SV = 2 | | | | | | |
| Calpain small subunit 1 OS = Homo sapiens | UniProtKB: P04632 | 3 | 71.08 | | | |
| GN = CAPNS1 PE = 1 SV = 1 | | | | | | |
| Calpain-1 catalytic subunit OS = Homo sapiens | UniProtKB: P07384 | 4 | 58.11 | | 1 | 1 |
| GN = CAPN1 PE = 1 SV = 1 | | | | | | |
| Calreticulin OS = Homo sapiens GN = CALR | UniProtKB: P27797 | 2 | 49.87 | | | |
| PE = 1 SV = 1 | | | | | | |
| cAMP and cAMP-inhibited cGMP 3′,5′-cyclic | UniProtKB: Q9Y233 | 5 | 30.18 | | | |
| phosphodiesterase 10A OS = Homo sapiens | | | | | | |
| GN = PDE10A PE = 1 SV = 1 | | | | | | |
| Caprin-1 OS = Homo sapiens GN = CAPRIN1 | UniProtKB: Q14444 | 6 | 84.46 | | | 1 |
| PE = 1 SV = 2 | | | | | | |
| Cardiotrophin-like cytokine factor 1 OS = Homo | UniProtKB: Q9UBD9 | 1 | 29.17 | | | |
| sapiens GN = CLCF1 PE = 1 SV = 1 | | | | | | |
| Casein kinase I isoform gamma-3 OS = Homo | UniProtKB: Q9Y6M4 | 3 | 27.55 | | 1 | 1 |
| sapiens GN = CSNK1G3 PE = 1 SV = 2 | | | | | | |
| Casein kinase II subunit alpha OS = Homo | UniProtKB: P68400 | 1 | 52.84 | | | |
| sapiens GN = CSNK2A1 PE = 1 SV = 1 | | | | | | |
| CASK_BOVIN | CASK_BOVIN | 1 | 30.17 | | | |
| Caspase-14 OS = Homo sapiens GN = CASP14 | UniProtKB: P31944 | 1 | 24.14 | | | |
| PE = 1 SV = 2 | | | | | | |
| CATD_HUMAN | CATD_HUMAN | 3 | 30.95 | | | |
| CD2 antigen cytoplasmic tail-binding protein 2 | UniProtKB: O95400 | 2 | 67.66 | | | |
| OS = Homo sapiens GN = CD2BP2 PE = 1 SV = 1 | | | | | | |
| CD9 antigen OS = Homo sapiens GN = CD9 PE = 1 | UniProtKB: P21926 | 2 | 48.92 | | | |
| SV = 4 | | | | | | |
| CD44 antigen OS = Homo sapiens GN = CD44 | UniProtKB: P16070 | 6 | 60.92 | | | |
| PE = 1 SV = 3 | | | | | | |
| Cell cycle progression protein 1 OS = Homo | UniProtKB: Q9ULG6 | 6 | 29.7 | | | |
| sapiens GN = CCPG1 PE = 1 SV = 3 | | | | | | |
| Cell growth-regulating nucleolar protein | UniProtKB: Q9NX58 | 4 | 24.75 | | | |
| OS = Homo sapiens GN = LYAR PE = 1 SV = 2 | | | | | | |
| Cellular nucleic acid-binding protein OS = Homo | UniProtKB: P62633 | 3 | 41.74 | | | |
| sapiens GN = CNBP PE = 1 SV = 1 | | | | | | |
| Cellular retinoic acid-binding protein 2 | UniProtKB: P29373 | 6 | 57.15 | | | |
| OS = Homo sapiens GN = CRABP2 PE = 1 SV = 2 | | | | | | |
| Chloride intracellular channel protein 1 | UniProtKB: O00299 | 2 | 86.28 | | | |
| OS = Homo sapiens GN = CLIC1 PE = 1 SV = 4 | | | | | | |
| Chromodomain-helicase-DNA-binding protein 3 | UniProtKB: Q12873 | 10 | 32.64 | | | |
| OS = Homo sapiens GN = CHD3 PE = 1 SV = 3 | | | | | | |
| Chromodomain-helicase-DNA-binding protein 6 | UniProtKB: Q8TD26 | 7 | 22.18 | | | |
| OS = Homo sapiens GN = CHD6 PE = 1 SV = 4 | | | | | | |
| Citrate synthase, mitochondrial OS = Homo | UniProtKB: O75390 | 6 | 72.32 | | | |
| sapiens GN = CS PE = 1 SV = 2 | | | | | | |
| Clathrin heavy chain 1 OS = Homo sapiens | UniProtKB: Q00610 | 4 | 67.25 | | | 1 |
| GN = CLTC PE = 1 SV = 5 | | | | | | |
| Clathrin heavy chain 2 OS = Homo sapiens | UniProtKB: P53675 | 2 | 21.4 | | | |
| GN = CLTCL1 PE = 1 SV = 2 | | | | | | |
| Clathrin light chain A OS = Homo sapiens | UniProtKB: P09496 | 4 | 41.95 | | | |
| GN = CLTA PE = 1 SV = 1 | | | | | | |
| Coactosin-like protein OS = Homo sapiens | UniProtKB: Q14019 | 2 | 30.1 | | | |
| GN = COTL1 PE = 1 SV = 3 | | | | | | |
| Coatomer subunit beta′ OS = Homo sapiens | UniProtKB: P35606 | 3 | 44.47 | | | |
| GN = COPB2 PE = 1 SV = 2 | | | | | | |
| Coatomer subunit gamma OS = Homo sapiens | UniProtKB: Q9Y678 | 1 | 32.67 | | | 1 |
| GN = COPG PE = 1 SV = 1 | | | | | | |
| Coatomer subunit zeta-1 OS = Homo sapiens | UniProtKB: P61923 | 1 | 29.96 | | | |
| GN = COPZ1 PE = 1 SV = 1 | | | | | | |
| Cofilin-1 OS = Homo sapiens GN = CFL1 PE = 1 | UniProtKB: P23528 | 5 | 30.76 | | | |
| SV = 3 | | | | | | |
| Coiled-coil domain-containing protein 27 | UniProtKB: Q2M243 | 1 | 14.62 | | 1 | |
| OS = Homo sapiens GN = CCDC27 PE = 2 SV = 2 | | | | | | |
| Coiled-coil domain-containing protein 34 | UniProtKB: Q96HJ3 | 10 | 39.48 | | | |
| OS = Homo sapiens GN = CCDC34 PE = 2 SV = 2 | | | | | | |
| Coiled-coil domain-containing protein 71 | UniProtKB: Q8IV32 | 5 | 27.6 | | | |
| OS = Homo sapiens GN = CCDC71 PE = 2 SV = 2 | | | | | | |
| Coiled-coil domain-containing protein 86 | UniProtKB: Q9H6F5 | 6 | 49.06 | | | |
| OS = Homo sapiens GN = CCDC86 PE = 1 SV = 1 | | | | | | |
| Coiled-coil domain-containing protein 147 | UniProtKB: Q5T655 | 5 | 35.09 | | | 2 |
| OS = Homo sapiens GN = CCDC147 PE = 2 SV = 1 | | | | | | |
| Coiled-coil domain-containing protein 158 | UniProtKB: Q5M9N0 | 9 | 22.28 | | | |
| OS = Homo sapiens GN = CCDC158 PE = 2 SV = 2 | | | | | | |
| Coilin OS = Homo sapiens GN = COIL PE = 1 | UniProtKB: P38432 | 2 | 37.36 | | | |
| SV = 1 | | | | | | |
| COMM domain-containing protein 9 OS = Homo | UniProtKB: Q9P000 | 1 | 41.66 | | | |
| sapiens GN = COMMD9 PE = 1 SV = 2 | | | | | | |
| Complement component 1 Q subcomponent- | UniProtKB: Q07021 | 2 | 92.45 | | 1 | 1 |
| binding protein, mitochondrial OS = Homo | | | | | | |
| sapiens GN = C1QBP PE = 1 SV = 1 | | | | | | |
| Copine-3 OS = Homo sapiens GN = CPNE3 PE = 1 | UniProtKB: O75131 | 2 | 26.41 | | | |
| SV = 1 | | | | | | |
| Copper-transporting ATPase 2 OS = Homo | UniProtKB: P35670 | 2 | 32.41 | | | |
| sapiens GN = ATP7B PE = 1 SV = 4 | | | | | | |
| Coproporphyrinogen-III oxidase, mitochondrial | UniProtKB: P36551 | 6 | 115.4 | | | |
| OS = Homo sapiens GN = CPOX PE = 1 SV = 3 | | | | | | |
| Coronin-1C OS = Homo sapiens GN = CORO1C | UniProtKB: Q9ULV4 | 5 | 30.19 | | | |
| PE = 1 SV = 1 | | | | | | |
| Craniofacial development protein 1 OS = Homo | UniProtKB: Q9UEE9 | 5 | 37.58 | | | |
| sapiens GN = CFDP1 PE = 1 SV = 1 | | | | | | |
| Creatine kinase B-type OS = Homo sapiens | UniProtKB: P12277 | 3 | 39.52 | | | |
| GN = CKB PE = 1 SV = 1 | | | | | | |
| Crumbs protein homolog 3 OS = Homo sapiens | UniProtKB: Q9BUF7 | 1 | 33.23 | | | 1 |
| GN = CRB3 PE = 1 SV = 3 | | | | | | |
| CTP synthase 1 OS = Homo sapiens GN = CTPS | UniProtKB: P17812 | 6 | 42.4 | | | |
| PE = 1 SV = 2 | | | | | | |
| CYC_HUMAN | CYC_HUMAN | 4 | 28.67 | | | |
| Cystatin-A OS = Homo sapiens GN = CSTA PE = 1 | UniProtKB: P01040 | 1 | 53.6 | | | |
| SV = 1 | | | | | | |
| Cysteine and histidine-rich domain-containing | UniProtKB: Q9UHD1 | 4 | 32.85 | | | |
| protein 1 OS = Homo sapiens GN = CHORDC1 | | | | | | |
| PE = 1 SV = 2 | | | | | | |
| Cytochrome b-c1 complex subunit 1, | UniProtKB: P31930 | 3 | 176.3 | | 1 | |
| mitochondrial OS = Homo sapiens GN = UQCRC1 | | | | | | |
| PE = 1 SV = 3 | | | | | | |
| Cytochrome b-c1 complex subunit 2, | UniProtKB: P22695 | 3 | 96.01 | | | |
| mitochondrial OS = Homo sapiens GN = UQCRC2 | | | | | | |
| PE = 1 SV = 3 | | | | | | |
| Cytochrome b-c1 complex subunit 6, | UniProtKB: P07919 | 2 | 24.06 | | | |
| mitochondrial OS = Homo sapiens GN = UQCRH | | | | | | |
| PE = 1 SV = 2 | | | | | | |
| Cytochrome b-c1 complex subunit 7 OS = Homo | UniProtKB: P14927 | 2 | 39.99 | | | |
| sapiens GN = UQCRB PE = 1 SV = 2 | | | | | | |
| Cytochrome c oxidase subunit 2 OS = Homo | UniProtKB: P00403 | 2 | 33.84 | | | |
| sapiens GN = MT-CO2 PE = 1 SV = 1 | | | | | | |
| Cytochrome c oxidase subunit 4 isoform 2, | UniProtKB: Q96KJ9 | 2 | 36.43 | | | |
| mitochondrial OS = Homo sapiens GN = COX4I2 | | | | | | |
| PE = 1 SV = 2 | | | | | | |
| Cytochrome c oxidase subunit 5A, | UniProtKB: P20674 | 3 | 83.81 | | | |
| mitochondrial OS = Homo sapiens GN = COX5A | | | | | | |
| PE = 1 SV = 2 | | | | | | |
| Cytochrome c oxidase subunit 5B, | UniProtKB: P10606 | 3 | 57.21 | | | |
| mitochondrial OS = Homo sapiens GN = COX5B | | | | | | |
| PE = 1 SV = 2 | | | | | | |
| Cytochrome c oxidase subunit 6A1, | UniProtKB: P12074 | 1 | 30.73 | | | |
| mitochondrial OS = Homo sapiens GN = COX6A1 | | | | | | |
| PE = 1 SV = 4 | | | | | | |
| Cytochrome c oxidase subunit 7A2, | UniProtKB: P14406 | 2 | 41.81 | | | |
| mitochondrial OS = Homo sapiens GN = COX7A2 | | | | | | |
| PE = 1 SV = 1 | | | | | | |
| Cytochrome P450 3A7 OS = Homo sapiens | UniProtKB: P24462 | 2 | 26.57 | | | |
| GN = CYP3A7 PE = 1 SV = 2 | | | | | | |
| Cytoplasmic dynein 1 heavy chain 1 OS = Homo | UniProtKB: Q14204 | 37 | 56.88 | | 2 | |
| sapiens GN = DYNC1H1 PE = 1 SV = 5 | | | | | | |
| Cytoplasmic dynein 2 heavy chain 1 OS = Homo | UniProtKB: Q8NCM8 | 14 | 45.77 | | | 2 |
| sapiens GN = DYNC2H1 PE = 1 SV = 4 | | | | | | |
| Cytoskeleton-associated protein 2 OS = Homo | UniProtKB: Q8WWK9 | 16 | 17.54 | | 1 | 1 |
| sapiens GN = CKAP2 PE = 1 SV = 1 | | | | | | |
| Cytoskeleton-associated protein 4 OS = Homo | UniProtKB: Q07065 | 6 | 47.21 | | | |
| sapiens GN = CKAP4 PE = 1 SV = 2 | | | | | | |
| Cytosolic acyl coenzyme A thioester hydrolase | UniProtKB: O00154 | 5 | 53.12 | | | |
| OS = Homo sapiens GN = ACOT7 PE = 1 SV = 3 | | | | | | |
| D-3-phosphoglycerate dehydrogenase | UniProtKB: O43175 | 7 | 32.7 | | | |
| OS = Homo sapiens GN = PHGDH PE = 1 SV = 4 | | | | | | |
| DDB1- and CUL4-associated factor 13 | UniProtKB: Q9NV06 | 2 | 57.27 | | | |
| OS = Homo sapiens GN = DCAF13 PE = 1 SV = 2 | | | | | | |
| Death-associated protein kinase 2 OS = Homo | UniProtKB: Q9UIK4 | 1 | 37.86 | | | |
| sapiens GN = DAPK2 PE = 1 SV = 1 | | | | | | |
| Deoxynucleotidyltransferase terminal- | UniProtKB: Q5QJE6 | 8 | 36.61 | | | |
| interacting protein 2 OS = Homo sapiens | | | | | | |
| GN = DNTTIP2 PE = 1 SV = 2 | | | | | | |
| Deoxyribonuclease gamma OS = Homo sapiens | UniProtKB: Q13609 | 2 | 23.22 | | | |
| GN = DNASE1L3 PE = 1 SV = 1 | | | | | | |
| Deoxyuridine 5′-triphosphate | UniProtKB: P33316 | 4 | 72.49 | | | |
| nucleotidohydrolase, mitochondrial OS = Homo | | | | | | |
| sapiens GN = DUT PE = 1 SV = 3 | | | | | | |
| Dermcidin OS = Homo sapiens GN = DCD PE = 1 | UniProtKB: P81605 | 1 | 51.62 | | | |
| SV = 2 | | | | | | |
| Desert hedgehog protein OS = Homo sapiens | UniProtKB: O43323 | 1 | 24.39 | | | |
| GN = DHH PE = 1 SV = 1 | | | | | | |
| Developmentally-regulated GTP-binding protein | UniProtKB: Q9Y295 | 3 | 117.9 | | | |
| 1 OS = Homo sapiens GN = DRG1 PE = 1 SV = 1 | | | | | | |
| DHE3_BOVIN | DHE3_BOVIN | 3 | 88.62 | | | |
| Dihydrolipoyl dehydrogenase, mitochondrial | UniProtKB: P09622 | 9 | 53.49 | | | |
| OS = Homo sapiens GN = DLD PE = 1 SV = 2 | | | | | | |
| Dihydrolipoyllysine-residue succinyltransferase | UniProtKB: P36957 | 2 | 59.93 | | | |
| component of 2-oxoglutarate dehydrogenase | | | | | | |
| complex, mitochondrial OS = Homo sapiens | | | | | | |
| GN = DLST PE = 1 SV = 3 | | | | | | |
| Dipeptidyl peptidase 2 OS = Homo sapiens | UniProtKB: Q9UHL4 | 2 | 24.91 | | | 1 |
| GN = DPP7 PE = 1 SV = 3 | | | | | | |
| Disheveled-associated activator of | UniProtKB: Q86T65 | 1 | 15.21 | | | |
| morphogenesis 2 OS = Homo sapiens | | | | | | |
| GN = DAAM2 PE = 2 SV = 3 | | | | | | |
| DmX-like protein 2 OS = Homo sapiens | UniProtKB: Q8TDJ6 | 2 | 31.72 | | 1 | |
| GN = DMXL2 PE = 1 SV = 2 | | | | | | |
| DNA cross-link repair 1A protein OS = Homo | UniProtKB: Q6PJP8 | 6 | 42.11 | | 1 | |
| sapiens GN = DCLRE1A PE = 1 SV = 3 | | | | | | |
| DNA helicase B OS = Homo sapiens GN = HELB | UniProtKB: Q8NG08 | 5 | 25 | | 1 | |
| PE = 1 SV = 2 | | | | | | |
| DNA mismatch repair protein Msh3 OS = Homo | UniProtKB: P20585 | 4 | 21.1 | | | |
| sapiens GN = MSH3 PE = 1 SV = 3 | | | | | | |
| DNA replication licensing factor MCM2 | UniProtKB: P49736 | 9 | 75.13 | | | |
| OS = Homo sapiens GN = MCM2 PE = 1 SV = 4 | | | | | | |
| DNA replication licensing factor MCM3 | UniProtKB: P25205 | 6 | 105.6 | | | |
| OS = Homo sapiens GN = MCM3 PE = 1 SV = 3 | | | | | | |
| DNA replication licensing factor MCM5 | UniProtKB: P33992 | 4 | 20.32 | | | |
| OS = Homo sapiens GN = MCM5 PE = 1 SV = 5 | | | | | | |
| DNA-(apurinic or apyrimidinic site) lyase | UniProtKB: P27695 | 5 | 47.75 | | | |
| OS = Homo sapiens GN = APEX1 PE = 1 SV = 2 | | | | | | |
| DNA-dependent protein kinase catalytic subunit | UniProtKB: P78527 | 34 | 49.83 | | 1 | |
| OS = Homo sapiens GN = PRKDC PE = 1 SV = 3 | | | | | | |
| DnaJ homolog subfamily A member 1 | UniProtKB: P31689 | 3 | 41.74 | | | |
| OS = Homo sapiens GN = DNAJA1 PE = 1 SV = 2 | | | | | | |
| Dolichyl-diphosphooligosaccharide--protein | UniProtKB: P39656 | 4 | 55.21 | | | |
| glycosyltransferase 48 kDa subunit OS = Homo | | | | | | |
| sapiens GN = DDOST PE = 1 SV = 4 | | | | | | |
| Dolichyl-diphosphooligosaccharide--protein | UniProtKB: P04843 | 9 | 113.3 | | | |
| glycosyltransferase subunit 1 OS = Homo sapiens | | | | | | |
| GN = RPN1 PE = 1 SV = 1 | | | | | | |
| Double-stranded RNA-binding protein Staufen | UniProtKB: Q9NUL3 | 4 | 21.19 | | | |
| homolog 2 OS = Homo sapiens GN = STAU2 | | | | | | |
| PE = 1 SV = 1 | | | | | | |
| Double-stranded RNA-specific editase B2 | UniProtKB: Q9NS39 | 2 | 13.61 | | | |
| OS = Homo sapiens GN = ADARB2 PE = 1 SV = 1 | | | | | | |
| Doublesex- and mab-3-related transcription | UniProtKB: Q8IXT2 | 2 | 33.58 | | | |
| factor C2 OS = Homo sapiens GN = DMRTC2 | | | | | | |
| PE = 2 SV = 2 | | | | | | |
| Dual oxidase 1 OS = Homo sapiens GN = DUOX1 | UniProtKB: Q9NRD9 | 7 | 21.29 | | | |
| PE = 1 SV = 1 | | | | | | |
| Dual specificity protein kinase CLK4 OS = Homo | UniProtKB: Q9HAZ1 | 2 | 27.83 | | | |
| sapiens GN = CLK4 PE = 1 SV = 1 | | | | | | |
| Dynein heavy chain 1, axonemal OS = Homo | UniProtKB: Q9P2D7 | 11 | 23.1 | | | |
| sapiens GN = DNAH1 PE = 1 SV = 3 | | | | | | |
| E3 SUMO-protein ligase PIAS2 OS = Homo | UniProtKB: O75928 | 4 | 30.34 | | | 1 |
| sapiens GN = PIAS2 PE = 1 SV = 3 | | | | | | |
| E3 ubiquitin-protein ligase BRE1A OS = Homo | UniProtKB: Q5VTR2 | 13 | 17.87 | | | |
| sapiens GN = RNF20 PE = 1 SV = 2 | | | | | | |
| E3 ubiquitin-protein ligase HUWE1 OS = Homo | UniProtKB: Q7Z6Z7 | 18 | 32.77 | | | 1 |
| sapiens GN = HUWE1 PE = 1 SV = 3 | | | | | | |
| E3 ubiquitin-protein ligase RAD18 OS = Homo | UniProtKB: Q9NS91 | 4 | 31.68 | | | |
| sapiens GN = RAD18 PE = 1 SV = 2 | | | | | | |
| E3 ubiquitin-protein ligase TRIM63 OS = Homo | UniProtKB: Q969Q1 | 1 | 27.11 | | | |
| sapiens GN = TRIM63 PE = 1 SV = 1 | | | | | | |
| E3 UFM1-protein ligase 1 OS = Homo sapiens | UniProtKB: O94874 | 4 | 31.52 | | | |
| GN = KIAA0776 PE = 1 SV = 2 | | | | | | |
| EH domain-binding protein 1 OS = Homo sapiens | UniProtKB: Q8NDI1 | 3 | 27.1 | | 2 | |
| GN = EHBP1 PE = 1 SV = 3 | | | | | | |
| Electron transfer flavoprotein subunit alpha, | UniProtKB: P13804 | 3 | 34.24 | | | |
| mitochondrial OS = Homo sapiens GN = ETFA | | | | | | |
| PE = 1 SV = 1 | | | | | | |
| Electron transfer flavoprotein subunit beta | UniProtKB: P38117 | 4 | 38.43 | | | |
| OS = Homo sapiens GN = ETFB PE = 1 SV = 3 | | | | | | |
| ELKS/Rab6-interacting/CAST family member 1 | UniProtKB: Q8IUD2 | 10 | 18.06 | | 4 | 1 |
| OS = Homo sapiens GN = ERC1 PE = 1 SV = 1 | | | | | | |
| Elongation factor 1-beta OS = Homo sapiens | UniProtKB: P24534 | 2 | 69.33 | | | |
| GN = EEF1B2 PE = 1 SV = 3 | | | | | | |
| Elongation factor 1-delta OS = Homo sapiens | UniProtKB: P29692 | 5 | 213.5 | | | |
| GN = EEF1D PE = 1 SV = 5 | | | | | | |
| Elongation factor 1-gamma OS = Homo sapiens | UniProtKB: P26641 | 3 | 84.28 | | | |
| GN = EEF1G PE = 1 SV = 3 | | | | | | |
| Elongation factor 2 OS = Homo sapiens | UniProtKB: P13639 | 19 | 177 | | | |
| GN = EEF2 PE = 1 SV = 4 | | | | | | |
| Elongation factor Tu, mitochondrial OS = Homo | UniProtKB: P49411 | 7 | 91.3 | | | |
| sapiens GN = TUFM PE = 1 SV = 2 | | | | | | |
| Endoplasmic reticulum resident protein 29 | UniProtKB: P30040 | 2 | 41.86 | | 2 | |
| OS = Homo sapiens GN = ERP29 PE = 1 SV = 4 | | | | | | |
| Enhancer of rudimentary homolog OS = Homo | UniProtKB: P84090 | 4 | 40.78 | | | |
| sapiens GN = ERH PE = 1 SV = 1 | | | | | | |
| Eukaryotic initiation factor 4A-I OS = Homo | UniProtKB: P60842 | 6 | 108.7 | | | |
| sapiens GN = EIF4A1 PE = 1 SV = 1 | | | | | | |
| Eukaryotic initiation factor 4A-II OS = Homo | UniProtKB: Q14240 | 11 | 206.8 | | | |
| sapiens GN = EIF4A2 PE = 1 SV = 2 | | | | | | |
| Eukaryotic initiation factor 4A-III OS = Homo | UniProtKB: P38919 | 5 | 53.14 | | | |
| sapiens GN = EIF4A3 PE = 1 SV = 4 | | | | | | |
| Eukaryotic peptide chain release factor GTP- | UniProtKB: Q8IYD1 | 5 | 21.63 | | | |
| binding subunit ERF3B OS = Homo sapiens | | | | | | |
| GN = GSPT2 PE = 1 SV = 2 | | | | | | |
| Eukaryotic translation initiation factor 2 subunit | UniProtKB: P05198 | 3 | 88.48 | | | 1 |
| 1 OS = Homo sapiens GN = EIF2S1 PE = 1 SV = 3 | | | | | | |
| Eukaryotic translation initiation factor 2-alpha | UniProtKB: Q9P2K8 | 10 | 45.35 | | | |
| kinase 4 OS = Homo sapiens GN = EIF2AK4 | | | | | | |
| PE = 1 SV = 3 | | | | | | |
| Eukaryotic translation initiation factor 3 subunit | UniProtKB: Q14152 | 25 | 74.54 | | | |
| A OS = Homo sapiens GN = EIF3A PE = 1 SV = 1 | | | | | | |
| Eukaryotic translation initiation factor 3 subunit | UniProtKB: P55884 | 9 | 119.6 | | | |
| B OS = Homo sapiens GN = EIF3B PE = 1 SV = 3 | | | | | | |
| Eukaryotic translation initiation factor 3 subunit | UniProtKB: Q99613 | 16 | 114.2 | | | |
| C OS = Homo sapiens GN = EIF3C PE = 1 SV = 1 | | | | | | |
| Eukaryotic translation initiation factor 3 subunit | UniProtKB: O15371 | 8 | 120.2 | | 1 | |
| D OS = Homo sapiens GN = EIF3D PE = 1 SV = 1 | | | | | | |
| Eukaryotic translation initiation factor 3 subunit | UniProtKB: P60228 | 6 | 47.88 | | | |
| E OS = Homo sapiens GN = EIF3E PE = 1 SV = 1 | | | | | | |
| Eukaryotic translation initiation factor 3 subunit | UniProtKB: O00303 | 3 | 41.55 | | | |
| F OS = Homo sapiens GN = EIF3F PE = 1 SV = 1 | | | | | | |
| Eukaryotic translation initiation factor 3 subunit | UniProtKB: O15372 | 8 | 52.88 | | | |
| H OS = Homo sapiens GN = EIF3H PE = 1 SV = 1 | | | | | | |
| Eukaryotic translation initiation factor 3 subunit | UniProtKB: Q13347 | 1 | 26.43 | | | |
| I OS = Homo sapiens GN = EIF3I PE = 1 SV = 1 | | | | | | |
| Eukaryotic translation initiation factor 3 subunit | UniProtKB: Q9Y262 | 9 | 57.9 | | | |
| L OS = Homo sapiens GN = EIF3L PE = 1 SV = 1 | | | | | | |
| Eukaryotic translation initiation factor 4B | UniProtKB: P23588 | 9 | 65.77 | | | |
| OS = Homo sapiens GN = EIF4B PE = 1 SV = 2 | | | | | | |
| Eukaryotic translation initiation factor 4E | UniProtKB: P06730 | 5 | 96.95 | | | |
| OS = Homo sapiens GN = EIF4E PE = 1 SV = 2 | | | | | | |
| Eukaryotic translation initiation factor 4H | UniProtKB: Q15056 | 5 | 36.64 | | | |
| OS = Homo sapiens GN = EIF4H PE = 1 SV = 5 | | | | | | |
| Eukaryotic translation initiation factor 5 | UniProtKB: P55010 | 6 | 27.7 | | | |
| OS = Homo sapiens GN = EIF5 PE = 1 SV = 2 | | | | | | |
| Eukaryotic translation initiation factor 5A-2 | UniProtKB: Q9GZV4 | 2 | 31.23 | | | |
| OS = Homo sapiens GN = EIF5A2 PE = 1 SV = 3 | | | | | | |
| Eukaryotic translation initiation factor 6 | UniProtKB: P56537 | 1 | 38.31 | | | |
| OS = Homo sapiens GN = EIF6 PE = 1 SV = 1 | | | | | | |
| Exosome complex exonuclease RRP45 | UniProtKB: Q06265 | 2 | 80 | | | |
| OS = Homo sapiens GN = EXOSC9 PE = 1 SV = 3 | | | | | | |
| Exportin-1 OS = Homo sapiens GN = XPO1 PE = 1 | UniProtKB: O14980 | 4 | 35.66 | | | |
| SV = 1 | | | | | | |
| Exportin-2 OS = Homo sapiens GN = CSE1L | UniProtKB: P55060 | 7 | 59.79 | | | |
| PE = 1 SV = 3 | | | | | | |
| F-actin-capping protein subunit beta OS = Homo | UniProtKB: P47756 | 7 | 38.22 | | | |
| sapiens GN = CAPZB PE = 1 SV = 4 | | | | | | |
| FACT complex subunit SSRP1 OS = Homo | UniProtKB: Q08945 | 5 | 30.16 | | | |
| sapiens GN = SSRP1 PE = 1 SV = 1 | | | | | | |
| Fanconi anemia group I protein OS = Homo | UniProtKB: Q9NVI1 | 9 | 22.68 | | 1 | |
| sapiens GN = FANCI PE = 1 SV = 4 | | | | | | |
| Fascin OS = Homo sapiens GN = FSCN1 PE = 1 | UniProtKB: Q16658 | 14 | 184.5 | | | |
| SV = 3 | | | | | | |
| Fatty acid synthase OS = Homo sapiens | UniProtKB: P49327 | 9 | 116.6 | | | |
| GN = FASN PE = 1 SV = 3 | | | | | | |
| Fibronectin type III and SPRY domain- | UniProtKB: A1L4K1 | 1 | 23.42 | | | |
| containing protein 2 OS = Homo sapiens | | | | | | |
| GN = FSD2 PE = 2 SV = 1 | | | | | | |
| Filamin-A OS = Homo sapiens GN = FLNA PE = 1 | UniProtKB: P21333 | 31 | 433.6 | | | |
| SV = 4 | | | | | | |
| FK506-binding protein 15 OS = Homo sapiens | UniProtKB: Q5T1M5 | 7 | 23.22 | | | |
| GN = FKBP15 PE = 1 SV = 2 | | | | | | |
| Flap endonuclease 1 OS = Homo sapiens | UniProtKB: P39748 | 2 | 28.48 | | | 1 |
| GN = FEN1 PE = 1 SV = 1 | | | | | | |
| FRAS1-related extracellular matrix protein 2 | UniProtKB: Q5SZK8 | 6 | 21.24 | | 1 | |
| OS = Homo sapiens GN = FREM2 PE = 1 SV = 2 | | | | | | |
| Friend of PRMT1 protein OS = Homo sapiens | UniProtKB: Q9Y3Y2 | 5 | 300.9 | | | |
| GN = C1orf77 PE = 1 SV = 2 | | | | | | |
| Fructose-bisphosphate aldolase C OS = Homo | UniProtKB: P09972 | 6 | 112.7 | | | |
| sapiens GN = ALDOC PE = 1 SV = 2 | | | | | | |
| Fumarate hydratase, mitochondrial OS = Homo | UniProtKB: P07954 | 4 | 77.76 | | | |
| sapiens GN = FH PE = 1 SV = 3 | | | | | | |
| G-protein coupled receptor family C group 5 | UniProtKB: Q9NQ84 | 2 | 26.22 | | | |
| member C OS = Homo sapiens GN = GPRC5C | | | | | | |
| PE = 1 SV = 2 | | | | | | |
| G-rich sequence factor 1 OS = Homo sapiens | UniProtKB: Q12849 | 3 | 36.29 | | | |
| GN = GRSF1 PE = 1 SV = 3 | | | | | | |
| Gametogenetin OS = Homo sapiens GN = GGN | UniProtKB: Q86UU5 | 1 | 31.54 | | | |
| PE = 2 SV = 2 | | | | | | |
| GDP-L-fucose synthase OS = Homo sapiens | UniProtKB: Q13630 | 1 | 40.58 | | | |
| GN = TSTA3 PE = 1 SV = 1 | | | | | | |
| Glucosidase 2 subunit beta OS = Homo sapiens | UniProtKB: P14314 | 7 | 28.87 | | | |
| GN = PRKCSH PE = 1 SV = 2 | | | | | | |
| Glutamate-rich WD repeat-containing protein 1 | UniProtKB: Q9BQ67 | 2 | 25.55 | | | |
| OS = Homo sapiens GN = GRWD1 PE = 1 SV = 1 | | | | | | |
| Glutamine and serine-rich protein 1 OS = Homo | UniProtKB: Q2KHR3 | 3 | 18.2 | | | |
| sapiens GN = QSER1 PE = 1 SV = 3 | | | | | | |
| Glutathione reductase, mitochondrial OS = Homo | UniProtKB: P00390 | 8 | 140.1 | | | |
| sapiens GN = GSR PE = 1 SV = 2 | | | | | | |
| Glutathione S-transferase omega-1 OS = Homo | UniProtKB: P78417 | 2 | 40.83 | | | |
| sapiens GN = GSTO1 PE = 1 SV = 2 | | | | | | |
| Glyceraldehyde-3-phosphate dehydrogenase, | UniProtKB: O14556 | 2 | 38.85 | | | |
| testis-specific OS = Homo sapiens GN = GAPDHS | | | | | | |
| PE = 1 SV = 2 | | | | | | |
| Glycylpeptide N-tetradecanoyltransferase 1 | UniProtKB: P30419 | 3 | 52.9 | | | |
| OS = Homo sapiens GN = NMT1 PE = 1 SV = 2 | | | | | | |
| Glyoxalase domain-containing protein 4 | UniProtKB: Q9HC38 | 4 | 81.39 | | | |
| OS = Homo sapiens GN = GLOD4 PE = 1 SV = 1 | | | | | | |
| Granulins OS = Homo sapiens GN = GRN PE = 1 | UniProtKB: P28799 | 1 | 69.77 | | 1 | |
| SV = 2 | | | | | | |
| Group of 14-3-3 protein beta/alpha OS = Homo | UniProtKB: P31946 +2 | 13 | 199.9 | | | |
| sapiens GN = YWHAB PE = 1 SV = 3 (+2) | | | | | | |
| Group of 26S protease regulatory subunit 4 | UniProtKB: P62191 +1 | 13 | 70.46 | | | |
| OS = Homo sapiens GN = PSMC1 PE = 1 SV = 1 | | | | | | |
| (+1) | | | | | | |
| Group of 40S ribosomal protein S10 OS = Homo | UniProtKB: P46783 +1 | 5 | 67.12 | | | |
| sapiens GN = RPS10 PE = 1 SV = 1 (+1) | | | | | | |
| Group of 60S acidic ribosomal protein P1 | UniProtKB: P05386 +1 | 2 | 105 | | | |
| OS = Homo sapiens GN = RPLP1 PE = 1 SV = 1 | | | | | | |
| (+1) | | | | | | |
| Group of 60S ribosomal protein L10 OS = Homo | UniProtKB: P27635 +1 | 4 | 92.27 | | | |
| sapiens GN = RPL10 PE = 1 SV = 4 (+1) | | | | | | |
| Group of 60S ribosomal protein L36a | UniProtKB: P83881 +1 | 3 | 54.71 | | | |
| OS = Homo sapiens GN = RPL36A PE = 1 SV = 2 | | | | | | |
| (+1) | | | | | | |
| Group of (Bos taurus) Glucose-6-phosphate | UniProtKB: CON_Q3ZBD7 +1 | 11 | 217.3 | | | |
| isomerase (+1) | | | | | | |
| Group of (Bos taurus) Glyceraldehyde-3- | UniProtKB: CON_P10096 +1 | 13 | 595.1 | | | |
| phosphate dehydrogenase (+1) | | | | | | |
| Group of (Bos taurus) Metallothionein-1A (+2) | UniProtKB: CON_P67983 +2 | 2 | 22.73 | | | |
| Group of (Bos taurus) Profilin-1 (+1) | UniProtKB: CON_P02584 +1 | 8 | 291.6 | | | |
| Group of (Bos taurus) Tropomyosin 2 (+1) | UniProtKB: CON_Q3SX28; | 20 | 157 | | | |
| | CON_Q5KR48 +1 | | | | | |
| Group of Acidic leucine-rich nuclear | UniProtKB: O43423 +3 | 5 | 110.2 | | | |
| phosphoprotein 32 family member C OS = Homo | | | | | | |
| sapiens GN = ANP32C PE = 2 SV = 1 (+3) | | | | | | |
| Group of ALBU_HUMAN (+2) | ALBU_HUMAN +2 | 4 | 162.8 | | | |
| Group of ALDOA_RABIT (+1) | ALDOA_RABIT +1 | 13 | 295 | | | |
| Group of Alpha-enolase OS = Homo sapiens | UniProtKB: P06733 +2 | 16 | 600.9 | | | |
| GN = ENO1 PE = 1 SV = 2 (+2) | | | | | | |
| Group of ATP-dependent RNA helicase DDX39 | UniProtKB: O00148 +1 | 6 | 156.5 | | | |
| OS = Homo sapiens GN = DDX39 PE = 1 SV = 2 | | | | | | |
| (+1) | | | | | | |
| Group of ATPase family AAA domain- | UniProtKB: Q5T2N8 +1 | 12 | 78.1 | | | |
| containing protein 3C OS = Homo sapiens | | | | | | |
| GN = ATAD3C PE = 1 SV = 2 (+1) | | | | | | |
| Group of Calponin-3 OS = Homo sapiens | UniProtKB: Q15417 +1 | 6 | 85.34 | | | |
| GN = CNN3 PE = 1 SV = 1 (+1) | | | | | | |
| Group of Cell division protein kinase 1 | UniProtKB: P06493 +1 | 7 | 49.64 | | | |
| OS = Homo sapiens GN = CDK1 PE = 1 SV = 2 (+1) | | | | | | |
| Group of Destrin OS = Homo sapiens GN = DSTN | UniProtKB: P60981 +1 | 5 | 118.8 | | | |
| PE = 1 SV = 3 (+1) | | | | | | |
| Group of DNA-binding protein A OS = Homo | UniProtKB: P16989 +1 | 5 | 97.41 | | | |
| sapiens GN = CSDA PE = 1 SV = 4 (+1) | | | | | | |
| Group of Eukaryotic translation initiation factor | UniProtKB: P41091 +1 | 10 | 120.5 | | | |
| 2 subunit 3 OS = Homo sapiens GN = EIF2S3 | | | | | | |
| PE = 1 SV = 3 (+1) | | | | | | |
| Group of Eukaryotic translation initiation factor | UniProtKB: O43432 +1 | 14 | 75.66 | | 1 | |
| 4 gamma 3 OS = Homo sapiens GN = EIF4G3 | | | | | | |
| PE = 1 SV = 2 (+1) | | | | | | |
| Group of Eukaryotic translation initiation factor | UniProtKB: P63241 +1 | 7 | 290.6 | | | |
| 5A-1 OS = Homo sapiens GN = EIF5A PE = 1 | | | | | | |
| SV = 2 (+1) | | | | | | |
| Group of F-actin-capping protein subunit alpha- | UniProtKB: P47755 +1 | 4 | 81.89 | | | |
| 2 OS = Homo sapiens GN = CAPZA2 PE = 1 SV = 3 | | | | | | |
| (+1) | | | | | | |
| Group of Far upstream element-binding protein | UniProtKB: Q92945 +2 | 14 | 203.9 | | | |
| 2 OS = Homo sapiens GN = KHSRP PE = 1 SV = 4 | | | | | | |
| (+2) | | | | | | |
| Group of Fragile X mental retardation | UniProtKB: P51114 +2 | 4 | 109.1 | | | |
| syndrome-related protein 1 OS = Homo sapiens | | | | | | |
| GN = FXR1 PE = 1 SV = 3 (+2) | | | | | | |
| Group of Heterogeneous nuclear | UniProtKB: O14979 +1 | 9 | 161.1 | | | |
| ribonucleoprotein D-like OS = Homo sapiens | | | | | | |
| GN = HNRPDL PE = 1 SV = 3 (+1) | | | | | | |
| Group of Heterogeneous nuclear | UniProtKB: P31943 +1 | 5 | 144.1 | | | |
| ribonucleoprotein H OS = Homo sapiens | | | | | | |
| GN = HNRNPH1 PE = 1 SV = 4 (+1) | | | | | | |
| Group of Histone H1.4 OS = Homo sapiens | UniProtKB: P10412 +1 | 8 | 90.12 | | | |
| GN = HIST1H1E PE = 1 SV = 2 (+1) | | | | | | |
| Group of Histone H2A.x OS = Homo sapiens | UniProtKB: P16104 +4 | 9 | 230.7 | | | |
| GN = H2AFX PE = 1 SV = 2 (+4) | | | | | | |
| Group of Hsc70-interacting protein OS = Homo | UniProtKB: P50502 +2 | 9 | 95.55 | | | |
| sapiens GN = ST13 PE = 1 SV = 2 (+2) | | | | | | |
| Group of Inorganic pyrophosphatase OS = Homo | UniProtKB: Q15181 +1 | 9 | 92.62 | | | |
| sapiens GN = PPA1 PE = 1 SV = 2 (+1) | | | | | | |
| Group of Inosine-5′-monophosphate | UniProtKB: P12268 +1 | 20 | 299.5 | | | |
| dehydrogenase 2 OS = Homo sapiens | | | | | | |
| GN = IMPDH2 PE = 1 SV = 2 (+1) | | | | | | |
| Group of Insulin-like growth factor 2 mRNA- | UniProtKB: Q9NZI8 +1 | 9 | 69.13 | | | |
| binding protein 1 OS = Homo sapiens | | | | | | |
| GN = IGF2BP1 PE = 1 SV = 2 (+1) | | | | | | |
| Group of K2M3_SHEEP (+3) | K2M3_SHEEP +3 | 9 | 60.11 | | | |
| Group of L-lactate dehydrogenase A chain | UniProtKB: P00338 +2 | 9 | 132.3 | | | |
| OS = Homo sapiens GN = LDHA PE = 1 SV = 2 | | | | | | |
| (+2) | | | | | | |
| Group of Leiomodin-3 OS = Homo sapiens | UniProtKB: Q0VAK6 +1 | 18 | 281.6 | | | |
| GN = LMOD3 PE = 2 SV = 1 (+1) | | | | | | |
| Group of Melanoma-associated antigen B2 | UniProtKB: O15479 +1 | 5 | 77.57 | | | |
| OS = Homo sapiens GN = MAGEB2 PE = 1 SV = 2 | | | | | | |
| (+1) | | | | | | |
| Group of Myosin light chain 6B OS = Homo | UniProtKB: P14649 +1 | 2 | 74.26 | | | |
| sapiens GN = MYL6B PE = 1 SV = 1 (+1) | | | | | | |
| Group of Myosin regulatory light chain 12A | UniProtKB: P19105 +1 | 5 | 65.92 | | | |
| OS = Homo sapiens GN = MYL12A PE = 1 SV = 2 | | | | | | |
| (+1) | | | | | | |
| Group of Myosin-9 OS = Homo sapiens | UniProtKB: P35579 +3 | 62 | 614.8 | | | |
| GN = MYH9 PE = 1 SV = 4 (+3) | | | | | | |
| Group of Nascent polypeptide-associated | UniProtKB: Q13765 +1 | 2 | 149.4 | | | |
| complex subunit alpha OS = Homo sapiens | | | | | | |
| GN = NACA PE = 1 SV = 1 (+1) | | | | | | |
| Group of Phosphoglycerate kinase 1 OS = Homo | UniProtKB: P00558 +1 | 11 | 60.8 | | | |
| sapiens GN = PGK1 PE = 1 SV = 3 (+1) | | | | | | |
| Group of Phosphoglycerate mutase 2 OS = Homo | UniProtKB: P15259 +1 | 7 | 206.4 | | | |
| sapiens GN = PGAM2 PE = 1 SV = 3 (+1) | | | | | | |
| Group of Poly(rC)-binding protein 3 OS = Homo | UniProtKB: P57721 +2 | 9 | 245.9 | | | |
| sapiens GN = PCBP3 PE = 1 SV = 2 (+2) | | | | | | |
| Group of POTE ankyrin domain family member | UniProtKB: A5A3E0 +2 | 7 | 157.4 | | | |
| F OS = Homo sapiens GN = POTEF PE = 1 SV = 2 | | | | | | |
| (+2) | | | | | | |
| Group of POTE ankyrin domain family member | UniProtKB: P0CG38 +4 | 12 | 584.6 | | | |
| I OS = Homo sapiens GN = POTEI PE = 3 SV = 1 | | | | | | |
| (+4) | | | | | | |
| Group of PPIA_HUMAN (+1) | PPIA_HUMAN +1 | 9 | 193.6 | | | |
| Group of PRDX1_HUMAN (+2) | PRDX1_HUMAN +2 | 6 | 125.3 | | | |
| Group of Putative annexin A2-like protein | UniProtKB: A6NMY6 +1 | 9 | 192.1 | | | 2 |
| OS = Homo sapiens GN = ANXA2P2 PE = 5 SV = 2 | | | | | | |
| (+1) | | | | | | |
| Group of Putative fatty acid-binding protein 5- | UniProtKB: A8MUU1 +1 | 6 | 222.2 | | | |
| like protein 3 OS = Homo sapiens GN = FABP5L3 | | | | | | |
| PE = 1 SV = 1 (+1) | | | | | | |
| Group of Putative heterogeneous nuclear | UniProtKB: P0C7M2 +1 | 6 | 79.13 | | | |
| ribonucleoprotein A1-like 3 OS = Homo sapiens | | | | | | |
| GN = HNRPA1L3 PE = 5 SV = 1 (+1) | | | | | | |
| Group of Putative high mobility group protein | UniProtKB: B2RPK0 +2 | 15 | 364.6 | | | 1 |
| B1-like 1 OS = Homo sapiens GN = HMGB1L1 | | | | | | |
| PE = 5 SV = 1 (+2) | | | | | | |
| Group of Putative nucleoside diphosphate kinase | UniProtKB: O60361 +2 | 7 | 121.8 | | | |
| OS = Homo sapiens GN = NME2P1 PE = 5 SV = 1 | | | | | | |
| (+2) | | | | | | |
| Group of Putative tubulin beta chain-like protein | UniProtKB: A6NKZ8 +4 | 8 | 195.9 | | | |
| ENSP00000290377 OS = Homo sapiens PE = 5 | | | | | | |
| SV = 2 (+4) | | | | | | |
| Group of Pyrroline-5-carboxylate reductase 1, | UniProtKB: P32322 +1 | 7 | 71.06 | | | |
| mitochondrial OS = Homo sapiens GN = PYCR1 | | | | | | |
| PE = 1 SV = 2 (+1) | | | | | | |
| Group of Pyruvate kinase isozymes M1/M2 | UniProtKB: P14618 +1 | 8 | 139.3 | | | |
| OS = Homo sapiens GN = PKM2 PE = 1 SV = 4 (+1) | | | | | | |
| Group of Ras GTPase-activating protein-binding | UniProtKB: Q13283 +1 | 6 | 132.3 | | | |
| protein 1 OS = Homo sapiens GN = G3BP1 PE = 1 | | | | | | |
| SV = 1 (+1) | | | | | | |
| Group of Ras-related C3 botulinum toxin | UniProtKB: P15153 +1 | 6 | 83.27 | | | |
| substrate 2 OS = Homo sapiens GN = RAC2 PE = 1 | | | | | | |
| SV = 1 (+1) | | | | | | |
| Group of Ras-related protein Rab-13 OS = Homo | UniProtKB: P51153 +4 | 6 | 121.3 | | | |
| sapiens GN = RAB13 PE = 1 SV = 1 (+4) | | | | | | |
| Group of Regulator of differentiation 1 | UniProtKB: O95758 +2 | 3 | 65.69 | | | |
| OS = Homo sapiens GN = ROD1 PE = 1 SV = 2 (+2) | | | | | | |
| Group of Serine/arginine-rich splicing factor 4 | UniProtKB: Q08170 +1 | 4 | 92.35 | | 6 | |
| OS = Homo sapiens GN = SRSF4 PE = 1 SV = 2 | | | | | | |
| (+1) | | | | | | |
| Group of Sodium/potassium-transporting | UniProtKB: P05023 +3 | 13 | 76.31 | | 1 | |
| ATPase subunit alpha-1 OS = Homo sapiens | | | | | | |
| GN = ATP1A1 PE = 1 SV = 1 (+3) | | | | | | |
| Group of Splicing factor U2AF 35 kDa subunit | UniProtKB: Q01081 +1 | 6 | 133.9 | | | |
| OS = Homo sapiens GN = U2AF1 PE = 1 SV = 3 | | | | | | |
| (+1) | | | | | | |
| Group of Stathmin OS = Homo sapiens | UniProtKB: P16949 +1 | 7 | 36.9 | | | |
| GN = STMN1 PE = 1 SV = 3 (+1) | | | | | | |
| Group of Triosephosphate isomerase OS = Homo | UniProtKB: P60174 +1 | 10 | 452.4 | | | 1 |
| sapiens GN = TPI1 PE = 1 SV = 2 (+1) | | | | | | |
| Group of Tubulin alpha chain-like 3 OS = Homo | UniProtKB: A6NHL2 +2 | 7 | 317.2 | | | |
| sapiens GN = TUBAL3 PE = 1 SV = 2 (+2) | | | | | | |
| Group of Tubulin alpha-1B chain OS = Homo | UniProtKB: P68363 +2 | 225 | 151.7 | | | |
| sapiens GN = TUBA1B PE = 1 SV = 1 (+2) | | | | | | |
| Group of Tubulin alpha-4A chain OS = Homo | UniProtKB: P68366 +2 | 2 | 61.41 | | | |
| sapiens GN = TUBA4A PE = 1 SV = 1 (+2) | | | | | | |
| Group of Tubulin beta-4 chain OS = Homo | UniProtKB: P04350 +5 | 5 | 178.7 | | | |
| sapiens GN = TUBB4 PE = 1 SV = 2 (+5) | | | | | | |
| Group of U4/U6 small nuclear ribonucleoprotein | UniProtKB: Q8WWY3 +1 | 3 | 60.46 | | | |
| Prp31 OS = Homo sapiens GN = PRPF31 PE = 1 | | | | | | |
| SV = 2 (+1) | | | | | | |
| Group of UBIQ_HUMAN (+1) | UBIQ_HUMAN +1 | 2 | 60.66 | | | |
| GrpE protein homolog 1, mitochondrial | UniProtKB: Q9HAV7 | 5 | 34.98 | | | |
| OS = Homo sapiens GN = GRPEL1 PE = 1 SV = 2 | | | | | | |
| GSTP1_HUMAN | GSTP1_HUMAN | 4 | 164.6 | | | |
| GTP-binding nuclear protein Ran OS = Homo | UniProtKB: P62826 | 8 | 438 | | | |
| sapiens GN = RAN PE = 1 SV = 3 | | | | | | |
| GTP-binding protein 1 OS = Homo sapiens | UniProtKB: O00178 | 3 | 15.74 | | | |
| GN = GTPBP1 PE = 1 SV = 3 | | | | | | |
| GTP-binding protein 2 OS = Homo sapiens | UniProtKB: Q9BX10 | 3 | 33.75 | | | |
| GN = GTPBP2 PE = 2 SV = 1 | | | | | | |
| GTP-binding protein 8 OS = Homo sapiens | UniProtKB: Q8N3Z3 | 4 | 87.47 | | | |
| GN = GTPBP8 PE = 2 SV = 1 | | | | | | |
| GTP-binding protein 10 OS = Homo sapiens | UniProtKB: A4D1E9 | 2 | 28.61 | | | |
| GN = GTPBP10 PE = 1 SV = 1 | | | | | | |
| GTP-binding protein era homolog OS = Homo | UniProtKB: O75616 | 5 | 78.11 | | | |
| sapiens GN = ERAL1 PE = 1 SV = 2 | | | | | | |
| Guanine nucleotide-binding protein | UniProtKB: P62873 | 3 | 51.73 | | | |
| G(I)/G(S)/G(T) subunit beta-1 OS = Homo | | | | | | |
| sapiens GN = GNB1 PE = 1 SV = 3 | | | | | | |
| Guanine nucleotide-binding protein subunit | UniProtKB: P63244 | 3 | 148.9 | | | |
| beta-2-like 1 OS = Homo sapiens GN = GNB2L1 | | | | | | |
| PE = 1 SV = 3 | | | | | | |
| H/ACA ribonucleoprotein complex subunit 1 | UniProtKB: Q9NY12 | 2 | 43.81 | | | |
| OS = Homo sapiens GN = GAR1 PE = 1 SV = 1 | | | | | | |
| H/ACA ribonucleoprotein complex subunit 4 | UniProtKB: O60832 | 2 | 29.02 | | | |
| OS = Homo sapiens GN = DKC1 PE = 1 SV = 3 | | | | | | |
| HBS1-like protein OS = Homo sapiens | UniProtKB: Q9Y450 | 22 | 697 | | | |
| GN = HBS1L PE = 1 SV = 1 | | | | | | |
| Heart- and neural crest derivatives-expressed | UniProtKB: P61296 | 1 | 21.06 | | | |
| protein 2 OS = Homo sapiens GN = HAND2 PE = 1 | | | | | | |
| SV = 1 | | | | | | |
| Heat shock 70 kDa protein 4 OS = Homo sapiens | UniProtKB: P34932 | 23 | 166 | | | |
| GN = HSPA4 PE = 1 SV = 4 | | | | | | |
| Heat shock 70 kDa protein 4L OS = Homo | UniProtKB: O95757 | 2 | 20.26 | | | |
| sapiens GN = HSPA4L PE = 1 SV = 3 | | | | | | |
| Heat shock 70 kDa protein 12B OS = Homo | UniProtKB: Q96MM6 | 2 | 21.55 | | | |
| sapiens GN = HSPA12B PE = 1 SV = 2 | | | | | | |
| Heat shock protein 105 kDa OS = Homo sapiens | UniProtKB: Q92598 | 3 | 26.76 | | 1 | |
| GN = HSPH1 PE = 1 SV = 1 | | | | | | |
| Heat shock protein beta-1 OS = Homo sapiens | UniProtKB: P04792 | 2 | 49.91 | | 1 | |
| GN = HSPB1 PE = 1 SV = 2 | | | | | | |
| Hematological and neurological expressed 1 | UniProtKB: Q9UK76 | 2 | 23.97 | | | |
| protein OS = Homo sapiens GN = HN1 PE = 1 | | | | | | |
| SV = 3 | | | | | | |
| Hepatoma-derived growth factor OS = Homo | UniProtKB: P51858 | 3 | 50.96 | | | |
| sapiens GN = HDGF PE = 1 SV = 1 | | | | | | |
| HERV-K_6q14.1 provirus ancestral Gag-Pol | UniProtKB: P63128 | 3 | 20.39 | | 1 | |
| polyprotein OS = Homo sapiens PE = 3 SV = 3 | | | | | | |
| HERV-K_11q22.1 provirus ancestral Pol protein | UniProtKB: P63136 | 6 | 22.23 | | 1 | |
| OS = Homo sapiens PE = 3 SV = 1 | | | | | | |
| Heterogeneous nuclear ribonucleoprotein A0 | UniProtKB: Q13151 | 1 | 62.01 | | | |
| OS = Homo sapiens GN = HNRNPA0 PE = 1 SV = 1 | | | | | | |
| Heterogeneous nuclear ribonucleoprotein A3 | UniProtKB: P51991 | 5 | 60.66 | | | |
| OS = Homo sapiens GN = HNRNPA3 PE = 1 SV = 2 | | | | | | |
| Heterogeneous nuclear ribonucleoprotein D0 | UniProtKB: Q14103 | 4 | 25.42 | | | |
| OS = Homo sapiens GN = HNRNPD PE = 1 SV = 1 | | | | | | |
| Heterogeneous nuclear ribonucleoprotein F | UniProtKB: P52597 | 6 | 95.05 | | 1 | |
| OS = Homo sapiens GN = HNRNPF PE = 1 SV = 3 | | | | | | |
| Heterogeneous nuclear ribonucleoprotein H3 | UniProtKB: P31942 | 4 | 67.97 | | | |
| OS = Homo sapiens GN = HNRNPH3 PE = 1 SV = 2 | | | | | | |
| Heterogeneous nuclear ribonucleoprotein K | UniProtKB: P61978 | 5 | 155.6 | | | |
| OS = Homo sapiens GN = HNRNPK PE = 1 SV = 1 | | | | | | |
| Heterogeneous nuclear ribonucleoprotein L | UniProtKB: P14866 | 3 | 34.21 | | | |
| OS = Homo sapiens GN = HNRNPL PE = 1 SV = 2 | | | | | | |
| Heterogeneous nuclear ribonucleoprotein L-like | UniProtKB: Q8WVV9 | 2 | 38.06 | | | |
| OS = Homo sapiens GN = HNRPLL PE = 1 SV = 1 | | | | | | |
| Heterogeneous nuclear ribonucleoprotein M | UniProtKB: P52272 | 14 | 252.6 | | | |
| OS = Homo sapiens GN = HNRNPM PE = 1 SV = 3 | | | | | | |
| Heterogeneous nuclear ribonucleoprotein U | UniProtKB: Q00839 | 12 | 179.6 | | | |
| OS = Homo sapiens GN = HNRNPU PE = 1 SV = 6 | | | | | | |
| Heterogeneous nuclear ribonucleoproteins | UniProtKB: P22626 | 10 | 89.88 | | | |
| A2/B1 OS = Homo sapiens GN = HNRNPA2B1 | | | | | | |
| PE = 1 SV = 2 | | | | | | |
| High mobility group protein 20A OS = Homo | UniProtKB: Q9NP66 | 2 | 31.86 | | | |
| sapiens GN = HMG20A PE = 1 SV = 1 | | | | | | |
| High mobility group protein B1 OS = Homo | UniProtKB: P09429 | 6 | 49.35 | | | |
| sapiens GN = HMGB1 PE = 1 SV = 3 | | | | | | |
| High mobility group protein HMG-I/HMG-Y | UniProtKB: P17096 | 4 | 208.6 | | | |
| OS = Homo sapiens GN = HMGA1 PE = 1 SV = 3 | | | | | | |
| Histamine N-methyltransferase OS = Homo | UniProtKB: P50135 | 1 | 27.06 | | 1 | 2 |
| sapiens GN = HNMT PE = 1 SV = 1 | | | | | | |
| Histidine triad nucleotide-binding protein 1 | UniProtKB: P49773 | 3 | 47.8 | | | |
| OS = Homo sapiens GN = HINT1 PE = 1 SV = 2 | | | | | | |
| Histone acetyltransferase MYST2 OS = Homo | UniProtKB: O95251 | 5 | 39.14 | | | |
| sapiens GN = MYST2 PE = 1 SV = 1 | | | | | | |
| Histone deacetylase complex subunit SAP18 | UniProtKB: O00422 | 8 | 229 | | | |
| OS = Homo sapiens GN = SAP18 PE = 1 SV = 1 | | | | | | |
| Histone H2A deubiquitinase MYSM1 | UniProtKB: Q5VVJ2 | 4 | 25.89 | | | |
| OS = Homo sapiens GN = MYSM1 PE = 1 SV = 1 | | | | | | |
| Histone H2A.J OS = Homo sapiens GN = H2AFJ | UniProtKB: Q9BTM1 | 6 | 133.7 | | 1 | |
| PE = 1 SV = 1 | | | | | | |
| Histone H2A.Z OS = Homo sapiens GN = H2AFZ | UniProtKB: P0C0S5 | 2 | 44.99 | | | |
| PE = 1 SV = 2 | | | | | | |
| Histone H2B type 1-B OS = Homo sapiens | UniProtKB: P33778 | 6 | 89.6 | | | |
| GN = HIST1H2BB PE = 1 SV = 2 | | | | | | |
| Histone H2B type 1-H OS = Homo sapiens | UniProtKB: Q93079 | 7 | 88.88 | | | 1 |
| GN = HIST1H2BH PE = 1 SV = 3 | | | | | | |
| Histone H2B type 1-L OS = Homo sapiens | UniProtKB: Q99880 | 6 | 110.8 | | | |
| GN = HIST1H2BL PE = 1 SV = 3 | | | | | | |
| Histone H2B type 2-F OS = Homo sapiens | UniProtKB: Q5QNW6 | 5 | 156.2 | | | |
| GN = HIST2H2BF PE = 1 SV = 3 | | | | | | |
| Histone H2B type F-S OS = Homo sapiens | UniProtKB: P57053 | 3 | 41.62 | | | 1 |
| GN = H2BFS PE = 1 SV = 2 | | | | | | |
| Histone H3.1t OS = Homo sapiens GN = HIST3H3 | UniProtKB: Q16695 | 4 | 54.4 | | | |
| PE = 1 SV = 3 | | | | | | |
| Histone H3.3 OS = Homo sapiens GN = H3F3A | UniProtKB: P84243 | 3 | 27.44 | | | |
| PE = 1 SV = 2 | | | | | | |
| Histone-lysine N-methyltransferase, H3 lysine- | UniProtKB: Q96L73 | 3 | 16.31 | | | |
| 36 and H4 lysine-20 specific OS = Homo sapiens | | | | | | |
| GN = NSD1 PE = 1 SV = 1 | | | | | | |
| Histone-lysine N-methyltransferase, H3 lysine- | UniProtKB: Q8TEK3 | 6 | 26.44 | | | |
| 79 specific OS = Homo sapiens GN = DOT1L | | | | | | |
| PE = 1 SV = 2 | | | | | | |
| Homeobox protein unc-4 homolog OS = Homo | UniProtKB: A6NJT0 | 3 | 36.34 | | | |
| sapiens GN = UNCX PE = 2 SV = 1 | | | | | | |
| Hornerin OS = Homo sapiens GN = HRNR PE = 1 | UniProtKB: Q86YZ3 | 2 | 70.27 | | | |
| SV = 2 | | | | | | |
| Hsp90 co-chaperone Cdc37 OS = Homo sapiens | UniProtKB: Q16543 | 9 | 86.5 | | | |
| GN = CDC37 PE = 1 SV = 1 | | | | | | |
| Hypoxanthine-guanine | UniProtKB: P00492 | 5 | 54.43 | | | |
| phosphoribosyltransferase OS = Homo sapiens | | | | | | |
| GN = HPRT1 PE = 1 SV = 2 | | | | | | |
| Hypoxia up-regulated protein 1 OS = Homo | UniProtKB: Q9Y4L1 | 12 | 65.65 | | | |
| sapiens GN = HYOU1 PE = 1 SV = 1 | | | | | | |
| Importin subunit alpha-2 OS = Homo sapiens | UniProtKB: P52292 | 11 | 155.4 | | | |
| GN = KPNA2 PE = 1 SV = 1 | | | | | | |
| Importin subunit beta-1 OS = Homo sapiens | UniProtKB: Q14974 | 8 | 133.4 | | | |
| GN = KPNB1 PE = 1 SV = 2 | | | | | | |
| InaD-like protein OS = Homo sapiens | UniProtKB: Q8NI35 | 3 | 24.42 | | | |
| GN = INADL PE = 1 SV = 3 | | | | | | |
| Inositol hexakisphosphate kinase 1 OS = Homo | UniProtKB: Q92551 | 3 | 24.65 | | | |
| sapiens GN = IP6K1 PE = 1 SV = 3 | | | | | | |
| Inositol-3-phosphate synthase 1 OS = Homo | UniProtKB: Q9NPH2 | 1 | 47.89 | | | |
| sapiens GN = ISYNA1 PE = 1 SV = 1 | | | | | | |
| Inositol-tetrakisphosphate 1-kinase OS = Homo | UniProtKB: Q13572 | 4 | 31.18 | | | |
| sapiens GN = ITPK1 PE = 1 SV = 2 | | | | | | |
| Insulin-like growth factor 2 mRNA-binding | UniProtKB: O00425 | 5 | 66.53 | | | |
| protein 3 OS = Homo sapiens GN = IGF2BP3 | | | | | | |
| PE = 1 SV = 2 | | | | | | |
| Integrin alpha-M OS = Homo sapiens | UniProtKB: P11215 | 5 | 31.11 | | 1 | 1 |
| GN = ITGAM PE = 1 SV = 2 | | | | | | |
| Integrin beta-1 OS = Homo sapiens GN = ITGB1 | UniProtKB: P05556 | 5 | 44.42 | | | |
| PE = 1 SV = 2 | | | | | | |
| Interferon-induced very large GTPase 1 | UniProtKB: Q7Z2Y8 | 1 | 21.28 | | | |
| OS = Homo sapiens GN = GVIN1 PE = 2 SV = 2 | | | | | | |
| Interleukin enhancer-binding factor 2 OS = Homo | UniProtKB: Q12905 | 3 | 232.1 | | | |
| sapiens GN = ILF2 PE = 1 SV = 2 | | | | | | |
| Interleukin-1 family member 9 OS = Homo | UniProtKB: Q9NZH8 | 2 | 21.06 | | | |
| sapiens GN = IL1F9 PE = 1 SV = 1 | | | | | | |
| IQ and AAA domain-containing protein 1 | UniProtKB: Q86XH1 | 8 | 33.52 | | | |
| OS = Homo sapiens GN = IQCA1 PE = 2 SV = 1 | | | | | | |
| Isocitrate dehydrogenase [NAD] subunit alpha, | UniProtKB: P50213 | 6 | 58.86 | | | |
| mitochondrial OS = Homo sapiens GN = IDH3A | | | | | | |
| PE = 1 SV = 1 | | | | | | |
| Isocitrate dehydrogenase [NADP] cytoplasmic | UniProtKB: O75874 | 6 | 77.18 | | | |
| OS = Homo sapiens GN = IDH1 PE = 1 SV = 2 | | | | | | |
| Isoleucyl-tRNA synthetase, cytoplasmic | UniProtKB: P41252 | 5 | 39.02 | | | |
| OS = Homo sapiens GN = IARS PE = 1 SV = 2 | | | | | | |
| Junction plakoglobin OS = Homo sapiens | UniProtKB: P14923 | 7 | 60.1 | | | |
| GN = JUP PE = 1 SV = 3 | | | | | | |
| K2M2_SHEEP | K2M2_SHEEP | 4 | 26.07 | | | |
| Keratin, hair, basic, 4 - Homo sapiens (Human). | UniProtKB: CON_Q6ISB0 | 3 | 23.64 | | | |
| Keratin, type I cytoskeletal 9 OS = Homo sapiens | UniProtKB: P35527 | 2 | 48.52 | | | |
| GN = KRT9 PE = 1 SV = 3 | | | | | | |
| KH domain-containing, RNA-binding, signal | UniProtKB: Q07666 | 2 | 29.52 | | | |
| transduction-associated protein 1 OS = Homo | | | | | | |
| sapiens GN = KHDRBS1 PE = 1 SV = 1 | | | | | | |
| KH domain-containing, RNA-binding, signal | UniProtKB: O75525 | 1 | 43.05 | | | |
| transduction-associated protein 3 OS = Homo | | | | | | |
| sapiens GN = KHDRBS3 PE = 1 SV = 1 | | | | | | |
| Kinesin-1 heavy chain OS = Homo sapiens | UniProtKB: P33176 | 16 | 42.11 | | 1 | 1 |
| GN = KIF5B PE = 1 SV = 1 | | | | | | |
| Kinesin-like protein KIF21A OS = Homo sapiens | UniProtKB: Q7Z456 | 7 | 22.09 | | | |
| GN = KIF21A PE = 1 SV = 2 | | | | | | |
| Kinesin-like protein KIFC3 OS = Homo sapiens | UniProtKB: Q9BVG8 | 1 | 46.98 | | | |
| GN = KIFC3 PE = 1 SV = 3 | | | | | | |
| Kinetochore protein NDC80 homolog | UniProtKB: O14777 | 7 | 21.54 | | | |
| OS = Homo sapiens GN = NDC80 PE = 1 SV = 1 | | | | | | |
| La-related protein 1 OS = Homo sapiens | UniProtKB: Q6PKG0 | 3 | 45.06 | | | |
| GN = LARP1 PE = 1 SV = 2 | | | | | | |
| La-related protein 7 OS = Homo sapiens | UniProtKB: Q4G0J3 | 7 | 25.24 | | | |
| GN = LARP7 PE = 1 SV = 1 | | | | | | |
| Lactoylglutathione lyase OS = Homo sapiens | UniProtKB: Q04760 | 8 | 74.9 | | | |
| GN = GLO1 PE = 1 SV = 4 | | | | | | |
| Lamina-associated polypeptide 2, isoforms | UniProtKB: P42167 | 3 | 50.35 | | | |
| beta/gamma OS = Homo sapiens GN = TMPO | | | | | | |
| PE = 1 SV = 2 | | | | | | |
| Large proline-rich protein BAT2 OS = Homo | UniProtKB: P48634 | 2 | 29.85 | | 1 | |
| sapiens GN = BAT2 PE = 1 SV = 3 | | | | | | |
| Large subunit GTPase 1 homolog OS = Homo | UniProtKB: Q9H089 | 5 | 76.52 | | | |
| sapiens GN = LSG1 PE = 1 SV = 2 | | | | | | |
| LETM1 domain-containing protein LETM2, | UniProtKB: Q2VYF4 | 2 | 23.18 | | | |
| mitochondrial OS = Homo sapiens GN = LETM2 | | | | | | |
| PE = 2 SV = 2 | | | | | | |
| Leucine-rich PPR motif-containing protein, | UniProtKB: P42704 | 18 | 213.2 | | | |
| mitochondrial OS = Homo sapiens GN = LRPPRC | | | | | | |
| PE = 1 SV = 3 | | | | | | |
| Leucine-rich repeat protein SHOC-2 OS = Homo | UniProtKB: Q9UQ13 | 10 | 29.38 | | | |
| sapiens GN = SHOC2 PE = 1 SV = 2 | | | | | | |
| Leucine-rich repeat-containing protein 59 | UniProtKB: Q96AG4 | 5 | 40.39 | | | |
| OS = Homo sapiens GN = LRRC59 PE = 1 SV = 1 | | | | | | |
| Leukotriene A-4 hydrolase OS = Homo sapiens | UniProtKB: P09960 | 5 | 58.59 | | | |
| GN = LTA4H PE = 1 SV = 2 | | | | | | |
| LIM and calponin homology domains- | UniProtKB: Q9UPQ0 | 2 | 25.28 | | | 1 |
| containing protein 1 OS = Homo sapiens | | | | | | |
| GN = LIMCH1 PE = 1 SV = 4 | | | | | | |
| LIM and SH3 domain protein 1 OS = Homo | UniProtKB: Q14847 | 1 | 77.53 | | | |
| sapiens GN = LASP1 PE = 1 SV = 2 | | | | | | |
| LIM domain and actin-binding protein 1 | UniProtKB: Q9UHB6 | 6 | 26.13 | | | |
| OS = Homo sapiens GN = LIMA1 PE = 1 SV = 1 | | | | | | |
| Lipoamide acyltransferase component of | UniProtKB: P11182 | 5 | 71.32 | | | |
| branched-chain alpha-keto acid dehydrogenase | | | | | | |
| complex, mitochondrial OS = Homo sapiens | | | | | | |
| GN = DBT PE = 1 SV = 3 | | | | | | |
| Luc7-like protein 3 OS = Homo sapiens | UniProtKB: O95232 | 5 | 27.52 | | | |
| GN = LUC7L3 PE = 1 SV = 2 | | | | | | |
| Lupus La protein OS = Homo sapiens GN = SSB | UniProtKB: P05455 | 10 | 23.09 | | 1 | |
| PE = 1 SV = 2 | | | | | | |
| LYSC_HUMAN | LYSC_HUMAN | 4 | 53.59 | | | |
| Lysyl-tRNA synthetase OS = Homo sapiens | UniProtKB: Q15046 | 9 | 25.19 | | | |
| GN = KARS PE = 1 SV = 3 | | | | | | |
| M-phase phosphoprotein 6 OS = Homo sapiens | UniProtKB: Q99547 | 4 | 22.09 | | | |
| GN = MPHOSPH6 PE = 1 SV = 2 | | | | | | |
| Macrophage migration inhibitory factor | UniProtKB: P14174 | 3 | 105.7 | | | |
| OS = Homo sapiens GN = MIF PE = 1 SV = 4 | | | | | | |
| Macrophage-capping protein OS = Homo sapiens | UniProtKB: P40121 | 2 | 62.52 | | | |
| GN = CAPG PE = 1 SV = 2 | | | | | | |
| MAGUK p55 subfamily member 3 OS = Homo | UniProtKB: Q13368 | 2 | 35.02 | | | 1 |
| sapiens GN = MPP3 PE = 1 SV = 2 | | | | | | |
| MAGUK p55 subfamily member 6 OS = Homo | UniProtKB: Q9NZW5 | 3 | 17.32 | | | |
| sapiens GN = MPP6 PE = 1 SV = 2 | | | | | | |
| Malate dehydrogenase, cytoplasmic OS = Homo | UniProtKB: P40925 | 11 | 252.3 | | | |
| sapiens GN = MDH1 PE = 1 SV = 4 | | | | | | |
| Malectin OS = Homo sapiens GN = MLEC PE = 1 | UniProtKB: Q14165 | 3 | 34.86 | | | 1 |
| SV = 1 | | | | | | |
| Mannose- 1-phosphate guanyltransferase alpha | UniProtKB: Q96IJ6 | 2 | 18.92 | | | |
| OS = Homo sapiens GN = GMPPA PE = 1 SV = 1 | | | | | | |
| MARCKS-related protein OS = Homo sapiens | UniProtKB: P49006 | 2 | 49.62 | | | |
| GN = MARCKSL1 PE = 1 SV = 2 | | | | | | |
| Matrin-3 OS = Homo sapiens GN = MATR3 PE = 1 | UniProtKB: P43243 | 18 | 41.58 | | | |
| SV = 2 | | | | | | |
| Max dimerization protein 1 OS = Homo sapiens | UniProtKB: Q05195 | 4 | 18.26 | | | |
| GN = MXD1 PE = 1 SV = 1 | | | | | | |
| Melanoma inhibitory activity protein 3 | UniProtKB: Q5JRA6 | 18 | 14.16 | | | |
| OS = Homo sapiens GN = MIA3 PE = 1 SV = 1 | | | | | | |
| Melanoma-associated antigen B10 OS = Homo | UniProtKB: Q96LZ2 | 2 | 30.99 | | | |
| sapiens GN = MAGEB10 PE = 2 SV = 4 | | | | | | |
| Melanoma-associated antigen D2 OS = Homo | UniProtKB: Q9UNF1 | 2 | 29.39 | | 1 | |
| sapiens GN = MAGED2 PE = 1 SV = 2 | | | | | | |
| Membrane progestin receptor beta OS = Homo | UniProtKB: Q8TEZ7 | 1 | 31.43 | | | |
| sapiens GN = PAQR8 PE = 2 SV = 1 | | | | | | |
| Mesencephalic astrocyte-derived neurotrophic | UniProtKB: P55145 | 6 | 92.87 | | | |
| factor OS = Homo sapiens GN = MANF PE = 1 | | | | | | |
| SV = 2 | | | | | | |
| Mesothelin-like protein OS = Homo sapiens | UniProtKB: Q96KJ4 | 3 | 32.2 | | | 1 |
| GN = MSLNL PE = 2 SV = 3 | | | | | | |
| Methylmalonic aciduria type A protein, | UniProtKB: Q8IVH4 | 5 | 68.26 | | | |
| mitochondrial OS = Homo sapiens GN = MMAA | | | | | | |
| PE = 1 SV = 1 | | | | | | |
| Methylosome subunit pICln OS = Homo sapiens | UniProtKB: P54105 | 1 | 84.16 | | | |
| GN = CLNS1A PE = 1 SV = 1 | | | | | | |
| MHC class I polypeptide-related sequence A | UniProtKB: Q29983 | 3 | 24.29 | | | |
| OS = Homo sapiens GN = MICA PE = 1 SV = 1 | | | | | | |
| MHC class II transactivator OS = Homo sapiens | UniProtKB: P33076 | 3 | 30.21 | | | |
| GN = CIITA PE = 1 SV = 2 | | | | | | |
| Microfibrillar-associated protein 1 OS = Homo | UniProtKB: P55081 | 8 | 36.21 | | | |
| sapiens GN = MFAP1 PE = 1 SV = 2 | | | | | | |
| Microtubule-actin cross-linking factor 1, | UniProtKB: Q96PK2 | 25 | 33.56 | | | 2 |
| isoform 4 OS = Homo sapiens GN = MACF1 | | | | | | |
| PE = 1 SV = 2 | | | | | | |
| Microtubule-associated protein 4 OS = Homo | UniProtKB: P27816 | 11 | 83.83 | | | |
| sapiens GN = MAP4 PE = 1 SV = 3 | | | | | | |
| Microtubule-associated protein RP/EB family | UniProtKB: Q15691 | 2 | 34.85 | | | |
| member 1 OS = Homo sapiens GN = MAPRE1 | | | | | | |
| PE = 1 SV = 3 | | | | | | |
| Microtubule-associated tumor suppressor 1 | UniProtKB: Q9ULD2 | 15 | 30.54 | | | |
| OS = Homo sapiens GN = MTUS1 PE = 1 SV = 2 | | | | | | |
| Mitochondrial antiviral-signaling protein | UniProtKB: Q7Z434 | 1 | 27.25 | | | |
| OS = Homo sapiens GN = MAVS PE = 1 SV = 2 | | | | | | |
| Mitochondrial GTPase 1 OS = Homo sapiens | UniProtKB: Q9BT17 | 2 | 32.62 | | | |
| GN = MTG1 PE = 1 SV = 2 | | | | | | |
| Mitochondrial inner membrane protein | UniProtKB: Q16891 | 11 | 74.11 | | | |
| OS = Homo sapiens GN = IMMT PE = 1 SV = 1 | | | | | | |
| Mitochondrial-processing peptidase subunit beta | UniProtKB: O75439 | 2 | 14.69 | | | |
| OS = Homo sapiens GN = PMPCB PE = 1 SV = 2 | | | | | | |
| Mitotic checkpoint protein BUB3 OS = Homo | UniProtKB: O43684 | 2 | 58.21 | | | |
| sapiens GN = BUB3 PE = 1 SV = 1 | | | | | | |
| mRNA cap guanine-N7 methyltransferase | UniProtKB: O43148 | 7 | 15 | | | |
| OS = Homo sapiens GN = RNMT PE = 1 SV = 1 | | | | | | |
| Msx2-interacting protein OS = Homo sapiens | UniProtKB: Q96T58 | 42 | 36.66 | | | |
| GN = SPEN PE = 1 SV = 1 | | | | | | |
| Mucin-16 OS = Homo sapiens GN = MUC16 | UniProtKB: Q8WXI7 | 4 | 23.75 | | 1 | 1 |
| PE = 1 SV = 2 | | | | | | |
| Multifunctional protein ADE2 OS = Homo | UniProtKB: P22234 | 10 | 86.23 | | | |
| sapiens GN = PAICS PE = 1 SV = 3 | | | | | | |
| Myb-binding protein 1A OS = Homo sapiens | UniProtKB: Q9BQG0 | 6 | 36.71 | | 2 | |
| GN = MYBBP1A PE = 1 SV = 2 | | | | | | |
| Myosin-binding protein C, cardiac-type | UniProtKB: Q14896 | 3 | 31.48 | | | |
| OS = Homo sapiens GN = MYBPC3 PE = 1 SV = 3 | | | | | | |
| Myosin-IXb OS = Homo sapiens GN = MYO9B | UniProtKB: Q13459 | 4 | 25.73 | | | |
| PE = 1 SV = 2 | | | | | | |
| Myosin-XV OS = Homo sapiens GN = MYO15A | UniProtKB: Q9UKN7 | 11 | 30.47 | | | |
| PE = 1 SV = 2 | | | | | | |
| Myotrophin OS = Homo sapiens GN = MTPN | UniProtKB: P58546 | 3 | 91.1 | | | |
| PE = 1 SV = 2 | | | | | | |
| N-acetyltransferase 10 OS = Homo sapiens | UniProtKB: Q9H0A0 | 6 | 25.77 | | | |
| GN = NAT10 PE = 1 SV = 2 | | | | | | |
| N-alpha-acetyltransferase 15, NatA auxiliary | UniProtKB: Q9BXJ9 | 5 | 36.16 | | | |
| subunit OS = Homo sapiens GN = NAA15 PE = 1 | | | | | | |
| SV = 1 | | | | | | |
| N-alpha-acetyltransferase 38, NatC auxiliary | UniProtKB: O95777 | 1 | 30.62 | | | |
| subunit OS = Homo sapiens GN = NAA38 PE = 1 | | | | | | |
| SV = 3 | | | | | | |
| NADH dehydrogenase [ubiquinone] 1 alpha | UniProtKB: O00483 | 2 | 26.85 | | | |
| subcomplex subunit 4 OS = Homo sapiens | | | | | | |
| GN = NDUFA4 PE = 1 SV = 1 | | | | | | |
| NADH dehydrogenase [ubiquinone] | UniProtKB: P19404 | 1 | 31.62 | | | |
| flavoprotein 2, mitochondrial OS = Homo sapiens | | | | | | |
| GN = NDUFV2 PE = 1 SV = 2 | | | | | | |
| Nesprin-2 OS = Homo sapiens GN = SYNE2 | UniProtKB: Q8WXH0 | 23 | 26.46 | | 1 | |
| PE = 1 SV = 3 | | | | | | |
| Neudesin OS = Homo sapiens GN = NENF PE = 1 | UniProtKB: Q9UMX5 | 1 | 24.3 | | | |
| SV = 1 | | | | | | |
| Neural cell adhesion molecule L1 OS = Homo | UniProtKB: P32004 | 5 | 24 | | | |
| sapiens GN = L1CAM PE = 1 SV = 2 | | | | | | |
| Neuroblast differentiation-associated protein | UniProtKB: Q09666 | 68 | 117.6 | | 1 | 1 |
| AHNAK OS = Homo sapiens GN = AHNAK | | | | | | |
| PE = 1 SV = 2 | | | | | | |
| Neuroblastoma breakpoint family member 3 | UniProtKB: Q9H094 | 4 | 28.34 | | | |
| OS = Homo sapiens GN = NBPF3 PE = 2 SV = 1 | | | | | | |
| Neurofilament medium polypeptide OS = Homo | UniProtKB: P07197 | 3 | 24.41 | | | |
| sapiens GN = NEFM PE = 1 SV = 3 | | | | | | |
| Neurogenic differentiation factor 4 OS = Homo | UniProtKB: Q9HD90 | 2 | 18.23 | | | 1 |
| sapiens GN = NEUROD4 PE = 2 SV = 2 | | | | | | |
| Neuroligin-4, X-linked OS = Homo sapiens | UniProtKB: Q8N0W4 | 2 | 26.85 | | | |
| GN = NLGN4X PE = 1 SV = 1 | | | | | | |
| Neurotensin receptor type 2 OS = Homo sapiens | UniProtKB: O95665 | 1 | 23.37 | | | |
| GN = NTSR2 PE = 2 SV = 2 | | | | | | |
| Neutral alpha-glucosidase AB OS = Homo | UniProtKB: Q14697 | 5 | 108.1 | | | |
| sapiens GN = GANAB PE = 1 SV = 3 | | | | | | |
| Neutral amino acid transporter B(0) OS = Homo | UniProtKB: Q15758 | 1 | 33.62 | | | |
| sapiens GN = SLC1A5 PE = 1 SV = 2 | | | | | | |
| Next to BRCA1 gene 1 protein OS = Homo | UniProtKB: Q14596 | 4 | 26.16 | | | |
| sapiens GN = NBR1 PE = 1 SV = 3 | | | | | | |
| NHP2-like protein 1 OS = Homo sapiens | UniProtKB: P55769 | 2 | 81.66 | | | |
| GN = NHP2L1 PE = 1 SV = 3 | | | | | | |
| Nicotinamide phosphoribosyltransferase | UniProtKB: P43490 | 2 | 33.67 | | | |
| OS = Homo sapiens GN = NAMPT PE = 1 SV = 1 | | | | | | |
| Nidogen-1 OS = Homo sapiens GN = NID1 PE = 1 | UniProtKB: P14543 | 2 | 13.98 | | | |
| SV = 3 | | | | | | |
| Nuclear apoptosis-inducing factor 1 OS = Homo | UniProtKB: Q69YI7 | 2 | 17.57 | | | |
| sapiens GN = NAIF1 PE = 1 SV = 1 | | | | | | |
| Nuclear autoantigenic sperm protein OS = Homo | UniProtKB: P49321 | 14 | 80.73 | | 1 | |
| sapiens GN = NASP PE = 1 SV = 2 | | | | | | |
| Nuclear migration protein nudC OS = Homo | UniProtKB: Q9Y266 | 10 | 60.2 | | | |
| sapiens GN = NUDC PE = 1 SV = 1 | | | | | | |
| Nuclear mitotic apparatus protein 1 OS = Homo | UniProtKB: Q14980 | 17 | 46.85 | | | |
| sapiens GN = NUMA1 PE = 1 SV = 2 | | | | | | |
| Nuclear receptor subfamily 5 group A member 2 | UniProtKB: O00482 | 3 | 38.55 | | | |
| OS = Homo sapiens GN = NR5A2 PE = 1 SV = 2 | | | | | | |
| Nucleolar complex protein 2 homolog | UniProtKB: Q9Y3T9 | 3 | 72.67 | | | |
| OS = Homo sapiens GN = NOC2L PE = 1 SV = 3 | | | | | | |
| Nucleolar GTP-binding protein 1 OS = Homo | UniProtKB: Q9BZE4 | 7 | 52.15 | | | |
| sapiens GN = GTPBP4 PE = 1 SV = 3 | | | | | | |
| Nucleolar GTP-binding protein 2 OS = Homo | UniProtKB: Q13823 | 9 | 27.68 | | | |
| sapiens GN = GNL2 PE = 1 SV = 1 | | | | | | |
| Nucleolar protein 56 OS = Homo sapiens | UniProtKB: O00567 | 6 | 64.08 | | | |
| GN = NOP56 PE = 1 SV = 4 | | | | | | |
| Nucleolar protein 58 OS = Homo sapiens | UniProtKB: Q9Y2X3 | 6 | 54.69 | | | |
| GN = NOP58 PE = 1 SV = 1 | | | | | | |
| Nucleophosmin OS = Homo sapiens GN = NPM1 | UniProtKB: P06748 | 11 | 239.9 | | | 1 |
| PE = 1 SV = 2 | | | | | | |
| Nucleoplasmin-3 OS = Homo sapiens GN = NPM3 | UniProtKB: O75607 | 1 | 17.79 | | | |
| PE = 1 SV = 3 | | | | | | |
| Nucleoprotein TPR OS = Homo sapiens | UniProtKB: P12270 | 12 | 26.45 | | | |
| GN = TPR PE = 1 SV = 3 | | | | | | |
| Nucleosome assembly protein 1-like 1 | UniProtKB: P55209 | 6 | 243.2 | | | |
| OS = Homo sapiens GN = NAP1L1 PE = 1 SV = 1 | | | | | | |
| Obg-like ATPase 1 OS = Homo sapiens | UniProtKB: Q9NTK5 | 7 | 105.8 | | | |
| GN = OLA1 PE = 1 SV = 2 | | | | | | |
| Oncostatin-M-specific receptor subunit beta | UniProtKB: Q99650 | 1 | 24.13 | | 1 | |
| OS = Homo sapiens GN = OSMR PE = 1 SV = 1 | | | | | | |
| Outer dense fiber protein 3 OS = Homo sapiens | UniProtKB: Q96PU9 | 1 | 35.94 | | | |
| GN = ODF3 PE = 2 SV = 1 | | | | | | |
| p21-activated protein kinase-interacting protein | UniProtKB: Q9NWT1 | 5 | 35.53 | | | |
| 1 OS = Homo sapiens GN = PAK1IP1 PE = 1 SV = 2 | | | | | | |
| p53-regulated apoptosis-inducing protein 1 | UniProtKB: Q9HCN2 | 1 | 19.96 | | | |
| OS = Homo sapiens GN = TP53AIP1 PE = 2 SV = 1 | | | | | | |
| PAP-associated domain-containing protein 5 | UniProtKB: Q8NDF8 | 1 | 60.3 | | | |
| OS = Homo sapiens GN = PAPD5 PE = 1 SV = 2 | | | | | | |
| Paralemmin OS = Homo sapiens GN = PALM | UniProtKB: O75781 | 2 | 28.08 | | | |
| PE = 1 SV = 2 | | | | | | |
| Parathymosin OS = Homo sapiens GN = PTMS | UniProtKB: P20962 | 1 | 49.49 | | | |
| PE = 1 SV = 2 | | | | | | |
| PCI domain-containing protein 2 OS = Homo | UniProtKB: Q5JVF3 | 4 | 45.29 | | | |
| sapiens GN = PCID2 PE = 1 SV = 2 | | | | | | |
| Peptidase M20 domain-containing protein 2 | UniProtKB: Q8IYS1 | 2 | 24.84 | | | |
| OS = Homo sapiens GN = PM20D2 PE = 1 SV = 2 | | | | | | |
| Peptidyl-prolyl cis-trans isomerase B OS = Homo | UniProtKB: P23284 | 7 | 122.7 | | | 1 |
| sapiens GN = PPIB PE = 1 SV = 2 | | | | | | |
| Peptidyl-prolyl cis-trans isomerase FKBP1A | UniProtKB: P62942 | 2 | 98.05 | | | |
| OS = Homo sapiens GN = FKBP1A PE = 1 SV = 2 | | | | | | |
| Peptidyl-prolyl cis-trans isomerase FKBP4 | UniProtKB: Q02790 | 10 | 63.11 | | | |
| OS = Homo sapiens GN = FKBP4 PE = 1 SV = 3 | | | | | | |
| Peptidylprolyl cis-trans isomerase A-like 4G | UniProtKB: A2BFH1 | 3 | 33.24 | | | |
| OS = Homo sapiens GN = PPIAL4GPE = 2 SV = 1 | | | | | | |
| Pericentrin OS = Homo sapiens GN = PCNT PE = 1 | UniProtKB: O95613 | 29 | 32.25 | | | 1 |
| SV = 4 | | | | | | |
| Perilipin-3 OS = Homo sapiens GN = PLIN3 PE = 1 | UniProtKB: O60664 | 3 | 26.34 | | | |
| SV = 2 | | | | | | |
| Periodic tryptophan protein 1 homolog | UniProtKB: Q13610 | 2 | 25.65 | | | |
| OS = Homo sapiens GN = PWP1 PE = 1 SV = 1 | | | | | | |
| Peroxiredoxin-5, mitochondrial OS = Homo | UniProtKB: P30044 | 2 | 34.53 | | | |
| sapiens GN = PRDX5 PE = 1 SV = 3 | | | | | | |
| Peroxiredoxin-6 OS = Homo sapiens | UniProtKB: P30041 | 8 | 221.4 | | | |
| GN = PRDX6 PE = 1 SV = 3 | | | | | | |
| Pescadillo homolog OS = Homo sapiens | UniProtKB: O00541 | 5 | 36.84 | | | 1 |
| GN = PES1 PE = 1 SV = 1 | | | | | | |
| Phenylalanyl-tRNA synthetase beta chain | UniProtKB: Q9NSD9 | 3 | 34.58 | | | |
| OS = Homo sapiens GN = FARSB PE = 1 SV = 3 | | | | | | |
| Phosphate carrier protein, mitochondrial | UniProtKB: Q00325 | 3 | 42.48 | | | |
| OS = Homo sapiens GN = SLC25A3 PE = 1 SV = 2 | | | | | | |
| Phosphatidylethanolamine-binding protein 1 | UniProtKB: P30086 | 3 | 230.2 | | | |
| OS = Homo sapiens GN = PEBP1 PE = 1 SV = 3 | | | | | | |
| Phosphoglycerate mutase 1 OS = Homo sapiens | UniProtKB: P18669 | 3 | 37.46 | | | |
| GN = PGAM1 PE = 1 SV = 2 | | | | | | |
| Phosphoserine aminotransferase OS = Homo | UniProtKB: Q9Y617 | 7 | 164.8 | | | |
| sapiens GN = PSAT1 PE = 1 SV = 2 | | | | | | |
| PIN2/TERF1-interacting telomerase inhibitor 1 | UniProtKB: Q96BK5 | 4 | 58.45 | | | |
| OS = Homo sapiens GN = PINX1 PE = 1 SV = 2 | | | | | | |
| Pinin OS = Homo sapiens GN = PNN PE = 1 SV = 4 | UniProtKB: Q9H307 | 18 | 250.7 | | | |
| Plasminogen activator inhibitor 1 RNA-binding | UniProtKB: Q8NC51 | 6 | 139.5 | | | |
| protein OS = Homo sapiens GN = SERBP1 PE = 1 | | | | | | |
| SV = 2 | | | | | | |
| Platelet-activating factor acetylhydrolase IB | UniProtKB: P68402 | 1 | 38.39 | | | |
| subunit beta OS = Homo sapiens | | | | | | |
| GN = PAFAH1B2 PE = 1 SV = 1 | | | | | | |
| Platelet-activating factor acetylhydrolase IB | UniProtKB: Q15102 | 2 | 55.26 | | | |
| subunit gamma OS = Homo sapiens | | | | | | |
| GN = PAFAH1B3 PE = 1 SV = 1 | | | | | | |
| Platelet-derived growth factor receptor-like | UniProtKB: Q15198 | 2 | 20.07 | | | |
| protein OS = Homo sapiens GN = PDGFRL PE = 1 | | | | | | |
| SV = 1 | | | | | | |
| Plectin OS = Homo sapiens GN = PLEC PE = 1 | UniProtKB: Q15149 | 18 | 34.13 | | | |
| SV = 3 | | | | | | |
| Pleiotropic regulator 1 OS = Homo sapiens | UniProtKB: O43660 | 4 | 46.46 | | | |
| GN = PLRG1 PE = 1 SV = 1 | | | | | | |
| Pogo transposable element with KRAB domain | UniProtKB: Q9P215 | 1 | 18.27 | | | |
| OS = Homo sapiens GN = POGK PE = 1 SV = 2 | | | | | | |
| Poly [ADP-ribose] polymerase 1 OS = Homo | UniProtKB: P09874 | 19 | 56.08 | | | 1 |
| sapiens GN = PARP1 PE = 1 SV = 4 | | | | | | |
| Poly(U)-binding-splicing factor PUF60 | UniProtKB: Q9UHX1 | 3 | 22.07 | | | |
| OS = Homo sapiens GN = PUF60 PE = 1 SV = 1 | | | | | | |
| Polycomb group RING finger protein 1 | UniProtKB: Q9BSM1 | 3 | 22.84 | | | 1 |
| OS = Homo sapiens GN = PCGF1 PE = 1 SV = 2 | | | | | | |
| Polycomb protein SUZ12 OS = Homo sapiens | UniProtKB: Q15022 | 2 | 20.7 | | | |
| GN = SUZ12 PE = 1 SV = 3 | | | | | | |
| Polymerase delta-interacting protein 3 | UniProtKB: Q9BY77 | 4 | 71.96 | | | |
| OS = Homo sapiens GN = POLDIP3 PE = 1 SV = 2 | | | | | | |
| Polymerase I and transcript release factor | UniProtKB: Q6NZI2 | 2 | 35.6 | | | |
| OS = Homo sapiens GN = PTRF PE = 1 SV = 1 | | | | | | |
| Pre-mRNA 3′-end-processing factor FIP1 | UniProtKB: Q6UN15 | 5 | 113.3 | | | |
| OS = Homo sapiens GN = FIP1L1 PE = 1 SV = 1 | | | | | | |
| Pre-mRNA branch site protein p14 OS = Homo | UniProtKB: Q9Y3B4 | 3 | 61.6 | | | |
| sapiens GN = SF3B14 PE = 1 SV = 1 | | | | | | |
| Pre-mRNA-processing factor 6 OS = Homo | UniProtKB: O94906 | 10 | 52.18 | | | |
| sapiens GN = PRPF6 PE = 1 SV = 1 | | | | | | |
| Pre-mRNA-processing factor 19 OS = Homo | UniProtKB: Q9UMS4 | 2 | 53.53 | | | |
| sapiens GN = PRPF19 PE = 1 SV = 1 | | | | | | |
| Pre-mRNA-processing factor 40 homolog A | UniProtKB: O75400 | 10 | 54.87 | | | |
| OS = Homo sapiens GN = PRPF40A PE = 1 SV = 2 | | | | | | |
| Pre-mRNA-processing-splicing factor 8 | UniProtKB: Q6P2Q9 | 20 | 257.2 | | 1 | |
| OS = Homo sapiens GN = PRPF8 PE = 1 SV = 2 | | | | | | |
| Pre-mRNA-splicing factor RBM22 OS = Homo | UniProtKB: Q9NW64 | 1 | 47.61 | | | |
| sapiens GN = RBM22 PE = 1 SV = 1 | | | | | | |
| Pre-mRNA-splicing factor SLU7 OS = Homo | UniProtKB: O95391 | 2 | 18.06 | | | |
| sapiens GN = SLU7 PE = 1 SV = 2 | | | | | | |
| Pre-mRNA-splicing factor SPF27 OS = Homo | UniProtKB: O75934 | 1 | 22.39 | | | |
| sapiens GN = BCAS2 PE = 1 SV = 1 | | | | | | |
| Pre-mRNA-splicing factor SYF1 OS = Homo | UniProtKB: Q9HCS7 | 1 | 34.37 | | | |
| sapiens GN = XAB2 PE = 1 SV = 2 | | | | | | |
| Pre-mRNA-splicing regulator WTAP OS = Homo | UniProtKB: Q15007 | 6 | 140.8 | | | |
| sapiens GN = WTAP PE = 1 SV = 2 | | | | | | |
| Prefoldin subunit 2 OS = Homo sapiens | UniProtKB: Q9UHV9 | 5 | 75.05 | | | |
| GN = PFDN2 PE = 1 SV = 1 | | | | | | |
| Prefoldin subunit 6 OS = Homo sapiens | UniProtKB: O15212 | 5 | 22.86 | | | |
| GN = PFDN6 PE = 1 SV = 1 | | | | | | |
| Probable ATP-dependent RNA helicase DDX28 | UniProtKB: Q9NUL7 | 2 | 64.77 | | | |
| OS = Homo sapiens GN = DDX28 PE = 2 SV = 2 | | | | | | |
| Probable ATP-dependent RNA helicase DDX41 | UniProtKB: Q9UJV9 | 5 | 24.87 | | | |
| OS = Homo sapiens GN = DDX41 PE = 1 SV = 2 | | | | | | |
| Probable ATP-dependent RNA helicase DDX46 | UniProtKB: Q7L014 | 17 | 30.79 | | | |
| OS = Homo sapiens GN = DDX46 PE = 1 SV = 2 | | | | | | |
| Probable ATP-dependent RNA helicase DDX47 | UniProtKB: Q9H0S4 | 6 | 65.73 | | | |
| OS = Homo sapiens GN = DDX47 PE = 1 SV = 1 | | | | | | |
| Probable ATP-dependent RNA helicase DDX58 | UniProtKB: O95786 | 7 | 28.68 | | | |
| OS = Homo sapiens GN = DDX58 PE = 1 SV = 2 | | | | | | |
| Probable dimethyladenosine transferase | UniProtKB: Q9UNQ2 | 4 | 39.69 | | | |
| OS = Homo sapiens GN = DIMT1L PE = 1 SV = 1 | | | | | | |
| Probable E3 ubiquitin-protein ligase MYCBP2 | UniProtKB: O75592 | 16 | 33.47 | | | |
| OS = Homo sapiens GN = MYCBP2 PE = 1 SV = 3 | | | | | | |
| Probable E3 ubiquitin-protein ligase TRIP12 | UniProtKB: Q14669 | 8 | 40.99 | | | |
| OS = Homo sapiens GN = TRIP12 PE = 1 SV = 1 | | | | | | |
| Probable methyltransferase TARBP1 OS = Homo | UniProtKB: Q13395 | 4 | 30.02 | | | |
| sapiens GN = TARBP1 PE = 1 SV = 1 | | | | | | |
| Probable phospholipid-transporting ATPase IH | UniProtKB: P98196 | 1 | 33.59 | | | |
| OS = Homo sapiens GN = ATP11A PE = 2 SV = 3 | | | | | | |
| Probable ribosome biogenesis protein RLP24 | UniProtKB: Q9UHA3 | 5 | 33.46 | | | |
| OS = Homo sapiens GN = RSL24D1 PE = 1 SV = 1 | | | | | | |
| Probable rRNA-processing protein EBP2 | UniProtKB: Q99848 | 14 | 110.4 | | | |
| OS = Homo sapiens GN = EBNA1BP2 PE = 1 | | | | | | |
| SV = 2 | | | | | | |
| Profilin-2 OS = Homo sapiens GN = PFN2 PE = 1 | UniProtKB: P35080 | 2 | 81.06 | | | |
| SV = 3 | | | | | | |
| Programmed cell death 6-interacting protein | UniProtKB: Q8WUM4 | 8 | 29.51 | | | |
| OS = Homo sapiens GN = PDCD6IP PE = 1 SV = 1 | | | | | | |
| Programmed cell death protein 5 OS = Homo | UniProtKB: O14737 | 5 | 91.91 | | | |
| sapiens GN = PDCD5 PE = 1 SV = 3 | | | | | | |
| Prohibitin OS = Homo sapiens GN = PHB PE = 1 | UniProtKB: P35232 | 1 | 68.65 | | | |
| SV = 1 | | | | | | |
| Prohibitin-2 OS = Homo sapiens GN = PHB2 | UniProtKB: Q99623 | 4 | 71.76 | | | |
| PE = 1 SV = 2 | | | | | | |
| Proliferating cell nuclear antigen OS = Homo | UniProtKB: P12004 | 2 | 85.19 | | | |
| sapiens GN = PCNA PE = 1 SV = 1 | | | | | | |
| Proliferation-associated protein 2G4 OS = Homo | UniProtKB: Q9UQ80 | 4 | 34.05 | | | |
| sapiens GN = PA2G4 PE = 1 SV = 3 | | | | | | |
| Proline-rich protein 25 OS = Homo sapiens | UniProtKB: Q96S07 | 2 | 52.69 | | 1 | |
| GN = PRR25 PE = 4 SV = 1 | | | | | | |
| Prostaglandin E synthase 3 OS = Homo sapiens | UniProtKB: Q15185 | 4 | 91.62 | | | |
| GN = PTGES3 PE = 1 SV = 1 | | | | | | |
| Proteasome activator complex subunit 2 | UniProtKB: Q9UL46 | 3 | 42.55 | | | |
| OS = Homo sapiens GN = PSME2 PE = 1 SV = 4 | | | | | | |
| Proteasome activator complex subunit 3 | UniProtKB: P61289 | 5 | 49.37 | | | |
| OS = Homo sapiens GN = PSME3 PE = 1 SV = 1 | | | | | | |
| Proteasome subunit alpha type-1 OS = Homo | UniProtKB: P25786 | 7 | 79.68 | | | |
| sapiens GN = PSMA1 PE = 1 SV = 1 | | | | | | |
| Proteasome subunit alpha type-2 OS = Homo | UniProtKB: P25787 | 3 | 44.36 | | | |
| sapiens GN = PSMA2 PE = 1 SV = 2 | | | | | | |
| Proteasome subunit alpha type-3 OS = Homo | UniProtKB: P25788 | 5 | 61.33 | | | |
| sapiens GN = PSMA3 PE = 1 SV = 2 | | | | | | |
| Proteasome subunit alpha type-5 OS = Homo | UniProtKB: P28066 | 2 | 78.53 | | | |
| sapiens GN = PSMA5 PE = 1 SV = 3 | | | | | | |
| Proteasome subunit alpha type-6 OS = Homo | UniProtKB: P60900 | 5 | 62.24 | | | |
| sapiens GN = PSMA6 PE = 1 SV = 1 | | | | | | |
| Proteasome subunit alpha type-7 OS = Homo | UniProtKB: O14818 | 8 | 131.2 | | | |
| sapiens GN = PSMA7 PE = 1 SV = 1 | | | | | | |
| Proteasome subunit beta type-1 OS = Homo | UniProtKB: P20618 | 1 | 61.21 | | | |
| sapiens GN = PSMB1 PE = 1 SV = 2 | | | | | | |
| Proteasome subunit beta type-2 OS = Homo | UniProtKB: P49721 | 4 | 42.5 | | | |
| sapiens GN = PSMB2 PE = 1 SV = 1 | | | | | | |
| Proteasome subunit beta type-5 OS = Homo | UniProtKB: P28074 | 2 | 150.2 | | | |
| sapiens GN = PSMB5 PE = 1 SV = 3 | | | | | | |
| Proteasome subunit beta type-6 OS = Homo | UniProtKB: P28072 | 2 | 35.89 | | | |
| sapiens GN = PSMB6 PE = 1 SV = 4 | | | | | | |
| Proteasome subunit beta type-7 OS = Homo | UniProtKB: Q99436 | 3 | 32.1 | | | |
| sapiens GN = PSMB7 PE = 1 SV = 1 | | | | | | |
| Protein arginine N-methyltransferase 1 | UniProtKB: Q99873 | 4 | 27.72 | | | |
| OS = Homo sapiens GN = PRMT1 PE = 1 SV = 2 | | | | | | |
| Protein artemis OS = Homo sapiens | UniProtKB: Q96SD1 | 2 | 28.2 | | | |
| GN = DCLRE1C PE = 1 SV = 2 | | | | | | |
| Protein BAT2-like 2 OS = Homo sapiens | UniProtKB: Q9Y520 | 19 | 27.93 | | 3 | |
| GN = BAT2L2 PE = 1 SV = 2 | | | | | | |
| Protein CutA OS = Homo sapiens GN = CUTA | UniProtKB: O60888 | 1 | 60.45 | | | |
| PE = 1 SV = 2 | | | | | | |
| Protein delta homolog 1 OS = Homo sapiens | UniProtKB: P80370 | 1 | 43.65 | | | |
| GN = DLK1 PE = 1 SV = 3 | | | | | | |
| Protein disulfide-isomerase A3 OS = Homo | UniProtKB: P30101 | 6 | 171.5 | | 1 | |
| sapiens GN = PDIA3 PE = 1 SV = 4 | | | | | | |
| Protein disulfide-isomerase A4 OS = Homo | UniProtKB: P13667 | 4 | 71.32 | | | |
| sapiens GN = PDIA4 PE = 1 SV = 2 | | | | | | |
| Protein disulfide-isomerase A6 OS = Homo | UniProtKB: Q15084 | 13 | 232.6 | | | |
| sapiens GN = PDIA6 PE = 1 SV = 1 | | | | | | |
| Protein DJ-1 OS = Homo sapiens GN = PARK7 | UniProtKB: Q99497 | 2 | 33.65 | | | |
| PE = 1 SV = 2 | | | | | | |
| Protein FAM3C OS = Homo sapiens | UniProtKB: Q92520 | 6 | 65.15 | | | |
| GN = FAM3C PE = 1 SV = 1 | | | | | | |
| Protein FAM38B OS = Homo sapiens | UniProtKB: Q9H5I5 | 2 | 28.02 | | | |
| GN = FAM38B PE = 2 SV = 1 | | | | | | |
| Protein FAM114A2 OS = Homo sapiens | UniProtKB: Q9NRY5 | 3 | 25.68 | | | |
| GN = FAM114A2 PE = 1 SV = 4 | | | | | | |
| Protein FAM118B OS = Homo sapiens | UniProtKB: Q9BPY3 | 1 | 21.16 | | 1 | 1 |
| GN = FAM118B PE = 1 SV = 1 | | | | | | |
| Protein FAM123B OS = Homo sapiens | UniProtKB: Q5JTC6 | 3 | 25.98 | | 2 | |
| GN = FAM123B PE = 1 SV = 2 | | | | | | |
| Protein FAM136A OS = Homo sapiens | UniProtKB: Q96C01 | 2 | 22.64 | | | |
| GN = FAM136A PE = 1 SV = 1 | | | | | | |
| Protein FAM184A OS = Homo sapiens | UniProtKB: Q8NB25 | 14 | 42.84 | | | |
| GN = FAM184A PE = 2 SV = 3 | | | | | | |
| Protein FAM186A OS = Homo sapiens | UniProtKB: A6NE01 | 5 | 20.35 | | | |
| GN = FAM186A PE = 2 SV = 3 | | | | | | |
| Protein fem-1 homolog A OS = Homo sapiens | UniProtKB: Q9BSK4 | 2 | 25.94 | | | |
| GN = FEM1A PE = 1 SV = 1 | | | | | | |
| Protein KIAA0649 OS = Homo sapiens | UniProtKB: Q5T8A7 | 7 | 21.93 | | | |
| GN = KIAA0649 PE = 1 SV = 1 | | | | | | |
| Protein kinase C eta type OS = Homo sapiens | UniProtKB: P24723 | 3 | 38.8 | | | |
| GN = PRKCH PE = 1 SV = 4 | | | | | | |
| Protein KRI1 homolog OS = Homo sapiens | UniProtKB: Q8N9T8 | 5 | 68.65 | | | |
| GN = KRI1 PE = 1 SV = 2 | | | | | | |
| Protein LSM12 homolog OS = Homo sapiens | UniProtKB: Q3MHD2 | 3 | 51.21 | | | |
| GN = LSM12 PE = 1 SV = 2 | | | | | | |
| Protein LYRIC OS = Homo sapiens GN = MTDH | UniProtKB: Q86UE4 | 2 | 47.69 | | 3 | 2 |
| PE = 1 SV = 2 | | | | | | |
| Protein mago nashi homolog 2 OS = Homo | UniProtKB: Q96A72 | 4 | 97.14 | | | |
| sapiens GN = MAGOHB PE = 1 SV = 1 | | | | | | |
| Protein RCC2 OS = Homo sapiens GN = RCC2 | UniProtKB: Q9P258 | 3 | 40.04 | | | |
| PE = 1 SV = 2 | | | | | | |
| Protein Red OS = Homo sapiens GN = IK PE = 1 | UniProtKB: Q13123 | 7 | 18.81 | | | |
| SV = 3 | | | | | | |
| Protein regulator of cytokinesis 1 OS = Homo | UniProtKB: O43663 | 3 | 31.14 | | | |
| sapiens GN = PRC1 PE = 1 SV = 2 | | | | | | |
| Protein RRP5 homolog OS = Homo sapiens | UniProtKB: Q14690 | 14 | 29.21 | | | |
| GN = PDCD11 PE = 1 SV = 3 | | | | | | |
| Protein S100-A4 OS = Homo sapiens | UniProtKB: P26447 | 3 | 44.59 | | | |
| GN = S100A4 PE = 1 SV = 1 | | | | | | |
| Protein S100-A6 OS = Homo sapiens | UniProtKB: P06703 | 2 | 49.5 | | | |
| GN = S100A6 PE = 1 SV = 1 | | | | | | |
| Protein S100-A8 OS = Homo sapiens | UniProtKB: P05109 | 6 | 54.13 | | | |
| GN = S100A8 PE = 1 SV = 1 | | | | | | |
| Protein S100-A9 OS = Homo sapiens | UniProtKB: P06702 | 5 | 28.57 | | | |
| GN = S100A9 PE = 1 SV = 1 | | | | | | |
| Protein S100-A11 OS = Homo sapiens | UniProtKB: P31949 | 2 | 28.92 | | | |
| GN = S100A11 PE = 1 SV = 2 | | | | | | |
| Protein SET OS = Homo sapiens GN = SET PE = 1 | UniProtKB: Q01105 | 6 | 70.94 | | | |
| SV = 3 | | | | | | |
| Protein Shroom3 OS = Homo sapiens | UniProtKB: Q8TF72 | 4 | 27.02 | | 1 | |
| GN = SHROOM3 PE = 1 SV = 2 | | | | | | |
| Protein SON OS = Homo sapiens GN = SON | UniProtKB: P18583 | 5 | 25.42 | | | |
| PE = 1 SV = 4 | | | | | | |
| Protein sprouty homolog 1 OS = Homo sapiens | UniProtKB: O43609 | 1 | 38.34 | | | |
| GN = SPRY1 PE = 1 SV = 2 | | | | | | |
| Protein transport protein Sec31B OS = Homo | UniProtKB: Q9NQW1 | 2 | 31.8 | | | |
| sapiens GN = SEC31B PE = 1 SV = 1 | | | | | | |
| Protein virilizer homolog OS = Homo sapiens | UniProtKB: Q69YN4 | 7 | 104.6 | | | |
| GN = KIAA1429 PE = 1 SV = 2 | | | | | | |
| Proteolipid protein 2 OS = Homo sapiens | UniProtKB: Q04941 | 1 | 44.87 | | | |
| GN = PLP2 PE = 1 SV = 1 | | | | | | |
| Prothymosin alpha OS = Homo sapiens | UniProtKB: P06454 | 3 | 193 | | | |
| GN = PTMA PE = 1 SV = 2 | | | | | | |
| Protocadherin Fat 2 OS = Homo sapiens | UniProtKB: Q9NYQ8 | 5 | 25.36 | | 3 | |
| GN = FAT2 PE = 1 SV = 2 | | | | | | |
| Puromycin-sensitive aminopeptidase OS = Homo | UniProtKB: P55786 | 7 | 57.62 | | 2 | |
| sapiens GN = NPEPPS PE = 1 SV = 2 | | | | | | |
| Putative 40S ribosomal protein S26-like 1 | UniProtKB: Q5JNZ5 | 2 | 32.73 | | | |
| OS = Homo sapiens GN = RPS26P11 PE = 5 SV = 1 | | | | | | |
| Putative adenosylhomocysteinase 2 OS = Homo | UniProtKB: O43865 | 5 | 33.86 | | | |
| sapiens GN = AHCYL1 PE = 1 SV = 2 | | | | | | |
| Putative elongation factor 1-alpha-like 3 | UniProtKB: Q5VTE0 | 12 | 184.6 | | | |
| OS = Homo sapiens GN = EEF1AL3 PE = 5 SV = 1 | | | | | | |
| Putative golgin subfamily A member 8I | UniProtKB: A6NC78 | 8 | 29.47 | | 1 | |
| OS = Homo sapiens GN = GOLGA8IP PE = 5 | | | | | | |
| SV = 2 | | | | | | |
| Putative nascent polypeptide-associated | UniProtKB: Q9BZK3 | 3 | 55.95 | | | |
| complex subunit alpha-like protein OS = Homo | | | | | | |
| sapiens GN = NACAP1 PE = 5 SV = 1 | | | | | | |
| Putative neuroblastoma breakpoint family | UniProtKB: P0C2Y1 | 6 | 34.27 | | | |
| member 7 OS = Homo sapiens GN = NBPF7 PE = 5 | | | | | | |
| SV = 1 | | | | | | |
| Putative olfactory receptor 56B2 OS = Homo | UniProtKB: Q8NGI1 | 1 | 15.57 | | | |
| sapiens GN = OR56B2P PE = 5 SV = 1 | | | | | | |
| Putative pituitary tumor-transforming gene 3 | UniProtKB: Q9NZH4 | 4 | 33.33 | | | |
| protein OS = Homo sapiens GN = PTTG3P PE = 5 | | | | | | |
| SV = 1 | | | | | | |
| Putative pre-mRNA-splicing factor ATP- | UniProtKB: O43143 | 3 | 33.59 | | | |
| dependent RNA helicase DHX15 OS = Homo | | | | | | |
| sapiens GN = DHX15 PE = 1 SV = 2 | | | | | | |
| Putative ribosomal RNA methyltransferase | UniProtKB: P46087 | 13 | 170.3 | | | |
| NOP2 OS = Homo sapiens GN = NOP2 PE = 1 | | | | | | |
| SV = 2 | | | | | | |
| Putative RNA-binding protein Luc7-like 1 | UniProtKB: Q9NQ29 | 2 | 46.99 | | | |
| OS = Homo sapiens GN = LUC7L PE = 1 SV = 1 | | | | | | |
| Putative RNA-binding protein Luc7-like 2 | UniProtKB: Q9Y383 | 5 | 52.23 | | | |
| OS = Homo sapiens GN = LUC7L2 PE = 1 SV = 2 | | | | | | |
| Putative rRNA methyltransferase 3 OS = Homo | UniProtKB: Q8IY81 | 10 | 31.49 | | | |
| sapiens GN = FTSJ3 PE = 1 SV = 2 | | | | | | |
| Putative small nuclear ribonucleoprotein | UniProtKB: Q5VYJ4 | 2 | 50.67 | | | |
| polypeptide E-like protein 1 OS = Homo sapiens | | | | | | |
| GN = SNRPEL1 PE = 5 SV = 1 | | | | | | |
| Putative tropomyosin alpha-3 chain-like protein | UniProtKB: A6NL28 | 4 | 63.04 | | | |
| OS = Homo sapiens PE = 5 SV = 2 | | | | | | |
| Putative ubiquitin-conjugating enzyme E2 N- | UniProtKB: Q5JXB2 | 3 | 21.72 | | | |
| like OS = Homo sapiens GN = UBE2NL PE = 5 | | | | | | |
| SV = 1 | | | | | | |
| Putative uncharacterized protein F1134945 | UniProtKB: Q8NAQ8 | 1 | 31.11 | | 1 | |
| OS = Homo sapiens PE = 2 SV = 1 | | | | | | |
| Putative UPF0607 protein ENSP00000383144 | UniProtKB: A8MX80 | 1 | 35.13 | | | |
| OS = Homo sapiens PE = 3 SV = 2 | | | | | | |
| Putative UPF0607 protein FLJ37424 OS = Homo | UniProtKB: Q8N9G6 | 3 | 33.7 | | | |
| sapiens PE = 2 SV = 1 | | | | | | |
| Rab GDP dissociation inhibitor alpha OS = Homo | UniProtKB: P31150 | 3 | 22.93 | | | |
| sapiens GN = GDI1 PE = 1 SV = 2 | | | | | | |
| Rab GDP dissociation inhibitor beta OS = Homo | UniProtKB: P50395 | 8 | 93.2 | | | |
| sapiens GN = GDI2 PE = 1 SV = 2 | | | | | | |
| Rab-3A-interacting protein OS = Homo sapiens | UniProtKB: Q96QF0 | 2 | 18.52 | | | |
| GN = RAB3IP PE = 1 SV = 1 | | | | | | |
| Rabphilin-3A OS = Homo sapiens GN = RPH3A | UniProtKB: Q9Y2J0 | 5 | 21.62 | | | |
| PE = 1 SV = 1 | | | | | | |
| Ran GTPase-activating protein 1 OS = Homo | UniProtKB: P46060 | 8 | 61.13 | | | |
| sapiens GN = RANGAP1 PE = 1 SV = 1 | | | | | | |
| Ran-specific GTPase-activating protein | UniProtKB: P43487 | 5 | 57.19 | | | |
| OS = Homo sapiens GN = RANBP1 PE = 1 SV = 1 | | | | | | |
| Ras association domain-containing protein 6 | UniProtKB: Q6ZTQ3 | 1 | 32.16 | | 1 | |
| OS = Homo sapiens GN = RASSF6 PE = 1 SV = 1 | | | | | | |
| Ras GTPase-activating-like protein IQGAP1 | UniProtKB: P46940 | 17 | 32.97 | | | |
| OS = Homo sapiens GN = IQGAP1 PE = 1 SV = 1 | | | | | | |
| Ras-related protein Rab-2A OS = Homo sapiens | UniProtKB: P61019 | 3 | 83.84 | | | |
| GN = RAB2A PE = 1 SV = 1 | | | | | | |
| Ras-related protein Rab-5C OS = Homo sapiens | UniProtKB: P51148 | 4 | 69.07 | | | |
| GN = RAB5C PE = 1 SV = 2 | | | | | | |
| Ras-related protein Rab-7a OS = Homo sapiens | UniProtKB: P51149 | 3 | 44.23 | | | |
| GN = RAB7A PE = 1 SV = 1 | | | | | | |
| Ras-related protein Rab-11A OS = Homo sapiens | UniProtKB: P62491 | 6 | 93.93 | | | |
| GN = RAB11A PE = 1 SV = 3 | | | | | | |
| Ras-related protein Rab-15 OS = Homo sapiens | UniProtKB: P59190 | 4 | 37.45 | | | |
| GN = RAB15 PE = 1 SV = 1 | | | | | | |
| Ras-related protein Rab-28 OS = Homo sapiens | UniProtKB: P51157 | 2 | 26.8 | | | |
| GN = RAB28 PE = 1 SV = 2 | | | | | | |
| Regulator of chromosome condensation | UniProtKB: P18754 | 6 | 122.9 | | | |
| OS = Homo sapiens GN = RCC1 PE = 1 SV = 1 | | | | | | |
| Regulator of G-protein signaling 14 OS = Homo | UniProtKB: O43566 | 1 | 29.33 | | | |
| sapiens GN = RGS14 PE = 1 SV = 4 | | | | | | |
| Replication factor C subunit 4 OS = Homo | UniProtKB: P35249 | 4 | 32.18 | | | |
| sapiens GN = RFC4 PE = 1 SV = 2 | | | | | | |
| Retinol-binding protein 1 OS = Homo sapiens | UniProtKB: P09455 | 2 | 76.61 | | | |
| GN = RBP1 PE = 1 SV = 2 | | | | | | |
| Rho GDP-dissociation inhibitor 1 OS = Homo | UniProtKB: P52565 | 5 | 127.9 | | | |
| sapiens GN = ARHGDIA PE = 1 SV = 3 | | | | | | |
| Rho-related GTP-binding protein RhoC | UniProtKB: P08134 | 4 | 43.94 | | | |
| OS = Homo sapiens GN = RHOC PE = 1 SV = 1 | | | | | | |
| Ribonuclease inhibitor OS = Homo sapiens | UniProtKB: P13489 | 3 | 32.93 | | | |
| GN = RNH1 PE = 1 SV = 2 | | | | | | |
| Ribosomal L1 domain-containing protein 1 | UniProtKB: O76021 | 43 | 1 | | 2 | |
| OS = Homo sapiens GN = RSL1D1 PE = 1 SV = 3 | | | | | | |
| Ribosomal RNA processing protein 1 homolog | UniProtKB: P56182 | 3 | 59.37 | | | |
| A OS = Homo sapiens GN = RRP1 PE = 1 SV = 1 | | | | | | |
| Ribosomal RNA processing protein 1 homolog | UniProtKB: Q14684 | 6 | 112.9 | | | |
| B OS = Homo sapiens GN = RRP1B PE = 1 SV = 3 | | | | | | |
| Ribosome biogenesis protein BOP1 OS = Homo | UniProtKB: Q14137 | 2 | 58.74 | | | |
| sapiens GN = BOP1 PE = 1 SV = 2 | | | | | | |
| Ribosome biogenesis protein NSA2 homolog | UniProtKB: O95478 | 7 | 40.2 | | | |
| OS = Homo sapiens GN = NSA2 PE = 1 SV = 1 | | | | | | |
| Ribosome biogenesis protein WDR12 | UniProtKB: Q9GZL7 | 1 | 18.2 | | | |
| OS = Homo sapiens GN = WDR12 PE = 1 SV = 2 | | | | | | |
| Ribosome biogenesis regulatory protein | UniProtKB: Q15050 | 4 | 50.42 | | | |
| homolog OS = Homo sapiens GN = RRS1 PE = 1 | | | | | | |
| SV = 2 | | | | | | |
| RNA methyltransferase-like protein 1 | UniProtKB: Q9HC36 | 2 | 67.12 | | | |
| OS = Homo sapiens GN = RNMTL1 PE = 1 SV = 2 | | | | | | |
| RNA polymerase II-associated protein 1 | UniProtKB: Q9BWH6 | 1 | 25.29 | | | |
| OS = Homo sapiens GN = RPAP1 PE = 1 SV = 3 | | | | | | |
| RNA-binding motif protein, X-linked-like-3 | UniProtKB: Q8N7X1 | 1 | 74.64 | | | |
| OS = Homo sapiens GN = RBMXL3 PE = 2 SV = 2 | | | | | | |
| RNA-binding protein 8A OS = Homo sapiens | UniProtKB: Q9Y5S9 | 2 | 68.97 | | | |
| GN = RBM8A PE = 1 SV = 1 | | | | | | |
| RNA-binding protein 14 OS = Homo sapiens | UniProtKB: Q96PK6 | 2 | 51.46 | | | |
| GN = RBM14 PE = 1 SV = 2 | | | | | | |
| RNA-binding protein 16 OS = Homo sapiens | UniProtKB: Q9UPN6 | 2 | 30.97 | | | |
| GN = RBM16 PE = 1 SV = 1 | | | | | | |
| RNA-binding protein 27 OS = Homo sapiens | UniProtKB: Q9P2N5 | 8 | 53.53 | | | 1 |
| GN = RBM27 PE = 1 SV = 2 | | | | | | |
| RNA-binding protein 39 OS = Homo sapiens | UniProtKB: Q14498 | 2 | 56.61 | | | |
| GN = RBM39 PE = 1 SV = 2 | | | | | | |
| RNA-binding protein 45 OS = Homo sapiens | UniProtKB: Q8IUH3 | 5 | 30.39 | | | |
| GN = RBM45 PE = 1 SV = 1 | | | | | | |
| RNA-binding protein with serine-rich domain 1 | UniProtKB: Q15287 | 5 | 71.6 | | 1 | |
| OS = Homo sapiens GN = RNPS1 PE = 1 SV = 1 | | | | | | |
| RNA-binding Raly-like protein OS = Homo | UniProtKB: Q86SE5 | 3 | 40.34 | | | |
| sapiens GN = RALYL PE = 1 SV = 2 | | | | | | |
| Rootletin OS = Homo sapiens GN = CROCC | UniProtKB: Q5TZA2 | 21 | 41.1 | | | |
| PE = 1 SV = 1 | | | | | | |
| rRNA 2′-O-methyltransferase fibrillarin | UniProtKB: P22087 | 2 | 24.22 | | | |
| OS = Homo sapiens GN = FBL PE = 1 SV = 2 | | | | | | |
| rRNA/tRNA 2′-O-methyltransferase fibrillarin- | UniProtKB: A6NHQ2 | 2 | 43.8 | | | |
| like protein 1 OS = Homo sapiens GN = FBLL1 | | | | | | |
| PE = 3 SV = 1 | | | | | | |
| RRP15-like protein OS = Homo sapiens | UniProtKB: Q9Y3B9 | 6 | 127.7 | | | |
| GN = RRP15 PE = 1 SV = 2 | | | | | | |
| RuvB-like 1 OS = Homo sapiens GN = RUVBL1 | UniProtKB: Q9Y265 | 5 | 85.46 | | | |
| PE = 1 SV = 1 | | | | | | |
| RuvB-like 2 OS = Homo sapiens GN = RUVBL2 | UniProtKB: Q9Y230 | 4 | 190 | | | |
| PE = 1 SV = 3 | | | | | | |
| S1 RNA-binding domain-containing protein 1 | UniProtKB: Q8N5C6 | 11 | 25.97 | | | |
| OS = Homo sapiens GN = SRBD1 PE = 1 SV = 2 | | | | | | |
| S-adenosylmethionine synthase isoform type-2 | UniProtKB: P31153 | 2 | 77.22 | | | |
| OS = Homo sapiens GN = MAT2A PE = 1 SV = 1 | | | | | | |
| S-formylglutathione hydrolase OS = Homo | UniProtKB: P10768 | 7 | 138.2 | | | |
| sapiens GN = ESD PE = 1 SV = 2 | | | | | | |
| S-methyl-5′-thioadenosine phosphorylase | UniProtKB: Q13126 | 4 | 65.98 | | | |
| OS = Homo sapiens GN = MTAP PE = 1 SV = 2 | | | | | | |
| SAFB-like transcription modulator OS = Homo | UniProtKB: Q9NWH9 | 17 | 71.31 | | | |
| sapiens GN = SLTM PE = 1 SV = 2 | | | | | | |
| SAP domain-containing ribonucleoprotein | UniProtKB: P82979 | 7 | 43.08 | | | |
| OS = Homo sapiens GN = SARNP PE = 1 SV = 3 | | | | | | |
| Septin-2 OS = Homo sapiens GN = SEPT2 PE = 1 | UniProtKB: Q15019 | 5 | 30.12 | | | |
| SV = 1 | | | | | | |
| Septin-7 OS = Homo sapiens GN = SEPT7 PE = 1 | UniProtKB: Q16181 | 8 | 37.3 | | | |
| SV = 2 | | | | | | |
| Septin-9 OS = Homo sapiens GN = SEPT9 PE = 1 | UniProtKB: Q9UHD8 | 10 | 35.92 | | | |
| SV = 2 | | | | | | |
| Serine hydroxymethyltransferase, mitochondrial | UniProtKB: P34897 | 12 | 210 | | | |
| OS = Homo sapiens GN = SHMT2 PE = 1 SV = 3 | | | | | | |
| Serine protease HTRA1 OS = Homo sapiens | UniProtKB: Q92743 | 7 | 28.16 | | | 2 |
| GN = HTRA1 PE = 1 SV = 1 | | | | | | |
| Serine-threonine kinase receptor-associated | UniProtKB: Q9Y3F4 | 7 | 163.3 | | | |
| protein OS = Homo sapiens GN = STRAP PE = 1 | | | | | | |
| SV = 1 | | | | | | |
| Serine/arginine repetitive matrix protein 2 | UniProtKB: Q9UQ35 | 15 | 197.6 | | | 1 |
| OS = Homo sapiens GN = SRRM2 PE = 1 SV = 2 | | | | | | |
| Serine/arginine-rich splicing factor 2 OS = Homo | UniProtKB: Q01130 | 3 | 118.7 | | | |
| sapiens GN = SRSF2 PE = 1 SV = 4 | | | | | | |
| Serine/arginine-rich splicing factor 5 OS = Homo | UniProtKB: Q13243 | 2 | 70.18 | | 1 | |
| sapiens GN = SRSF5 PE = 1 SV = 1 | | | | | | |
| Serine/arginine-rich splicing factor 7 OS = Homo | UniProtKB: Q16629 | 3 | 64.34 | | 2 | |
| sapiens GN = SRSF7 PE = 1 SV = 1 | | | | | | |
| Serine/arginine-rich splicing factor 8 OS = Homo | UniProtKB: Q9BRL6 | 2 | 31.22 | | | |
| sapiens GN = SRSF8 PE = 1 SV = 1 | | | | | | |
| Serine/arginine-rich splicing factor 10 | UniProtKB: O75494 | 7 | 64.39 | | | |
| OS = Homo sapiens GN = SRSF10 PE = 1 SV = 1 | | | | | | |
| Serine/arginine-rich splicing factor 11 | UniProtKB: Q05519 | 3 | 36.03 | | | |
| OS = Homo sapiens GN = SRSF11 PE = 1 SV = 1 | | | | | | |
| Serine/arginine-rich splicing factor 12 | UniProtKB: Q8WXF0 | 3 | 56.57 | | | |
| OS = Homo sapiens GN = SRSF12 PE = 1 SV = 1 | | | | | | |
| Serine/threonine-protein kinase D3 OS = Homo | UniProtKB: O94806 | 1 | 18.64 | | | |
| sapiens GN = PRKD3 PE = 1 SV = 1 | | | | | | |
| Serine/threonine-protein kinase Kist OS = Homo | UniProtKB: Q8TAS1 | 1 | 16.58 | | | |
| sapiens GN = UHMK1 PE = 1 SV = 2 | | | | | | |
| Serine/threonine-protein kinase RIO1 | UniProtKB: Q9BRS2 | 5 | 27.06 | | | |
| OS = Homo sapiens GN = RIOK1 PE = 1 SV = 2 | | | | | | |
| Serine/threonine-protein kinase SRPK1 | UniProtKB: Q96SB4 | 2 | 55.21 | | | |
| OS = Homo sapiens GN = SRPK1 PE = 1 SV = 2 | | | | | | |
| Serine/threonine-protein kinase SRPK2 | UniProtKB: P78362 | 3 | 43.58 | | | |
| OS = Homo sapiens GN = SRPK2 PE = 1 SV = 3 | | | | | | |
| Serine/threonine-protein phosphatase 2A 65 kDa | UniProtKB: P30153 | 10 | 148.2 | | | |
| regulatory subunit A alpha isoform OS = Homo | | | | | | |
| sapiens GN = PPP2R1A PE = 1 SV = 4 | | | | | | |
| Serine/threonine-protein phosphatase 2A | UniProtKB: P62714 | 4 | 51.66 | | | |
| catalytic subunit beta isoform OS = Homo | | | | | | |
| sapiens GN = PPP2CB PE = 1 SV = 1 | | | | | | |
| Serine/threonine-protein phosphatase PGAM5, | UniProtKB: Q96HS1 | 3 | 57.78 | | | |
| mitochondrial OS = Homo sapiens GN = PGAM5 | | | | | | |
| PE = 1 SV = 2 | | | | | | |
| Serpin H1 OS = Homo sapiens GN = SERPINH1 | UniProtKB: P50454 | 7 | 160.6 | | | |
| PE = 1 SV = 2 | | | | | | |
| Serum response factor-binding protein 1 | UniProtKB: Q8NEF9 | 5 | 27.36 | | | |
| OS = Homo sapiens GN = SRFBP1 PE = 1 SV = 1 | | | | | | |
| Seryl-tRNA synthetase, cytoplasmic OS = Homo | UniProtKB: P49591 | 4 | 39.09 | | | |
| sapiens GN = SARS PE = 1 SV = 3 | | | | | | |
| Seryl-tRNA synthetase, mitochondrial | UniProtKB: Q9NP81 | 5 | 44.22 | | | |
| OS = Homo sapiens GN = SARS2 PE = 1 SV = 1 | | | | | | |
| SH3 domain-binding glutamic acid-rich-like | UniProtKB: Q9H299 | 1 | 43.54 | | | |
| protein 3 OS = Homo sapiens GN = SH3BGRL3 | | | | | | |
| PE = 1 SV = 1 | | | | | | |
| Signal recognition particle 9 kDa protein | UniProtKB: P49458 | 1 | 77.98 | | | |
| OS = Homo sapiens GN = SRP9 PE = 1 SV = 2 | | | | | | |
| Signal recognition particle 14 kDa protein | UniProtKB: P37108 | 1 | 29.95 | | | |
| OS = Homo sapiens GN = SRP14 PE = 1 SV = 2 | | | | | | |
| Signal recognition particle receptor subunit | UniProtKB: P08240 | 6 | 60.89 | | | |
| alpha OS = Homo sapiens GN = SRPR PE = 1 | | | | | | |
| SV = 2 | | | | | | |
| Signal recognition particle receptor subunit beta | UniProtKB: Q9Y5M8 | 3 | 68.23 | | | |
| OS = Homo sapiens GN = SRPRB PE = 1 SV = 3 | | | | | | |
| Signal-regulatory protein beta-2 OS = Homo | UniProtKB: Q5JXA9 | 3 | 24.96 | | | |
| sapiens GN = SIRPB2 PE = 2 SV = 1 | | | | | | |
| Single-stranded DNA-binding protein, | UniProtKB: Q04837 | 3 | 56.33 | | | |
| mitochondrial OS = Homo sapiens GN = SSBP1 | | | | | | |
| PE = 1 SV = 1 | | | | | | |
| Small nuclear ribonucleoprotein E OS = Homo | UniProtKB: P62304 | 2 | 108.1 | | | |
| sapiens GN = SNRPE PE = 1 SV = 1 | | | | | | |
| Small nuclear ribonucleoprotein G OS = Homo | UniProtKB: P62308 | 3 | 31.32 | | | |
| sapiens GN = SNRPG PE = 1 SV = 1 | | | | | | |
| Small nuclear ribonucleoprotein Sm D1 | UniProtKB: P62314 | 2 | 49.94 | | | |
| OS = Homo sapiens GN = SNRPD1 PE = 1 SV = 1 | | | | | | |
| Small nuclear ribonucleoprotein-associated | UniProtKB: P63162 | 8 | 42.9 | | | |
| protein N OS = Homo sapiens GN = SNRPN PE = 1 | | | | | | |
| SV = 1 | | | | | | |
| Small nuclear ribonucleoprotein-associated | UniProtKB: P14678 | 3 | 24.82 | | | |
| proteins B and B′ OS = Homo sapiens | | | | | | |
| GN = SNRPB PE = 1 SV = 2 | | | | | | |
| Small ubiquitin-related modifier 3 OS = Homo | UniProtKB: P55854 | 1 | 97.76 | | | |
| sapiens GN = SUMO3 PE = 1 SV = 2 | | | | | | |
| Small ubiquitin-related modifier 4 OS = Homo | UniProtKB: Q6EEV6 | 3 | 59.12 | | | |
| sapiens GN = SUMO4 PE = 1 SV = 2 | | | | | | |
| SODC_HUMAN | SODC_HUMAN | 4 | 116.6 | | | |
| Sodium-coupled monocarboxylate transporter 2 | UniProtKB: Q1EHB4 | 2 | 16.17 | | | |
| OS = Homo sapiens GN = SLC5A12 PE = 2 SV = 2 | | | | | | |
| Sodium/potassium-transporting ATPase subunit | UniProtKB: P54709 | 3 | 49.28 | | | |
| beta-3 OS = Homo sapiens GN = ATP1B3 PE = 1 | | | | | | |
| SV = 1 | | | | | | |
| Solute carrier family 2, facilitated glucose | UniProtKB: Q8TD20 | 2 | 42.32 | | | |
| transporter member 12 OS = Homo sapiens | | | | | | |
| GN = SLC2A12 PE = 2 SV = 1 | | | | | | |
| Solute carrier family 12 member 3 OS = Homo | UniProtKB: P55017 | 3 | 21.68 | | | |
| sapiens GN = SLC12A3 PE = 1 SV = 3 | | | | | | |
| Solute carrier organic anion transporter family | UniProtKB: Q9H2Y9 | 2 | 21.26 | | | |
| member 5A1 OS = Homo sapiens GN = SLCO5A1 | | | | | | |
| PE = 2 SV = 2 | | | | | | |
| Something about silencing protein 10 OS = Homo | UniProtKB: Q9NQZ2 | 3 | 65.06 | | | |
| sapiens GN = UTP3 PE = 1 SV = 1 | | | | | | |
| Sorting nexin-3 OS = Homo sapiens GN = SNX3 | UniProtKB: O60493 | 3 | 23.35 | | | |
| PE = 1 SV = 3 | | | | | | |
| SPATS2-like protein OS = Homo sapiens | UniProtKB: Q9NUQ6 | 2 | 35.31 | | | |
| GN = SPATS2L PE = 1 SV = 2 | | | | | | |
| Spectrin alpha chain, erythrocyte OS = Homo | UniProtKB: P02549 | 7 | 40.26 | | | |
| sapiens GN = SPTA1 PE = 1 SV = 5 | | | | | | |
| Spermatid perinuclear RNA-binding protein | UniProtKB: Q96SI9 | 2 | 35 | | | |
| OS = Homo sapiens GN = STRBP PE = 1 SV = 1 | | | | | | |
| Spermatogenesis-associated protein 7 | UniProtKB: Q9P0W8 | 1 | 19.28 | | 1 | |
| OS = Homo sapiens GN = SPATA7 PE = 2 SV = 3 | | | | | | |
| Spermatogenesis-associated serine-rich protein 2 | UniProtKB: Q86XZ4 | 1 | 43.6 | | | |
| OS = Homo sapiens GN = SPATS2 PE = 1 SV = 1 | | | | | | |
| Spermidine synthase OS = Homo sapiens | UniProtKB: P19623 | 3 | 33.38 | | | |
| GN = SRM PE = 1 SV = 1 | | | | | | |
| Spermine synthase OS = Homo sapiens GN = SMS | UniProtKB: P52788 | 3 | 50.21 | | | |
| PE = 1 SV = 2 | | | | | | |
| Splicing factor 3A subunit 3 OS = Homo sapiens | UniProtKB: Q12874 | 3 | 33.62 | | | |
| GN = SF3A3 PE = 1 SV = 1 | | | | | | |
| Splicing factor 3B subunit 2 OS = Homo sapiens | UniProtKB: Q13435 | 7 | 48.2 | | | |
| GN = SF3B2 PE = 1 SV = 2 | | | | | | |
| Splicing factor 3B subunit 3 OS = Homo sapiens | UniProtKB: Q15393 | 4 | 49.19 | | | |
| GN = SF3B3 PE = 1 SV = 4 | | | | | | |
| Splicing factor 3B subunit 4 OS = Homo sapiens | UniProtKB: Q15427 | 2 | 78.58 | | | |
| GN = SF3B4 PE = 1 SV = 1 | | | | | | |
| SRA stem-loop-interacting RNA-binding | UniProtKB: Q9GZT3 | 2 | 79.73 | | | |
| protein, mitochondrial OS = Homo sapiens | | | | | | |
| GN = SURP PE = 1 SV = 1 | | | | | | |
| SRSF2-interacting protein OS = Homo sapiens | UniProtKB: Q99590 | 5 | 67.97 | | | |
| GN = SRSF2IP PE = 1 SV = 2 | | | | | | |
| Staphylococcal nuclease domain-containing | UniProtKB: Q7KZF4 | 9 | 102.8 | | | 1 |
| protein 1 OS = Homo sapiens GN = SND1 PE = 1 | | | | | | |
| SV = 1 | | | | | | |
| Stomatin-like protein 2 OS = Homo sapiens | UniProtKB: Q9UJZ1 | 2 | 45.85 | | | |
| GN = STOML2 PE = 1 SV = 1 | | | | | | |
| Streptavidin without signal peptide (AAs 1-24) | UniProtKB: CON_P22629 | 4 | 142.3 | | | |
| OS = Streptomyces avidinii PE = 1 SV = 1 | | | | | | |
| Stress-induced-phosphoprotein 1 OS = Homo | UniProtKB: P31948 | 7 | 70.42 | | | |
| sapiens GN = STIP1 PE = 1 SV = 1 | | | | | | |
| Succinyl-CoA ligase [GDP-forming] subunit | UniProtKB: P53597 | 1 | 27.45 | | | |
| alpha, mitochondrial OS = Homo sapiens | | | | | | |
| GN = SUCLG1 PE = 1 SV = 4 | | | | | | |
| SUMO1_HUMAN | SUMO1_HUMAN | 3 | 34.61 | | | |
| Superkiller viralicidic activity 2-like 2 | UniProtKB: P42285 | 6 | 31.06 | | | |
| OS = Homo sapiens GN = SKIV2L2 PE = 1 SV = 3 | | | | | | |
| Suppressor of G2 allele of SKP1 homolog | UniProtKB: Q9Y2Z0 | 5 | 45.99 | | | |
| OS = Homo sapiens GN = SUGT1 PE = 1 SV = 3 | | | | | | |
| Suppressor of SWI4 1 homolog OS = Homo | UniProtKB: Q9NQ55 | 5 | 61.62 | | 1 | |
| sapiens GN = PPAN PE = 1 SV = 1 | | | | | | |
| Survival motor neuron protein OS = Homo | UniProtKB: Q16637 | 2 | 48.1 | | | |
| sapiens GN = SMN1 PE = 1 SV = 1 | | | | | | |
| SYH_HUMAN | SYH_HUMAN | 5 | 33.45 | | | |
| Synaptic vesicle membrane protein VAT-1 | UniProtKB: Q99536 | 7 | 62.8 | | | |
| homolog OS = Homo sapiens GN = VAT1 PE = 1 | | | | | | |
| SV = 2 | | | | | | |
| T-complex protein 1 subunit alpha OS = Homo | UniProtKB: P17987 | 16 | 497.1 | | | |
| sapiens GN = TCP1 PE = 1 SV = 1 | | | | | | |
| T-complex protein 1 subunit beta OS = Homo | UniProtKB: P78371 | 4 | 94.02 | | | |
| sapiens GN = CCT2 PE = 1 SV = 4 | | | | | | |
| T-complex protein 1 subunit delta OS = Homo | UniProtKB: P50991 | 2 | 69.12 | | | |
| sapiens GN = CCT4 PE = 1 SV = 4 | | | | | | |
| T-complex protein 1 subunit epsilon OS = Homo | UniProtKB: P48643 | 22 | 341.8 | | | |
| sapiens GN = CCT5 PE = 1 SV = 1 | | | | | | |
| T-complex protein 1 subunit eta OS = Homo | UniProtKB: Q99832 | 2 | 43.64 | | | |
| sapiens GN = CCT7 PE = 1 SV = 2 | | | | | | |
| T-complex protein 1 subunit gamma OS = Homo | UniProtKB: P49368 | 8 | 151 | | | |
| sapiens GN = CCT3 PE = 1 SV = 4 | | | | | | |
| T-complex protein 1 subunit theta OS = Homo | UniProtKB: P50990 | 7 | 179.1 | | | |
| sapiens GN = CCT8 PE = 1 SV = 4 | | | | | | |
| T-complex protein 1 subunit zeta OS = Homo | UniProtKB: P40227 | 7 | 52.98 | | | |
| sapiens GN = CCT6A PE = 1 SV = 3 | | | | | | |
| T-complex protein 1 subunit zeta-2 OS = Homo | UniProtKB: Q92526 | 9 | 112.1 | | | |
| sapiens GN = CCT6B PE = 1 SV = 4 | | | | | | |
| Target of EGR1 protein 1 OS = Homo sapiens | UniProtKB: Q96GM8 | 4 | 20.88 | | | |
| GN = TOE1 PE = 1 SV = 1 | | | | | | |
| TATA-binding protein-associated factor 2N | UniProtKB: Q92804 | 3 | 54.84 | | | |
| OS = Homo sapiens GN = TAF15 PE = 1 SV = 1 | | | | | | |
| Tax_Id = 10090 Gene Symbol = Krt20 Keratin, | UniProtKB: CON_Q9D312 | 2 | 26.68 | | | |
| type I cytoskeletal 20 | | | | | | |
| Tax_Id = 10090 Gene Symbol = Krt72 Type-II | UniProtKB: CON_Q6IME9 | 5 | 40.06 | | | |
| keratin Kb35 | | | | | | |
| THIO_HUMAN | THIO_HUMAN | 3 | 60.04 | | | |
| Thioredoxin domain-containing protein 5 | UniProtKB: Q8NBS9 | 8 | 50.89 | | | |
| OS = Homo sapiens GN = TXNDC5 PE = 1 SV = 2 | | | | | | |
| Thioredoxin domain-containing protein 11 | UniProtKB: Q6PKC3 | 1 | 37.9 | | | |
| OS = Homo sapiens GN = TXNDC11 PE = 1 SV = 2 | | | | | | |
| Thioredoxin domain-containing protein 17 | UniProtKB: Q9BRA2 | 3 | 26.73 | | | |
| OS = Homo sapiens GN = TXNDC17 PE = 1 SV = 1 | | | | | | |
| Thioredoxin OS = Homo sapiens GN = TXN PE = 1 | UniProtKB: P10599 | 4 | 58.04 | | | |
| SV = 3 | | | | | | |
| Thioredoxin-dependent peroxide reductase, | UniProtKB: P30048 | 2 | 58.26 | | | |
| mitochondrial OS = Homo sapiens GN = PRDX3 | | | | | | |
| PE = 1 SV = 3 | | | | | | |
| Thioredoxin-like protein 4A OS = Homo sapiens | UniProtKB: P83876 | 1 | 43.94 | | | |
| GN = TXNL4A PE = 1 SV = 1 | | | | | | |
| Threonyl-tRNA synthetase, cytoplasmic | UniProtKB: P26639 | 5 | 41.54 | | | |
| OS = Homo sapiens GN = TARS PE = 1 SV = 3 | | | | | | |
| Thymidylate kinase OS = Homo sapiens | UniProtKB: P23919 | 9 | 201.5 | | | |
| GN = DTYMK PE = 1 SV = 4 | | | | | | |
| Thyroid transcription factor 1-associated protein | UniProtKB: Q9P031 | 6 | 25.61 | | | |
| 26 OS = Homo sapiens GN = CCDC59 PE = 1 | | | | | | |
| SV = 2 | | | | | | |
| Tigger transposable element-derived protein 1 | UniProtKB: Q96MW7 | 8 | 22.42 | | | 1 |
| OS = Homo sapiens GN = TIGD1 PE = 2 SV = 1 | | | | | | |
| Titin OS = Homo sapiens GN = TTN PE = 1 SV = 2 | UniProtKB: Q8WZ42 | 61 | 16.46 | | | 1 |
| Toll-like receptor 10 OS = Homo sapiens | UniProtKB: Q9BXR5 | 3 | 32.05 | | 1 | 1 |
| GN = TLR10 PE = 1 SV = 2 | | | | | | |
| Transaldolase OS = Homo sapiens GN = TALDO1 | UniProtKB: P37837 | 12 | 148.3 | | | |
| PE = 1 SV = 2 | | | | | | |
| Transcription elongation factor A protein 1 | UniProtKB: P23193 | 9 | 30.2 | | | |
| OS = Homo sapiens GN = TCEA1 PE = 1 SV = 2 | | | | | | |
| Transcription elongation factor B polypeptide 1 | UniProtKB: Q15369 | 1 | 47.42 | | | |
| OS = Homo sapiens GN = TCEB1 PE = 1 SV = 1 | | | | | | |
| Transcription elongation factor B polypeptide 2 | UniProtKB: Q15370 | 1 | 35.03 | | | |
| OS = Homo sapiens GN = TCEB2 PE = 1 SV = 1 | | | | | | |
| Transcription factor BTF3 homolog 2 | UniProtKB: Q13891 | 5 | 58.27 | | | |
| OS = Homo sapiens GN = BTF3L2 PE = 2 SV = 1 | | | | | | |
| Transcription factor BTF3 OS = Homo sapiens | UniProtKB: P20290 | 8 | 171.3 | | | |
| GN = BTF3 PE = 1 SV = 1 | | | | | | |
| Transcription factor HIVEP2 OS = Homo sapiens | UniProtKB: P31629 | 3 | 35.74 | | | |
| GN = HIVEP2 PE = 1 SV = 2 | | | | | | |
| Transcription intermediary factor 1-beta | UniProtKB: Q13263 | 3 | 29.43 | | | 1 |
| OS = Homo sapiens GN = TRIM28 PE = 1 SV = 5 | | | | | | |
| Transcriptional regulator ERG OS = Homo | UniProtKB: P11308 | 2 | 19.52 | | | 1 |
| sapiens GN = ERG PE = 1 SV = 2 | | | | | | |
| Transferrin receptor protein 1 OS = Homo | UniProtKB: P02786 | 13 | 39.37 | | | |
| sapiens GN = TFRC PE = 1 SV = 2 | | | | | | |
| Transformer-2 protein homolog alpha | UniProtKB: Q13595 | 3 | 156.9 | | 8 | |
| OS = Homo sapiens GN = TRA2A PE = 1 SV = 1 | | | | | | |
| Transformer-2 protein homolog beta OS = Homo | UniProtKB: P62995 | 5 | 63.9 | | 2 | |
| sapiens GN = TRA2B PE = 1 SV = 1 | | | | | | |
| Transgelin-2 OS = Homo sapiens GN = TAGLN2 | UniProtKB: P37802 | 7 | 78.78 | | | |
| PE = 1 SV = 3 | | | | | | |
| Transitional endoplasmic reticulum ATPase | UniProtKB: P55072 | 19 | 302.5 | | | |
| OS = Homo sapiens GN = VCP PE = 1 SV = 4 | | | | | | |
| Transketolase OS = Homo sapiens GN = TKT | UniProtKB: P29401 | 3 | 24.96 | | | |
| PE = 1 SV = 3 | | | | | | |
| Transmembrane emp24 domain-containing | UniProtKB: Q9BVK6 | 5 | 33.42 | | | |
| protein 9 OS = Homo sapiens GN = TMED9 PE = 1 | | | | | | |
| SV = 2 | | | | | | |
| Transmembrane emp24 domain-containing | UniProtKB: P49755 | 3 | 62.64 | | | |
| protein 10 OS = Homo sapiens GN = TMED10 | | | | | | |
| PE = 1 SV = 2 | | | | | | |
| Transmembrane glycoprotein NMB OS = Homo | UniProtKB: Q14956 | 2 | 26.19 | | 1 | |
| sapiens GN = GPNMB PE = 1 SV = 2 | | | | | | |
| Transportin-1 OS = Homo sapiens GN = TNP01 | UniProtKB: Q92973 | 7 | 63.45 | | | |
| PE = 1 SV = 2 | | | | | | |
| Trifunctional purine biosynthetic protein | UniProtKB: P22102 | 10 | 150.9 | | | |
| adenosine-3 OS = Homo sapiens GN = GART | | | | | | |
| PE = 1 SV = 1 | | | | | | |
| tRNA dimethylallyltransferase, mitochondrial | UniProtKB: Q9H3H1 | 6 | 22.11 | | 2 | |
| OS = Homo sapiens GN = TRIT1 PE = 1 SV = 1 | | | | | | |
| tRNA methyltransferase 112 homolog | UniProtKB: Q9UI30 | 2 | 48.77 | | | |
| OS = Homo sapiens GN = TRMT112 PE = 1 SV = 1 | | | | | | |
| tRNA-specific adenosine deaminase 1 | UniProtKB: Q9BUB4 | 3 | 34.55 | | 1 | |
| OS = Homo sapiens GN = ADAT1 PE = 2 SV = 1 | | | | | | |
| tRNA-splicing endonuclease subunit Sen2 | UniProtKB: Q8NCE0 | 4 | 22.23 | | | |
| OS = Homo sapiens GN = TSEN2 PE = 1 SV = 2 | | | | | | |
| Tryptophanyl-tRNA synthetase, cytoplasmic | UniProtKB: P23381 | 6 | 66.64 | | | |
| OS = Homo sapiens GN = WARS PE = 1 SV = 2 | | | | | | |
| Tubulin-specific chaperone A OS = Homo | UniProtKB: O75347 | 9 | 158.3 | | | |
| sapiens GN = TBCA PE = 1 SV = 3 | | | | | | |
| Tudor domain-containing protein 3 OS = Homo | UniProtKB: Q9H7E2 | 5 | 35.18 | | | |
| sapiens GN = TDRD3 PE = 1 SV = 1 | | | | | | |
| Tumor protein D52 OS = Homo sapiens | UniProtKB: P55327 | 7 | 65.35 | | | |
| GN = TPD52 PE = 1 SV = 2 | | | | | | |
| Tumor protein D54 OS = Homo sapiens | UniProtKB: O43399 | 5 | 88.33 | | | |
| GN = TPD52L2 PE = 1 SV = 2 | | | | | | |
| U1 small nuclear ribonucleoprotein 70 kDa | UniProtKB: P08621 | 1 | 30.54 | | | |
| OS = Homo sapiens GN = SNRNP70 PE = 1 SV = 2 | | | | | | |
| U1 small nuclear ribonucleoprotein A | UniProtKB: P09012 | 3 | 32.62 | | | |
| OS = Homo sapiens GN = SNRPA PE = 1 SV = 3 | | | | | | |
| U2 small nuclear ribonucleoprotein B″ | UniProtKB: P08579 | 4 | 52.32 | | | |
| OS = Homo sapiens GN = SNRPB2 PE = 1 SV = 1 | | | | | | |
| U3 small nucleolar ribonucleoprotein protein | UniProtKB: O00566 | 3 | 67.31 | | | |
| MMP10 OS = Homo sapiens GN = MPHOSPH10 | | | | | | |
| PE = 1 SV = 2 | | | | | | |
| U3 small nucleolar RNA-associated protein 14 | UniProtKB: Q9BVJ6 | 4 | 34.78 | | | |
| homolog A OS = Homo sapiens GN = UTP14A | | | | | | |
| PE = 1 SV = 1 | | | | | | |
| U3 small nucleolar RNA-associated protein 14 | UniProtKB: Q5TAP6 | 11 | 28.04 | | | |
| homolog C OS = Homo sapiens GN = UTP14C | | | | | | |
| PE = 1 SV = 1 | | | | | | |
| U3 small nucleolar RNA-associated protein 18 | UniProtKB: Q9Y5J1 | 3 | 52.2 | | | |
| homolog OS = Homo sapiens GN = UTP18 PE = 1 | | | | | | |
| SV = 3 | | | | | | |
| U3 small nucleolar RNA-interacting protein 2 | UniProtKB: O43818 | 3 | 72.89 | | 1 | |
| OS = Homo sapiens GN = RRP9 PE = 1 SV = 1 | | | | | | |
| U4/U6 small nuclear ribonucleoprotein Prp3 | UniProtKB: O43395 | 6 | 58.75 | | | |
| OS = Homo sapiens GN = PRPF3 PE = 1 SV = 2 | | | | | | |
| U4/U6 small nuclear ribonucleoprotein Prp4 | UniProtKB: O43172 | 2 | 56.41 | | | |
| OS = Homo sapiens GN = PRPF4 PE = 1 SV = 2 | | | | | | |
| U4/U6.U5 tri-snRNP-associated protein 1 | UniProtKB: O43290 | 17 | 638.5 | | | |
| OS = Homo sapiens GN = SART1 PE = 1 SV = 1 | | | | | | |
| U4/U6.U5 tri-snRNP-associated protein 2 | UniProtKB: Q53GS9 | 1 | 23.32 | | 4 | 1 |
| OS = Homo sapiens GN = USP39 PE = 1 SV = 2 | | | | | | |
| U5 small nuclear ribonucleoprotein 40 kDa | UniProtKB: Q96DI7 | 1 | 66.32 | | | |
| protein OS = Homo sapiens GN = SNRNP40 PE = 1 | | | | | | |
| SV = 1 | | | | | | |
| U5 small nuclear ribonucleoprotein 200 kDa | UniProtKB: O75643 | 13 | 365.6 | | | 1 |
| helicase OS = Homo sapiens GN = SNRNP200 | | | | | | |
| PE = 1 SV = 2 | | | | | | |
| U6 snRNA-associated Sm-like protein LSm2 | UniProtKB: Q9Y333 | 2 | 44.08 | | | |
| OS = Homo sapiens GN = LSM2 PE = 1 SV = 1 | | | | | | |
| U6 snRNA-associated Sm-like protein LSm3 | UniProtKB: P62310 | 2 | 41.03 | | | |
| OS = Homo sapiens GN = LSM3 PE = 1 SV = 2 | | | | | | |
| U6 snRNA-associated Sm-like protein LSm4 | UniProtKB: Q9Y4Z0 | 3 | 42.76 | | | |
| OS = Homo sapiens GN = LSM4 PE = 1 SV = 1 | | | | | | |
| U6 snRNA-associated Sm-like protein LSm6 | UniProtKB: P62312 | 2 | 62.56 | | | |
| OS = Homo sapiens GN = LSM6 PE = 1 SV = 1 | | | | | | |
| U6 snRNA-associated Sm-like protein LSm7 | UniProtKB: Q9UK45 | 1 | 44 | | | |
| OS = Homo sapiens GN = LSM7 PE = 1 SV = 1 | | | | | | |
| UBE2I_HUMAN | UBE2I_HUMAN | 5 | 54.33 | | | |
| Ubiquilin-4 OS = Homo sapiens GN = UBQLN4 | UniProtKB: Q9NRR5 | 3 | 38.04 | | | |
| PE = 1 SV = 2 | | | | | | |
| Ubiquitin carboxyl-terminal hydrolase 5 | UniProtKB: P45974 | 3 | 86.74 | | | |
| OS = Homo sapiens GN = USP5 PE = 1 SV = 2 | | | | | | |
| Ubiquitin carboxyl-terminal hydrolase 37 | UniProtKB: Q86T82 | 7 | 21.46 | | 1 | 1 |
| OS = Homo sapiens GN = USP37 PE = 1 SV = 2 | | | | | | |
| Ubiquitin-associated protein 2-like OS = Homo | UniProtKB: Q14157 | 4 | 42.92 | | | |
| sapiens GN = UBAP2L PE = 1 SV = 2 | | | | | | |
| Ubiquitin-conjugating enzyme E2 D3 | UniProtKB: P61077 | 1 | 38.54 | | | |
| OS = Homo sapiens GN = UBE2D3 PE = 1 SV = 1 | | | | | | |
| Ubiquitin-like modifier-activating enzyme 1 | UniProtKB: P22314 | 18 | 321.9 | | | |
| OS = Homo sapiens GN = UBA1 PE = 1 SV = 3 | | | | | | |
| UBX domain-containing protein 2A OS = Homo | UniProtKB: P68543 | 3 | 33.28 | | | |
| sapiens GN = UBXN2A PE = 2 SV = 1 | | | | | | |
| UDP-glucuronosyltransferase 2B7 OS = Homo | UniProtKB: P16662 | 1 | 21.35 | | 1 | |
| sapiens GN = UGT2B7 PE = 1 SV = 1 | | | | | | |
| UHRF1-binding protein 1-like OS = Homo | UniProtKB: A0JNW5 | 4 | 28.64 | | | |
| sapiens GN = UHRF1BP1L PE = 1 SV = 2 | | | | | | |
| Uncharacterized protein C1orf103 OS = Homo | UniProtKB: Q5T3J3 | 10 | 17.02 | | | |
| sapiens GN = C1orf103 PE = 1 SV = 1 | | | | | | |
| Uncharacterized protein C1orf106 OS = Homo | UniProtKB: Q3KP66 | 3 | 30.82 | | | |
| sapiens GN = C1orf106 PE = 2 SV = 1 | | | | | | |
| Uncharacterized protein C2orf42 OS = Homo | UniProtKB: Q9NWW7 | 9 | 29.5 | | 2 | 1 |
| sapiens GN = C2orf42 PE = 2 SV = 1 | | | | | | |
| Uncharacterized protein C8orf42 OS = Homo | UniProtKB: Q86YL5 | 1 | 31.15 | | | |
| sapiens GN = C8orf42 PE = 2 SV = 2 | | | | | | |
| Uncharacterized protein C10orf71 OS = Homo | UniProtKB: Q711Q0 | 5 | 18.88 | | | |
| sapiens GN = C10orf71 PE = 2 SV = 2 | | | | | | |
| Uncharacterized protein C12orf43 OS = Homo | UniProtKB: Q96C57 | 2 | 108.5 | | | |
| sapiens GN = C12orf43 PE = 1 SV = 2 | | | | | | |
| Uncharacterized protein C14orf43 OS = Homo | UniProtKB: Q6PJG2 | 2 | 34.72 | | | |
| sapiens GN = C14orf43 PE = 1 SV = 2 | | | | | | |
| Uncharacterized protein C14orf166B OS = Homo | UniProtKB: Q0VAA2 | 1 | 32.67 | | | |
| sapiens GN = C14orf166B PE = 2 SV = 2 | | | | | | |
| Uncharacterized protein C17orf47 OS = Homo | UniProtKB: Q8NEP4 | 1 | 22.87 | | | |
| sapiens GN = C17orf47 PE = 1 SV = 3 | | | | | | |
| Uncharacterized protein C19orf34 OS = Homo | UniProtKB: Q8NCQ2 | 2 | 30.12 | | | |
| sapiens GN = C19orf34 PE = 2 SV = 2 | | | | | | |
| Uncharacterized protein C20orf194 OS = Homo | UniProtKB: Q5TEA3 | 4 | 20.54 | | 3 | |
| sapiens GN = C20orf194 PE = 2 SV = 1 | | | | | | |
| Uncharacterized protein DKFZp781G0119 | UniProtKB: Q68DL7 | 5 | 17.09 | | | |
| OS = Homo sapiens PE = 2 SV = 2 | | | | | | |
| Uncharacterized protein KIAA1683 OS = Homo | UniProtKB: Q9H0B3 | 21 | 25.42 | | | 1 |
| sapiens GN = KIAA1683 PE = 1 SV = 1 | | | | | | |
| Uncharacterized protein KIAA2026 OS = Homo | UniProtKB: Q5HYC2 | 3 | 30.08 | | | |
| sapiens GN = KIAA2026 PE = 2 SV = 2 | | | | | | |
| Usher syndrome type-1C protein-binding | UniProtKB: Q8N6Y0 | 1 | 32.53 | | | |
| protein 1 OS = Homo sapiens GN = USHBP1 | | | | | | |
| PE = 1 SV = 1 | | | | | | |
| Valyl-tRNA synthetase OS = Homo sapiens | UniProtKB: P26640 | 12 | 80.07 | | | |
| GN = VARS PE = 1 SV = 4 | | | | | | |
| Vam6/Vps39-like protein OS = Homo sapiens | UniProtKB: Q96JC1 | 1 | 27.18 | | | |
| GN = VPS39 PE = 1 SV = 2 | | | | | | |
| Vinculin OS = Homo sapiens GN = VCL PE = 1 | UniProtKB: P18206 | 17 | 34.36 | | | |
| SV = 4 | | | | | | |
| Voltage-dependent anion-selective channel | UniProtKB: P21796 | 3 | 52.37 | | | |
| protein 1 OS = Homo sapiens GN = VDAC1 PE = 1 | | | | | | |
| SV = 2 | | | | | | |
| Voltage-dependent anion-selective channel | UniProtKB: P45880 | 3 | 93.34 | | | |
| protein 2 OS = Homo sapiens GN = VDAC2 PE = 1 | | | | | | |
| SV = 2 | | | | | | |
| Voltage-dependent anion-selective channel | UniProtKB: Q9Y277 | 3 | 50.67 | | | |
| protein 3 OS = Homo sapiens GN = VDAC3 PE = 1 | | | | | | |
| SV = 1 | | | | | | |
| WAS protein family homolog 2 OS = Homo | UniProtKB: Q6VEQ5 | 4 | 36.15 | | | |
| sapiens GN = WASH2P PE = 2 SV = 2 | | | | | | |
| WD repeat-containing protein 1 OS = Homo | UniProtKB: O75083 | 11 | 50.98 | | | |
| sapiens GN = WDR1 PE = 1 SV = 4 | | | | | | |
| WD repeat-containing protein 33 OS = Homo | UniProtKB: Q9C0J8 | 3 | 19.52 | | | |
| sapiens GN = WDR33 PE = 1 SV = 2 | | | | | | |
| WD repeat-containing protein 43 OS = Homo | UniProtKB: Q15061 | 1 | 37.12 | | | |
| sapiens GN = WDR43 PE = 1 SV = 3 | | | | | | |
| WD repeat-containing protein 46 OS = Homo | UniProtKB: O15213 | 6 | 48.38 | | | |
| sapiens GN = WDR46 PE = 1 SV = 3 | | | | | | |
| WD repeat-containing protein 74 OS = Homo | UniProtKB: Q6RFH5 | 3 | 71.89 | | | 1 |
| sapiens GN = WDR74 PE = 1 SV = 1 | | | | | | |
| WD repeat-containing protein 81 OS = Homo | UniProtKB: Q562E7 | 3 | 13.72 | | | |
| sapiens GN = WDR81 PE = 2 SV = 1 | | | | | | |
| WD repeat-containing protein 87 OS = Homo | UniProtKB: Q6ZQQ6 | 44 | 36.09 | | | |
| sapiens GN = WDR87 PE = 2 SV = 3 | | | | | | |
| X-ray repair cross-complementing protein 5 | UniProtKB: P13010 | 3 | 56.19 | | | |
| OS = Homo sapiens GN = XRCC5 PE = 1 SV = 3 | | | | | | |
| YTH domain-containing protein 1 OS = Homo | UniProtKB: Q96MU7 | 5 | 70.6 | | 1 | |
| sapiens GN = YTHDC1 PE = 1 SV = 3 | | | | | | |
| Zinc finger and BTB domain-containing protein | UniProtKB: O95625 | 7 | 31.65 | | 7 | 1 |
| 11 OS = Homo sapiens GN = ZBTB11 PE = 1 | | | | | | |
| SV = 2 | | | | | | |
| Zinc finger CCCH domain-containing protein 13 | UniProtKB: Q5T200 | 5 | 65.46 | | | |
| OS = Homo sapiens GN = ZC3H13 PE = 1 SV = 1 | | | | | | |
| Zinc finger CCCH domain-containing protein 15 | UniProtKB: Q8WU90 | 16 | 141.8 | | | |
| OS = Homo sapiens GN = ZC3H15 PE = 1 SV = 1 | | | | | | |
| Zinc finger CCCH-type with G patch domain- | UniProtKB: Q8N5A5 | 6 | 69.64 | | | 1 |
| containing protein OS = Homo sapiens | | | | | | |
| GN = ZGPAT PE = 1 SV = 3 | | | | | | |
| Zinc finger protein 292 OS = Homo sapiens | UniProtKB: O60281 | 13 | 15.87 | | | |
| GN = ZNF292 PE = 1 SV = 3 | | | | | | |
| Zinc finger protein 326 OS = Homo sapiens | UniProtKB: Q5BKZ1 | 6 | 80.66 | | | |
| GN = ZNF326 PE = 1 SV = 2 | | | | | | |
| Zinc finger protein 729 OS = Homo sapiens | UniProtKB: A6NN14 | 2 | 17.17 | | | |
| GN = ZNF729 PE = 2 SV = 3 | | | | | | |
| Zinc finger protein 768 OS = Homo sapiens | UniProtKB: Q9H5H4 | 2 | 48.1 | | | |
| GN = ZNF768 PE = 1 SV = 2 | | | | | | |
| Zinc finger protein 771 OS = Homo sapiens | UniProtKB: Q7L3S4 | 1 | 37.12 | | | |
| GN = ZNF771 PE = 1 SV = 1 | | | | | | |
| Zinc finger protein 784 OS = Homo sapiens | UniProtKB: Q8NCA9 | 3 | 24.06 | | | |
| GN = ZNF784 PE = 2 SV = 1 | | | | | | |
| Zinc finger protein 800 OS = Homo sapiens | UniProtKB: Q2TB10 | 10 | 21.07 | | | |
| GN = ZNF800 PE = 1 SV = 1 | | | | | | |
| Zinc finger protein GLI2 OS = Homo sapiens | UniProtKB: P10070 | 2 | 27.03 | | | |
| GN = GLI2 PE = 1 SV = 4 | | | | | | |
| Zinc finger with UFM1-specific peptidase | UniProtKB: Q96AP4 | 5 | 37.49 | | | |
| domain protein OS = Homo sapiens GN = ZUFSP | | | | | | |
| PE = 1 SV = 1 | | | | | | |

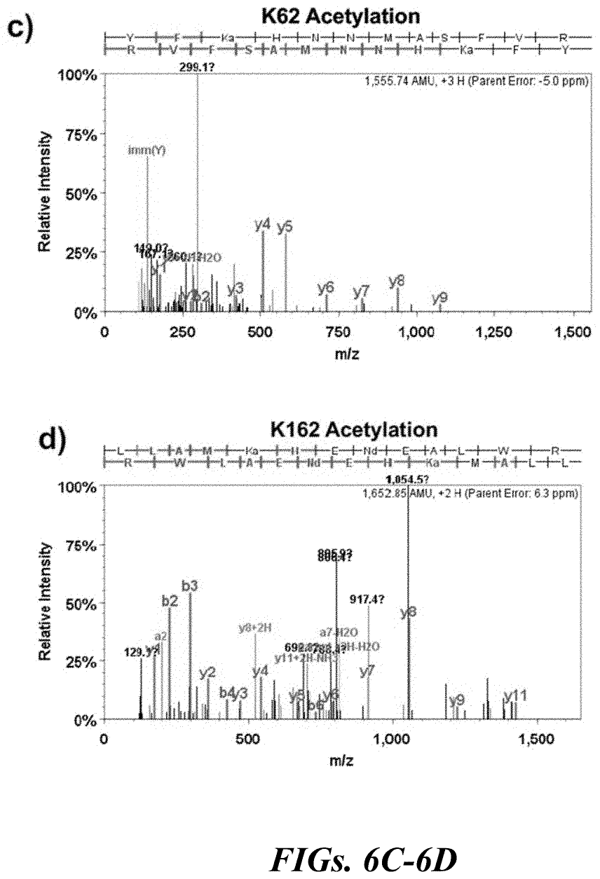

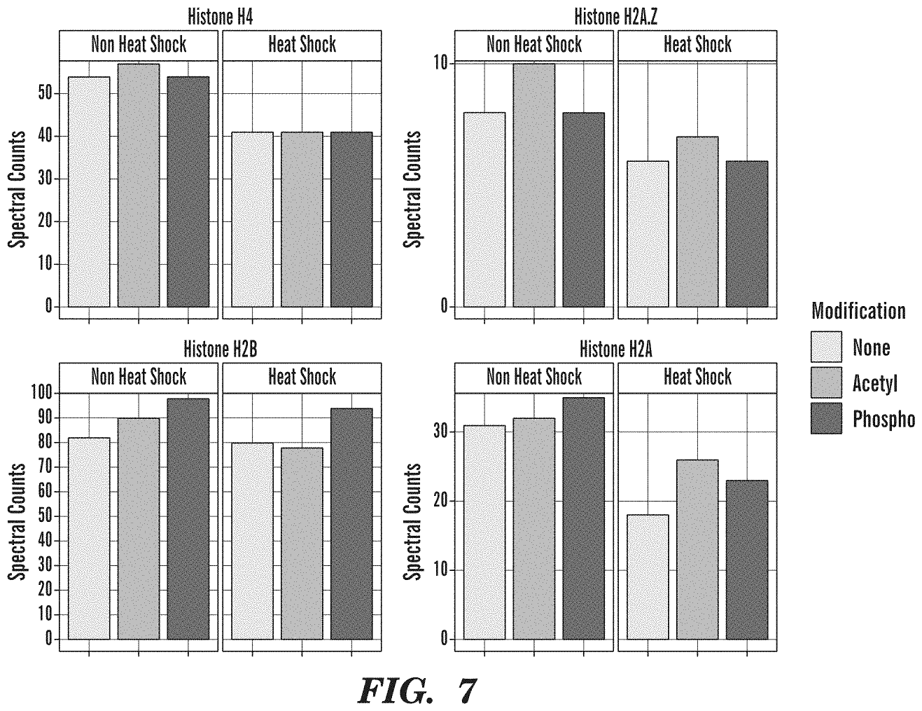

MS1 quantification in Skyline (Pino et al., “The Skyline Ecosystem: Informatics for Quantitative Mass Spectrometry Proteomics,” Mass Spectrom. Rev. 39:229-244 (2017), which is hereby incorporated by reference in its entirety) was used to validate the quality of the modified peptides (FIG. 6A). Scaffold PTM (Vincent-Maloney et al., “Probabilistically Assigning Sites of Protein Modification with Scaffold PTM,” J. Biomol. Tech. 22:S36-S37 (2011), which is hereby incorporated by reference in its entirety) and the MS2 spectra verified the PTM assignments on HSF1 peptides (FIGS. 6B-6D). In addition, two acetylated lysine residues were identified, K62 and K162, that were not reported previously (FIG. 4B, FIG. 6C-6D). AptA-MS also identified PTMs (acetylated and phosphorylated residues) on HSF1-associated histones in both NHS and HS conditions (FIG. 7, Table 2). These PTMs were identified consistently but with relatively low spectral counts, providing an explanation for why they were not identified previously in the presence of abundant interfering signal from contaminating peptides. This association of HSF1 with acetylated histones is consistent with the observation that HSF1 preferentially binds to sites in open chromatin, in particular those that contain acetylated histones (Vihervaara et al., “Transcriptional response to stress is pre-wired by promoter and enhancer architecture,” Nat. Commun. 8:255 (2017); Guertin et al., “Mechanisms by Which Transcription Factors Gain Access to Target Sequence Elements in Chromatin,” Curr. Opin. Genet. Dev. 23:116-123 (2013), which are hereby incorporated by reference in their entirety).

### Example 4—Multiple Classes of Proteins Show Increased HSF1 Interaction in Heat Shock Cells

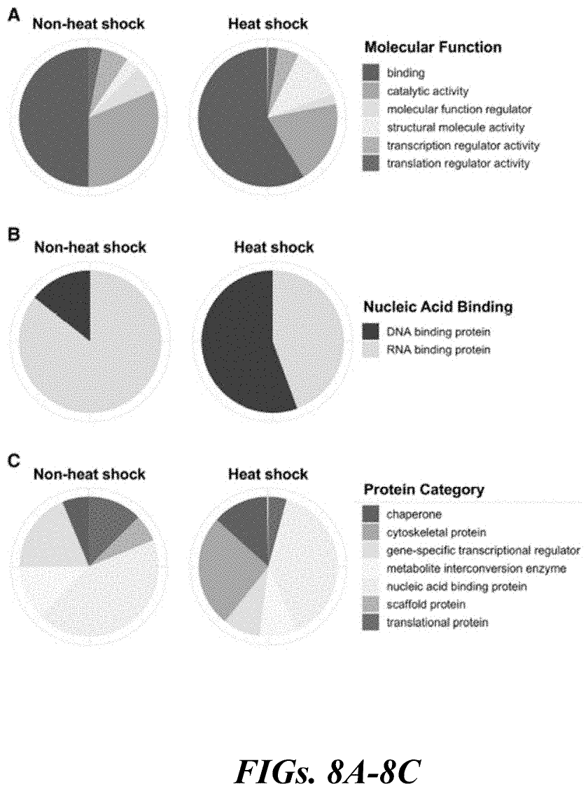

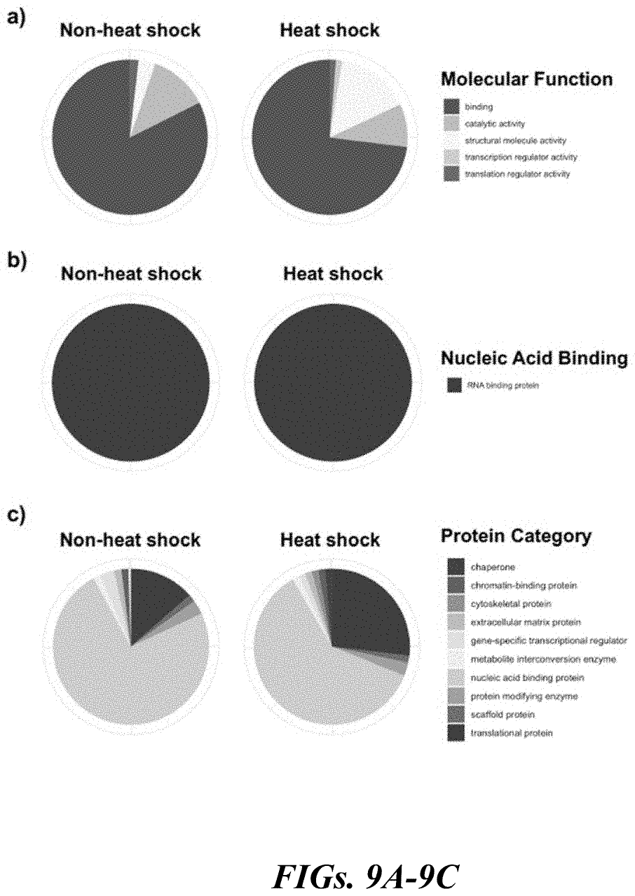

32 proteins were identified in NHS cells expressing HSF1-GFP that are enriched compared to GFP expressing cells upon pull-down with the GFP-aptamer based on a Fisher's exact test using a P value cutoff of <0.05. In the same pulldowns from the HS cells, 42 enriched proteins were identified. Earlier interaction studies have not identified histones as co-precipitants in HSF1 immunoprecipitations or affinity purifications, potentially due to large variation in protein abundances in immunoprecipitated samples. Two histone proteins were found to be enriched in NHS cells, whereas six histone proteins were enriched in HS cells (Table 5). This illustrates that the chromatin landscape changes in response to HS, and that these changes are associated with HSF1-containing complexes. This fact is further demonstrated by the increase in proteins with ‘binding’ activity in HSF1 pull-downs from HS cells, as shown by GO analysis (FIG. 8A). This is in concordance with a dramatic increase in the proportion of DNA binding proteins upon HS (FIG. 8B).

AptA-MS recapitulated published observations that HSF1 engages with chaperone proteins such as HSP70. It is found that AptA-MS pull-downs contain a large proportion of chaperone proteins in NHS and HS conditions (FIG. 4A). Interestingly, a higher proportion of chaperone proteins is seen co-purifying with HSF1 after HS, which suggests that chaperones might have a dynamic role in both maintaining inactive HSF1 during NHS and modulating the level of activation after HS (FIG. 8C).

Cytoskeletal reorganization and increased levels of transcription of cytoskeletal proteins have been observed after HS in some cell types, but their role and possible interplay with active HSF1 have not been documented (Walter et al., “Heat Shock Causes the Collapse of the Intermediate Filament Cytoskeleton in Drosophila Embryos,” Dev. Genet. 11:270-279 (1990), which is hereby incorporated by reference in its entirety). Upon HS an increase in HSF1-associated proteins with ‘structural molecule activity’ (FIG. 8A) is observed, which includes cytoskeletal proteins. Indeed, it is identified that cytoskeletal proteins are enriched upon GFP aptamer pulldown in the HSF1-GFP tagged HS cells, indicating that cytoskeletal protein networks may be connected more directly to modulating HSF1 activity than previously thought (FIG. 8C). Microtubules are particularly enriched as interactors with HSF1 following HS.

### Example 5—RNA Affinity Contaminants Experimental Repository (RACER) Provides a Database of Non-Specifically Binding Proteins for AptA-MS Experiments

The rigorously controlled study identified proteins that bind to free GFP and to a control (NELF)-aptamer selected for Drosophila NELF-E (dNELF-E) (Pagano et al., “Defining NELF-E RNA Binding in HIV-1 and Promoter-proximal Pause Regions,” PLoS Genet. 10:e1004090 (2014); Tome et al., “Comprehensive Analysis of RNA-protein Interactions by High-throughput Sequencing-RNA Affinity Profiling,” Nat. Methods 11:683-688 (2014), which are hereby incorporated by reference in their entirety) with no predicted binding specificity in humans. A Fisher's exact test was used to statistically compare proteins enriched by the GFP-aptamer in cells expressing free GFP compared to cells expressing HSF1-GFP (Table 3).

TABLE 3

| | | P Value | |
|---|---|---|---|
| | | (Fishers | Quantitative |
| Protein | Accession Number | Exact Test) | Profile |
| Nucleolar RNA helicase 2 OS = Homo sapiens GN = DDX21 PE = 1 | UniProtKB: Q9NR30 | 0.00085 | EGFP NHS GFP |
| SV = 5 | | | high, HSF1 NHS |
| | | | GFP low |
| Green fluorescent protein OS = Aequorea victoria OX = 6100 | GFP_AEQVI | 0.00014 | EGFP NHS GFP |
| GN = GFP PE = 1 SV = 1 | | | high, HSF1 NHS |
| | | | GFP low |
| Bc1-2-associated transcription factor 1 OS = Homo sapiens | UniProtKB: Q9NYF8 | <0.00010 | EGFP NHS GFP |
| GN = BCLAF1 PE = 1 SV = 2 | | | high, HSF1 NHS |
| | | | GFP low |
| Thyroid hormone receptor-associated protein 3 OS = Homo sapiens | UniProtKB: Q9Y2W1 | <0.00010 | EGFP NHS GFP |
| GN = THRAP3 PE = 1 SV = 2 | | | high, HSF1 NHS |
| | | | GFP low |
| Polyadenylate-binding protein 1 OS = Homo sapiens GN = PABPC1 | UniProtKB: P11940 | <0.00010 | EGFP NHS GFP |
| PE = 1 SV = 2 | | | high, HSF1 NHS |
| | | | GFP low |
| 60S ribosomal protein L4 OS = Homo sapiens GN = RPL4 PE = 1 | UniProtKB: P36578 | <0.00010 | EGFP NHS GFP |
| SV = 5 | | | high, HSF1 NHS |
| | | | GFP low |
| 60S ribosomal protein L6 OS = Homo sapiens GN = RPL6 PE = 1 | UniProtKB: Q02878 | <0.00010 | EGFP NHS GFP |
| SV = 3 | | | high, HSF1 NHS |
| | | | GFP low |
| Ribosomal L1 domain-containing protein 1 OS = Homo sapiens | UniProtKB: O76021 | <0.00010 | EGFP NHS GFP |
| GN = RSL1D1 PE = 1 SV = 3 | | | high, HSF1 NHS |
| | | | GFP low |
| 60S ribosomal protein L5 OS = Homo sapiens GN = RPL5 PE = 1 | UniProtKB: P46777 | <0.00010 | EGFP NHS GFP |
| SV = 3 | | | high, HSF1 NHS |
| | | | GFP low |
| Nucleophosmin OS = Homo sapiens GN = NPM1 PE = 1 SV = 2 | UniProtKB: P06748 | 0.0059 | EGFP NHS GFP |
| | | | high, HSF1 NHS |
| | | | GFP low |
| Treacle protein OS = Homo sapiens GN = TCOF1 PE = 1 SV = 3 | UniProtKB: Q13428 | <0.00010 | EGFP NHS GFP |
| | | | high, HSF1 NHS |
| | | | GFP low |
| 60S ribosomal protein L7a OS = Homo sapiens GN = RPL7A PE = 1 | UniProtKB: P62424 | <0.00010 | EGFP NHS GFP |
| SV = 2 | | | high, HSF1 NHS |
| | | | GFP low |
| 60S ribosomal protein L3 OS = Homo sapiens GN = RPL3 PE = 1 | UniProtKB: P39023 | <0.00010 | EGFP NHS GFP |
| SV = 2 | | | high, HSF1 NHS |
| | | | GFP low |
| (Bos taurus) Bovine serum albumin precursor | UniProtKB: | 0.02 | EGFP NHS GFP |
| | CON_P02769 | | high, HSF1 NHS |
| | (+1) | | GFP low |
| Splicing factor, proline- and glutamine-rich OS = Homo sapiens | UniProtKB: P23246 | 0.00037 | EGFP NHS GFP |
| GN = SFPQ PE = 1 SV = 2 | | | high, HSF1 NHS |
| | | | GFP low |
| Serine/arginine repetitive matrix protein 2 OS = Homo sapiens | UniProtKB: Q9UQ35 | 0.0009 | EGFP NHS GFP |
| GN = SRRM2 PE = 1 SV = 2 | | | high, HSF1 NHS |
| | | | GFP low |
| 40S ribosomal protein S3a OS = Homo sapiens GN = RPS3A PE = 1 | UniProtKB: P61247 | 0.0014 | EGFP NHS GFP |
| SV = 2 | | | high, HSF1 NHS |
| | | | GFP low |
| 40S ribosomal protein S19 OS = Homo sapiens GN = RPS19 PE = 1 | UniProtKB: P39019 | 0.00075 | EGFP NHS GFP |
| SV = 2 | | | high, HSF1 NHS |
| | | | GFP low |
| 60S ribosomal protein L15 OS = Homo sapiens GN = RPL15 PE = 1 | UniProtKB: P61313 | 0.00092 | EGFP NHS GFP |
| SV = 2 | | | high, HSF1 NHS |
| | | | GFP low |
| 60S ribosomal protein L10a OS = Homo sapiens GN = RPL10A | UniProtKB: P62906 | 0.001 | EGFP NETS GFP |
| PE = 1 SV = 2 | | | high, HSF1 NHS |
| | | | GFP low |
| Protein disulfide-isomerase OS = Homo sapiens GN = P4HB PE = 1 | UniProtKB: P07237 | 0.022 | EGFP NHS GFP |
| SV = 3 | | | high, HSF1 NHS |
| | | | GFP low |
| 40S ribosomal protein S2 OS = Homo sapiens GN = RPS2 PE = 1 | UniProtKB: P15880 | 0.0031 | EGFP NHS GFP |
| SV = 2 | | | high, HSF1 NHS |
| | | | GFP low |
| Putative ribosomal RNA methyltransferase NOP2 OS = Homo | UniProtKB: P46087 | <0.00010 | EGFP NHS GFP |
| sapiens GN = NOP2 PE = 1 SV = 2 | | | high, HSF1 NHS |
| | | | GFP low |
| Non-POU domain-containing octamer-binding protein OS = Homo | UniProtKB: Q15233 | 0.0037 | EGFP NHS GFP |
| sapiens GN = NONO PE = 1 SV = 4 | | | high, HSF1 NHS |
| | | | GFP low |
| Transformer-2 protein homolog beta OS = Homo sapiens | UniProtKB: P62995 | 0.0061 | EGFP NHS GFP |
| GN = TRA2B PE = 1 SV = 1 | | | high, HSF1 NHS |
| | | | GFP low |
| 60S ribosomal protein L13 OS = Homo sapiens GN = RPL13 PE = 1 | UniProtKB: P26373 | <0.00010 | EGFP NHS GFP |
| SV = 4 | | | high, HSF1 NHS |
| | | | GFP low |
| Heterogeneous nuclear ribonucleoprotein U OS = Homo sapiens | UniProtKB: Q00839 | 0.031 | EGFP NHS GFP |
| GN = HNRNPU PE = 1 SV6 | | | high, HSF1 NHS |
| | | | GFP low |
| 60S ribosomal protein L27 OS = Homo sapiens GN = RPL27 PE = 1 | UniProtKB: P61353 | 0.0021 | EGFP NHS GFP |
| SV = 2 | | | high, HSF1 NHS |
| | | | GFP low |
| Transformer-2 protein homolog alpha OS = Homo sapiens | UniProtKB: Q13595 | 0.014 | EGFP NHS GFP |
| GN = TRA2A PE = 1 SV = 1 | | | high, HSF1 NHS |
| | | | GFP low |
| 40S ribosomal protein S4, X isoform OS = Homo sapiens | UniProtKB: P62701 | 0.0031 | EGFP NETS GFP |
| GN = RPS4X PE = 1 SV = 2 | | | high, HSF1 NHS |
| | | | GFP low |
| 60S ribosomal protein L14 OS = Homo sapiens GN = RPL14 PE = 1 | UniProtKB: P50914 | <0.00010 | EGFP NHS GFP |
| SV = 4 | | | high, HSF1 NHS |
| | | | GFP low |
| 60S ribosomal protein L12 OS = Homo sapiens GN = RPL12 PE = 1 | UniProtKB: P30050 | 0.0031 | EGFP NHS GFP |
| SV = 1 | | | high, HSF1 NHS |
| | | | GFP low |
| 60S ribosomal protein L8 OS = Homo sapiens GN = RPL8 PE = 1 | UniProtKB: P62917 | <0.00010 | EGFP NHS GFP |
| SV = 2 | | | high, HSF1 NHS |
| | | | GFP low |
| 40S ribosomal protein S6 OS = Homo sapiens GN = RPS6 PE = 1 | UniProtKB: P62753 | 0.00061 | EGFP NHS GFP |
| SV = 1 | | | high, HSF1 NHS |
| | | | GFP low |
| Endoplasmin OS = Homo sapiens GN = HSP90B1 PE = 1 SV = 1 | UniProtKB: P14625 | 0.031 | EGFP NHS GFP |
| | | | high, HSF1 NHS |
| | | | GFP low |
| 60S ribosomal protein L34 OS = Homo sapiens GN = RPL34 PE = 1 | UniProtKB: P49207 | 0.0061 | EGFP NHS GFP |
| SV = 3 | | | high, HSF1 NHS |
| | | | GFP low |
| Heterogeneous nuclear ribonucleoproteins A2/B1 OS = Homo | UniProtKB: P22626 | 0.027 | EGFP NHS GFP |
| sapiens GN = HNRNPA2B1 PE = 1 SV = 2 | | | high, HSF1 NHS |
| | | | GFP low |
| 60S ribosomal protein L7 OS = Homo sapiens GN = RPL7 PE = 1 | UniProtKB: P18124 | 0.0024 | EGFP NHS GFP |
| SV = 1 | | | high, HSF1 NHS |
| | | | GFP low |
| 60S ribosomal protein L18 OS = Homo sapiens GN = RPL18 PE = 1 | UniProtKB: Q07020 | 0.00043 | EGFP NHS GFP |
| SV = 2 | | | high, HSF1 NHS |
| | | | GFP low |
| 40S ribosomal protein SA OS = Homo sapiens GN = RPSA PE = 1 | UniProtKB: P08865 | 0.041 | EGFP NHS GFP |
| SV = 4 | | | high, HSF1 NHS |
| | | | GFP low |
| 40S ribosomal protein S11 OS = Homo sapiens GN = RPS11 PE = 1 | UniProtKB: P62280 | 0.039 | EGFP NHS GFP |
| SV = 3 | | | high, HSF1 NHS |
| | | | GFP low |
| Elongation factor 1-gamma OS = Homo sapiens GN = EEF1GPE = 1 | UniProtKB: P26641 | 0.019 | EGFP NHS GFP |
| SV = 3 | | | high, HSF1 NHS |
| | | | GFP low |
| 60S ribosomal protein L13a OS = Homo sapiens GN = RPL13A | UniProtKB: P40429 | 0.014 | EGFP NHS GFP |
| PE = 1 SV = 2 | | | high, HSF1 NHS |
| | | | GFP low |
| 60S ribosomal protein L17 OS = Homo sapiens GN = RPL17 PE = 1 | UniProtKB: P18621 | 0.0016 | EGFP NHS GFP |
| SV = 3 | | | high, HSF1 NHS |
| | | | GFP low |
| 40S ribosomal protein S18 OS = Homo sapiens GN = RPS18 PE = 1 | UniProtKB: P62269 | 0.029 | EGFP NHS GFP |
| SV = 3 | | | high, HSF1 NHS |
| | | | GFP low |
| 60S ribosomal protein L11 OS = Homo sapiens GN = RPL11 PE = 1 | UniProtKB: P62913 | 0.01 | EGFP NHS GFP |
| SV = 2 | | | high, HSF1 NHS |
| | | | GFP low |
| 60S ribosomal protein L23 OS = Homo sapiens GN = RPL23 PE = 1 | UniProtKB: P62829 | 0.016 | EGFP NHS GFP |
| SV = 1 | | | high, HSF1 NHS |
| | | | GFP low |
| Serine/arginine repetitive matrix protein 1 OS = Homo sapiens | UniProtKB: Q8IYB3 | 0.041 | EGFP NHS GFP |
| GN = SRRNI1 PE = 1 SV = 2 | | | high, HSF1 NHS |
| | | | GFP low |
| 40S ribosomal protein S23 OS = Homo sapiens GN = RPS23 PE = 1 | UniProtKB: P62266 | 0.0035 | EGFP NHS GFP |
| SV = 3 | | | high, HSF1 NHS |
| | | | GFP low |
| 60S ribosomal protein L18a OS = Homo sapiens GN = RPL18A | UniProtKB: Q02543 | 0.00052 | EGFP NETS GFP |
| PE = 1 SV = 2 | | | high, HSF1 NETS |
| | | | GFP low |
| Polyadenylate-binding protein 4 OS = Homo sapiens GN = PABPC4 | UniProtKB: Q13310 | <0.00010 | EGFP NETS GFP |
| PE = 1 SV = 1 | | | high, HSF1 NETS |
| | | | GFP low |
| RNA-binding protein 14 OS = Homo sapiens GN = RBM14 PE = 1 | UniProtKB: Q96PK6 | 0.0093 | EGFP NETS GFP |
| SV = 2 | | | high, HSF1 NETS |
| | | | GFP low |
| 40S ribosomal protein S25 OS = Homo sapiens GN = RPS25 PE = 1 | UniProtKB: P62851 | 0.019 | EGFP NETS GFP |
| SV = 1 | | | high, HSF1 NETS |
| | | | GFP low |
| 60S ribosomal protein L21 OS = Homo sapiens GN = RPL21 PE = 1 | UniProtKB: P46778 | 0.03 | EGFP NETS GFP |
| SV = 2 | | | high, HSF1 NETS |
| | | | GFP low |
| Eukaryotic translation initiation factor 6 OS = Homo sapiens | UniProtKB: P56537 | 0.017 | EGFP NETS GFP |
| GN = EIF6 PE = 1 SV = 1 | | | high, HSF1 NETS |
| | | | GFP low |
| 40S ribosomal protein S21 OS = Homo sapiens GN = RPS21 PE = 1 | UniProtKB: P63220 | 0.024 | EGFP NETS GFP |
| SV = 1 | | | high, HSF1 NETS |
| | | | GFP low |
| 40S ribosomal protein S5 OS = Homo sapiens GN = RPS5 PE = 1 | UniProtKB: P46782 | 0.045 | EGFP NETS GFP |
| SV = 4 | | | high, HSF1 NETS |
| | | | GFP low |
| 40S ribosomal protein S10 OS = Homo sapiens GN = RPS10 PE = 1 | UniProtKB: P46783 | 0.041 | EGFP NETS GFP |
| SV = 1 | | | high, HSF1 NETS |
| | | | GFP low |
| 60S ribosomal protein L27a OS = Homo sapiens GN = RPL27A | UniProtKB: P46776 | 0.019 | EGFP NETS GFP |
| PE = 1 SV = 2 | | | high, HSF1 NETS |
| | | | GFP low |
| 40S ribosomal protein S14 OS = Homo sapiens GN = RPS14 PE = 1 | UniProtKB: P62263 | 0.013 | EGFP NHS GFP |
| SV = 3 | | | high, HSF1 NHS |
| | | | GFP low |
| Heterogeneous nuclear ribonucleoprotein A1 OS = Homo sapiens | UniProtKB: P09651 (+1) | 0.0056 | EGFP NHS GFP |
| GN = HNRNPA1 PE = 1 SV = 5 | | | high, HSF1 NHS |
| | | | GFP low |
| Elongation factor 1-beta OS = Homo sapiens GN = EEF1B2 PE = 1 | UniProtKB: P24534 | 0.041 | EGFP NHS GFP |
| SV = 3 | | | high, HSF1 NHS |
| | | | GFP low |
| RRP15-like protein OS = Homo sapiens GN = RRP15 PE = 1 SV = 2 | UniProtKB: Q9Y3B9 | 0.02 | EGFP NHS GFP |
| | | | high, HSF1 NHS |
| | | | GFP low |
| Ribosome production factor 2 homolog OS = Homo sapiens | UniProtKB: Q9H7B2 | <0.00010 | EGFP NHS GFP |
| GN = RPF2 PE = 1 SV = 2 | | | high, HSF1 NHS |
| | | | GFP low |
| 40S ribosomal protein S28 OS = Homo sapiens GN = RPS28 PE = 1 | UniProtKB: P62857 | 0.036 | EGFP NHS GFP |
| SV = 1 | | | high, HSF1 NHS |
| | | | GFP low |
| Guanine nucleotide-binding protein subunit beta-2-like 1 | UniProtKB: P63244 | 0.024 | EGFP NHS GFP |
| OS = Homo sapiens GN = GNB2L1 PE = 1 SV = 3 | | | high, HSF1 NHS |
| | | | GFP low |
| Nucleolar GTP-binding protein 1 OS = Homo sapiens GN = GTPBP4 | UniProtKB: Q9BZE4 | <0.00010 | EGFP NHS GFP |
| PE = 1 SV = 3 | | | high, HSF1 NHS |
| | | | GFP low |
| Ribosome biogenesis regulatory protein homolog OS = Homo | UniProtKB: Q15050 | 0.0043 | EGFP NHS GFP |
| sapiens GN = RRS1 PE = 1 SV = 2 | | | high, HSF1 NHS |
| | | | GFP low |
| 60S ribosomal protein L28 OS = Homo sapiens GN = RPL28 PE = 1 | UniProtKB: P46779 | 0.03 | EGFP NHS GFP |
| SV = 3 | | | high, HSF1 NHS |
| | | | GFP low |
| Ribosome biogenesis protein BOP1 OS = Homo sapiens GN = B0P1 | UniProtKB: Q14137 | <0.00010 | EGFP NHS GFP |
| PE = 1 SV = 2 | | | high, HSF1 NHS |
| | | | GFP low |
| Probable rRNA-processing protein EBP2 OS = Homo sapiens | UniProtKB: Q99848 | <0.00010 | EGFP NHS GFP |
| GN = EBNA1BP2 PE = 1 SV = 2 | | | high, HSF1 NHS |
| | | | GFP low |
| Eukaryotic translation initiation factor 5B OS = Homo sapiens | UniProtKB: O60841 | 0.049 | EGFP NHS GFP |
| GN = EIF5B PE = 1 SV = 4 | | | high, HSF1 NHS |
| | | | GFP low |
| Pescadillo homolog OS = Homo sapiens GN = PES1 PE = 1 SV = 1 | UniProtKB: O00541 | 0.0043 | EGFP NHS GFP |
| | | | high, HSF1 NHS |
| | | | GFP low |
| Heterogeneous nuclear ribonucleoprotein A3 OS = Homo sapiens | UniProtKB: P51991 | 0.031 | EGFP NHS GFP |
| GN = HNRNPA3 PE = 1 SV = 2 | | | high, HSF1 NHS |
| | | | GFP low |
| Putative rRNA methyltransferase 3 OS = Homo sapiens GN = FTSJ3 | UniProtKB: Q8IY81 | 0.0009 | EGFP NHS GFP |
| PE = 1 SV = 2 | | | high, HSF1 NHS |
| | | | GFP low |
| Ribosome biogenesis protein WDR12 OS = Homo sapiens | UniProtKB: Q9GZL7 | 0.044 | EGFP NHS GFP |
| GN = WDR12 PE = 1 SV = 2 | | | high, HSF1 NHS |
| | | | GFP low |
| Myb-binding protein 1A OS = Homo sapiens GN = MYBBP1A | UniProtKB: Q9BQG0 | 0.002 | EGFP NHS GFP |
| PE = 1 SV = 2 | | | high, HSF1 NHS |
| | | | GFP low |
| Antigen KI-67 OS = Homo sapiens GN = MKI67 PE = 1 SV = 2 | UniProtKB: P46013 | 0.02 | EGFP NHS GFP |
| | | | high, HSF1 NHS |
| | | | GFP low |
| Ribosomal RNA processing protein 1 homolog A OS = Homo | UniProtKB: P56182 | 0.0093 | EGFP NHS GFP |
| sapiens GN = RRP1 PE = 1 SV = 1 | | | high, HSF1 NHS |
| | | | GFP low |
| Ribosome biogenesis protein NSA2 homolog OS = Homo sapiens | UniProtKB: O95478 | 0.0093 | EGFP NHS GFP |
| GN = NSA2 PE = 1 SV = 1 | | | high, HSF1 NHS |
| | | | GFP low |
| Galectin-1 OS = Homo sapiens GN = LGALS1 PE = 1 SV = 2 | UniProtKB: P09382 | 0.044 | EGFP NHS GFP |
| | | | high, HSF1 NHS |
| | | | GFP low |
| Dermcidin OS = Homo sapiens GN = DCD PE = 1 SV = 2 | UniProtKB: P81605 | 0.044 | EGFP NHS GFP |
| | | | high, HSF1 NHS |
| | | | GFP low |
| Probable ATP-dependent RNA helicase DDX27 OS = Homo | UniProtKB: Q96GQ7 | 0.044 | EGFP NHS GFP |
| sapiens GN = DDX27 PE = 1 SV = 2 | | | high, HSF1 NHS |
| | | | GFP low |
| Pumilio domain-containing protein KIAA0020 OS = Homo sapiens | UniProtKB: Q15397 | 0.044 | EGFP NHS GFP |
| GN = KIAA0020 PE = 1 SV = 3 | | | high, HSF1 NHS |
| | | | GFP low |
| Poly(rC)-binding protein 3 OS = Homo sapiens GN = PCBP3 PE = 1 | UniProtKB: P57721 | 0.0093 | EGFP NHS GFP |
| SV = 2 | | | high, HSF1 NHS |
| | | | GFP low |
| Histone H2A.x OS = Homo sapiens GN = H2AFX PE = 1 SV = 2 | UniProtKB: P16104 | 0.044 | EGFP NHS GFP |
| | | | high, HSF1 NHS |
| | | | GFP low |
| Heterogeneous nuclear ribonucleoproteins C1/C2 OS = Homo | UniProtKB: P07910 | <0.00010 | HSF1 NHS GFP |
| sapiens GN = HNRNPC PE = 1 SV = 4 | | | low, HSF1 NHS |
| | | | NELF high |
| Nucleolar RNA helicase 2 OS = Homo sapiens GN = DDX21 PE = 1 | UniProtKB: Q9NR30 | <0.00010 | HSF1 NHS GFP |
| SV = 5 | | | low, HSF1 NHS |
| | | | NELF high |
| Lipoamide acyltransferase component of branched-chain alpha- | UniProtKB: P11182 | <0.00010 | HSF1 NHS GFP |
| keto acid dehydrogenase complex, mitochondrial OS = Homo | | | low, HSF1 NHS |
| sapiens GN = DBT PE = 1 SV = 3 | | | NELF high |
| U4/U6.U5 tri-snRNP-associated protein 2 OS = Homo sapiens | UniProtKB: Q53GS9 | <0.00010 | HSF1 NETS GFP |
| GN = USP39 PE = 1 SV = 2 | | | low, HSF1 NHS |
| | | | NELF high |
| rRNA 2′-O-methyltransferase fibrillarin OS = Homo sapiens | UniProtKB: P22087 | 0.025 | HSF1 NHS GFP |
| GN = FBL PE = 1 SV = 2 | | | low, HSF1 NHS |
| | | | NELF high |
| Splicing factor U2AF 35 kDa subunit OS = Homo sapiens | UniProtKB: Q01081 | 0.0054 | HSF1 NHS GFP |
| GN = U2AF1 PE = 1 SV = 3 | | | low, HSF1 NHS |
| | | | NELF high |
| THO complex subunit 4 OS = Homo sapiens GN = THOC4 PE = 1 | UniProtKB: Q86V81 | <0.00010 | HSF1 NHS GFP |
| SV = 3 | | | low, HSF1 NHS |
| | | | NELF high |
| Serine/arginine-rich splicing factor 6 OS = Homo sapiens | UniProtKB: Q13247 | <0.00010 | HSF1 NHS GFP |
| GN = SRSF6 PE = 1 SV = 2 | | | low, HSF1 NHS |
| | | | NELF high |
| Transformer-2 protein homolog beta OS = Homo sapiens | UniProtKB: P62995 | <0.00010 | HSF1 NHS GFP |
| GN = TRA2B PE = 1 SV = 1 | | | low, HSF1 NHS |
| | | | NELF high |
| CD2 antigen cytoplasmic tail-binding protein 2 OS = Homo sapiens | UniProtKB: O95400 | <0.00010 | HSF1 NHS GFP |
| GN = CD2BP2 PE = 1 SV = 1 | | | low, HSF1 NHS |
| | | | NELF high |
| 60S ribosomal protein L6 OS = Homo sapiens GN = RPL6 PE = 1 | UniProtKB: Q02878 | 0.023 | HSF1 NHS GFP |
| SV = 3 | | | low, HSF1 NHS |
| | | | NELF high |
| Nucleophosmin OS = Homo sapiens GN = NPM1 PE = 1 SV = 2 | UniProtKB: P06748 | 0.0044 | HSF1 NHS GFP |
| | | | low, HSF1 NHS |
| | | | NELF high |
| Small nuclear ribonucleoprotein Sm D3 OS = Homo sapiens | UniProtKB: P62318 | 0.00013 | HSF1 NHS GFP |
| GN = SNRPD3 PE = 1 SV1 | | | low, HSF1 NHS |
| | | | NELF high |
| 60S ribosomal protein L4 OS = Homo sapiens GN = RPL4 PE = 1 | UniProtKB: P36578 | 0.039 | HSF1 NHS GFP |
| SV = 5 | | | low, HSF1 NHS |
| | | | NELF high |
| 60S ribosomal protein L15 OS = Homo sapiens GN = RPL15 PE = 1 | UniProtKB: P61313 | 0.0037 | HSF1 NHS GFP |
| SV = 2 | | | low, HSF1 NHS |
| | | | NELF high |
| Heterogeneous nuclear ribonucleoprotein U OS = Homo sapiens | UniProtKB: Q00839 | 0.00051 | HSF1 NHS GFP |
| GN = HNRNPU PE = 1 SV6 | | | low, HSF1 NHS |
| | | | NELF high |
| Serine/arginine-rich splicing factor 8 OS = Homo sapiens | UniProtKB: Q9BRL6 | 0.017 | HSF1 NHS GFP |
| GN = SRSF8 PE = 1 SV = 1 | | | low, HSF1 NHS |
| | | | NELF high |
| Tax_Id = 10090 Gene_Symbol = Krt19 Keratin, type I cytoskeletal | UniProtKB: | 0.011 | HSF1 NHS GFP |
| 19 | CON_P19001 | | low, HSF1 NHS |
| | | | NELF high |
| Serine/arginine-rich splicing factor 4 OS = Homo sapiens | UniProtKB: Q08170 | <0.00010 | HSF1 NHS GFP |
| GN = SRSF4 PE = 1 SV = 2 | | | low, HSF1 NHS |
| | | | NELF high |
| Serine/arginine-rich splicing factor 5 OS = Homo sapiens | UniProtKB: Q13243 | <0.00010 | HSF1 NHS GFP |
| GN = SRSF5 PE = 1 SV = 1 | | | low, HSF1 NHS |
| | | | NELF high |
| Treacle protein OS = Homo sapiens GN = TC0F1 PE = 1 SV = 3 | UniProtKB: Q13428 | <0.00010 | HSF1 NHS GFP |
| | | | low, HSF1 NHS |
| | | | NELF high |
| Heterogeneous nuclear ribonucleoprotein G OS = Homo sapiens | UniProtKB: P38159 | <0.00010 | HSF1 NHS GFP |
| GN = RBMX PE = 1 SV = 3 | | | low, HSF1 NHS |
| | | | NELF high |
| Transformer-2 protein homolog alpha OS = Homo sapiens | UniProtKB: Q13595 | <0.00010 | HSF1 NHS GFP |
| GN = TRA2A PE = 1 SV = 1 | | | low, HSF1 NHS |
| | | | NELF high |
| Heterogeneous nuclear ribonucleoprotein Q OS = Homo sapiens | UniProtKB: O60506 | 0.00036 | HSF1 NETS GFP |
| GN = SYNCRIP PE = 1 SV = 2 | | | low, HSF1 NHS |
| | | | NELF high |
| Heterogeneous nuclear ribonucleoprotein R OS = Homo sapiens | UniProtKB: O43390 | <0.00010 | HSF1 NETS GFP |
| GN = HNRNPR PE = 1 SV = 1 | | | low, HSF1 NHS |
| | | | NELF high |
| Putative ATP-dependent RNA helicase DHX30 OS = Homo sapiens | UniProtKB: Q7L2E3 | <0.00010 | HSF1 NETS GFP |
| GN = DHX30 PE = 1 SV = 1 | | | low, HSF1 NHS |
| | | | NELF high |
| U5 small nuclear ribonucleoprotein 200 kDa helicase OS = Homo | UniProtKB: O75643 | <0.00010 | HSF1 NETS GFP |
| sapiens GN = SNRNP200 PE = 1 SV = 2 | | | low, HSF1 NHS |
| | | | NELF high |
| Interleukin enhancer-binding factor 3 OS = Homo sapiens | UniProtKB: Q12906 | <0.00010 | HSF1 NETS GFP |
| GN = ILF3 PE = 1 SV = 3 | | | low, HSF1 NHS |
| | | | NELF high |
| Glutamate-rich WD repeat-containing protein 1 OS = Homo sapiens | UniProtKB: Q9BQ67 | 0.0046 | HSF1 NETS GFP |
| GN = GRWD1 PE = 1 SV = 1 | | | low, HSF1 NHS |
| | | | NELF high |
| RNA-binding Raly-like protein OS = Homo sapiens GN = RALYL | UniProtKB: Q865E5 | 0.02 | HSF1 NETS GFP |
| PE = 1 SV = 2 | | | low, HSF1 NHS |
| | | | NELF high |
| Polyadenylate-binding protein 1 OS = Homo sapiens GN = PABPC1 | UniProtKB: P11940 | <0.00010 | HSF1 NETS GFP |
| PE = 1 SV = 2 | | | low, HSF1 NHS |
| | | | NELF high |
| Polyadenylate-binding protein 4 OS = Homo sapiens GN = PABPC4 | UniProtKB: Q13310 | <0.00010 | HSF1 NETS GFP |
| PE = 1 SV = 1 | | | low, HSF1 NHS |
| | | | NELF high |
| Ezrin OS = Homo sapiens GN = EZR PE = 1 SV = 4 | UniProtKB: P15311 | 0.0058 | HSF1 NETS GFP |
| | | | low, HSF1 NHS |
| | | | NELF high |
| Bcl-2-associated transcription factor 1 OS = Homo sapiens | UniProtKB: Q9NYF8 | <0.00010 | HSF1 NETS GFP |
| GN = BCLAF1 PE = 1 SV = 2 | | | low, HSF1 NHS |
| | | | NELF high |
| 40S ribosomal protein S19 OS = Homo sapiens GN = RPS19 PE = 1 | UniProtKB: P39019 | 0.0044 | HSF1 NHS GFP |
| SV = 2 | | | low, HSF1 NHS |
| | | | NELF high |
| ATP-dependent RNA helicase A OS = Homo sapiens GN = DHX9 | UniProtKB: Q08211 | <0.00010 | HSF1 NHS GFP |
| PE = 1 SV = 4 | | | low, HSF1 NHS |
| | | | NELF high |
| Splicing factor, proline- and glutamine-rich OS = Homo sapiens | UniProtKB: P23246 | <0.00010 | HSF1 NHS GFP |
| GN = SFPQ PE = 1 SV = 2 | | | low, HSF1 NHS |
| | | | NELF high |
| Putative ribosomal RNA methyltransferase NOP2 OS = Homo | UniProtKB: P46087 | <0.00010 | HSF1 NHS GFP |
| sapiens GN = NOP2 PE = 1 SV = 2 | | | low, HSF1 NHS |
| | | | NELF high |
| Serine/arginine repetitive matrix protein 2 OS = Homo sapiens | UniProtKB: Q9UQ35 | <0.00010 | HSF1 NHS GFP |
| GN = SRRM2 PE = 1 SV = 2 | | | low, HSF1 NHS |
| | | | NELF high |
| 60S ribosomal protein L7a OS = Homo sapiens GN = RPL7A PE = 1 | UniProtKB: P62424 | 0.0018 | HSF1 NHS GFP |
| SV = 2 | | | low, HSF1 NHS |
| | | | NELF high |
| 60S ribosomal protein L10a OS = Homo sapiens GN = RPL10A | UniProtKB: P62906 | 0.037 | HSF1 NHS GFP |
| PE = 1 SV = 2 | | | low, HSF1 NHS |
| | | | NELF high |
| Non-POU domain-containing octamer-binding protein OS = Homo | UniProtKB: Q15233 | <0.00010 | HSF1 NHS GFP |
| sapiens GN = NONO PE = 1 SV = 4 | | | low, HSF1 NHS |
| | | | NELF high |
| Thyroid hormone receptor-associated protein 3 OS = Homo sapiens | UniProtKB: Q9Y2W1 | <0.00010 | HSF1 NHS GFP |
| GN = THRAP3 PE = 1 SV = 2 | | | low, HSF1 NHS |
| | | | NELF high |
| RNA-binding protein 14 OS = Homo sapiens GN = RBM14 PE = 1 | UniProtKB: Q96PK6 | <0.00010 | HSF1 NHS GFP |
| SV = 2 | | | low, HSF1 NHS |
| | | | NELF high |
| 60S ribosomal protein L12 OS = Homo sapiens GN = RPL12 PE = 1 | UniProtKB: P30050 | 0.0061 | HSF1 NHS GFP |
| SV = 1 | | | low, HSF1 NHS |
| | | | NELF high |
| 60S ribosomal protein L13 OS = Homo sapiens GN = RPL13 PE = 1 | UniProtKB: P26373 | 0.0024 | HSF1 NHS GFP |
| SV = 4 | | | low, HSF1 NHS |
| | | | NELF high |
| 60S ribosomal protein L34 OS = Homo sapiens GN = RPL34 PE = 1 | UniProtKB: P49207 | 0.011 | HSF1 NHS GFP |
| SV = 3 | | | low, HSF1 NHS |
| | | | NELF high |
| Serine/arginine repetitive matrix protein 1 OS = Homo sapiens | UniProtKB: Q8IYB3 | 0.0023 | HSF1 NHS GFP |
| GN = SRRM1 PE = 1 SV = 2 | | | low, HSF1 NHS |
| | | | NELF high |
| 60S ribosomal protein L30 OS = Homo sapiens GN = RPL30 PE = 1 | UniProtKB: P62888 | 0.0011 | HSF1 NHS GFP |
| SV = 2 | | | low, HSF1 NHS |
| | | | NELF high |
| 60S ribosomal protein L5 OS = Homo sapiens GN = RPL5 PE = 1 | UniProtKB: P46777 | 0.00011 | HSF1 NHS GFP |
| SV = 3 | | | low, HSF1 NHS |
| | | | NELF high |
| 60S ribosomal protein L27 OS = Homo sapiens GN = RPL27 PE = 1 | UniProtKB: P61353 | 0.041 | HSF1 NHS GFP |
| SV = 2 | | | low, HSF1 NHS |
| | | | NELF high |
| 60S ribosomal protein L31 OS = Homo sapiens GN = RPL31 PE = 1 | UniProtKB: P62899 | 0.00013 | HSF1 NHS GFP |
| SV = 1 | | | low, HSF1 NHS |
| | | | NELF high |
| 40S ribosomal protein S10 OS = Homo sapiens GN = RPS10 PE = 1 | UniProtKB: P46783 | 0.017 | HSF1 NHS GFP |
| SV = 1 | | | low, HSF1 NHS |
| | | | NELF high |
| Small nuclear ribonucleoprotein Sm D2 OS = Homo sapiens | UniProtKB: P62316 | <0.00010 | HSF1 NETS GFP |
| GN = SNRPD2 PE = 1 SV = 1 | | | low, HSF1 NHS |
| | | | NELF high |
| RNA-binding protein with serine-rich domain 1 OS = Homo sapiens | UniProtKB: Q15287 | <0.00010 | HSF1 NHS GFP |
| GN = RNP S1 PE = 1 SV = 1 | | | low, HSF1 NHS |
| | | | NELF high |
| Ras GTPase-activating protein-binding protein 2 OS = Homo | UniProtKB: Q9UN86 | <0.00010 | HSF1 NHS GFP |
| sapiens GN = G3BP2 PE = 1 SV = 2 | | | low, HSF1 NHS |
| | | | NELF high |
| YTH domain-containing protein 1 OS = Homo sapiens | UniProtKB: Q96MU7 | <0.00010 | HSF1 NHS GFP |
| GN = YTHDC1 PE = 1 SV = 3 | | | low, HSF1 NHS |
| | | | NELF high |
| Ribosome biogenesis protein BOP1 OS = Homo sapiens GN = BOP1 | UniProtKB: Q14137 | 0.011 | HSF1 NHS GFP |
| PE = 1 SV = 2 | | | low, HSF1 NHS |
| | | | NELF high |
| Pre-mRNA branch site protein p14 OS = Homo sapiens | UniProtKB: Q9Y3B4 | 0.00075 | HSF1 NHS GFP |
| GN = SF3B14 PE = 1 SV = 1 | | | low, HSF1 NHS |
| | | | NELF high |
| Heterogeneous nuclear ribonucleoprotein A0 OS = Homo sapiens | UniProtKB: Q13151 | 0.027 | HSF1 NHS GFP |
| GN = HNRNPA0 PE = 1 SV1 | | | low, HSF1 NHS |
| | | | NELF high |
| Probable ATP-dependent RNA helicase DDX5 OS = Homo sapiens | UniProtKB: P17844 | <0.00010 | HSF1 NHS GFP |
| GN = DDX5 PE = 1 SV = 1 | | | low, HSF1 NHS |
| | | | NELF high |
| Ras GTPase-activating protein-binding protein 1 OS = Homo | UniProtKB: Q13283 | <0.00010 | HSF1 NHS GFP |
| sapiens GN = G3BP1 PE = 1 SV = 1 | | | low, HSF1 NHS |
| | | | NELF high |
| Small nuclear ribonucleoprotein E OS = Homo sapiens GN = SNRPE | UniProtKB: P62304 | 0.001 | HSF1 NHS GFP |
| PE = 1 SV = 1 | | | low, HSF1 NHS |
| | | | NELF high |
| 116 kDa U5 small nuclear ribonucleoprotein component | UniProtKB: Q15029 | <0.00010 | HSF1 NHS GFP |
| OS = Homo sapiens GN = EFTUD2 PE = 1 SV = 1 | | | low, HSF1 NHS |
| | | | NELF high |
| Serine/arginine-rich splicing factor 7 OS = Homo sapiens | UniProtKB: Q16629 | <0.00010 | HSF1 NHS GFP |
| GN = SRSF7 PE = 1 SV = 1 | | | low, HSF1 NHS |
| | | | NELF high |
| Eukaryotic initiation factor 4A-III OS = Homo sapiens GN = EIF4A3 | UniProtKB: P38919 | <0.00010 | HSF1 NHS GFP |
| PE = 1 SV = 4 | | | low, HSF1 NHS |
| | | | NELF high |
| Polyadenylate-binding protein 1-like OS = Homo sapiens | UniProtKB: Q4VXU2 | <0.00010 | HSF1 NHS GFP |
| GN = PABPC1L PE = 2 SV = 1 | | | low, HSF1 NHS |
| | | | NELF high |
| Small nuclear ribonucleoprotein-associated proteins B and B′ | UniProtKB: P14678 | 0.011 | HSF1 NHS GFP |
| OS = Homo sapiens GN = SNRPB PE = 1 SV = 2 | | | low, HSF1 NHS |
| | | | NELF high |
| Caprin-1 OS = Homo sapiens GN = CAPRIN1 PE = 1 SV = 2 | UniProtKB: Q14444 | 0.00027 | HSF1 NHS GFP |
| | | | low, HSF1 NHS |
| | | | NELF high |
| Splicing factor 3B subunit 2 OS = Homo sapiens GN = SF3B2 | UniProtKB: Q13435 | 0.0046 | HSF1 NHS GFP |
| PE = 1 SV = 2 | | | low, HSF1 NHS |
| | | | NELF high |
| Eukaryotic translation initiation factor 6 OS = Homo sapiens | UniProtKB: P56537 | 0.0033 | HSF1 NHS GFP |
| GN = EIF6 PE = 1 SV = 1 | | | low, HSF1 NHS |
| | | | NELF high |
| Interleukin enhancer-binding factor 2 OS = Homo sapiens | UniProtKB: Q12905 | <0.00010 | HSF1 NHS GFP |
| GN = ILF2 PE = 1 SV = 2 | | | low, HSF1 NHS |
| | | | NELF high |
| U6 snRNA-associated Sm-like protein LSm3 OS = Homo sapiens | UniProtKB: P62310 | 0.0033 | HSF1 NHS GFP |
| GN = LSM3 PE = 1 SV = 2 | | | low, HSF1 NHS |
| | | | NELF high |
| Enhancer of rudimentary homolog OS = Homo sapiens GN = ERH | UniProtKB: P84090 | 0.0033 | HSF1 NHS GFP |
| PE = 1 SV = 1 | | | low, HSF1 NHS |
| | | | NELF high |
| Signal recognition particle 14 kDa protein OS = Homo sapiens | UniProtKB: P37108 | 0.0016 | HSF1 NHS GFP |
| GN = SRP14 PE = 1 SV = 2 | | | low, HSF1 NHS |
| | | | NELF high |
| Pre-mRNA-processing-splicing factor 8 OS = Homo sapiens | UniProtKB: Q6P2Q9 | <0.00010 | HSF1 NHS GFP |
| GN = PRPF 8 PE = 1 SV = 2 | | | low, HSF1 NHS |
| | | | NELF high |
| Serine/arginine-rich splicing factor 3 OS = Homo sapiens | UniProtKB: P84103 | <0.00010 | HSF1 NHS GFP |
| GN = SRSF3 PE = 1 SV = 1 | | | low, HSF1 NHS |
| | | | NELF high |
| Serine/arginine-rich splicing factor 9 OS = Homo sapiens | UniProtKB: Q13242 | <0.00010 | HSF1 NHS GFP |
| GN = SRSF9 PE = 1 SV = 1 | | | low, HSF1 NHS |
| | | | NELF high |
| Splicing factor 3B subunit 3 OS = Homo sapiens GN = SF3B3 | UniProtKB: Q15393 | 0.00087 | HSF1 NHS GFP |
| PE = 1 SV = 4 | | | low, HSF1 NHS |
| | | | NELF high |
| RNA-binding protein Raly OS = Homo sapiens GN = RALY PE = 1 | UniProtKB: Q9UKM9 | 0.0043 | HSF1 NHS GFP |
| SV = 1 | | | low, HSF1 NHS |
| | | | NELF high |
| Protein mago nashi homolog OS = Homo sapiens GN = MAGOH | UniProtKB: P61326 | 0.0095 | HSF1 NHS GFP |
| PE = 1 SV = 1 | | | low, HSF1 NHS |
| | | | NELF high |
| Putative RNA-binding protein Luc7-like 2 OS = Homo sapiens | UniProtKB: Q9Y383 | 0.0095 | HSF1 NHS GFP |
| GN = LUC7L2 PE = 1 SV = 2 | | | low, HSF1 NHS |
| | | | NELF high |
| Protein LYRIC OS = Homo sapiens GN = MTDH PE = 1 SV = 2 | UniProtKB: Q86UE4 | 0.044 | HSF1 NHS GFP |
| | | | low, HSF1 NHS |
| | | | NELF high |
| High mobility group protein HMG-I/HMG-Y OS = Homo sapiens | UniProtKB: P17096 | 0.044 | HSF1 NETS GFP |
| GN = HMGA1 PE = 1 SV = 3 | | | low, HSF1 NHS |
| | | | NELF high |
| N-alpha-acetyltransferase 38, NatC auxiliary subunit OS = Homo | UniProtKB: O95777 | 0.021 | HSF1 NETS GFP |
| sapiens GN = NAA38 PE = 1 SV = 3 | | | low, HSF1 NHS |
| | | | NELF high |
| U4/U6.U5 tri-snRNP-associated protein 1 OS = Homo sapiens | UniProtKB: O43290 | <0.00010 | HSF1 NETS GFP |
| GN = SART1 PE = 1 SV = 1 | | | low, HSF1 NHS |
| | | | NELF high |
| Ribosomal L1 domain-containing protein 1 OS = Homo sapiens | UniProtKB: O76021 | <0.00010 | HSF1 NETS GFP |
| GN = RSL1D1 PE = 1 SV = 3 | | | low, HSF1 NHS |
| | | | NELF high |
| ATP-dependent RNA helicase DDX50 OS = Homo sapiens | UniProtKB: Q9BQ39 | <0.00010 | HSF1 NETS GFP |
| GN = DDX50 PE = 1 SV = 1 | | | low, HSF1 NHS |
| | | | NELF high |
| Pinin OS = Homo sapiens GN = PNN PE = 1 SV = 4 | UniProtKB: Q9H307 | <0.00010 | HSF1 NETS GFP |
| | | | low, HSF1 NHS |
| | | | NELF high |
| Squamous cell carcinoma antigen recognized by T-cells 3 | UniProtKB: Q15020 | <0.00010 | HSF1 NETS GFP |
| OS = Homo sapiens GN = SART3 PE = 1 SV = 1 | | | low, HSF1 NHS |
| | | | NELF high |
| Probable ATP-dependent RNA helicase DDX23 OS = Homo | UniProtKB: Q9BUQ8 | <0.00010 | HSF1 NETS GFP |
| sapiens GN = DDX23 PE = 1 SV = 3 | | | low, HSF1 NHS |
| | | | NELF high |
| Apoptotic chromatin condensation inducer in the nucleus | UniProtKB: Q9UKV3 | <0.00010 | HSF1 NETS GFP |
| OS = Homo sapiens GN = ACIN1 PE = 1 SV = 2 | | | low, HSF1 NHS |
| | | | NELF high |
| Histone deacetylase complex subunit SAP18 OS = Homo sapiens | UniProtKB: O00422 | <0.00010 | HSF1 NETS GFP |
| GN = SAP18 PE = 1 SV = 1 | | | low, HSF1 NHS |
| | | | NELF high |
| U4/U6 small nuclear ribonucleoprotein Prp31 OS = Homo sapiens | UniProtKB: Q8WWY3 | <0.00010 | HSF1 NETS GFP |
| GN = PRPF31 PE = 1 SV = 2 | | | low, HSF1 NHS |
| | | | NELF high |
| Fragile X mental retardation syndrome-related protein 2 | UniProtKB: P51116 | <0.00010 | HSF1 NHS GFP |
| OS = Homo sapiens GN = FXR2 PE = 1 SV = 2 | | | low, HSF1 NHS |
| | | | NELF high |
| Fragile X mental retardation syndrome-related protein 1 | UniProtKB: P51114 | <0.00010 | HSF1 NHS GFP |
| OS = Homo sapiens GN = FXR1 PE = 1 SV = 3 | | | low, HSF1 NHS |
| | | | NELF high |
| Friend of PRMT1 protein OS = Homo sapiens GN = C1orf77 | UniProtKB: Q9Y3Y2 | <0.00010 | HSF1 NHS GFP |
| PE = 1 SV = 2 | | | low, HSF1 NHS |
| | | | NELF high |
| RNA-binding motif protein, X-linked-like-2 OS = Homo sapiens | UniProtKB: O75526 | <0.00010 | HSF1 NHS GFP |
| GN = RBMXL2 PE = 1 SV = 3 | | | low, HSF1 NHS |
| | | | NELF high |
| U4/U6 small nuclear ribonucleoprotein Prp4 OS = Homo sapiens | UniProtKB: O43172 | <0.00010 | HSF1 NHS GFP |
| GN = PRPF4 PE = 1 SV = 2 | | | low, HSF1 NHS |
| | | | NELF high |
| Fragile X mental retardation 1 protein OS = Homo sapiens | UniProtKB: Q06787 | <0.00010 | HSF1 NHS GFP |
| GN = FMR1 PE = 1 SV1 | | | low, HSF1 NHS |
| | | | NELF high |
| Nucleolar and coiled-body phosphoprotein 1 OS = Homo sapiens | UniProtKB: Q14978 | 0.00012 | HSF1 NHS GFP |
| GN = NOLC1 PE = 1 SV = 2 | | | low, HSF1 NHS |
| | | | NELF high |
| La-related protein 7 OS = Homo sapiens GN = LARP7 PE = 1 | UniProtKB: Q4G0J3 | <0.00010 | HSF1 NHS GFP |
| SV = 1 | | | low, HSF1 NHS |
| | | | NELF high |
| RRP15-like protein OS = Homo sapiens GN = RRP15 PE = 1 | UniProtKB: Q9Y3B9 | 0.00012 | HSF1 NHS GFP |
| SV = 2 | | | low, HSF1 NHS |
| | | | NELF high |
| Ribosome production factor 2 homolog OS = Homo sapiens | UniProtKB: Q9H7B2 | 0.00031 | HSF1 NETS GFP |
| GN = RPF2 PE = 1 SV = 2 | | | low, HSF1 NHS |
| | | | NELF high |
| Ribosome biogenesis protein BMS1 homolog OS = Homo sapiens | UniProtKB: Q14692 | 0.00031 | HSF1 NHS GFP |
| GN = BMS1 PE = 1 SV = 1 | | | low, HSF1 NHS |
| | | | NELF high |
| Nucleolar GTP-binding protein 1 OS = Homo sapiens GN = GTPBP4 | UniProtKB: Q9BZE4 | 0.011 | HSF1 NHS GFP |
| PE = 1 SV = 3 | | | low, HSF1 NHS |
| | | | NELF high |
| ATP-dependent RNA helicase DHX8 OS = Homo sapiens | UniProtKB: Q14562 | 0.027 | HSF1 NHS GFP |
| GN = DHX8 PE = 1 SV = 1 | | | low, HSF1 NHS |
| | | | NELF high |
| Suppressor of SWI4 1 homolog OS = Homo sapiens GN = PPAN | UniProtKB: Q9NQ55 | 0.0019 | HSF1 NHS GFP |
| PE = 1 SV = 1 | | | low, HSF1 NHS |
| | | | NELF high |
| U4/U6 small nuclear ribonucleoprotein Prp3 OS = Homo sapiens | UniProtKB: O43395 | 0.0019 | HSF1 NHS GFP |
| GN = PRPF3 PE = 1 SV = 2 | | | low, HSF1 NHS |
| | | | NELF high |
| Zinc finger CCCH domain-containing protein 13 OS = Homo | UniProtKB: Q5T200 | 0.00075 | HSF1 NHS GFP |
| sapiens GN = ZC3H13 PE = 1 SV = 1 | | | low, HSF1 NHS |
| | | | NELF high |
| Something about silencing protein 10 OS = Homo sapiens | UniProtKB: Q9NQZ2 | 0.00031 | HSF1 NHS GFP |
| GN = UTP3 PE = 1 SV = 1 | | | low, HSF1 NHS |
| | | | NELF high |
| RNA-binding protein 8A OS = Homo sapiens GN = RBM8A PE = 1 | UniProtKB: Q9Y559 | 0.00075 | HSF1 NHS GFP |
| SV = 1 | | | low, HSF1 NHS |
| | | | NELF high |
| SPATS2-like protein OS = Homo sapiens GN = SPATS2L PE = 1 | UniProtKB: Q9NUQ6 | 0.0019 | HSF1 NHS GFP |
| SV = 2 | | | low, HSF1 NHS |
| | | | NELF high |
| RNA-binding protein 28 OS = Homo sapiens GN = RBM28 PE = 1 | UniProtKB: Q9NW13 | 0.0046 | HSF1 NHS GFP |
| SV = 3 | | | low, HSF1 NHS |
| | | | NELF high |
| Activator of basal transcription 1 OS = Homo sapiens GN = ABT1 | UniProtKB: Q9ULW3 | 0.00031 | HSF1 NHS GFP |
| PE = 1 SV = 1 | | | low, HSF1 NHS |
| | | | NELF high |
| U3 small nucleolar RNA-interacting protein 2 OS = Homo sapiens | UniProtKB: O43818 | 0.0019 | HSF1 NHS GFP |
| GN = RRP9 PE = 1 SV = 1 | | | low, HSF1 NHS |
| | | | NELF high |
| Probable dimethyladenosine transferase OS = Homo sapiens | UniProtKB: Q9UNQ2 | 0.0046 | HSF1 NHS GFP |
| GN = DIMT1L PE = 1 SV = 1 | | | low, HSF1 NHS |
| | | | NELF high |
| Ribosome biogenesis regulatory protein homolog OS = Homo | UniProtKB: Q15050 | 0.0019 | HSF1 NHS GFP |
| sapiens GN = RRS1 PE = 1 SV = 2 | | | low, HSF1 NHS |
| | | | NELF high |
| Splicing factor 3B subunit 1 OS = Homo sapiens GN = SF3B1 | UniProtKB: O75533 | 0.027 | HSF1 NHS GFP |
| PE = 1 SV = 3 | | | low, HSF1 NHS |
| | | | NELF high |
| Nuclear fragile X mental retardation-interacting protein 2 | UniProtKB: Q7Z417 | 0.027 | HSF1 NHS GFP |
| OS = Homo sapiens GN = NUFIP2 PE = 1 SV = 1 | | | low, HSF1 NHS |
| | | | NELF high |
| Coiled-coil domain-containing protein 86 OS = Homo sapiens | UniProtKB: Q9H6F5 | 0.0046 | HSF1 NHS GFP |
| GN = CCDC86 PE = 1 SV = 1 | | | low, HSF1 NHS |
| | | | NELF high |
| Ribosomal RNA processing protein 1 homolog B OS = Homo | UniProtKB: Q14684 | 0.011 | HSF1 NHS GFP |
| sapiens GN = RRP1B PE = 1 SV = 3 | | | low, HSF1 NHS |
| | | | NELF high |
| BUD13 homolog OS = Homo sapiens GN = BUD13 PE = 1 SV = 1 | UniProtKB: Q9BRD0 | 0.0046 | HSF1 NHS GFP |
| | | | low, HSF1 NHS |
| | | | NELF high |
| Pre-mRNA-processing factor 6 OS = Homo sapiens GN = PRPF6 | UniProtKB: O94906 | 0.027 | HSF1 NHS GFP |
| PE = 1 SV = 1 | | | low, HSF1 NHS |
| | | | NELF high |
| Pre-mRNA-splicing regulator WTAP OS = Homo sapiens | UniProtKB: Q15007 | 0.027 | HSF1 NHS GFP |
| GN = WTAP PE = 1 SV = 2 | | | low, HSF1 NHS |
| | | | NELF high |
| Serine/threonine-protein kinase SRPK1 OS = Homo sapiens | UniProtKB: Q96SB4 | 0.0046 | HSF1 NHS GFP |
| GN = SRPK1 PE = 1 SV = 2 | | | low, HSF1 NHS |
| | | | NELF high |
| Serine/arginine-rich splicing factor 10 OS = Homo sapiens | UniProtKB: O75494 | 0.0019 | HSF1 NHS GFP |
| GN = SRSF 10 PE = 1 SV = 1 | | | low, HSF1 NHS |
| | | | NELF high |
| Polymerase delta-interacting protein 3 OS = Homo sapiens | UniProtKB: Q9BY77 | 0.027 | HSF1 NHS GFP |
| GN = POLDIP3 PE = 1 SV = 2 | | | low, HSF1 NHS |
| | | | NELF high |
| Pescadillo homolog OS = Homo sapiens GN = PES1 PE = 1 SV = 1 | UniProtKB: O00541 | 0.027 | HSF1 NHS GFP |
| | | | low, HSF1 NHS |
| | | | NELF high |
| RNA methyltransferase-like protein 1 OS = Homo sapiens | UniProtKB: Q9HC36 | 0.027 | HSF1 NHS GFP |
| GN = RNIVITL1 PE = 1 SV = 2 | | | low, HSF1 NHS |
| | | | NELF high |
| 3′-5′ exoribonuclease 1 OS = Homo sapiens GN = ERI1 | UniProtKB: Q8IV48 | 0.011 | HSF1 NHS GFP |
| PE = 1 SV = 3 | | | low, HSF1 NHS |
| | | | NELF high |
| U6 snRNA-associated Sm-like protein LSm4 OS = Homo sapiens | UniProtKB: Q9Y4Z0 | 0.027 | HSF1 NHS GFP |
| GN = LSM4 PE = 1 SV = 1 | | | low, HSF1 NHS |
| | | | NELF high |
| Probable ATP-dependent RNA helicase DDX47 OS = Homo | UniProtKB: Q9H054 | 0.027 | HSF1 NHS GFP |
| sapiens GN = DDX47 PE = 1 SV = 1 | | | low, HSF1 NHS |
| | | | NELF high |

Similarly, proteins were identified that are statistically enriched in pull-downs from HSF1-GFP expressing cells using the control-aptamer, compared to the GFP-aptamer (Table 3). These datasets are highly informative as they provide a list of common contaminants for an AptA-MS experiment. The first comparison generates a list of proteins that bind to GFP nonspecifically that can be used for future experiments using a GFP-tag. GO analysis of proteins enriched by the NELF-aptamer shows that the majority of these proteins function in binding, primarily to nucleic acids (FIGS. 9A-9C). All of these nucleic acid binding proteins are RNA-binding and is not unexpected as this aptamer recognizes the RNA binding domain of dNELF-E (Pagano et al., “Defining NELF-E RNA Binding in HIV-1 and Promoter-proximal Pause Regions,” PLoS Genet. 10:e1004090 (2014), which is hereby incorporated by reference in its entirety) (FIG. 9B). These datasets not only provide a control for GFP pull-downs, but also a control for non-specific RNA-binding proteins, analogous to the common contaminants repository (CRAPome) used for proteomics (Mellacheruvu et al., “The CRAPome: a Contaminant Repository for Affinity Purification-mass Spectrometry Data,” Nat. Methods 10:730-736 (2013), which is hereby incorporated by reference in its entirety). This database has been constructed, designated in RACER, and made available on Mascot for future AptA-MS experiments.

### Example 6—Broad Applicability of AptA-MS

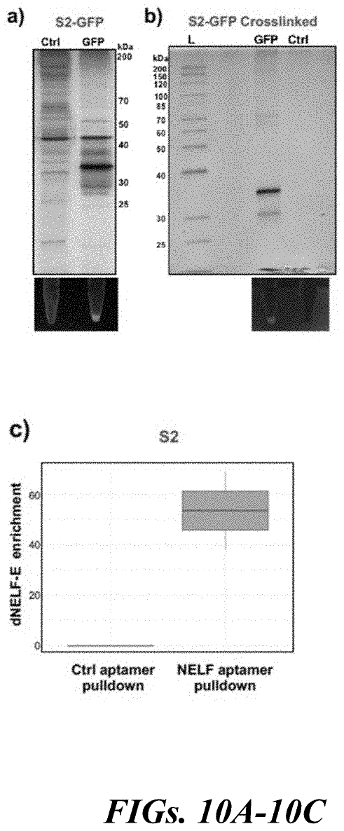

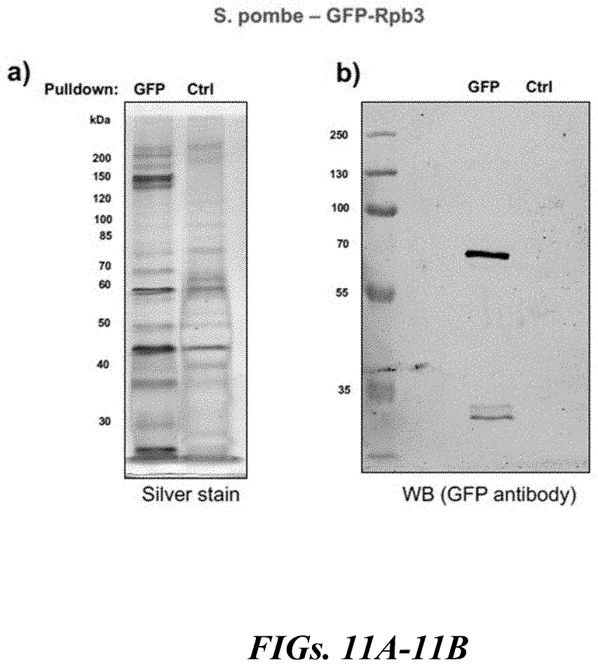

To test the broad applicability of AptA-MS, GFP was affinity enriched from Drosophila S2 cells expressing GFP with or without formaldehyde crosslinking (FIGS. 10A-10B). The ability of the GFP-aptamer to enrich crosslinked GFP makes it usable for identifying transient interactions. In addition, the NELF-aptamer has been utilized to purify the NELF complex from Drosophila S2 nuclear extracts and −50-fold enrichment of dNELF-E in NELF-aptamer pull-down was observed relative to the control (GFP)-aptamer pull-down (FIG. 10C), thereby making AptA-MS applicable for other aptamers as well. To demonstrate that proteins expressed from their endogenous promoters (and not just overexpressed proteins) can be purified by AptA-MS, a S. pombe line expressing endogenously GFP-tagged Rpb3, a subunit of Pol II, has been utilized. Affinity purification with the GFP-aptamer successfully enriched GFP-Rpb3 along with potential interactors and possibly other Pol II subunits from these cells (FIG. 11). The results clearly indicate that AptA-MS can be of general use to molecular and cellular biology applications in different model organisms.

### Discussion of Examples 1-6

Conventional affinity purification strategies rely upon the use of antibodies against a specific protein or epitope tag for successful enrichment of the bait protein and identification of its interacting partners (Bauer et al., “Affinity Purification-mass Spectrometry. Powerful Tools for the Characterization of Protein Complexes,” Eur. J. Biochem. 270:570-578 (2003), which is hereby incorporated by reference in its entirety). Nucleic acid-dependent affinity purification methods provide critical advantages over immunoprecipitations by limiting the amount of contaminating peptides that mask the detection of low-abundant interactors. Although relatively limited, aptamer-based purification strategies have been previously implemented for protein purification from biological sources (Perret et al., “Aptamer-based Affinity Chromatography for Protein Extraction and Purification,” In: Adv. Biochem. Eng. Biotechnol. Springer, Berlin, Heidelberg, pp. 1-47 (2019); Srisawat et al., “Streptavidin Aptamers: Affinity Tags for the Study of RNAs and Ribonucleoproteins,” RNA 7:632-641 (2001); Lonne et al., “Development of an Aptamer-based Affinity Purification Method for Vascular Endothelial Growth Factor,” Biotechnol. Rep. (Amst.) 8:16-23 (2015)). In addition, the Thrombin, IgE and ATP aptamers have been used for target isolation and detection by MS (Dick et al., “Aptamer-enhanced laser desorption/ionization for affinity mass spectrometry,” Anal. Chem. 76:3037-3041 (2004); Cole et al., “Affinity capture and detection of immunoglobulin E in human serum using an aptamer-modified surface in matrix-assisted laser desorption/ionization mass spectrometry,” Anal. Chem. 79:273-279 (2007); Ocsoy et al., “Aptamer-conjugated multifunctional nanoflowers as a platform for targeting, capture, and detection in laser desorption ionization mass spectrometry,” ACS Nano 7:417-427 (2013)). In one study, the EGFR and INSR aptamers were utilized to detect some of the interactors of the target proteins by western blot (Kim, et al., “Efficient Isolation and Elution of Cellular Proteins using Aptamer-mediated Protein Precipitation Assay,” Biochem. Biophys. Res. Commun. 448:114-119 (2014), which is hereby incorporated by reference in its entirety). However, aptamers have not been broadly used for exploring biological questions pertaining to protein-protein interactions utilizing MS. Furthermore, the previous aptamer-based analytical assays were restricted to aptamers against specific proteins of a particular species or small molecules, lacking the versatility and high sensitivity that is achievable by AptA-MS, as the latter takes advantage of GFP as an affinity tag. The GFP-aptamer was utilized to develop AptA-MS for the following reasons. First, GFP is a widely-used protein tag that has been applied in cellular imaging for decades. Therefore, an affinity purification approach targeting GFP would potentially serve as a common strategy for purifying hundreds of GFP-tagged proteins thereby minimizing technical variation and background. Second, GFP has no significant sequence similarity in human cells or commonly used model organisms making it less prone to non-specific interactions with other cellular components. Third, GFP has no known propensity for nucleic acids, which provides an explanation for the previous unsuccessful attempts of selecting an aptamer against it (Stanlis et al., “Single-strand DNA Aptamers as Probes for Protein Localization in Cells,” J. Histochem. Cytochem. 51:797-808 (2003), which is hereby incorporated by reference in its entirety). Therefore, the high-affinity aptamer against GFP should make it ‘less-sticky’ to other cellular proteins. Although, aptamers are considered to be ‘high-affinity’ reagents, the ligand binding affinities range from picomolar to micromolar dissociation constants (Kd) (Ilgu et al., “Aptamers in Analytics,” Analyst 141:1551-1568 (2016), which is hereby incorporated by reference in its entirety). Many of these aptamers cannot be used for affinity purification, as it is believed that the target-affinity requirement for such application should be at least in the low nanomolar range. In this aspect, the GFP-aptamer stands out due to its strong binding affinity (Kd=2.4-4.2 nM) (Tome et al., “Comprehensive Analysis of RNA-protein Interactions by High-throughput Sequencing-RNA Affinity Profiling,” Nat. Methods 11:683-688 (2014), which is hereby incorporated by reference in its entirety) and therefore, is suitable to serve the purpose. A good affinity-purification strategy requires reagents that are not only high-affinity, but also highly-specific and with broad utility. The GFP-tag and GFP-aptamer combination satisfies all these criteria and serves as a tool to study protein-protein interactions with high confidence, and is widely applicable in different species, tissues, and cell types.

To demonstrate the practical utility of the method, HSF1 was targeted, a critical regulator of HS response in mammals. HSF1 mediated activation of gene expression is associated with its binding to HSEs at pre-established transcriptional regulatory elements and release of paused PolII into productive elongation (Vihervaara et al., “Transcriptional Response to Stress is Pre-wired by Promoter and Enhancer Architecture,” Nat. Commun. 8:255 (2017); Mahat et al., “Mammalian Heat Shock Response and Mechanisms Underlying its Genome-wide Transcriptional Regulation,” Mol. Cell, 62:63-78 (2016); Ray et al., “Chromatin Conformation Remains Stable Upon Extensive Transcriptional Changes Driven by Heat Shock,” Proc. Natl Acad. Sci. U.S.A., 116:19431-19439 (2019), which are hereby incorporated by reference in their entirety). In normal conditions, HSF1 is constitutively expressed and remains inactive but upon stress it is converted to a transcriptionally active state, coordinated by PTMs and interactions with other proteins (Akerfelt et al., “Heat Shock Factors: Integrators of Cell Stress, Development and Lifespan,” Nat. Rev. Mol. Cell Biol. 11:545-555 (2010), which is hereby incorporated by reference in its entirety). Epitope-tagged HSF1 has been previously expressed in human cells to identify its PTMs and interacting partners upon immunoprecipitation followed by MS (Raychaudhuri et al., “Interplay of Acetyltransferase EP300 and the Proteasome System in Regulating Heat Shock Transcription Factor 1,” Cell 156:975-985 (2014); Fujimoto et al., “RPA assists HSF1 Access to Nucleosomal DNA by Recruiting Histone Chaperone FACT,” Mol. Cell 48:182-194 (2012), which are hereby incorporated by reference in their entirety). With AptA-MS some of the strong interactors and PTMs have been verified that have previously been detected but also have identified a few novel interacting partners/co-precipitates, clearly demonstrating the potential of this technology.

Differential protein purification analysis shows a major response to HS that is coordinated by HSF1. Like previous studies, chaperone proteins are detected that are HS responsive and interact with HSF1 to induce transcriptional events (Raychaudhuri et al., “Interplay of Acetyltransferase EP300 and the Proteasome System in Regulating Heat Shock Transcription Factor 1,” Cell 156:975-985 (2014), which is hereby incorporated by reference in its entirety). The functional classes associated with this induced interaction are illustrated via GO analysis showing an increased proportion of chaperone proteins and proteins with ‘binding’ activity to be interacting with HSF1 after HS. Furthermore, nucleic acid-binding proteins are more enriched after HS, and these proteins shift from primarily RNA-binding to mostly DNA-binding proteins in HS cells. This may reflect HSF1's increased DNA binding activity upon HS.

Expression of cytoskeletal genes has been shown to be up-regulated early during HS (Mahat et al., “Mammalian Heat Shock Response and Mechanisms Underlying its Genome-wide Transcriptional Regulation,” Mol. Cell 62:63-78 (2016), which is hereby incorporated by reference in its entirety), and interestingly an increased enrichment of cytoskeletal proteins copurifying with HSF1 is observed in these conditions. In particular, high-confidence interactions are demonstrated between HSF1 and cytoskeletal proteins including tubulin and keratin only after HS. It is speculated that these interactions could be a component of a feedback regulation that keeps the HS response modulated.

The chromatin environment of cells is also dramatically changed by stress. Alteration of histone PTM levels have been shown to be associated with HSF1 occupancy on chromatin upon HS (Vihervaara et al., “Transcriptional Response to Stress is Pre-wired by Promoter and Enhancer Architecture,” Nat. Commun 8:255 (2017); Kusch et al., “Histone H3 Lysine 4 trimethylation Regulates Cotranscriptional H2A Variant Exchange by Tip60 Complexes to Maximize Gene Expression,” Proc. Natl Acad. Sci. U.S.A., 111:4850-4855 (2014), which are hereby incorporated by reference in their entirety). Histone methyltransferases particularly targeting histone H3 lysine 4 have been shown to contribute to the HS response (Raychaudhuri et al., “Interplay of Acetyltransferase EP300 and the Proteasome System in Regulating Heat Shock Transcription Factor 1,” Cell 156:975-985 (2014); Ardehali et al., “Drosophila Set1 is the Major Histone H3 Lysine 4 Trimethyltransferase with Role in Transcription,” EMBO J. 30:2817-2828 (2011), which are hereby incorporated by reference in their entirety). While these histone modifiers may not directly interact with HSF1, a HS-induced copurification of histone H3 with HSF1 is shown, indicating that HSF1 is binding near transcriptionally active H3-containing DNA. In addition, induced interactions are detected between HSF1 and histones H2B and H4. The method can likely pick up these low-frequency interactions by virtue of its low background signal, and shows that HSF1 is binding differentially to transcriptionally-active chromatin as previously reported (Vihervaara et al., “Transcriptional Response to Stress is Pre-wired by Promoter and Enhancer Architecture,” Nat. Commun 8:255 (2017), which is hereby incorporated by reference in its entirety).

HSF1 has been shown to undergo extensive PTMs during its regulation. HSF1 is ubiquitinated during recovery from HS and when overexpressed in cells, and we find an interaction between HSF1 and a ubiquitin-40S protein, likely reflecting this modification process. Multiple lysines of HSF1 were found to be acetylated even in the NHS condition (Raychaudhuri et al., “Interplay of Acetyltransferase EP300 and the Proteasome System in Regulating Heat Shock Transcription Factor 1,” Cell 156:975-985 (2014), which is hereby incorporated by reference in its entirety), while acetylation of specific lysine residues was shown to be critical to the HS response. Acetylation of K80 and K118, was shown to be crucial for the release of HSF1 trimers from the HSEs and inhibited chromatin binding of HSF1 (Raychaudhuri et al., “Interplay of Acetyltransferase EP300 and the Proteasome System in Regulating Heat Shock Transcription Factor 1,” Cell 156:975-985 (2014); Westerheide et al., “Stress-inducible Regulation of Heat Shock Factor 1 by the Deacetylase SIRT1,” Science 323:1063-1066 (2009), which are hereby incorporated by reference in their entirety). In addition, acetylation of K208 by EP300 modulates HSF1 function and protein turnover. Using AptA-MS, acetylation has been detected at each of these essential residues in both NHS and HS conditions in addition to K62 and K162, providing an opportunity for further investigation to elucidate their roles in HSF1 regulation.

The HS response varies by tissue and cell type (Guisbert et al., “Identification of a Tissue-selective Heat Shock Response Regulatory Network,” PLoS Genet. 9:e1003466 (2013); van Oosten-Hawle, et al., “Organismal Proteostasis: Role of Cell-nonautonomous Regulation and Transcellular Chaperone Signaling,” Genes Dev. 28:1533-1543 (2014), which are hereby incorporated by reference in their entirety). Not only does AptA-MS provide a robust method to investigate MCs and their interactions, but it also gives a snapshot of the HSF1-mediated response to HS in HCT116 cells. These data complement previous work done in other cell lines and also give a unique insight into a cell-specific process that requires interaction of translation elongation factors and cytoskeletal proteins with HSF1 during HS response.

AptA-MS provides a detailed view of HSF1 interactions and modifications before and after HS. In addition, control pull-downs also reveal essential information. Pull-downs using GFP only cells with GFP-aptamer or HSF1-GFP cells with a structured control RNA aptamer (selected against dNELF-E) with no predicted binding partners in HCT116 cells provide a proteomic profile for nonspecific interactions between RNA and proteins. The proteins identified from GFP only cells subjected to GFP-aptamer pull-down are not only informative about the nonspecific binders of the GFP/GFP-aptamer in human cells, but also in combination with the control RNA aptamer-enriched proteins from HSF1-GFP cells, allow us to calculate an enrichment factor for each identified protein and to identify high-confidence HSF1 interactors using the most stringent criteria (see Materials and methods). GO analysis of proteins enriched by the control aptamer shows that the vast majority of these proteins are devoted to binding nucleic acid. These nucleic acid-binding proteins are RNA binding proteins. It is predicted that these proteins are likely to bind RNA non-specifically, and proposed that they serve as a resource in RACER, analogous to the common contaminants repository used for antibody-based pull-downs (Mellacheruvu, et al., “The CRAPome: a Contaminant Repository for Affinity Purification-mass Spectrometry Data,” Nat. Methods 10:730-736 (2013), which is hereby incorporated by reference in its entirety). In addition, the non-specific proteins enriched from the GFP only cells by the GFP-aptamer also provide a list of contaminants for AptA-MS studies using GFP-fusions. RACER is publicly available and can be continually updated for use in further AptA-MS experiments.

Knock-in cell lines with GFP-tagged proteins generated by CRISPR/Cas9 are proving to be critical for imaging macromolecules in living cells (Steurer et al., “Live-cell Analysis of Endogenous GFP-RPB1 Uncovers Rapid Turnover of Initiating and Promoter-paused RNA Polymerase II,” Proc. Natl. Acad. Sci. U.S.A., 115:E4368-E4376 (2018), which is hereby incorporated by reference in its entirety). They also can be used directly for analysis by AptA-MS with the GFP aptamer, as this method is capable of purifying GFP and GFP-tagged proteins from various sources. Additionally, large libraries of GFP-tagged proteins are available, and any member of such libraries could also be used directly in AptA-MS, thereby allowing identification of the associated factors along with their PTMs in a single assay (Roberts, et al., “Systematic Gene Tagging Using CRISPR/Cas9 in Human Stem Cells to Illuminate Cell Organization,” Mol. Biol. Cell 28:2854-2874 (2017); Harikumar et al., “An Endogenously Tagged Fluorescent Fusion Protein Library in Mouse Embryonic Stem Cells,” Stem Cell Rep. 9:1304-1314 (2017), which are hereby incorporated by reference in their entirety). These findings would complement optical studies of cellular dynamics and co-localization with other proteins in vivo. The GFP-aptamer has been shown to bind to other derivatives of GFP, making it applicable to precipitate proteins tagged with similar fluorescent proteins (Shui et al., “RNA Aptamers that Functionally Interact with Green Fluorescent Protein and its Derivatives,” Nucleic Acids Res. 40:e39 (2012), which is hereby incorporated by reference in its entirety). Aptamers provide many advantages as affinity reagents: they can be selected against toxic proteins, are amenable for chemical modifications, are cost effective to synthesize, and can be made without the use of animals in any molecular biology lab in unlimited quantities. These advantages, in addition to its broad applicability, make AptA-MS a highly sensitive and simple tool that could significantly transform protein—protein interaction studies and provide deeper and more comprehensive insights in understanding the composition of MCs.

### Example 7—GFP-Aptamer Affinity Purification of PolII from Yeast Cells for Cryo-EM Analysis

### Materials and Methods

Lysate preparation. S. pombe strain used: ade6-m216, ura4-D18, leu1, gfp-rpb3 (Source: National Bio Resource Project, Japan). Lysate was prepared from 500 ml of culture (˜5 billion Yeast cells) expressing GFP-Rpb3 and was used for GFP-aptamer pulldown. Rpb3 is a subunit of RNA polymerase II (Pol II). Cells were cultured to OD600=1 (1.0×10⁷ cells/ml) in 500 ml of YES media at 30° C. Cells were collected in 50 ml of culture into 50 ml conical tubes and centrifuged at 1100×g for 5 min. Medium was discarded and pellets were frozen in liquid N2 and stored at −20° C. Pellets were thawed by adding 1 ml of PBS and transferred into a 2 ml screw cap tube. Cells were centrifuged at 11,000×g for 5 min at 4° C. Pellets were resuspended in 800 μL of complete Lysis buffer (1× PBS+0.2% NP-40+1× PIC+ 1/50 0.1M PMSF). PIC=Protease inhibitor cocktail-EDTA free. Sample was centrifuged at 11,000×g for 5 min at 4° C. Supernatant was discarded and pellets were resuspended in 400 μL of complete Lysis buffer. Zirconia beads were added up to 80% of the 2 ml screw cap tube. Samples were Beadsbeaten by FastPres-24 (MB products) for 4 cycles with the settings below.

| | | |
|---|---|---|
| RPM | 5.0 | M/s |
| ON | 15 | sec |
| OFF | 60 | sec |

Tubes were punctured at the bottom of the 2 ml tube by a heated needle. The 2 ml tube was placed in a 7 ml polypropylene tube and centrifuged at 1100×g for 1 min at 4° C. The lysate pass through the puncture and was collected in the 7 ml tube. The crude lysate was transferred into a new 2 ml tube and centrifuge at 15,000×g for 10 min at 4° C. The supernatant was transferred to a 1.5 ml tube. Lysates were combine in one tube. And diluted 1:4 with Dilution buffer (1× PBS+250 ng/μL ssDNA+250 ng/μL yRNA+7 mM MgCl₂).

RNA preparation and immobilization on beads. 100 pmole of polyadenylated GFP aptamer was annealed to desthiobiotin-oligodT-20 in 200 μL of 1× Annealing buffer (10 mM Tris-HCl pH 7.5, 50 mM NaCl) by heating at 95° C. for 3 min and slow cooling to room temperature over >1 hr. For pulldown 1 mg of Dynabeads MyOne Streptavidin C1 (Thermo) magnetic beads were washed once with 1 ml and twice with 0.1 ml of Tween wash buffer (5 mM Tris-HCl pH 7.5, 0.5 mM EDTA, 1M NaCl, 0.05% Tween-20) by placing on a magnetic separator for 2 min and removing the supernatant. To eliminate possible RNase activity, beads were washed once with 0.1 ml of 0.1 M NaOH, 0.05 M NaCl followed by two washes of 0.1 ml of 0.1 M NaCl with changing tubes in between washes. The beads were resuspended in 200 μL of 2× Binding buffer (10 mM Tris-HCl pH 7.5, 1 mM EDTA, 2 M NaCl) supplemented with 4 units/ml SUPERase IN. The resulting bead slurry was mixed with the annealed RNA aptamer and incubated on a thermomixer for 1 hr at 23° C. with shaking. Aptamer bound beads were washed twice with 0.4 ml of Bead wash buffer (1× PBS, 0.05% NP40, 5 mM MgCl₂), supplemented with 4 units/ml SUPERase IN with changing tubes in between washes.

Binding to lysate, washing & elution. The GFP-aptamer bound beads were resuspended in the diluted cellular lysate and incubated at 4° C. with rotation for 2 hr. The beads were placed on a magnet and supernatant was removed. The beads were washed four times with Bead wash buffer and once with 1× PBS, 5 mM MgCl₂, with changing tubes in between washes. Beads were resuspended in 50 μL of fresh elution buffer (5 mM Biotin, 50 mM Ammonium phosphate, pH 7.5) and incubated in a Thermomixer at 37° C. with shaking for 1 hr followed by collection of the eluate into a fresh tube.

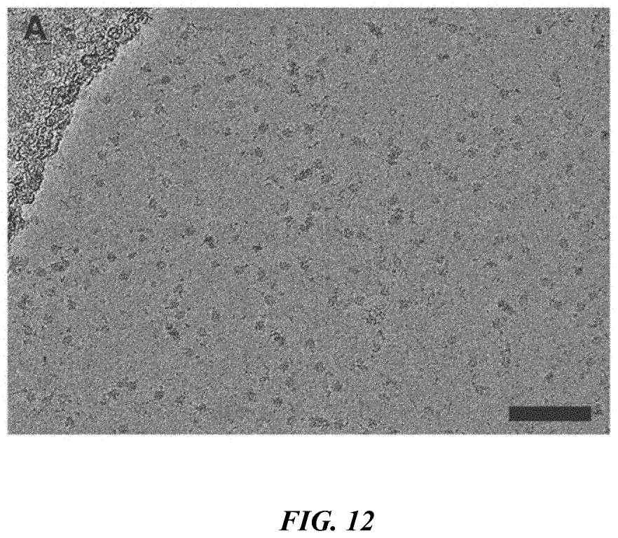

Cryo-EM structure determination. The eluate was prepared for Cryo-EM analysis and the structure was determined. The electron micrograph of a graphene oxide grid below shows that the eluate was enriched in particles consistent with the expected size of Pol II (FIG. 12).

### Example 8—Chromatin Aptamer-Precipitation Sequencing (ChAP-Seq)

### Materials and Methods

Human HCT116 cells transfected with pEGFP of pHSF1-GFPN3 (Addgene #32538) plasmid were resuspended in 0.5 ml ice cold cellular lysis buffer (1× PBS+0.2% NP40+1× EDTA-free Protease inhibitor cocktail). Cells were incubated on ice for 30 min followed by sonication with Bioruptor Diagenode at High setting (30 s ON/30 s OFF) for 5 min. The lysate was centrifuged at 20,000×g for 10 min in 4° C. and the resulting supernatant was transferred to a new tube. The cleared lysate was diluted to a final buffer containing 1× PBS, 0.05% NP40, 5.25 mM MgCl₂, 187.5 ng/μL yeast RNA, 187.5 ng/μL sheared salmon sperm DNA, 200 units SUPERase IN/ml.

RNA preparation and immobilization on beads. Around 200 pmole of polyadenylated GFP- or the control (NELF)-aptamer was annealed to equimolar Desthiobiotin-oligodT (20) in 200 μL of 1× Annealing buffer (10 mM Tris-HCl pH 7.5, 50 mM NaCl) by heating at 95° C. for 3 min and slow cooling to room temperature over >1 hr. For each pulldown 1 mg of Dynabeads MyOne Streptavidin C1 (Thermo) magnetic beads were washed once with 1 ml and twice with 0.1 ml of Tween wash buffer (5 mM Tris-HCl pH 7.5, 0.5 mM EDTA, 1M NaCl, 0.05% Tween-20) by placing on a magnetic separator for 2 min and removing the supernatant. To eliminate possible RNase activity, beads were washed once with 0.1 ml of 0.1 M NaOH, 0.05 M NaCl followed by two washes of 0.1 ml of 0.1 M NaCl with changing tubes in between washes. The beads were resuspended in 200 μL of 2× Binding buffer (10 mM Tris-HCl pH 7.5, 1 mM EDTA, 2M NaCl) supplemented with 4 units/ml SUPERase IN. The resulting bead slurry was mixed with the annealed RNA aptamer and incubated on a thermomixer for 1 hr at 23° C. with shaking. Aptamer bound beads were washed twice with 0.4 ml of Bead wash buffer (1× PBS, 0.05% NP40, 5 mM MgCl₂), 4 units/ml SUPERase IN with changing tubes in between washes.

Binding to lysate, washing & elution. The GFP- or control-aptamer bound beads were resuspended in the diluted cellular lysate and incubated at 4° C. with rotation for 2 hr. The beads were placed on a magnet and supernatant was removed. The beads were washed twice with Bead wash buffer and twice with 1× PBS, 5 mM MgCl₂, with changing tubes in between washes. Beads were resuspended in 50 μL of fresh elution buffer (5 mM Biotin, 50 mM Ammonium phosphate, pH 7.5) and incubated in a Thermomixer at 37° C. with shaking for 1 hr followed by collection of the eluate into a fresh tube.

DNA purification & library preparation for sequencing. Eluates were treated with 2 μL of RNase cocktail (RNase A/T1, Thermo) and incubated at 37° C. for 20 min. DNA was purified by Phenol/Chloroform, chloroform extraction followed by Ethanol precipitation and finally resuspended in 25 μL of 10 mM Tris-HCl, pH 8.5. Library was prepared by Tn5-tagmentation where 10 μL of DNA was treated with Tn5 reaction buffer (10 mM Tris-HCl, pH 7.5, 5 mM MgCl₂, 10% DMF), 0.5 μL Tn5 complex with adaptors to a volume of 25 μL and incubated at 55° C. for 5 min. Reactions were quenched with 0.1% SDS and kept on ice. Tagmented DNA was PCR amplified with Nextera adaptors and Phusion Polymerase for 13 cycles. The amplicons were purified by Ampure beads and sequenced with pair-end sequencing using Illumina NextSeq500.

### Results

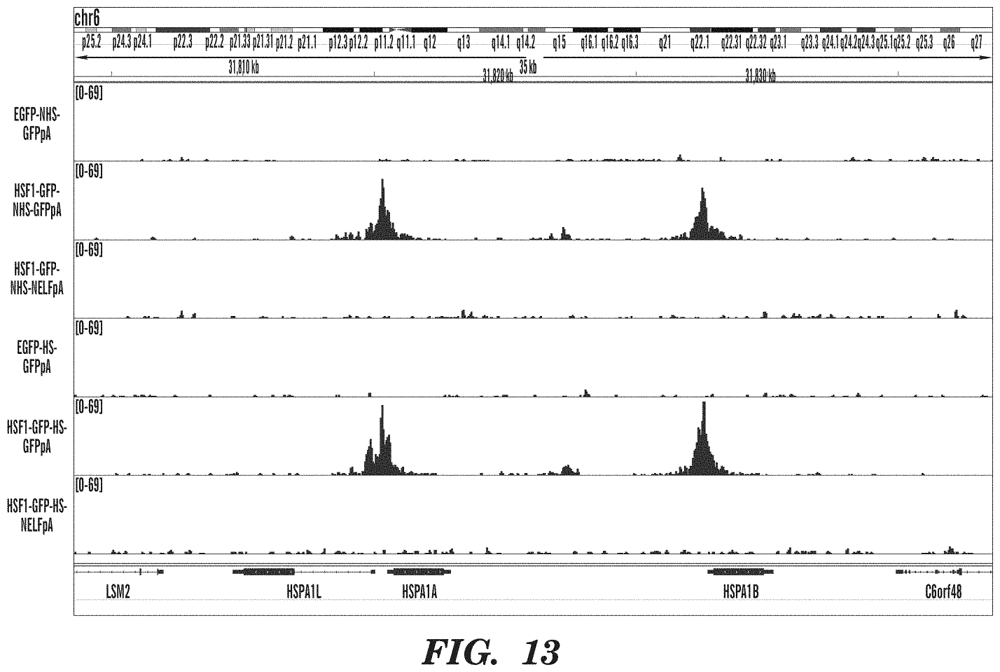

The GFP aptamer-based pulldown strategy could be implemented to identify DNA binding sites of a particular protein genome-wide. The aptamer pulldown methodology as described in FIG. 1 has been performed and instead of analyzing the samples with mass spectrometry (MS), DNA has been purified from the resulting eluates and sequenced by Next generation sequencing. This method is called ChAP-seq as this provides an alternative to ChIP-seq that utilizes antibodies to achieve the same goal. To demonstrate this method, ChAP-seq from human HCT116 cells expressing HSF1-GFP in non-heat shock (NHS) or heat shock (HS) condition has been performed. A representative example is shown in FIG. 13.

ChAP-seq clearly enriches for HSF1 binding sites genome-wide only in HSF1-GFP cells but not in the controls. Many of these peaks overlap with previously identified HSF1 peaks from ChlPseq data obtained from crosslinked human K562 cells. In summary, the aptamer pulldown strategy provides a simple, native method in which samples from a single precipitation technique can be processed for both AptA-MS as well as ChAP-seq thereby providing both protein and DNA interactor information from a single assay.

### Example 9—Chromatin Aptamer-Precipitation from Formaldehyde Crosslinked Cells

### Materials and Methods

Human HCT116 cells expressing GFP-Rpb1 were grown in 15 cm dishes to confluency in McCoy's 5A Media+10% FBS+Penn/Strep. Media was removed and the cells were rinsed with 1× PBS. Cells were crosslinked with 0.5% formaldehyde for 5 min at room temperature with shaking. Crosslinking was quenched with 200 mM Glycine for 5 min at room temperature with shaking. Cells were collected and washed with 1× PBS. Cell pellets were flash frozen in liquid nitrogen and stored at −80° C. Crosslinked cell pellet was resuspended in 10 ml ice cold lysis buffer (10 mM Tris pH 8, 10 mM NaCl, 0.5% NP40+1× EDTA-free Protease inhibitor cocktail). Cells were incubated on ice for 15 min. Cells were spun at 500×g for 5 min at 4° C. and supernatant was removed. The pellet was resuspended in 10 ml ice cold lysis buffer and incubated on ice for 5 min. Cells were spun at 500×g for 5 min at 4° C. and supernatant was removed. Pellet was resuspended in 1× pellet volume of Nuclear lysis buffer (50 mM Tris pH 8+0.2% SDS) and transferred to a new tube. The tube was shaken in a thermomixer at room temperature for 5 min. Equal volume of SDS Quenching buffer (2× PBS+1% Triton X-100) was added. The suspension was divided into 1.5 ml tubes and sonicated with Bioruptor Diagenode at High setting (30 s ON/30 s OFF) for 15 min in ice cold water at 4° C. The lysate was centrifuged at 20,000×g for 10 min in 4° C. and the resulting supernatant was transferred to a new tube. The cleared lysate was diluted to a final buffer containing 1× PBS, 0.01% SDS, 0.1% Triton X-100, 5 mM MgCl₂, 200 ng/μL yeast RNA, 0.1 mM Dextran Sulfate, 100 units SUPERase IN/ml.

RNA preparation and immobilization on beads. Around 225 pmole of polyadenylated GFP (C58U)- or the control (NELF)-aptamer was annealed to 200 pmole of DesthiobiotinoligodT (20) in 200 μL of 1× Annealing buffer (10 mM Tris-HCl pH 7.5, 50 mM NaCl) by heating at 95° C. for 5 min and slow cooling to room temperature over >1 hr. For each pulldown 0.5 mg of Dynabeads MyOne Streptavidin C1 (Thermo) magnetic beads were washed once with 0.5 ml and twice with 50 μL of Tween wash buffer (5 mM Tris-HCl pH 7.5, 0.5 mM EDTA, 1M NaCl, 0.05% Tween-20) by placing on a magnetic separator for 2 min and removing the supernatant. To eliminate possible RNase activity, beads were washed once with 50 μL of 0.1 M NaOH, 0.05 M NaCl followed by two washes of 50 μL of 0.1 M NaCl with changing tubes in between washes. The beads were resuspended in 200 μL of 2× Binding buffer (10 mM Tris-HCl pH 7.5, 1 mM EDTA, 2M NaCl) supplemented with 4 units/ml SUPERase IN. The resulting bead slurry was mixed with the annealed RNA aptamer and incubated on a thermomixer for 1 hr at 23° C. with shaking. Aptamer bound beads were washed twice with 0.4 ml of Bead wash buffer (1× PBS, 0.05% NP40, 5 mM MgCl2), 4 units/ml SUPERase IN with changing tubes in between washes.

Binding to lysate, washing & elution. The GFP- or control-aptamer bound beads were resuspended in the diluted lysate and incubated at 4° C. with rotation for 2 hr. The beads were placed on a magnet and supernatant was removed. The beads were washed with Wash buffer 1 (1× PBS, 0.1% NP40, 5 mM MgCl₂, 0.1 mM DxSO4, SUPERase IN) twice with Wash buffer 2 (1× PBS, 0.1% NP40, 5 mM MgCl₂, 200 mM NaCl, SUPERase IN) by shaking in a Thermomixer for 5 min at room temperature, once with Wash buffer 3 (1× PBS, 0.1% NP40, 5 mM MgCl₂, SUPERase IN) and finally with 1× PBS, 5 mM MgCl₂, with changing tubes in between washes. Beads were resuspended in 50 μL of fresh elution buffer (5 mM Biotin, 50 mM Ammonium phosphate, pH 7.5, 4 μL RNase A/T1 cocktail) and incubated in a Thermomixer at room temperature with shaking for 30 min followed by collection of the eluate into a fresh tube.

DNA purification, qPCR, & sequencing. DNA was purified by Qiagen Minelute Reaction cleanup kit. qPCR was performed with target site (GAPDH primers from Erickson et al, Genes &. Dev (2018), which is hereby incorporated by reference in its entirety) and non-target site primers (2 Kb upstream of GAPDH transcription start site) using Roche Lightcycler qPCR instrument.

For DNA sequencing the library was prepared by Tn5-tagmentation where 10 μL of DNA was treated with Tn5 reaction buffer (10 mM Tris-HCl, pH 7.5, 5 mM MgCl₂, 10% DMF), 0.5 μL Tn5 complex with adaptors to a volume of 25 μL and incubated at 55° C. for 5 min. Reactions were quenched with 0.1% SDS and kept on ice. Tagmented DNA was PCR amplified with Nextera adaptors and Phusion Polymerase for 13 cycles. The amplicons were purified by Ampure beads and sequenced with pair-end sequencing using Illumina HiSeq system.

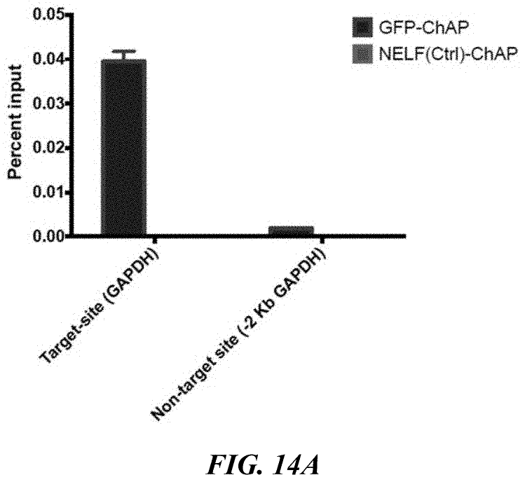

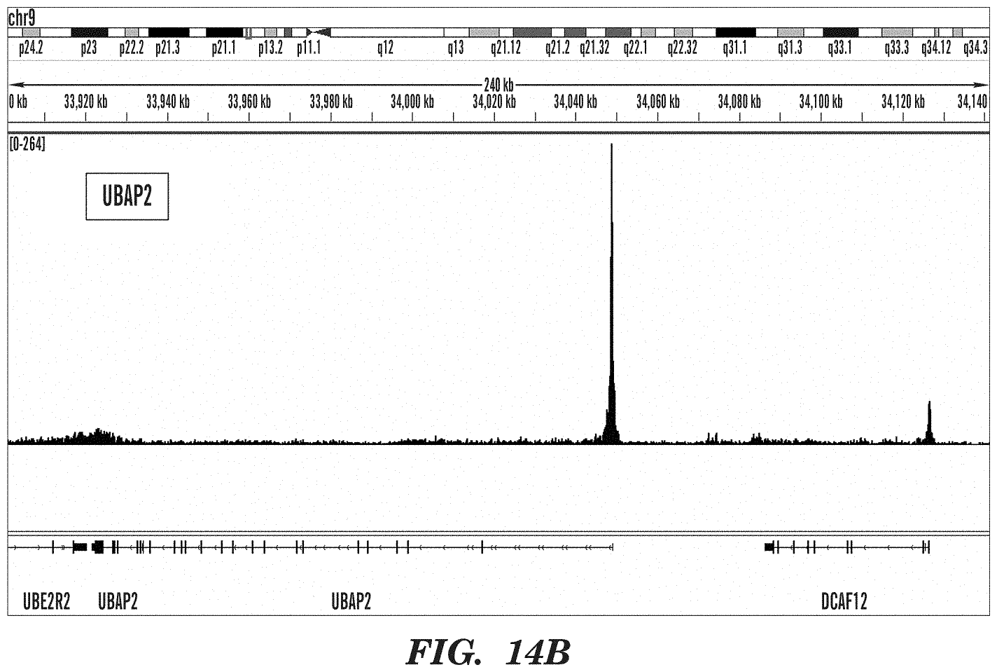

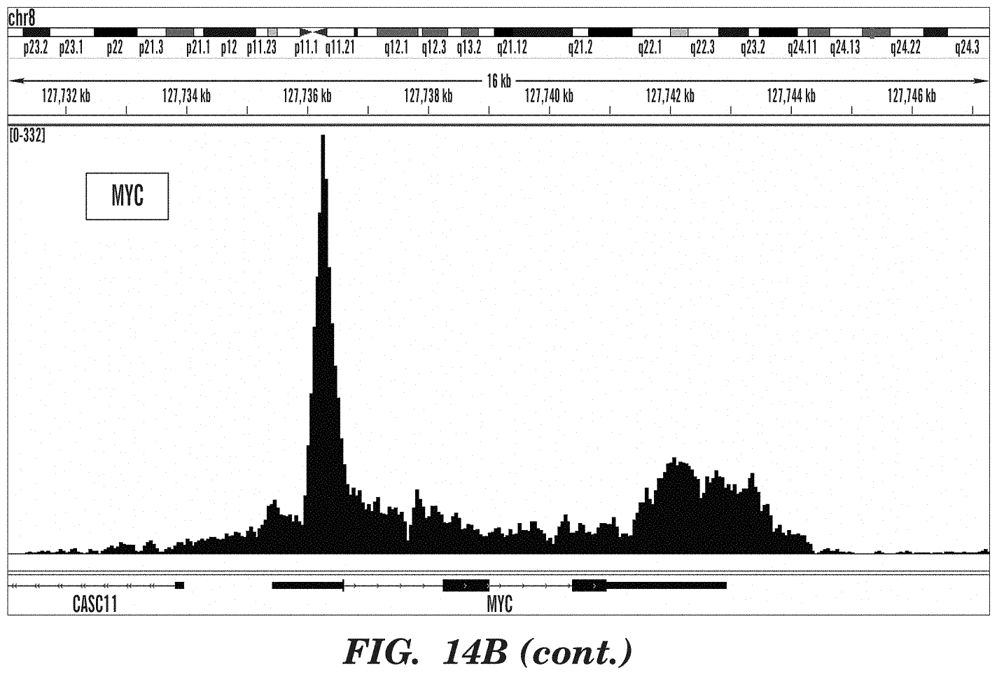

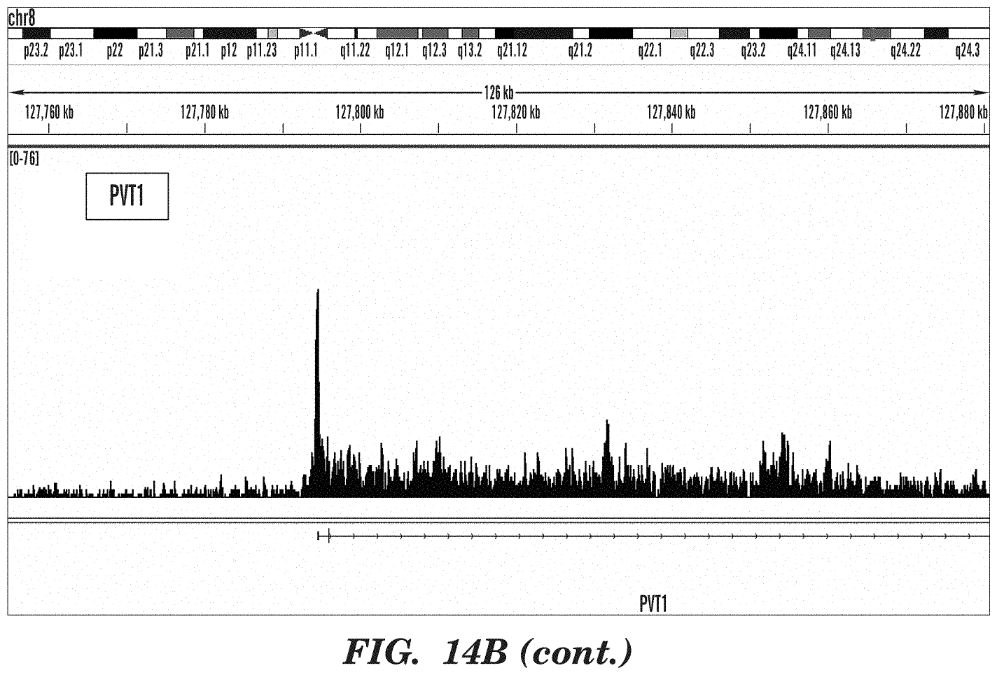

It is shown that the GFP-aptamer can be utilized to purify a protein of interest from native cellular lysates and identify the DNA sequences bound to the protein by Next generation sequencing. Here, this Chromatin-Aptamer Precipitation (ChAP) strategy was further implemented to identify DNA-binding sites of a particular protein of interest from formaldehyde (FA) crosslinked cells, an extended method that is referred hereby as FA-ChAP (FIG. 14A-14B). To demonstrate this method, the largest subunit (Rpb1) of RNA polymerase II (Pol II) has been GFP-tagged in human HCT116 cells by CRISPR knock-in and specific DNA sequences bound to Pol II have been purified utilizing the GFP-aptamer and analyzed by qPCR (FIG. 14A) or Next generation DNA sequencing (FIG. 14B).

FA-ChAP provides a highly specific approach to enrich and identify DNA sequences bound to a GFP-tagged protein of interest upon formaldehyde fixation. Formaldehyde has been used for decades to capture transient macromolecular interactions of many proteins including the transcription factors that are loosely bound to the underlying DNA. Standard Chromatin immunoprecipitation (ChIP) utilizes formaldehyde to crosslink the cells before performing the immunoprecipitation step in order to stabilize the DNA-protein complex. Although many antibodies perform well for native detection/precipitation methods, all are not suited for ChIP assays. The FA-ChAP results demonstrate that the GFP-aptamer is highly suitable for precipitating a GFP-tagged protein even upon formaldehyde crosslinking. The high signal-noise ratio detected by DNA sequencing of the pulled down material makes FA-ChAP an inexpensive and rapid method to identify the DNA binding sites of the tagged protein with high sensitivity, thereby allowing identification of potential novel binding sites.

### Example 10—Sequential Affinity-Purification Using the GFP-Aptamer and Factor-Specific Antibody (ChAP-ChIP-Seq)

The idea of a sequential affinity-purification where two proteins (one of them being GFP-tagged) co-occupying a sequence of DNA can be purified by their respective affinity reagents and the underlying DNA can be isolated and analyzed is shown in FIG. 15. FIG. 15 depicts the use of the GFP-aptamer that can be utilized for purifying a macromolecular complex from a crosslinked cellular lysate that consists of GFP-tagged RNA Polymerase II, any associated factor (could be any protein) and histone proteins all decorated on a DNA sequence. Following a series of washes the aptamer bound complex can be eluted with Biotin and RNase cocktail and the same eluate can be used to capture once more with an antibody against any associated factor or histones. The material is washed, and the DNA is purified to be analyzed by sequencing.

The same idea could be reversed where the first capture is done with the antibody and the second one is done with the GFP-aptamer (FIG. 16). Either of these two strategies could be employed depending on the tagged factor. This method of sequential affinity purification (ChAP-ChIP) allows the identification of DNA sequences co-occupied by two proteins of interest that are directly or indirectly bound to DNA. The advantage of this technique over conventional re-ChIP is that the later relies on the use of two antibodies, one against each factor and thereby suffers from cross-contamination issues that could negatively influence the biological relevance of the final result. ChAP-ChIP circumvents this issue by replacing one of the antibody-based purification steps with an aptamer and therefore is free from cross-contamination of antibodies.

Although preferred embodiments have been depicted and described in detail herein, it will be apparent to those skilled in the relevant art that various modifications, additions, substitutions, and the like can be made without departing from the spirit of the present application and these are therefore considered to be within the scope of the present application as defined in the claims which follow.

What is claimed:

1. A method for analyzing a molecular target in a sample, said method comprising:
providing an aptamer, wherein said aptamer is a high affinity binding partner to at least a portion of the molecular target in the sample;
contacting said aptamer with said sample containing the molecular target under conditions effective for the molecular target and aptamer to bind to each other;
separating the molecular target from the sample to form a molecular target enriched sample; and
analyzing the separated molecular target of the enriched sample.

2. The method of claim 1, wherein said analyzing comprises a method selected from mass spectrometry, cryo-electron microscopy, and nucleotide sequencing.

3. The method of claim 1, wherein the molecular target comprises one or more biomolecules.

4. The method of claim 3, wherein the one or more biomolecules is selected from a protein, polypeptide, peptide, ribonucleic acid (RNA), deoxyribonucleic acid (DNA), lipid, carbohydrate, and any combination thereof.

5. The method of claim 1, further comprising;
providing, after said separating, a binding agent that binds to a different portion of the molecular target than bound by the aptamer;
contacting the molecular target enriched sample with the binding agent under conditions effective for the molecular target and binding agent to bind to each other;
immunoprecipitating the binding agent to isolate the molecular target from the enriched sample, whereby said isolated molecular target is subjected to said analyzing.

6. The method of claim 5, wherein the binding agent is an antibody or binding fragment thereof.

7. The method of claim 1, wherein the sample is crosslinked prior to said contacting with said aptamer.

8. The method of claim 1, further comprising:
providing a binding agent that binds to a different portion of the second molecular target than bound by the aptamer;
introducing the binding agent into the sample, prior to said contacting with said aptamer, under conditions effective for the molecular target and binding agent to bind to each other;
immunoprecipitating the binding agent to isolate the molecular target from the sample, whereby said isolated molecular target is subjected to said contacting with said aptamer.

9. The method of claim 8, wherein said sample is crosslinked prior to introducing the binding agent.

10. The method of any one of claim 8, wherein the binding agent is an antibody or binding fragment thereof.

11. The method of claim 1, wherein the molecular target comprises a tag moiety, and the aptamer is a high affinity binding partner to at least a portion of the tag moiety of the molecular target.

12. The method of claim 11, wherein the tag moiety is selected from a fluorescent protein or fragment thereof, a poly-His tag, a maltose binding protein tag, an albumin-binding protein tag, a calmodulin binding peptide tag, a glutathione S-transferase tag, a chitin binding protein tag, FLAG-tag, HA-tag, Protein A tag, and combinations thereof.

13. The method of claim 1, wherein the aptamer is selected from a non-naturally occurring aptamer and a naturally occurring aptamer.

14. The method of claim 1, wherein the aptamer is selected from a peptide aptamer, a DNA aptamer, and an RNA aptamer.

15. The method of claim 1, wherein said aptamer is an RNA aptamer comprising:
a core region comprising a nucleotide sequence of any one of SEQ ID NOs:1-13.

16. The method of claim 1, wherein the aptamer binds to a portion of a fluorescent protein selected from GFP, eGFP, CFP, eCFP, YFP, and eYFP.

17. The method of claim 1, wherein the aptamer is immobilized on a solid support.

18. The method of claim 1, wherein said molecular target comprises two or more associated biomolecules, and said analyzing comprises:
identifying each of the two or more associated biomolecules of the molecular target.

19. The method of claim 18, wherein said identifying comprises:
subjecting the molecular target to mass spectrometry to detect each of the two or more associated biomolecules of the molecular target.

20. The method of claim 1, wherein said analyzing comprises:
subjecting the molecular target to mass spectrometry to detect one or more posttranslational modifications of said molecular target.

21. The method of claim 20, wherein the one or more posttranslational modifications is selected from methylation, acetylation, phosphorylation, sumoylation, or combinations thereof.

22. The method of claim 1, wherein said molecular target comprises nucleotide oligomers and said analyzing comprises:
isolating the nucleotide oligomers from the molecular target and
subjecting said isolated nucleotide oligomers to an amplification reaction, a sequencing reaction, or a combination thereof to identify the isolated nucleotide oligomers of the molecular target.

23. The method of claim 1, wherein said analyzing comprises:
subjecting the molecular target to cryo-electron microscopy to determine the three-dimensional structure of the molecular target.

24. The method of claim 23, wherein said molecular target comprises two or more associated biomolecules, and said subjecting is carried out to determine the three-dimensional structure of the two or more associated biomolecules of the molecular target.

25. The method of claim 1, wherein the sample is a cell or tissue lysate, a plasma sample, a serum sample, a blood sample, an exosome sample, or other biological sample.


SEQUENCE LISTING (15 sequences)

SEQ ID NO: 1 - RNA, 50 nt, Artificial
  aptamer
  misc_feature (11)..(12): n is a, c, g, or u
  misc_feature (33)..(33): n is a, c, g, or u
  misc_feature (38)..(38): n is a, c, g, or u
  misc_feature (42)..(42): n is a, c, g, or u
aucgaauugunngguagaaaguccuuugagagnaaccngggnggauacug

SEQ ID NO: 2 - RNA, 50 nt, Artificial
  aptamer
aucgaauuguuagguagaaaguccuuugagaggaaccugggaggauacug

SEQ ID NO: 3 - RNA, 50 nt, Artificial
  aptamer
aucgaauugugugguagaaaguccuuugagagaaaccaggggggauacug

SEQ ID NO: 4 - RNA, 50 nt, Artificial
  aptamer
  misc_feature (1)..(1): n is a, c, g, or u
  misc_feature (3)..(3): n is a, c, g, or u
  misc_feature (10)..(10): n is a, c, g, or u
  misc_feature (25)..(25): n is a, c, g, or u
  misc_feature (39)..(39): n is a, c, g, or u
ngngaauugnguggggaaaguccunaaaagagggccacngccgaaacgcc

SEQ ID NO: 5 - RNA, 50 nt, Artificial
  aptamer
ugugaauuggguggggaaaguccugaaaagagggccaccgccgaaacgcc

SEQ ID NO: 6 - RNA, 50 nt, Artificial
  aptamer
ugcgaauuggguggggaaaguccugaaaagagggccaccgccgaaacgcc

SEQ ID NO: 7 - RNA, 50 nt, Artificial
  aptamer
ugugaauugaguggggaaaguccugaaaagagggccacugccgaaacgcc

SEQ ID NO: 8 - RNA, 50 nt, Artificial
  aptamer
ugugaauuggguggggaaaguccuuaaaagagggccaccgccgaaacgcc

SEQ ID NO: 9 - RNA, 50 nt, Artificial
  aptamer
ggugaauuggguggggaaaguccuuaaaagagggccaccgccgaaacgcc

SEQ ID NO: 10 - RNA, 50 nt, Artificial
  aptamer
ugugaauugaguggggaaaguccugaaaagagggccaccgccgaaacgcc

SEQ ID NO: 11 - RNA, 50 nt, Artificial
  aptamer
ugugaauugaguggggaaaguccugaaaagagggccacagccgaaacgcc

SEQ ID NO: 12 - RNA, 50 nt, Artificial
  aptamer
uuggggugggguggggaaaguccuuaaaagagggccaccacagaagcaau

SEQ ID NO: 13 - RNA, 50 nt, Artificial; identical to SEQ ID NO: 12
  aptamer

SEQ ID NO: 14 - DNA, 86 bp, Artificial
  aptamer
gggagcttctggactgcgatgggagcacgaaacgtcgtggcgcaattgggtggggaaagtccttaaaagagggccaccacagaagc

SEQ ID NO: 15 - DNA, 120 bp, Artificial
  aptamer
gggaatggatccacatctacgaattcccaacgactgccgagcgagattacgcttgagcgccccactgaggatgcccacgggcgattggggcacggcttcactgcagacttgacgaagctt